(19) United States
(12) Patent Application Publication
(10) Pub. No.: US 2016/0194722 A1
(43) Pub. Date: Jul. 7, 2016

(54) METHODS AND NUCLEIC ACIDS FOR ANALYSES OF CELL PROLIFERATIVE DISORDERS

(71) Applicant: Epigenomics AG, Berlin (DE)

(72) Inventors: Dimo Dietrich, Berlin (DE); Volker Liebenberg, Berlin (DE); Reimo Tetzner, Berlin (DE); Juergen Distler, Berlin (DE); Joern Lewin, Berlin (DE); Thomas Schlegel, Berlin (DE)

(21) Appl. No.: 14/978,642

(22) Filed: Dec. 22, 2015

Related U.S. Application Data
(62) Division of application No. 12/747,584, filed on Sep. 13, 2010, now abandoned.

(30) Foreign Application Priority Data
Dec. 11, 2007 (EP) 07122844.9
Jan. 23, 2008 (EP) 08150557.0

(51) Int. Cl. C12Q 1/68

(52) U.S. Cl. CPC C12Q 1/6886; C12Q 2600/154; C12Q 2600/158; C12Q 2600/16

(57) ABSTRACT

The invention provides methods, nucleic acids and kits for detecting lung carcinoma. The invention discloses genomic sequences the methylation patterns of which have utility for the improved detection of said disorder, thereby enabling the improved diagnosis and treatment of patients.

### FIELD OF THE INVENTION

The present invention relates to genomic DNA sequences that exhibit altered expression patterns in disease states relative to normal. Particular embodiments provide methods, nucleic acids, nucleic acid arrays and kits useful for detecting, or for diagnosing cell proliferative disorders.

### BACKGROUND

### CpG Island Methylation.

Apart from mutations aberrant methylation of CpG islands has been shown to lead to the transcriptional silencing of certain genes that have been previously linked to the pathogenesis of various cell proliferative disorders, including cancer. CpG islands are short sequences which are rich in CpG dinucleotides and can usually be found in the 5′ region of approximately 50% of all human genes. Methylation of the cytosines in these islands leads to the loss of gene expression and has been reported in the inactivation of the X chromosome and genomic imprinting.

### Development of Medical Tests.

Two key evaluative measures of any medical screening or diagnostic test are its sensitivity and specificity, which measure how well the test performs to accurately detect all affected individuals without exception, and without falsely including individuals who do not have the target disease (predicitive value). Historically, many diagnostic tests have been criticized due to poor sensitivity and specificity.

A true positive (TP) result is where the test is positive and the condition is present. A false positive (FP) result is where the test is positive but the condition is not present. A true negative (TN) result is where the test is negative and the condition is not present. A false negative (FN) result is where the test is negative but the condition is not present. In this context: Sensitivity=TP/(TP+FN); Specificity=TN/(FP+TN); and Predictive value=TP/(TP+FP).

Sensitivity is a measure of a test's ability to correctly detect the target disease in an individual being tested. A test having poor sensitivity produces a high rate of false negatives, i.e., individuals who have the disease but are falsely identified as being free of that particular disease. The potential danger of a false negative is that the diseased individual will remain undiagnosed and untreated for some period of time, during which the disease may progress to a later stage wherein treatments, if any, may be less effective. An example of a test that has low sensitivity is a protein-based blood test for HIV. This type of test exhibits poor sensitivity because it fails to detect the presence of the virus until the disease is well established and the virus has invaded the bloodstream in substantial numbers. In contrast, an example of a test that has high sensitivity is viral-load detection using the polymerase chain reaction (PCR). High sensitivity is achieved because this type of test can detect very small quantities of the virus. High sensitivity is particularly important when the consequences of missing a diagnosis are high.

Specificity, on the other hand, is a measure of a test's ability to identify accurately patients who are free of the disease state. A test having poor specificity produces a high rate of false positives, i.e., individuals who are falsely identified as having the disease. A drawback of false positives is that they force patients to undergo unnecessary medical procedures treatments with their attendant risks, emotional and financial stresses, and which could have adverse effects on the patient's health. A feature of diseases which makes it difficult to develop diagnostic tests with high specificity is that disease mechanisms, particularly in cell proliferative disorders, often involve a plurality of genes and proteins. Additionally, certain proteins may be elevated for reasons unrelated to a disease state. Specificity is important when the cost or risk associated with further diagnostic procedures or further medical intervention are very high.

### SUMMARY OF THE INVENTION

The present invention provides a method for detecting or differentiating cell proliferative disorders, preferably those according to Table 2, and most preferably lung carcinomas, in a subject comprising determining the expression levels wherein determining expression levels also includes determining methylation levels and patterns of at least one gene or genomic sequence selected from the group consisting of FOXL-2, ONECUT1, TFAP2E, EN2-2, EN2-3, SHOX2-2, and BARHL2 in a biological sample isolated from said subject wherein hyper-methylation and/or under-expression is indicative of the presence of said disorder. Various aspects of the present invention provide an efficient and unique genetic marker, whereby expression analysis of said marker enables the detection of cell proliferative disorders, preferably those according to Table 2 with a particularly high sensitivity, specificity and/or predictive value. Preferred is that the lung cancer is selected from the group consisting of Lung adenocarcinoma; Large cell lung cancer; Squamous cell lung carcinoma and Small cell lung carcinoma.

In one embodiment the invention provides a method for detecting cell proliferative disorders, preferably those according to Table 2 (most preferably lung carcinoma), in a subject comprising determining the expression levels of at least one gene or genomic sequence selected from the group consisting of FOXL-2, ONECUT1, TFAP2E, EN2-2, EN2-3, SHOX2-2 and BARHL2 in a biological sample isolated from said subject wherein under-expression and/or CpG methylation is indicative of the presence of said disorder. In one embodiment said expression level is determined by detecting the presence, absence or level of mRNA transcribed from said gene. In a further embodiment said expression level is determined by detecting the presence, absence or level of a polypeptide encoded by said gene or sequence thereof.

In a further preferred embodiment said expression is determined by detecting the presence or absence or level of CpG methylation within said gene, wherein under-expression, which is understood as indicated by presence of CpG methylation, or by presence of a certain level of methylation, indicates the presence of cell proliferative disorders, preferably those according to Table 2 (most preferably lung carcinoma).

Said method comprises the following steps: i) contacting genomic DNA isolated from a biological sample (preferably selected from the group consisting of cells or cell lines, histological slides, biopsies, paraffin-embedded tissue, body fluids, ejaculate, urine, blood plasma, blood serum, whole blood, isolated blood cells, sputum and biological matter derived from bronchoscopy (including, but not limited to, bronchial lavage, bronchial alveolar lavage, bronchial brushing, and bronchial abrasion) obtained from the subject, preferably a human subject, with at least one reagent, or series of reagents that distinguishes between methylated and non-methylated CpG dinucleotides within at least one target region of the genomic DNA, wherein the target region is the region which is investigated and wherein the nucleotide sequence of said target region comprises at least one CpG dinucleotide sequence of at least one gene or genomic sequence selected from the group consisting of FOXL-2, ONECUT1, TFAP2E (including promoter or regulatory elements thereof) and EN2-2, EN2-3, SHOX2-2 and BARHL2- and ii) detecting cell proliferative disorders, preferably those according to (most preferably lung carcinoma), at least in part. Preferably the target region is located within a genomic sequences selected from the group mentioned above. It is preferred that the target region comprises, or hybridizes under stringent conditions to a sequence of at least 16 contiguous nucleotides of SEQ ID NO: 1 to SEQ ID NO: 7.

Preferably, the sensitivity of said detection is from about 75% to about 96%, or from about 80% to about 90%, or from about 80% to about 85%. Preferably, the specificity is from about 75% to about 96%, or from about 80% to about 90%, or from about 80% to about 85%.

Said use of the gene may be enabled by means of any analysis of the expression of the gene, by means of mRNA expression analysis or protein expression analysis. However, in the most preferred embodiment of the invention the detection of cell proliferative disorders, preferably those according to (most preferably lung carcinoma), is enabled by means of analysis of the methylation status of at least one gene or genomic sequence selected from the group consisting of FOXL-2; ONECUT1; TFAP2E (including promoter or regulatory elements thereof) and EN2-2, EN2-3, SHOX2-2, and BARHL2.

The invention provides a method for the analysis of biological samples for features associated with the development of cell proliferative disorders, preferably those according to (most preferably lung carcinoma), the method characterized in that the nucleic acid, or a fragment thereof of SEQ ID NO: 1 to SEQ ID NO: 7 is contacted with a reagent or series of reagents capable of distinguishing between methylated and non methylated CpG dinucleotides within the genomic sequence.

The present invention provides a method for ascertaining epigenetic parameters of genomic DNA associated with the development of cell proliferative disorders, preferably those according to (most preferably lung carcinoma). The method has utility for the improved detection and diagnosis of said disease.

Preferably, the source of the test sample is selected from the group consisting of cells or cell lines, histological slides, biopsies, paraffin-embedded tissue, body fluids, ejaculate, urine, blood plasma, blood serum, whole blood, isolated blood cells, sputum and biological matter derived from bronchoscopy (including, but not limited to, lavage, bronchial alveolar lavage, bronchial brushing, bronchial abrasion, and combinations thereof. More preferably the sample type is selected from the group consisting of blood plasma, sputum and biological matter derived from bronchoscopy (including, but not limited to, bronchial lavage, bronchial alveolar lavage, bronchial brushing, and bronchial abrasion) and all possible combinations thereof.

Specifically, the present invention provides a method for detecting cell proliferative disorders, preferably those according to Table 2 (most preferably lung carcinoma) suitable for use in a diagnostic tool, comprising: obtaining a biological sample comprising genomic nucleic acid(s); contacting the nucleic acid(s), or a fragment thereof, with a reagent or a plurality of reagents sufficient for distinguishing between methylated and non methylated CpG dinucleotide sequences within a target sequence of the subject nucleic acid, wherein the target sequence comprises, or hybridises under stringent conditions to, a sequence comprising at least 16 contiguous nucleotides of SEQ ID NO: 1 to SEQ ID NO: 7, said contiguous nucleotides comprising at least one CpG dinucleotide sequence; and determining, based at least in part on said distinguishing, the methylation state of at least one CpG dinucleotide within said target sequence, or an average, or a value reflecting an average methylation state of a plurality of CpG dinucleotides within said target sequence of the subject nucleic acid, wherein the target sequence comprises, or hybridises under stringent conditions to a sequence comprising at least 16 contiguous nucleotides of SEQ ID NO: 1 to SEQ ID NO: 7, said contiguous nucleotides comprising at least one CpG dinucleotide sequence.

Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NO: 8 to SEQ ID NO: 35 and contiguous regions thereof corresponding to the target sequence.

Additional embodiments provide a method for the detection of cell proliferative disorders, preferably those according to Table 2 (most preferably lung carcinoma) comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting SEQ ID NO: 8 to SEQ ID NO: 35 and complements thereof, wherein the treated DNA or the fragment thereof is either amplified to produce an amplificate, or is not amplified; and determining, based on a presence or absence of, or on a property of said amplificate, the methylation state or an average, or a value reflecting an average of the methylation level of at least one, but more preferably a plurality of CpG dinucleotides of SEQ ID NO: 1 to SEQ ID NO: 7.

Preferably, determining comprises use of at least one method selected from the group consisting of: i) hybridizing at least one nucleic acid molecule comprising a contiguous sequence at least 9 nucleotides in length that is complementary to, or hybridizes under moderately stringent or stringent conditions to a sequence selected from the group consisting of SEQ ID NO: 8 to SEQ ID NO: 35 and complements thereof; ii) hybridizing at least one nucleic acid molecule, bound to a solid phase, comprising a contiguous sequence at least 9 nucleotides in length that is complementary to, or hybridizes under moderately stringent or stringent conditions to a sequence selected from the group consisting of SEQ ID NO: 8 to SEQ ID NO: 35 and complements thereof; iii) hybridizing at least one nucleic acid molecule comprising a contiguous sequence at least 9 nucleotides in length that is complementary to, or hybridizes under moderately stringent or stringent conditions to a sequence selected from the group consisting of SEQ ID NO: 8 to SEQ ID NO: 35 and complements thereof, and extending at least one such hybridized nucleic acid molecule by at least one nucleotide base; and iv) sequencing of the amplificate.

Further embodiments provide a method for the analysis (i.e. detection or diagnosis) of cell proliferative disorders, preferably those according to Table 2 (most preferably lung carcinoma), comprising: obtaining a biological sample having subject genomic DNA; extracting the genomic DNA; contacting the genomic DNA, or a fragment thereof, comprising one or more sequences selected from the group consisting of SEQ ID NO: 1 to SEQ ID NO: 7; or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of SEQ ID NO: 1 to SEQ ID NO: 7; or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within SEQ ID NO: 1 to SEQ ID NO: 7.

Additional embodiments provide novel analytical assays, as well as specific favourable combinations of primers and blockers or primers and probes, resulting in especially well performing diagnostic or analytical tests.

### DETAILED DESCRIPTION OF THE INVENTION

### Definitions

The term “Observed/Expected Ratio” (“O/E Ratio”) refers to the frequency of CpG dinucleotides within a particular DNA sequence, and corresponds to the [number of CpG sites/(number of C bases×number of G bases)]/band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 KB, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a double stranded DNA wherein only one strand thereof is methylated.

The term ‘AUC’ as used herein is an abbreviation for the area under a curve. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylation. Further epigenetic parameters include, for example, the acetylation of histones which, however, cannot be directly analysed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state or methylation level of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridisation” is to be understood as a bond of an oligonucleotide to a complementary sequence along the lines of the Watson-Crick base pairings in the sample DNA, forming a duplex structure.

“Stringent hybridisation conditions,” as defined herein, involve hybridising at 68° C. in 5×SSC/5×Denhardt's solution/1.0% SDS, and washing in 0.2×SSC/0.1% SDS at room temperature, or involve the art-recognized equivalent thereof (e.g., conditions in which a hybridisation is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The terms “Methylation-specific restriction enzymes” or “methylation-sensitive restriction enzymes” shall be taken to mean an enzyme that selectively digests a nucleic acid dependend on the methylation state of its recognition site. In the case of such restriction enzymes which specifically cut if the recognition site is not methylated or hemimethylated, the cut will not take place, or with a significantly reduced efficiency, if the recognition site is methylated. In the case of such restriction enzymes which specifically cut if the recognition site is methylated, the cut will not take place, or with a significantly reduced efficiency if the recognition site is not methylated. Preferred are methylation-specific restriction enzymes, the recognition sequence of which contains a CG dinucleotide (for instance cgcg or cccggg). Further preferred for some embodiments are restriction enzymes that do not cut if the cytosine in this dinucleotide is methylated at the carbon atom C5.

“Non-methylation-specific restriction enzymes” or “non-methylation-sensitive restriction enzymes” are restriction enzymes that cut a nucleic acid sequence irrespective of the methylation state with nearly identical efficiency. They are also called “methylation-unspecific restriction enzymes.”

The term “at least one gene or genomic sequence selected from the group consisting of ONECUT1; FOXL-2 and TFAP2E; EN2-2, EN2-3, SHOX2-2 and BARHL2 shall be taken to include any transcript variant thereof. Furthermore as a plurality of SNPs are known within said genes the term shall be taken to include all sequence variants thereof.

If within the present specification the genomic regions EN2-2, EN2-3 and SHOX2-2 are mentioned these terms are referring to the genomic sequences as presented in the sequence protocol (as listed in Table 1). These regions represent CpG islands associated with the genes EN2 or SHOX2.

The sample types which may be analysed with any of the methods according to the invention may be any from the group comprising cells or cell lines, histological slides, biopsies, paraffin-embedded tissue, body fluids, ejaculate, urine, blood plasma, blood serum, whole blood, isolated blood cells, sputum and biological matter derived from bronchoscopy (including, but not limited to, bronchial lavage, bronchial alveolar lavage, bronchial brushing, bronchial abrasion, and combinations thereof. More preferably the sample type is selected from the group consisting of blood plasma, sputum and biological matter derived from bronchoscopy (including, but not limited to, bronchial lavage, bronchial alveolar lavage, bronchial brushing, and bronchial abrasion) and all possible combinations thereof.

The sample types which may be analysed with any of the methods according to the invention preferably belong to the group of fluids which are derived from the bloodstream.

The sample types which may be analysed with any of the methods according to the invention also preferably belong to the group of biological samples derived from the lung. The term “biological samples derived from the lung” shall therefore comprise fluids and/or cells obtained from the bronchial system of the lung. Such biological samples derived from the lung may be taken from a subject (e.g. a patient) without adding an external fluid, in which case typical sample types are sputum, tracheal or bronchial fluid, exhaled fluid, brushings or biopsies. Such fluids from the bronchial system however may also be taken after adding or rinsing with external fluid, in which case the typical sample would be e.g. induced sputum, bronchial lavage or bronchoalveolar lavage. Such biological samples derived from the lung may be taken by use of instruments (suction catheters, bronchoscope, brushes, forceps, Water absorbing trap) or without using instruments. The method may also be employed to analyse DNA already obtained from any such material.

The bronchial system (also called “airways”) is to be understood as the system of organs involved in the intake and exchange of air (especially oxygen and carbon dioxide) between an organism and the environment, e.g. trachea, bronchi, bronchioles, alveolar duct, alveoli).

The terms Bronchial lavage (BL) or Bronchoalveolar lavage (BAL) are to be understood as the types of fluids which are collected when the according medical procedures BL and BAL have been performed. BL and BAL are medical procedures in which a bronchoscope is passed through the mouth or nose into the lungs and fluid is squirted into a small part of the lung and then recollected for examination. BL/BAL is typically performed to diagnose lung disease. In particular, BAL is commonly used to diagnose infections in people with immune system problems, pneumonia in people on ventilators, some types of lung cancer, and scarring of the lung (interstitial lung disease). BAL is the most common manner to sample the components of the epithelial lining fluid (ELF) and to determine the protein composition of the pulmonary airways, and it is often used in immunological research as a means of sampling cells or pathogen levels in the lung. Examples of these include T-cell populations and influenza viral levels.

BL and BAL differ in the area (segment) of the bronchial system rinsed and the amount of fluid used:
BL focusses on the bronchi using approximately 10 ml of fluid.
BAL reaches further towards bronchioli and alveolar ducts using a higher amount of fluid (about 100 ml).

The term Bronchoscopy is understood to comprise a medical test to view the airways and diagnose lung disease. It may also be used during the treatment of some lung conditions.

Biological samples derived from the lung may also be achieved with a suction catheter for the trachea and the bronchial system, for example tubular, flexible suction catheter may be used for insertion into the trachea and the bronchial system, containing at least one continuous lumen for suction of fluids from the lungs.

The term lung carcinoma shall be taken to comprise lung adenocarcinoma; large cell lung cancer; squamous cell lung carcinoma and small cell lung carcinoma, as well as other forms of rare carcinoma types, which may be identified in a tumor which is located in the lung, whenever the specification refers to detection of lung carcinoma or diagnosis of lung carcinoma.

The term “methylation” is meant to be understood as cytosine methylation or CpG methylation. These terms are used to describe methylation at the C5 atom of the cytosine within a CpG context.

The present invention provides a method for detecting cell proliferative disorders, preferably those according to Table 2 (most preferably lung carcinoma) in a subject comprising determining the expression or methylation levels of at least one gene or genomic sequence selected from the group consisting of FOXL-2; ONECUT1; TFAP2E (including promoter or regulatory elements thereof) and EN2-2, EN2-3, SHOX2-2 and BARHL2 in a biological sample isolated from said subject wherein hyper-methylation and/or under-expression is indicative of the presence of said disorder. Said markers may be used for the diagnosis of cell proliferative disorders, preferably those according to Table 2 (most preferably lung carcinoma).

### Bisulfite Modification of DNA is an Art-Recognized Tool Used to Assess CpG Methylation Status.

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analysed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyse individual cells for methylation status, illustrating the utility and sensitivity of the method. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyse individual cytosine positions, or treated by enzymatic digestion (Xiong & Laird, Nucleic Acids Res., 25:2532-4, 1997). Detection by hybridisation has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 97/46705 and WO 95/15373).

The present invention provides for the use of the bisulfite technique, in combination with one or more methylation assays, for determination of the methylation status of CpG dinucleotide sequences within SEQ ID NO: 1 to SEQ ID NO: 7. Genomic CpG dinucleotides can be methylated or unmethylated (alternatively known as up- and down-methylated respectively). However the methods of the present invention are suitable for the analysis of biological samples of a heterogeneous nature e.g. a low concentration of tumor cells within a background of body fluid analyte, such as for example biological samples derived from the lung, such as sputum or bronchial lavage or bronchoalveolar lavage. Accordingly, when analyzing the methylation status of a CpG position within such a sample the person skilled in the art may use a quantitative assay for determining the level (e.g. percent, fraction, ratio, proportion or degree) of methylation at a particular CpG position as opposed to a methylation state. Accordingly the term methylation status or methylation state should also be taken to mean a value reflecting the degree of methylation at a CpG position, in other words the methylation level. Unless specifically stated the terms “hypermethylated” or “upmethylated” shall be taken to mean a methylation level above that of a specified cut-off point, wherein said cut-off may be a value representing the average or median methylation level for a given population, or is preferably an optimized cut-off level. The “cut-off” is also referred herein as a “threshold”. In the context of the present invention the terms “methylated”, “hypermethylated” or “upmethylated” shall be taken to include a methylation level above the cut-off be zero (0) % (or equivalents thereof) methylation for all CpG positions within and associated with (e.g. in promoter or regulatory regions) at least one gene or genomic sequence selected from the group consisting of FOXL-2; ONECUT1; TFAP2E (including promoter or regulatory elements thereof) and EN2-2, EN2-3, SHOX2-2 and BARHL2.

According to the present invention, determination of the methylation status of CpG dinucleotide sequences within SEQ ID NO: 1 to SEQ ID NO: 7 have utility in the diagnosis and detection of cell proliferative disorders, preferably those according to Table 2 (most preferably lung carcinoma).

### Methylation Assay Procedures.

Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

### COBRA.

COBRA™ analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific gene (or bisulfite treated DNA sequence or CpG island); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., cell proliferative disorders, preferably those according to Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., cell proliferative disorders, preferably those according to Cancer Res. 59:2307-12, 1999) are used alone or in combination with other of these methods.

The “HeavyMethyl™” assay, technique is a quantitative method for assessing methylation differences based on methylation specific amplification of bisulfite treated DNA. Methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight □-based kit) for HeavyMethyl™ analysis may include, but are not limited to: PCR primers for specific genes (or bisulfite treated DNA sequence or CpG island); blocking oligonucleotides; optimized PCR buffers and deoxynucleotides; and Taq polymerase.

### MSP.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylation-specific and unmethylation-specific PCR primers for specific gene(s) (or bisulfite treated DNA sequence or CpG island), optimized PCR buffers and deoxynucleotides, and specific probes.

### TSP Method.

The method was performed as described in the application EP08159227.1 (see p 29-28, under Examples). In brief, the DNA restriction Enzyme Tsp509I is used instead of the blocking oligonucleotides. This enzyme specifically cuts unmethylated DNA during amplicfication after bisulfite-treatment. As a result, unmethylated DNA is prevented from being amplified.

### MethyLight™.

The MethyLight™ assay is a high-throughput quantitative methylation assay that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” (with PCR primers that overlap known CpG dinucleotides) reaction. Sequence discrimination can occur both at the level of the amplification process and at the level of the fluorescence detection process.

The MethyLight™ assay may be used as a quantitative test for methylation patterns in the genomic DNA sample, wherein sequence discrimination occurs at the level of probe hybridization. In this quantitative version, the PCR reaction provides for a methylation specific amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the HeavyMethyl™ and MSP techniques), or with oligonucleotides covering potential methylation sites.

The MethyLight™ process can by used with any suitable probes e.g. “TaqMan®”, Lightcycler®, Scorpion™, etc. . . . . For example, double-stranded genomic DNA is treated with sodium bisulfite and subjected to one of two sets of PCR reactions using TaqMan® probes; e.g., with MSP primers and/or HeavyMethyl blocker oligonucleotides and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight□-based kit) for MethyLight™ analysis may include, but are not limited to: PCR primers for specific gene (or bisulfite treated DNA sequence or CpG island); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the HeavyMethyl™ and MSP techniques), or with oligonucleotides covering potential methylation sites.

The QM™ process can by used with any suitable probes e.g. “TaqMan®”, Lightcycler®, Scorpion®, etc. in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical QM™-based kit) for QM™ analysis may include, but are not limited to: PCR primers for specific gene (or bisulfite treated DNA sequence or CpG island); TaqMan® or Lightcycler® probes; optimized PCR buffers and deoxynucleotides; and Taq polymerase.

### Ms-SNuPE.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific gene (or bisulfite treated DNA sequence or CpG island); optimized PCR buffers and deoxynucleotides; gel extraction kit; positive control primers; Ms-SNuPE™ primers for specific gene; reaction buffer (for the Ms-SNuPE reaction); and labelled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

The genomic sequence(s) according to SEQ ID NO: 1 TO SEQ ID NO: 7 and non-naturally occurring treated variants thereof according to SEQ ID NO: 8 TO SEQ ID NO: 35 were determined to have novel utility for the detection of cell proliferative disorders, preferably those according to Table 2 (most preferably lung carcinoma). This utility has been exemplified in the specific assays described within the specification, especially in the examples.

The Scorpion® technique (generally described in patent application EP 9812768.1) has been adapted for the analysis of CpG methylation as described in detail within the published EP patent EP 1 654 388.

In one embodiment the method of the invention comprises the following steps: i) determining the expression of at least one gene or genomic sequence selected from the group consisting of ONECUT1; FOXL-2 and TFAP2E and ii) determining the presence or absence of a subject's risk or increased risk of suffering from a cell proliferative disorder, or detecting a cell proliferative disorder preferably those according to Table 2 (most preferably lung carcinoma). Preferred is the detection of a lung cancer selected from the group consisting of lung adenocarcinoma; large cell lung cancer; squamous cell lung carcinoma and small cell lung carcinoma.

The method of the invention may be enabled by means of any analysis of the expression of an RNA transcribed therefrom or polypeptide or protein translated from said RNA, preferably by means of mRNA expression analysis or polypeptide expression analysis. However, in the most preferred embodiment of the invention the detection of cell proliferative disorders, preferably those according to Table 2 (most preferably lung carcinoma), is enabled by means of analysis of the methylation status or methylation level of at least one gene or genomic sequence selected from the group consisting of FOXL-2; ONECUT1; TFAP2E (including promoter or regulatory elements thereof) and EN2-2, EN2-3, SHOX2-2 and BARHL2.

Accordingly the present invention also provides diagnostic assays and methods, both quantitative and qualitative for detecting the expression of at least one gene or genomic sequence selected from the group consisting of ONECUT1; FOXL-2 and TFAP2E in a subject and determining therefrom upon the presence or absence of a subject's risk or increased risk to suffer from a cell proliferative disorders, or to detect a cell proliferative disorder preferably those according to Table 2 (most preferably lung carcinoma) in said subject. Particularly preferred is that the cell proliferative disorder is lung cancer and particularly preferred that it is selected from the group consisting of lung adenocarcinoma; large cell lung cancer; squamous cell lung carcinoma and small cell lung carcinoma.

Aberrant expression of mRNA transcribed from at least one gene or genomic sequence selected from the group consisting of ONECUT1; FOXL-2 and TFAP2E is associated with the presence of cell proliferative disorders, preferably those according to Table 2 (most preferably lung carcinoma) in a subject. Particularly preferred is that the cell proliferative disorder is a lung cancer, preferably a lung cancer selected from the group consisting of lung adenocarcinoma, large cell lung cancer, squamous cell lung carcinoma and small cell lung carcinoma.

According to the present invention, hyper-methylation and for under-expression is associated with the presence of cell proliferative disorders, in particular those according to Table 2 (most preferably lung carcinoma).

To detect the presence of mRNA encoding a gene or genomic sequence, a sample is obtained from a patient. The sample may be any suitable sample comprising cellular matter of the tumor. Suitable sample types include cells or cell lines, histological slides, biopsies, paraffin-embedded tissue, body fluids, ejaculate, urine, blood plasma, blood serum, whole blood, isolated blood cells, sputum and biological matter derived from bronchoscopy (including but not limited to bronchial lavage, bronchial alveolar lavage, bronchial brushing, bronchial abrasion, and all possible combinations thereof. More preferably the sample type is selected form the group consisting of blood plasma, sputum and biological matter derived from bronchoscopy (including but not limited to bronchial lavage, bronchial alveolar lavage, bronchial brushing, and bronchial abrasion), and all possible combinations thereof.

The sample may be treated to extract the RNA contained therein. The resulting nucleic acid from the sample is then analysed. Many techniques are known in the state of the art for determining absolute and relative levels of gene expression, commonly used techniques suitable for use in the present invention include in situ hybridisation (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerisation chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligonucleotide dT primer and/or random hexamer primers. The cDNA thus produced is then amplified by means of PCR. (Belyaysky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridisation probes (e.g. TaqMan, Lightcycler, Molecular Beacons & Scorpion) or SYBR green. The detected signal from the probes or SYBR green is then quantitated either by reference to a standard curve or by comparing the Ct values to that of a calibration standard. Analysis of housekeeping genes is often used to normalize the results.

In Northern blot analysis total or poly(A)+mRNA is run on a denaturing agarose gel and detected by hybridisation to a labelled probe in the dried gel itself or on a membrane. The resulting signal is proportional to the amount of target RNA in the RNA population.

Comparing the signals from two or more cell populations or tissues reveals relative differences in gene expression levels. Absolute quantitation can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labelled (e.g. radioactive labels, mass labels or fluorescent labels). The size of the target RNA, not the probe, will determine the size of the detected band, so methods such as random-primed labelling, which generates probes of variable lengths, are suitable for probe synthesis. The specific activity of the probe will determine the level of sensitivity, so it is preferred that probes with high specific activities, are used.

In an RNase protection assay, the RNA target and an RNA probe of a defined length are hybridised in solution. Following hybridisation, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantitation of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al. 2003), particularly preferred are probes with high specific activities.

Particularly preferred is the use of microarrays. The microarray analysis process can be divided into two main parts. First is the immobilization of known gene sequences onto glass slides or other solid support followed by hybridisation of the fluorescently labelled cDNA (comprising the sequences to be interrogated) to the known genes immobilized on the glass slide (or other solid phase). After hybridisation, arrays are scanned using a fluorescent microarray scanner. Analysing the relative fluorescent intensity of different genes provides a measure of the differences in gene expression.

DNA arrays can be generated by immobilizing presynthesized oligonucleotides onto prepared glass slides or other solid surfaces. In this case, representative gene sequences are manufactured and prepared using standard oligonucleotide synthesis and purification methods. These synthesized gene sequences are complementary to the RNA transcript(s) of at least one gene or genomic sequence selected from the group consisting of ONECUT1; FOXL-2 and TFAP2E and tend to be shorter sequences in the range of 25-70 nucleotides. Alternatively, immobilized oligos can be chemically synthesized in situ on the surface of the slide. In situ oligonucleotide synthesis involves the consecutive addition of the appropriate nucleotides to the spots on the microarray; spots not receiving a nucleotide are protected during each stage of the process using physical or virtual masks. Preferably said synthesized nucleic acids are locked nucleic acids.

In expression profiling microarray experiments, the RNA templates used are representative of the transcription profile of the cells or tissues under study. RNA is first isolated from the cell populations or tissues to be compared. Each RNA sample is then used as a template to generate fluorescently labelled cDNA via a reverse transcription reaction. Fluorescent labelling of the cDNA can be accomplished by either direct labelling or indirect labelling methods. During direct labelling, fluorescently modified nucleotides (e.g., Cy®3- or Cy®5-dCTP) are incorporated directly into the cDNA during the reverse transcription. Alternatively, indirect labelling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabelled, but may be detectable by specific binding with a ligand which is labelled, either directly or indirectly. Suitable labels and methods for labelling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labelled with Cy®3. The resulting labelled cDNA is purified to remove unincorporated nucleotides, free dye and residual RNA. Following purification, the labelled cDNA samples are hybridised to the microarray. The stringency of hybridisation is determined by a number of factors during hybridisation and during the washing procedure, including temperature, ionic strength, length of time and concentration of formamide. These factors are outlined in, for example, Sambrook et al. (Molecular Cloning: A Laboratory Manual, 2nd ed., 1989). The microarray is scanned post-hybridisation using a fluorescent microarray scanner. The fluorescent intensity of each spot indicates the level of expression of the analysed gene; bright spots correspond to strongly expressed genes, while dim spots indicate weak expression.

Once the images are obtained, the raw data must be analysed. First, the background fluorescence must be subtracted from the fluorescence of each spot. The data is then normalized to a control sequence, such as exogenously added nucleic acids (preferably RNA or DNA), or a housekeeping gene panel to account for any non-specific hybridisation, array imperfections or variability in the array set-up, cDNA labelling, hybridisation or washing. Data normalization allows the results of multiple arrays to be compared.

Another aspect of the invention relates to a kit for use in diagnosis of cell proliferative disorders, preferably those according to Table 2 (most preferably lung carcinoma and further preferred is a lung cancer selected from the group consisting of lung adenocarcinoma; large cell lung cancer; squamous cell lung carcinoma; small cell lung carcinoma.) in a subject according to the methods of the present invention, said kit comprising: a means for measuring the level of transcription of at least one gene or genomic sequence selected from the group consisting of ONECUT1; FOXL-2 and TFAP2E. In a preferred embodiment the means for measuring the level of transcription comprise oligonucleotides or polynucleotides able to hybridise under stringent or moderately stringent conditions to the transcription products of at least one gene or genomic sequence selected from the group consisting of FOXL-2; ONECUT1; TFAP2E (including promoter or regulatory elements thereof) and EN2-2, EN2-3, SHOX2-2 and BARHL2. In a most preferred embodiment the level of transcription is determined by techniques selected from the group of Northern Blot analysis, reverse transcriptase PCR, real-time PCR, RNAse protection, and microarray. In another embodiment of the invention the kit further comprises means for obtaining a biological sample of the patient. Preferred is a kit, which further comprises a container which is most preferably suitable for containing the means for measuring the level of transcription and the biological sample of the patient, and most preferably further comprises instructions for use and interpretation of the kit results.

In a preferred embodiment the kit comprises (a) a plurality of oligonucleotides or polynucleotides able to hybridise under stringent or moderately stringent conditions to the transcription products of at least one gene or genomic sequence selected from the group consisting of FOXL-2; ONECUT1; TFAP2E (including promoter or regulatory elements thereof) and EN2-2, EN2-3, SHOX2-2 and BARHL2; (b) a container, preferably suitable for containing the oligonucleotides or polynucleotides and a biological sample of the patient comprising the transcription products wherein the oligonucleotides or polynucleotides can hybridise under stringent or moderately stringent conditions to the transcription products, (c) means to detect the hybridisation of (b); and optionally, (d) instructions for use and interpretation of the kit results.

The kit may also contain other components such as hybridisation buffer (where the oligonucleotides are to be used as a probe) packaged in a separate container. Alternatively, where the oligonucleotides are to be used to amplify a target region, the kit may contain, packaged in separate containers, a polymerase and a reaction buffer optimised for primer extension mediated by the polymerase, such as PCR. Preferably said polymerase is a reverse transcriptase. It is further preferred that said kit further contains an Rnase reagent.

The present invention further provides for methods for the detection of the presence of the polypeptide encoded by said gene sequences in a sample obtained from a patient.

Aberrant levels of polypeptide expression of the polypeptides encoded at least one gene or genomic sequence selected from the group consisting of ONECUT1; FOXL-2 and TFAP2E are associated with the presence of cell proliferative disorders, preferably those according to Table 2 (most preferably lung carcinoma). Particularly preferred is a lung cancer selected from the group consisting of lung adenocarcinoma; large cell lung cancer; squamous cell lung carcinoma; small cell lung carcinoma.

According to the present invention under-expression of said polypeptides is associated with the presence of cell proliferative disorders, preferably those according to Table 2 (most preferably lung carcinoma). It is particularly preferred that the cell proliferative disorder is lung cancer and that it is selected from the group consisting of lung adenocarcinoma; large cell lung cancer; squamous cell lung carcinoma and small cell lung carcinoma.

Any method known in the art for detecting polypeptides can be used. Such methods include, but are not limited to masss-spectrometry, immunodiffusion, immunoelectrophoresis, immunochemical methods, binder-ligand assays, immunohistochemical techniques, agglutination and complement assays (e.g., see Basic and Clinical Immunology, Sites and Terr, eds., Appleton & Lange, Norwalk, Conn. pp 217-262, 1991 which is incorporated by reference). Preferred are binder-ligand immunoassay methods including reacting antibodies with an epitope or epitopes and competitively displacing a labelled polypeptide or derivative thereof.

Certain embodiments of the present invention comprise the use of antibodies specific to the polypeptide(s) encoded by at least one gene or genomic sequence selected from the group consisting of ONECUT1; FOXL-2 and TFAP2E.

Such antibodies are useful for cell proliferative disorders, preferably of those diseases according to Table 2, and most preferably in the diagnosis of lung carcinoma. Particularly preferred is a lung cancer selected from the group consisting of lung adenocarcinoma; large cell lung cancer; squamous cell lung carcinoma; small cell lung carcinoma. In certain embodiments production of monoclonal or polyclonal antibodies can be induced by the use of an epitope encoded by a polypeptide of at least one gene or genomic sequence selected from the group consisting of ONECUT1; FOXL-2 and TFAP2E as an antigene. Such antibodies may in turn be used to detect expressed polypeptides as markers for cell proliferative disorders, preferably those according to Table 2 and most preferably the diagnosis of lung carcinoma. Particularly preferred is a lung cancer selected from the group consisting of lung adenocarcinoma; large cell lung cancer; squamous cell lung carcinoma; small cell lung carcinoma. The levels of such polypeptides present may be quantified by conventional methods. Antibody-polypeptide binding may be detected and quantified by a variety of means known in the art, such as labelling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabelled, for example as used in agglutination tests, or labelled for use a wide variety of assay methods. Labels that can be used include radionuclides, enzymes, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spacial separation achieved by electrophoresis. The membrane is then incubated with a blocking agent to bind remaining sticky places on the membrane, commonly used agents include generic protein (e.g. milk protein). An antibody specific to the protein of interest is then added, said antibody being detectably labelled for example by dyes or enzymatic means (e.g. alkaline phosphatase or horseradish peroxidase). The location of the antibody on the membrane is then detected.

In an alternative embodiment of the method the proteins may be detected by means of immunohistochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to coloured deposits at the antibody-antigen binding sites. There is a wide range of suitable sample types, antigen-antibody affinity, antibody types, and detection enhancement methods. Thus optimal conditions for immunohistochemical or immunocytochemical detection must be determined by the person skilled in the art for each individual case.

One approach for preparing antibodies to a polypeptide is the selection and preparation of an amino acid sequence of all or part of the polypeptide, chemically synthesising the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference in its entirety). Methods for preparation of the polypeptides or epitopes thereof include, but are not limited to chemical synthesis, recombinant DNA techniques or isolation from biological samples.

In the final step of the method, the diagnosis of the patient is determined, whereby under-expression (of mRNA or polypeptides) is indicative of the presence of cell proliferative disorders, preferably those according to Table 2 (most preferably lung carcinoma). Particularly preferred it is a lung cancer, preferably selected from the group consisting of lung adenocarcinoma; large cell lung cancer; squamous cell lung carcinoma and small cell lung carcinoma. The term under-expression shall be taken to mean expression at a detected level less than a pre-determined cut off which may be selected from the group consisting of the mean, median or an optimised threshold value. The term over-expression shall be taken to mean expression at a detected level greater than a pre-determined cut off which may be selected from the group consisting of the mean, median or an optimised threshold value.

Another aspect of the invention provides a kit for use in diagnosis of cell proliferative disorders, preferably those according to Table 2 (most preferably lung carcinoma) in a subject according to the methods of the present invention, comprising: a means for detecting polypeptides of at least one gene or genomic sequence selected from the group consisting of ONECUT1; FOXL-2 and TFAP2E. The means for detecting the polypeptides comprise preferably antibodies, antibody derivatives, or antibody fragments. The polypeptides are most preferably detected by means of Western Blotting utilizing a labelled antibody. In another embodiment of the invention the kit further comprising means for obtaining a biological sample of the patient. Preferred is a kit, which further comprises a container suitable for containing the means for detecting the polypeptides in the biological sample of the patient, and most preferably further comprises instructions for use and interpretation of the kit results. In a preferred embodiment the kit comprises: (a) a means for detecting polypeptides of at least one gene or genomic sequence selected from the group consisting of ONECUT1; FOXL-2 and TFAP2E; (b) a container suitable for containing the said means and the biological sample of the patient comprising the polypeptides wherein the means can form complexes with the polypeptides; (c) a means to detect the complexes of (b); and optionally (d) instructions for use and interpretation of the kit results.

The kit may also contain other components such as buffers or solutions suitable for blocking, washing or coating, packaged in a separate container.

Particular embodiments of the present invention provide a novel application of the analysis of methylation status, methylation levels and/or patterns within at least one gene or genomic sequence selected from the group consisting of FOXL-2; ONECUT1; TFAP2E (including promoter or regulatory elements thereof) and EN2-2, EN2-3, SHOX2-2 and BARHL2. that enables a precise detection, characterisation, assessment of risk to suffer from cell proliferative disorders, preferably those according to Table 2 (most preferably lung carcinoma). It is particularly preferred that this lung cancer is selected from the group consisting of lung adenocarcinoma; large cell lung cancer; squamous cell lung carcinoma and small cell lung carcinoma. Early detection of cell proliferative disorders, in particular lung carcinoma, is directly linked with disease prognosis, and the disclosed method thereby enables the physician and patient to make better and more informed treatment decisions. Therefore it is preferred that the method of the invention which allows detection of disease in an early stage is performed as a screening tool, or as an additional diagnostic test, whenever a first diagnosis is unclear.

The preferred sample type used within the method of the invention is sputum or biological samples derived from the lung, preferably, bronchial fluid, bronchial lavage and bronchoalveolar lavage. This sample type has the advantage that it is a sample which is currently used in common practice and obtainable by established and routine diagnostic procedures of lung disease as part of the standard care (e.g. histology procedures and/or cytology procedures). The advantage of using available samples is that additional information from the same sample can be achieved. The second advantage is, that these samples can be obtained non-invasively (for example sputum) or with low risk to the subject or patient.

Another important advantage of using samples which are collected from the bronchial system is, that the marker that can be used for a specific diagnosis of lung cancer or risk assessment of lung cancer may be less specific in terms cancer type. It would not harm, if the same marker is also detecting other cancer types (if tested on other sample types, for example blood).

In the most preferred embodiment of the method, the presence or absence of risk or increased risk of a subject to suffer from a cell proliferative disorder, or detecting of a cell proliferative disorder, preferably those according to Table 2 (most preferably lung carcinoma, in particular a lung cancer selected from the group consisting of lung adenocarcinoma; large cell lung cancer; squamous cell lung carcinoma and small cell lung carcinoma.) is determined by analysis of the methylation status or level of one or more CpG dinucleotides of at least one gene or genomic sequence selected from the group consisting of FOXL-2; ONECUT1; TFAP2E (including promoter or regulatory elements thereof) and EN2-2, EN2-3, SHOX2-2 and BARHL2.

In one embodiment the invention of said method comprises the following steps: i) contacting genomic DNA (preferably isolated from body fluids) obtained from the subject with at least one reagent, or series of reagents that distinguishes between methylated and non-methylated CpG dinucleotides within at least one gene or genomic sequence selected from the group consisting of FOXL-2; ONECUT1; TFAP2E (including promoter or regulatory elements thereof) and EN2-2, EN2-3, SHOX2-2 and BARHL2 and ii) detecting cell proliferative disorders, preferably those according to Table 2 (most preferably lung carcinoma). Particularly preferred is a lung cancer selected from the group consisting of lung adenocarcinoma; large cell lung cancer; squamous cell lung carcinoma and small cell lung carcinoma.

It is preferred that said one or more CpG dinucleotides of at least one gene or genomic sequence selected from the group consisting of FOXL-2; ONECUT1; TFAP2E (including promoter or regulatory elements thereof) and EN2-2, EN2-3, SHOX2-2 and BARHL2 are comprised within a respective genomic target sequence thereof as provided in SEQ ID NO: 1 to SEQ ID NO: 7 and complements thereof. The present invention further provides a method for ascertaining genetic and/or epigenetic parameters of at least one gene or genomic sequence selected from the group consisting of FOXL-2; ONECUT1; TFAP2E (including promoter or regulatory elements thereof) and EN2-2, EN2-3, SHOX2-2 and BARHL2 and/or the genomic sequence according to SEQ ID NO: 1 to SEQ ID NO: 7 within a subject by analysing cytosine methylation. Said method comprising contacting a nucleic acid comprising SEQ ID NO: 1 to SEQ ID NO: 7 in a biological sample obtained from said subject with at least one reagent or a series of reagents, wherein said reagent or series of reagents, distinguishes between methylated and non-methylated CpG dinucleotides within the target nucleic acid.

In a preferred embodiment, said method comprises the following steps: In the first step, a sample of the tissue to be analysed is obtained. The source may be any suitable source, such as cells or cell lines, histological slides, biopsies, paraffin-embedded tissue, body fluids, ejaculate, urine, blood plasma, blood serum, whole blood, isolated blood cells, sputum, biological samples derived from the lung, preferably biological matter derived from bronchoscopy including but not limited to bronchial lavage, bronchial alveolar lavage, bronchial brushing, bronchial abrasion, and all possible combinations thereof. More preferably the sample type is selected form the group consisting of blood plasma, sputum, biological samples derived from the lung, preferably biological matter derived from bronchoscopy (including, but not limited to, bronchial lavage, bronchial alveolar lavage, bronchial brushing, and bronchial abrasion) and all possible combinations thereof. It is a preferred embodiment of the method of the invention that the sample type is selected from the group consisting of sputum and biological samples derived from the lung (as described earlier), most preferably this biological matter is derived from bronchoscopy (including but not limited to bronchial lavage, bronchial alveolar lavage, bronchial brushing, and bronchial abrasion).

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Wherein the sample DNA is not enclosed in a membrane (e.g. circulating DNA from a blood sample) methods standard in the art for the isolation and/or purification of DNA may be employed. Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. The person skilled in the art may also make use of devices such as filter devices e.g. ultrafiltration, silica surfaces or membranes, magnetic particles, polystyrol particles, polystyrol surfaces, positively charged surfaces, and positively charged membrane, charged membranes, charged surfaces, charged switch membranes, charged switched surfaces.

Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pre-treatment’ or ‘treatment’ herein.

This explicit order of steps is only one embodiment of the method of the invention, because it is also possible and sometimes advantageous to omit the DNA isolation step prior to the bisulfite treatment. In that case the bisulfite treatment (see in detail below) is performed before the DNA is isolated and/or purified, for example if the sample DNA is not enclosed in a membrane. Hence the bisulfite treatment may be performed on a crude sample, i.e. the biological material itself. In some cases, the presence of a surfactant, such as for example SDS, may be needed.

This is preferably achieved by means of treatment with a bisulfite reagent. The term “bisulfite reagent” refers to a reagent comprising bisulfite, disulfite, hydrogen sulfite or combinations thereof, useful as disclosed herein to distinguish between methylated and unmethylated CpG dinucleotide sequences. Methods of said treatment are known in the art (e.g. PCT/EP2004/011715, which is incorporated by reference in its entirety). It is preferred that the bisulfite treatment is conducted in the presence of denaturing solvents such as but not limited to n-alkylenglycol, particularly diethylene glycol dimethyl ether (DME), or in the presence of dioxane or dioxane derivatives. In a preferred embodiment the denaturing solvents are used in concentrations between 1% and 35% (v/v). It is also preferred that the bisulfite reaction is carried out in the presence of scavengers such as but not limited to chromane derivatives, e.g., 6-hydroxy-2,5,7,8,-tetramethylchromane-2-carboxylic acid or trihydroxybenzoe acid and derivates thereof, e.g. Gallic acid (see: PCT/EP2004/011715 which is incorporated by reference in its entirety). The bisulfite conversion is preferably carried out at a reaction temperature between 30° C. and 70° C., whereby the temperature is increased to over 85° C. for short periods of times during the reaction (see: PCT/EP2004/011715 which is incorporated by reference in its entirety). The bisulfite treated DNA is preferably purified priori to the quantification. This may be conducted by any means known in the art, such as but not limited to ultrafiltration, preferably carried out by means of Microcon̂™ columns (manufactured by Milliporê™). The purification is carried out according to a modified manufacturer's protocol (see: PCT/EP2004/011715 which is incorporated by reference in its entirety).

In the third step of the method, fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). Preferably said amplificates are 100 to 2,000 base pairs in length. The set of primer oligonucleotides includes at least two oligonucleotides whose sequences are each reverse complementary, identical, or hybridise under stringent or highly stringent conditions to an at least 16-base-pair long segment of the base sequences of one of SEQ ID NO: 8 to SEQ ID NO: 35 and sequences complementary thereto.

In an alternate embodiment of the method, the methylation status or level of pre-selected CpG positions within at least one gene or genomic sequence selected from the group consisting of FOXL-2; ONECUT1; TFAP2E (including promoter or regulatory elements thereof) and EN2-2, EN2-3, SHOX2-2 and BARHL2 and preferably within the nucleic acid sequences according to SEQ ID NO: 1 to SEQ ID NO: 7 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer which hybridises to a bisulfite treated CpG dinucleotide. Therefore, the sequence of said primers comprises at least one CpG dinucleotide. MSP primers specific for non-methylated DNA contain a “T” at the position of the C position in the CpG. Preferably, therefore, the base sequence of said primers is required to comprise a sequence having a length of at least 9 nucleotides which hybridises to a treated nucleic acid sequence according to one of SEQ ID NO: 8 to SEQ ID NO: 35 and sequences complementary thereto, wherein the base sequence of said oligomers comprises at least one CpG dinucleotide. A further preferred embodiment of the method comprises the use of blocker oligonucleotides (the HeavyMethyl™ assay). The use of such blocker oligonucleotides has been described by Yu et al., BioTechniques 23:714-720, 1997. Blocking probe oligonucleotides are hybridised to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a CpA′ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridises to a treated nucleic acid sequence according to one of SEQ ID NO: 8 to SEQ ID NO: 35 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analysed by means of based-based methods such as, but not limited to, array technology and probe based technologies as well as by means of techniques such as sequencing and template directed extension.

In one embodiment of the method, the amplificates synthesised in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG TpG or CpA dinucleotide. The hybridizing portion of the hybridizing nucleic acids is typically at least 9, 15, 20, 25, 30 or 35 nucleotides in length. However, longer molecules have inventive utility, and are thus within the scope of the present invention.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within a sequence selected from the group consisting SEQ ID NO: 1 to SEQ ID NO: 7, and the equivalent positions within SEQ ID NO: 8 to SEQ ID NO: 35. Said oligonucleotides may also be present in the form of peptide nucleic acids. The non-hybridised amplificates are then removed. The hybridised amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes (as detailed above) that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which, in preferred embodiments, is designed to hybridise to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethyLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

In the most preferred embodiment of the method the genomic nucleic acids are isolated and treated according to the first three steps of the method outlined above, namely:

a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridization properties; and wherein
d) amplifying subsequent to treatment in c) is carried out in a methylation specific manner, namely by use of methylation specific primers or methylation specific blocking oligonucleotides, and further wherein
e) detecting of the amplificates is carried out by means of a real-time detection probe, as described above.

Preferably, where the subsequent amplification of d) is carried out by means of methylation specific primers, as described above, said methylation specific primers comprise a sequence having a length of at least 9 nucleotides which hybridises to a treated nucleic acid sequence according to one of SEQ ID NO: 8 to SEQ ID NO: 35 and sequences complementary thereto, wherein the base sequence of said oligomers comprises at least one CpG dinucleotide, but preferably two or three.

Step e) of the method, namely the detection of the specific amplificates indicative of the methylation status of one or more CpG positions according to SEQ ID NO: 1 to SEQ ID NO: 7 is carried out by means of real-time detection methods as described above.

Additional embodiments of the invention provide a method for the analysis of the methylation status of the at least one gene or genomic sequence selected from the group consisting of FOXL-2; ONECUT1; TFAP2E (including promoter or regulatory elements thereof) and EN2-2, EN2-3, SHOX2-2 and BARHL2 (preferably SEQ ID NO: 1 to SEQ ID NO: 7 and complements thereof) without the need for bisulfite conversion. Methods are known in the art wherein a methylation sensitive restriction enzyme reagent, or a series of restriction enzyme reagents comprising methylation sensitive restriction enzyme reagents that distinguishes between methylated and non-methylated CpG dinucleotides within a target region are utilized in determining methylation, for example but not limited to DMH.

In the first step of such additional embodiments, the genomic DNA sample is isolated from tissue or cellular sources. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. All clinical sample types comprising neoplastic or potentially neoplastic matter are suitable for use in the present method, preferred are cells or cell lines, histological slides, biopsies, paraffin-embedded tissue, body fluids, ejaculate, urine, blood plasma, blood serum, whole blood, isolated blood cells, and biological samples derived from the lung, such as sputum and biological matter derived from bronchoscopy (including but not limited to bronchial lavage, bronchial alveolar lavage, bronchial brushing, bronchial abrasion, and combinations thereof. More preferably the sample type is selected form the group consisting of blood plasma, sputum and biological matter derived from bronchoscopy (including but not limited to bronchial lavage, bronchial alveolar lavage, bronchial brushing, bronchial abrasion) and all possible combinations thereof.

Once the nucleic acids have been extracted, the genomic double-stranded DNA is used in the analysis.

In a preferred embodiment, the DNA may be cleaved prior to treatment with methylation sensitive restriction enzymes. Such methods are known in the art and may include both physical and enzymatic means. Particularly preferred is the use of one or a plurality of restriction enzymes which are not methylation sensitive, and whose recognition sites are AT rich and do not comprise CG dinucleotides. The use of such enzymes enables the conservation of CpG islands and CpG rich regions in the fragmented DNA. The non-methylation-specific restriction enzymes are preferably selected from the group consisting of MseI, BfaI, Csp6I, Tru1I, Tvu1I, Tru9I, Tvu9I, MaeI and XspI. Particularly preferred is the use of two or three such enzymes. Particularly preferred is the use of a combination of MseI, BfaI and Csp6I.

The fragmented DNA may then be ligated to adaptor oligonucleotides in order to facilitate subsequent enzymatic amplification. The ligation of oligonucleotides to blunt and sticky ended DNA fragments is known in the art, and is carried out by means of dephosphorylation of the ends (e.g. using calf or shrimp alkaline phosphatase) and subsequent ligation using ligase enzymes (e.g. T4 DNA ligase) in the presence of dATPs. The adaptor oligonucleotides are typically at least 18 base pairs in length.

In the third step, the DNA (or fragments thereof) is then digested with one or more methylation sensitive restriction enzymes. The digestion is carried out such that hydrolysis of the DNA at the restriction site is informative of the methylation status of a specific CpG dinucleotide of at least one gene or genomic sequence selected from the group consisting of FOXL-2; ONECUT1; TFAP2E (including promoter or regulatory elements thereof) and EN2-2, EN2-3, SHOX2-2 and BARHL2.

Preferably, the methylation-specific restriction enzyme is selected from the group consisting of Bsi E1, Hga I HinPl, Hpy99I, Ava I, Bce AI, Bsa HI, BisI, BstUI, BshI236I, AccII, BstFNI, McrBC, GlaI, MvnI, HpaII (HapII), HhaI, AciI, SmaI, HinP1I, HpyCH4IV, EagI and mixtures of two or more of the above enzymes. Preferred is a mixture containing the restriction enzymes BstUI, HpaII, HpyCH4IV and HinP1I.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels. Particularly preferred is amplification by means of an amplification enzyme and at least two primers comprising, in each case a contiguous sequence at least 16 nucleotides in length that is complementary to, or hybridizes under moderately stringent or stringent conditions to a sequence selected from the group consisting SEQ ID NO: 1 to SEQ ID NO: 7, and complements thereof. Preferably said contiguous sequence is at least 16, 20 or 25 nucleotides in length. In an alternative embodiment said primers may be complementary to any adaptors linked to the fragments.

In the fifth step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis. Preferably said detection is carried out by hybridisation to at least one nucleic acid or peptide nucleic acid comprising in each case a contiguous sequence at least 16 nucleotides in length that is complementary to, or hybridizes under moderately stringent or stringent conditions to a sequence selected from the group consisting of SEQ ID NO: 1 to SEQ ID NO: 7, and complements thereof. Preferably said contiguous sequence is at least 16, 20 or 25 nucleotides in length.

Subsequent to the determination of the methylation state or methylation level of the genomic nucleic acids obtained from a subject's sample, the risk or increased risk of a subject to suffer from a cell proliferative disorder, preferably those according to Table 2 (most preferably lung carcinoma), or the presence of such a cell proliferative disorder is deduced based upon the methylation state or level of at least one CpG dinucleotide sequence of SEQ ID NO: 1 to SEQ ID NO: 7, or an average, or a value reflecting an average methylation state of a plurality of CpG dinucleotide sequences of SEQ ID NO: 1 to SEQ ID NO: 7 wherein methylation is associated with the presence of cell proliferative disorders, preferably those according to Table 2 (most preferably lung carcinoma). Wherein said methylation is determined by quantitative means the cut-off point for determining said presence of methylation is preferably zero (i.e. wherein a sample displays any degree of methylation it is determined as having a methylated status at the analyzed CpG position). Nonetheless, it is foreseen that the person skilled in the art may wish to adjust said cut-off value in order to provide an assay of a particularly preferred sensitivity or specificity. Accordingly said cut-off value may be increased (thus increasing the specificity), said cut off value may be within a range selected form the group consisting of 0%-5%, 5%-10%, 10%45%, 15%-20%, 20%-30% and 30%-50%. Particularly preferred are the cut-offs 10%, 15%, 25%, and 30%.

Upon determination of the methylation and/or expression of at least one gene or genomic sequence selected from the group consisting of FOXL-2; ONECUT1; TFAP2E (including promoter or regulatory elements thereof) and EN2-2, EN2-3, SHOX2-2 and BARHL2 the presence or absence of a cell proliferative disorder or an increased risk of a subject to suffer from a cell proliferative disorder, preferably those according to Table 2 (most preferably lung carcinoma) is determined, wherein hyper-methylation and/or under-expression indicates the presence of cell proliferative disorders and/or the presence of an increased risk of the subject to suffer from such a disorder, preferably those according to Table 2 (most preferably lung carcinoma) and hypo-methylation and for over-expression indicates the absence of cell proliferative disorders within the subject, and/or the absence of an increased risk of the subject to suffer from such a disorder, preferably those according to Table 2 (most preferably lung carcinoma). It is particularly preferred that said proliferative disorder is a lung cancer selected from the group consisting of lung adenocarcinoma; large cell lung cancer; squamous cell lung carcinoma and small cell lung carcinoma.

An increased risk is to be understood as a risk that is at least two fold higher than the average risk of the population with the same gender in the same age group (wherein subjects belong to the same age group if they are not more than 5 years older or younger than the subject analysed.

### Further Improvements

The disclosed invention provides treated nucleic acids, derived from genomic SEQ ID NO: 1 to SEQ ID NO: 7, wherein the treatment is suitable to convert at least one unmethylated cytosine base of the genomic DNA sequence to uracil or another base that is detectably dissimilar to cytosine in terms of hybridization. The genomic sequences in question may comprise one, or more consecutive methylated CpG positions. Said treatment preferably comprises use of a reagent selected from the group consisting of bisulfite, hydrogen sulfite, disulfite, and combinations thereof. Said treatment may however also comprise an appropriate enzymatic treatment (instead of the bisulfite treatment), resulting in conversion of the unmethylated cytosines into base pairs with a different base pairing behavious. In a preferred embodiment of the invention, the invention provides a non-naturally occurring modified nucleic acid comprising a sequence of at least 16 contiguous nucleotide bases in length of a sequence selected from the group consisting of SEQ ID NO: 8 TO SEQ ID NO: 35. In further preferred embodiments of the invention said nucleic acid is at least 50, 100, 150, 200, 250 or 500 base pairs in length of a segment of the nucleic acid sequence disclosed in SEQ ID NO: 8 to SEQ ID NO: 35. Particularly preferred is a nucleic acid molecule that is identical or complementary to all or a portion of the sequences SEQ ID NO: 8 to SEQ ID NO: 35 but not to SEQ ID NO: 1 to SEQ ID NO: 7 or other naturally occurring DNA.

It is preferred that said sequence comprises at least one CpG, TpA or CpA dinucleotide and sequences complementary thereto. The sequences of SEQ ID NO: 8 TO SEQ ID NO: 35 provide non-naturally occurring modified versions of the nucleic acid according to SEQ ID NO: 1 TO SEQ ID NO: 7, wherein the modification of each genomic sequence results in the synthesis of a nucleic acid having a sequence that is unique and distinct from said genomic sequence as follows. For each sense strand genomic DNA, e.g., SEQ ID NO: 1 to SEQ ID NO: 7, four converted versions are disclosed. A first version wherein “C” is converted to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequences of SEQ ID NO: 1 to SEQ ID NO: 7 correspond to SEQ ID NO: 8 to SEQ ID NO: 21. A third chemically converted version of each genomic sequences is provided, wherein “C” is converted to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO: 1 to SEQ ID NO: 7 correspond to SEQ ID NO: 19 to SEQ ID NO: 30.

Significantly, heretofore, the nucleic acid sequences and molecules according to SEQ ID NO: 8 to SEQ ID NO: 35 were not implicated in or connected with the detection or diagnosis of cell proliferative disorders, preferably those according to Table 2 (most preferably lung carcinoma). It is particularly preferred that the cell proliferative disorder is a lung cancer selected from the group consisting of lung adenocarcinoma; large cell lung cancer; squamous cell lung carcinoma and small cell lung carcinoma.

In an alternative preferred embodiment, the invention further provides oligonucleotides or oligomers suitable for use in the methods of the invention for detecting the cytosine methylation state within genomic or treated (chemically modified) DNA, according to SEQ ID NO: 1 to SEQ ID NO: 35 Said oligonucleotide or oligomer nucleic acids provide novel diagnostic means. Said oligonucleotide or oligomer comprising a nucleic acid sequence having a length of at least nine (9) nucleotides which is identical to, hybridizes, under moderately stringent or stringent conditions (as defined herein above), to a treated nucleic acid sequence according to SEQ ID NO: 8 to SEQ ID NO: 35 and/or sequences complementary thereto, or to a genomic sequence according to SEQ ID NO: 1 to SEQ ID NO: 7; and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NO: 1 to SEQ ID NO: 35 or to the complements thereof. Particularly preferred is a nucleic acid molecule that hybridizes under moderately stringent and/or stringent hybridization conditions to all or a portion of the sequences SEQ ID NO: 8 to SEQ ID NO: 35 but not to SEQ ID NO: 1 to SEQ ID NO: 7 or other human genomic DNA.

The identical or hybridizing portion of the hybridizing nucleic acids is typically at least 9, 16, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO: 8 to SEQ ID NO: 35, or to the complements thereof.

Hybridizing nucleic acids of the type described herein can be used, for example, as a primer (e.g., a PCR primer), or a diagnostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO: 1 to SEQ ID NO: 7 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO: 1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y−(X−1));

where Y equals the length (nucleotides or base pairs) of SEQ ID NO: 1 (3905);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO 1 of length Y is equal to Y−(X−1). For example Z=3905−19=3886 for either sense or antisense sets of SEQ ID NO: 1, where X=20.

Preferably, the set is limited to those oligomers that comprise at least one CpG, TpG or CpA dinucleotide, and thus hybridise in any case to a region of the converted target DNA, that comprises at least one (methylated or unmethylated) CpG in its unconverted version.

Examples of inventive 20-mer oligonucleotides include the following set of 3905 oligomers (and the antisense set complementary thereto), indicated by polynucleotide positions with reference to SEQ ID NO: 1:

1-20, 2-21, 3-22, 4-23, 5-24, . . . and 3886-3905

Preferably, the set is limited to those oligomers that comprise at least one CpG, TpG or CpA dinucleotide and thus hybridise in any case to a region of the converted target DNA, that comprises at least one (methylated or unmethylated) CpG in its unconverted version.

Likewise, examples of inventive 25-mer oligonucleotides include the following set of 3881 oligomers (and the antisense set complementary thereto), indicated by polynucleotide positions with reference to SEQ ID NO: 1:

1-25, 2-26, 3-27, 4-28, 5-29, . . . and 3881-3905.

Preferably, the set is limited to those oligomers that comprise at least one CpG, TpG or CpA dinucleotide and thus hybridise in any case to a region of the converted target DNA, that comprises at least one (methylated or unmethylated) CpG in its unconverted version.

The present invention encompasses, for each of SEQ ID NO: 1 to SEQ ID NO: 35 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO: 1 to SEQ ID NO: 7. Preferred sets of such oligonucleotides or modified oligonucleotides of length X are those consecutively overlapping sets of oligomers corresponding to SEQ ID NO: 1 to SEQ ID NO: 35 (and to the complements thereof). Preferably, said oligomers comprise at least one CpG; TpG or CpA dinucleotide and thus hybridise in any case to a region of the converted target DNA, that comprises at least one (methylated or unmethylated) CpG in its unconverted version.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of a genomic sequence or parts thereof selected from the group consisting of SEQ ID NO: 1 to SEQ ID NO: 7 and sequences complementary thereto, or to the corresponding CpG, TpG or CpA dinucleotide within a sequence of the treated nucleic acids according to SEQ ID NO: 8 to SEQ ID NO: 35 and sequences complementary thereto. However, it is anticipated that for economic or other factors it may be preferable to analyse a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in treated genomic DNA (SEQ ID NO: 8 to SEQ ID NO: 35), or in genomic DNA (SEQ ID NO: 1 to SEQ ID NO: 7 and sequences complementary thereto). These probes enable diagnosis and detection of cell proliferative disorders, preferably those according to Table 2 (most preferably lung carcinoma). It is particularly preferred that it is a lung cancer selected from the group consisting of lung adenocarcinoma; large cell lung cancer; squamous cell lung carcinoma and small cell lung carcinoma. The set of oligomers may also be used for detecting single nucleotide polymorphisms (SNPs) in treated genomic DNA (SEQ ID NO: 8 to SEQ ID NO: 35), or in genomic DNA (SEQ ID NO: 1 to SEQ ID NO: 7 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO: 1 to SEQ ID NO: 35 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labelled probes are often used for the scanning of immobilized DNA arrays. The simple attachment of Cy3 and Cy5 dyes to the 5′-OH of the specific probe are particularly suitable for fluorescence labels. The detection of the fluorescence of the hybridised probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003), which is hereby incorporated by reference. In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilised for detecting, or for diagnosing cell proliferative disorders, preferably those according to Table 2 (most preferably lung carcinoma) or for detecting the presence or absence of an increased risk of a subject to suffer from a cell proliferative disorder, preferably those according to Table 2 (most preferably lung carcinoma). It is particularly preferred that the disorder is a lung cancer and that it is selected from the group consisting of lung adenocarcinoma; large cell lung cancer; squamous cell lung carcinoma and small cell lung carcinoma.

### Kits

Moreover, an additional aspect of the present invention is a kit comprising: a means for determining the expression or methylation status or levels of at least one gene or genomic sequence selected from the group consisting of FOXL-2; ONECUT1; TFAP2E (including promoter or regulatory elements thereof) and EN2-2, EN2-3, SHOX2-2 and BARHL2. The means for determining the expression or methylation status or levels of at least one gene or genomic sequence selected from the group consisting of FOXL-2; ONECUT1; TFAP2E (including promoter or regulatory elements thereof) and EN2-2, EN2-3, SHOX2-2 and BARHL2 preferably comprise a bisulfite-containing reagent; one or a plurality of oligonucleotides wherein the sequences thereof are identical, are complementary, or hybridise under stringent or highly stringent conditions to a 9 or more preferably 18 base long segment of a sequence selected from SEQ ID NO: 8 to SEQ ID NO: 35; and optionally instructions for carrying out and evaluating the described method of methylation analysis. In one embodiment the base sequence of said oligonucleotides comprises at least one CpG, CpA or TpG dinucleotide.

In a further embodiment, said kit may further comprise standard reagents for performing a CpG position-specific methylation analysis, wherein said analysis comprises one or more of the following techniques: MS-SNuPE, MSP, MethyLight™, HeavyMethyl, COBRA, and nucleic acid sequencing. However, a kit along the lines of the present invention can also contain only part of the aforementioned components.

In a preferred embodiment the kit may comprise additional bisulfite conversion reagents selected from the group consisting: DNA denaturation buffer; sulfonation buffer; DNA recovery reagents or kits (e.g., precipitation, ultrafiltration, affinity column); desulfonation buffer; and DNA recovery components.

In a further alternative embodiment, the kit may contain, packaged in separate containers, a polymerase and a reaction buffer optimised for primer extension mediated by the polymerase, such as PCR. In another embodiment of the invention the kit further comprising means for obtaining a biological sample of the patient. Preferred is a kit, which further comprises a container suitable for containing the means for determining methylation of at least one gene or genomic sequence selected from the group consisting of FOXL-2; ONECUT1; TFAP2E (including promoter or regulatory elements thereof) and EN2-2, EN2-3, SHOX2-2 and BARHL2 in the biological sample of the patient, and most preferably further comprises instructions for use and interpretation of the kit results. In a preferred embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridise under stringent or highly stringent conditions to a 9 or more preferably 18 base long segment of a sequence selected from SEQ ID NO: 8 to SEQ ID NO: 35; and optionally (d) instructions for use and interpretation of the kit results. In an alternative preferred embodiment the kit comprises: (a) a bisulfite reagent; (b) a container suitable for containing the said bisulfite reagent and the biological sample of the patient; (c) at least one oligonucleotides and/or PNA-oligomer having a length of at least 9 or 16 nucleotides which is identical to or hybridises to a pre-treated nucleic acid sequence according to one of SEQ ID NO: 8 to SEQ ID NO: 35 and sequences complementary thereto; and optionally (d) instructions for use and interpretation of the kit results.

In an alternative embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridise under stringent or highly stringent conditions to a 9 or more preferably 18 base long segment of a sequence selected from SEQ ID NO: 8 to SEQ ID NO: 35; (d) at least one oligonucleotides and/or PNA-oligomer having a length of at least 9 or 16 nucleotides which is identical to or hybridises to a pre-treated nucleic acid sequence according to one of SEQ ID NO: 8 to SEQ ID NO: 35 and sequences complementary thereto; and optionally (e) instructions for use and interpretation of the kit results.

The kit may also contain other components such as buffers or solutions suitable for blocking, washing or coating, packaged in a separate container.

Another aspect of the invention relates to a kit for use in determining the presence of and/or diagnosing cell proliferative disorders, preferably those according to Table 2 (most preferably lung carcinoma). Particularly preferred is a lung cancer selected from the group consisting of lung adenocarcinoma; large cell lung cancer; squamous cell lung carcinoma; small cell lung carcinoma.

Said kit prefereably comprises: a means for measuring the level of transcription of at least one gene or genomic sequence selected from the group consisting of ONECUT1; FOXL-2 and TFAP2E and a means for determining methylation status or level of at least one gene or genomic sequence selected from the group consisting of FOXL-2; ONECUT1; TFAP2E (including promoter or regulatory elements thereof) and EN2-2, EN2-3, SHOX2-2 and BARHL2.

Typical reagents (e.g., as might be found in a typical COBRA™-based kit) for COBRA™ analysis may include, but are not limited to: PCR primers for at least one gene or genomic sequence selected from the group consisting of FOXL-2; ONECUT1; TFAP2E (including promoter or regulatory elements thereof) and EN2-2, EN2-3, SHOX2-2 and BARHL2 and/or their bisulfite converted sequences; restriction enzyme and appropriate buffer; gene-hybridization oligo; control hybridization oligo; kinase labeling kit for oligo probe; and labeled nucleotides. Typical reagents (e.g., as might be found in a typical MethyLight™-based kit) for MethyLight™ analysis may include, but are not limited to: PCR primers for the bisulfite converted sequence of at least one gene or genomic sequence selected from the group consisting of ONECUT1; FOXL-2 and TFAP2E (including promoter or regulatory elements thereof) and EN2-2, EN2-3, SHOX2-2 and BARHL2; bisulfite specific probes (e.g. TaqMan™ or Lightcycler™); optimized PCR buffers and deoxynucleotides; and Taq polymerase.

Typical reagents (e.g., as might be found in a typical Ms-SNuPE™-based kit) for Ms-SNuPE™ analysis may include, but are not limited to: PCR primers for specific gene (or bisulfite treated DNA sequence or CpG island); optimized PCR buffers and deoxynucleotides; gel extraction kit; positive control primers; Ms-SNuPE™ primers for the bisulfite converted sequence of at least one gene or genomic sequence selected from the group consisting of ONECUT1; FOXL-2 and TFAP2E (including promoter or regulatory elements thereof) and EN2-2, EN2-3, SHOX2-2 and BARHL2; reaction buffer (for the Ms-SNuPE reaction); and labelled nucleotides.

Typical reagents (e.g., as might be found in a typical MSP-based kit) for MSP analysis may include, but are not limited to: methylation-specific and unmethylation-specific PCR primers for the bisulfite converted sequence of at least one gene or genomic sequence selected from the group consisting of ONECUT1; FOXL-2 and TFAP2E (including promoter or regulatory elements thereof) and EN2-2, EN2-3, SHOX2-2 and BARHL2, optimized PCR buffers and deoxynucleotides, and specific probes.

Moreover, an additional aspect of the present invention is an alternative kit comprising a means for determining methylation (status or level) of at least one gene or genomic sequence selected from the group consisting of ONECUT1; FOXL-2 and TFAP2E (including promoter or regulatory elements thereof) and EN2-2, EN2-3, SHOX2-2 and BARHL2, wherein said means comprise preferably at least one methylation specific restriction enzyme; one or a plurality of primer oligonucleotides (preferably one or a plurality of primer pairs) suitable for the amplification of a sequence comprising at least one CpG dinucleotide of a sequence selected from SEQ ID NO: 1 to SEQ ID NO: 7 and optionally instructions for carrying out and evaluating the described method of methylation analysis. In one embodiment the base sequence of said oligonucleotides are identical, are complementary, or hybridise under stringent or highly stringent conditions to an at least 18 base long segment of a sequence selected from SEQ ID NO: 1 to SEQ ID NO: 7.

In a further embodiment said kit may comprise one or a plurality of oligonucleotide probes for the analysis of the digest fragments, preferably said oligonucleotides are identical, are complementary, or hybridise under stringent or highly stringent conditions to an at least 16 base long segment of a sequence selected from SEQ ID NO: 1 to SEQ ID NO: 7.

In a preferred embodiment the kit may comprise additional reagents selected from the group consisting: buffer (e.g. restriction enzyme, PCR, storage or washing buffers); DNA recovery reagents or kits (e.g., precipitation, ultrafiltration, affinity column) and DNA recovery components.

In a further alternative embodiment, the kit may contain, packaged in separate containers, a polymerase and a reaction buffer optimised for primer extension mediated by the polymerase, such as PCR. In another embodiment of the invention the kit further comprising means for obtaining a biological sample of the patient. In a preferred embodiment the kit comprises: (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridise under stringent or highly stringent conditions to an at least 9 base long segment of a sequence selected from SEQ ID NO: 1 to SEQ ID NO: 7 and optionally (d) instructions for use and interpretation of the kit results.

In an alternative preferred embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from SEQ ID NO: 1 to SEQ ID NO: 7 and optionally (d) instructions for use and interpretation of the kit results.

In an alternative embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from SEQ ID NO: 1 to SEQ ID NO: 7 (d) at least one set of oligonucleotides one or a plurality of nucleic acids or peptide nucleic acids which are identical, are complementary, or hybridise under stringent or highly stringent conditions to an at least 9 base long segment of a sequence selected from SEQ ID NO: 1 to SEQ ID NO: 7 and optionally (e) instructions for use and interpretation of the kit results.

The kit may also contain other components such as buffers or solutions suitable for blocking, washing or coating, packaged in a separate container.

The invention further relates to a kit for use in providing a diagnosis of the presence or absence of cell proliferative disorders, preferably those according to Table 2 (most preferably lung carcinoma), in a subject by means of methylation-sensitive restriction enzyme analysis. Said kit comprises a container and a DNA microarray component. Said DNA microarray component being a surface upon which a plurality of oligonucleotides are immobilized at designated positions and wherein the oligonucleotide comprises at least one CpG methylation site. At least one of said oligonucleotides is specific for at least one gene or genomic sequence selected from the group consisting of ONECUT1; FOXL-2 and TFAP2E (including promoter or regulatory elements thereof) and EN2-2, EN2-3, SHOX2-2 and BARHL2 and comprises a sequence of at least 15 base pairs in length but no more than 200 by of a sequence according to one of SEQ ID NO: 1 to SEQ ID NO: 7. Preferably said sequence is at least 15 base pairs in length but no more than 80 bp of a sequence according to one of SEQ ID NO: 1 to SEQ ID NO: 7. It is further preferred that said sequence is at least 20 base pairs in length but no more than 30 bp of a sequence according to one of SEQ ID NO: 1 to SEQ ID NO: 7.

Said test kit preferably further comprises a restriction enzyme component comprising one or a plurality of methylation-sensitive restriction enzymes.

In a further embodiment said test kit is further characterized in that it comprises at least one methylation-specific restriction enzyme, and wherein the oligonucleotides comprise a restriction site of said at least one methylation specific restriction enzymes.

The kit may further comprise one or several of the following components, which are known in the art for DNA enrichment: a protein component, said protein binding selectively to methylated DNA; a triplex-forming nucleic acid component, one or a plurality of linkers, optionally in a suitable solution; substances or solutions for performing a ligation e.g. ligases, buffers; substances or solutions for performing a column chromatography; substances or solutions for performing an immunology based enrichment (e.g. immunoprecipitation); substances or solutions for performing a nucleic acid amplification e.g. PCR; a dye or several dyes, if applicable with a coupling reagent, if applicable in a solution; substances or solutions for performing a hybridization; and/or substances or solutions for performing a washing step.

The described invention further provides a composition of matter useful for detecting, or for diagnosing cell proliferative disorders, preferably those according to Table 2 (most preferably lung carcinoma). Particularly preferred is a lung cancer selected from the group consisting of lung adenocarcinoma; large cell lung cancer; squamous cell lung carcinoma; small cell lung carcinoma.

Said composition preferably comprises at least one nucleic acid 18 base pairs in length of a segment of the nucleic acid sequence disclosed in SEQ ID NO: 8 to SEQ ID NO: 35, and one or more substances taken from the group comprising:

1-5 mM Magnesium Chloride, 100-500 μM dNTP, 0.5-5 units of taq polymerase, bovine serum albumen, an oligomer in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a pretreated genomic DNA according to one of the SEQ ID NO: 8 to SEQ ID NO: 35 and sequences complementary thereto. It is preferred that said composition of matter comprises a buffer solution appropriate for the stabilization of said nucleic acid in an aqueous solution and enabling polymerase based reactions within said solution. Suitable buffers are known in the art and commercially available.

In further preferred embodiments of the invention said at least one nucleic acid is at least 50, 100, 150, 200, 250 or 500 base pairs in length of a segment of the nucleic acid sequence disclosed in SEQ ID NO: 8 to SEQ ID NO: 35.

TABLE 1

| | | Pretreated | Pretreated | Pretreated | Pretreated |
|---|---|---|---|---|---|
| | | methylated | methylated | unmethylated | unmethylated |
| | | sequence | strand | sequence | sequence |
| | Genomic | (sense) | (antisense) | (sense) | (antisense) |
| Gene | SEQ ID NO: | SEQ ID NO: | SEQ ID NO: | SEQ ID NO: | SEQ ID NO: |
| SHOX2-2 | 1 | 8 | 9 | 22 | 23 |
| EN2-2 | 2 | 10 | 11 | 24 | 25 |
| Second CpG island | | | | | |
| associated with | | | | | |
| Homeobox protein | | | | | |
| engrailed-2 | | | | | |
| (Hu-En-2); EN2; | | | | | |
| HME2 | | | | | |
| EN2-3 | 3 | 12 | 13 | 26 | 27 |
| Third CpG island | | | | | |
| associated with | | | | | |
| Homeobox protein | | | | | |
| engrailed-2 | | | | | |
| (Hu-En-2); EN2; | | | | | |
| HME2 | | | | | |
| ONECUT 1 | 4 | 14 | 15 | 28 | 29 |
| FOXL2 | 5 | 16 | 17 | 30 | 31 |
| TFAP2E | 6 | 18 | 19 | 32 | 33 |
| BARHL2 | 7 | 20 | 21 | 34 | 35 |

TABLE 2

| Gene | Preferred disorder |
|---|---|
| SHOX2-2 | Cancer, preferably lung |
| EN2-2 | Cancer, preferably lung |
| EN2-3 | Cancer, preferably lung |
| ONECUT 1 | Cancer, preferably lung |
| FOXL-2 | Cancer, preferably lung |
| TFAP2E | Cancer, preferably lung |
| BARHL2 | Cancer, preferably lung |

TABLE 3A
MSP Assays

| Gene/ | MSP- | Forward | Reverse | |
|---|---|---|---|---|
| Genomic | Amplicon/ | Primer/ | Primer/ | Probe/ |
| region | SEQ ID NO: | SEQ ID NO: | SEQ ID NO: | SEQ ID NO: |
| ONECUT1 | gttttgaaat | gttttgaaat | ctttctaaaa | tacggacgtt |
| | ttattagaat | ttattagaat | ataaccgaac | cgcgggtcgt |
| | aacgacgttt | aacgacgtt/ | tatactacga | t/43 |
| | taaaaataaa | 41 | c/42 | |
| | ggcgtagtaa | | | |
| | gtattttttt | | | |
| | tttcgttgtc | | | |
| | gcgggttgaa | | | |
| | ttacggacgt | | | |
| | tcgcgggtcg | | | |
| | tttagtttcg | | | |
| | acggttcgta | | | |
| | gggggcgcgc | | | |
| | gtcgtagtcg | | | |
| | tagtatagtt | | | |
| | cggttatttt | | | |
| | tagaaag/36 | | | |
| TFAP2E | tttagaagcg | tttagaagcg | ccgaacgctt | ttgcggtggg |
| | gttttcgtat | gttttcgtat | acctacaat | cgttttcggg |
| | cgttgcggtg | c/52 | c/53 | tt/54 |
| | ggcgttttcg | | | |
| | ggtttcgatt | | | |
| | tcgttagcgt | | | |
| | cgcggggtag | | | |
| | aggtatttgg | | | |
| | agttcgtagg | | | |
| | gtttagattt | | | |
| | gggttggaaa | | | |
| | agtttcgttg | | | |
| | attgtaggta | | | |
| | agcgttcgg/ | | | |
| | 37 | | | |

TABLE 3B
Heavy Methyl Assays

| | | Forward | Reverse | Forward | Reverse | |
|---|---|---|---|---|---|---|
| Gene/ | | Primer/ | Primer/ | Blocker/ | Blocker/ | Probe/ |
| Genomic | HM-Amplicon/ | SEQ ID | SEQ ID | SEQ ID | SEQ ID | SEQ ID |
| region | SEQ ID NO: | NO: | NO: | NO: | NO: | NO: |
| FOXL-2 | ccaagacctggg | ccaaaac | gagaggg | tacaacac | ttgggaag | ccgccg |
| | cttgcagcgccg | ctaaact | gttagta | caccaaca | attttggt | aaaaca |
| | ccaacaggcccg | tacaac/ | gt/45 | aacccaaa | ttggagt/ | cgaaac |
| | gggacacgaggc | 44 | | aacacaa/ | 47 | ggcggg |
| | gctccaggccgg | | | 46 | | agaggg |
| | ggtcttcccggc | | | | | gttagt |
| | tgctggcccctc | | | | | agt/48; |
| | tcgctccccacc | | | | | cccggg |
| | cgctggcggcgc | | | | | aagatt |
| | ctcggtcgcccg | | | | | ttggtt |
| | caattgacccaa | | | | | tggagc |
| | cccgcttcctgc | | | | | ccgggc |
| | gtttgcccctca | | | | | caaaac |
| | ggtttcc/39 | | | | | ctaaac |
| | | | | | | ttacaa |
| | | | | | | c/49; |
| | | | | | | ctccaa |
| | | | | | | accaaa |
| | | | | | | atcttc |
| | | | | | | cc/50; |
| | | | | | | ccgaaa |
| | | | | | | acacga |
| | | | | | | aacgct |
| | | | | | | c/51 |
| TFAP2E | aaacccaaacct | aaaccca | ggaagtg | gtaaagtg | / | aaaaac |
| | aaattaaaaaaa | aacctaa | tgtggta | ttggggtt | | ttcgct |
| | cttcgctaacta | attaaa/ | aag/56 | ttgtttgg | | aactac |
| | caaacaaacgtc | 55 | | ttgttt/ | | aaacaa |
| | cgaaaaaaacga | | | 57 | | ac/58 |
| | ccaaacgaaacc | | | | | |
| | ccgacgctttac | | | | | |
| | cacacacttcc/ | | | | | |
| | 40 | | | | | |

TABLE 3C
TSP assays

| Gene/ | | | | |
|---|---|---|---|---|
| Genomic | TSP PCR | Primer 1/ | Primer 2/ | Probe/ |
| region | Amplicon | SEQ ID NO: | SEQ ID NO: | SEQ ID NO: |
| BARHL2 | attgtttg | gttttgaaat | acatataaca | ttggattatt |
| | ttagtttt | ttattagaat | aatatatttt | ttaaatgtgg |
| | taagttaa | aacgacgtt/ | atccaac/60 | ttaaaa/61 |
| | tcgtagta | 59 | | |
| | ataatcgt | | | |
| | tggattat | | | |
| | tttaaatg | | | |
| | tggttaaa | | | |
| | atcgacgt | | | |
| | tggataaa | | | |
| | attaattt | | | |
| | gttatatg | | | |
| | t/38 | | | |

TABLE 4

| Gene/Marker | AUC | Sensitivity | Specificity |
|---|---|---|---|
| FOXL2 | 0.911 | 0.575 | 0.957 |
| BARHL2 | 0.766 | 0.400 | 0.957 |

### EXAMPLES

The following analysis was performed to examine the methylation status of FOXL2 and BARHL2 gene markers. DNA was first extracted from bronchial lavage samples and bisulfite treated. The treated DNA was analyzed using HeavyMethyl-based real-time PCR on the ABI PRISM 7900HT platform.

### Preanalytics

### DNA Extraction

Genomic DNA from unfixed bronchial lavage specimens was isolated using a QIAamp DNA Micro Kit (Qiagen, Hilden, Germany). The viscosity of the bronchial lavage samples was reduced, before DNA extraction, by adding 1,4-Dithiothreitol (DTT, Carl Roth, Germany) to a final concentration of 0.225% and incubating the samples at room temperature for at least 30 minutes or until the desired fluidity was obtained. After centrifugation at 3200×g for 12 minutes, the pellet was processed using a QIAamp DNA Micro Kit according to the manufacturer's protocol.

### Bisulfite Treatment

Bisulfite treatment of extracted sample DNA was performed using an EpiTect Kit (Qiagen, Hilden, Germany) according to the manufacturer's instructions with the following modifications. A fixed volume of 15 μl DNA from sample extractions was mixed with 5 μl water, 85 μl bisulfite mix and 35 μl protection buffer. Two elution steps were performed using 25 μl elution buffer each time.

### Analytics

### Principle

The quantification of the methylation of a specific locus is achieved via two PCRs. The first PCR is comprised of two gene specific primers and and a gene specific probe which detects DNA irrespective of its methylation state (qualification of total DNA). The second PCR is comprised of the same primers but contains a probe specific for methylated DNA and two blockers to supress the amplification of unmethylated DNA.

For FOXL2 (SEQ ID NO: 123)
Forward primer
SEQ ID NO: 44
ccaaaacctaaacttacaac
Reverse primer
SEQ ID NO: 45
gagaggggttagtagt
Forward blocker
SEQ ID NO: 46
tacaacaccaccaacaaacccaaaaacacaa
Reverse blocker
SEQ ID NO: 47
ttgggaagattttggtttggagt

In one assay (for BARHL2) the DNA restriction Enzyme Tsp509I is used instead of the blocking oligonucleotides. This enzyme specifically cuts unmethylated DNA after bisulfite-treatment leading to methylation specific amplification.

For BARHL2 (SEQ ID NO: 125)
Primer
SEQ ID NO: 59
gttttgaaatttattagaataacgacgtt
Primer
SEQ ID NO: 60
acatataacaaatatattttatccaac

### Biomarkers/Assays

The following assays were performed with Scorpion probes: FOXL2,

Probe
SEQ ID NO: 48:
ccgccgaaaacacgaaacggcgggagaggggttagtagt
Probe
SEQ ID NO: 49
cccgggaagattttggtttggagcccgggccaaaacctaaacttacaac

The following assays were performed with TaqMan probes: BARHL2

Probe
SEQ ID NO: 61
ttggattattttaaatgtggttaaaa

### Heavy Methyl Based Real-Time PCR

Real-time PCR experiments were performed using the Applied Biosystems ABI PRISM 7900HT instrument. Each real-time assay for one biomarker consisted of two independend reactions: a reference reaction for quantification of total input DNA and a HM-reaction for quantification of methylated target template. The reference assay was composed of two methylation-unspecific oligonucleotides and a methylation unspecific probe, whereas the HM-assay consisted of the same two methylation-unspecific primers, but in addition two methylation-specific blockers (one for each primer) and a methylation-specific probe. For the biomarker BARHL2, the DNA restriction Enzyme Tsp509I is used instead of the blocking oligonucleotides. This enzyme specifically cuts unmethylated DNA during amplicfication after bisulfite-treatment. As a result, unmethylated DNA is prevented from being amplified.

Two different probe systems were used for RT-PCR analysis, depending on the biomarker/assay. For FOXL2, Scorpion® probes consisting of a methylation-unspecific primer part and a methylation-specific probe part were used. The Scorpion® probes contained BHQ1 as quencher and 6-FAM as fluorescent reporter. For the markers BARHL2 TaqMan probes with BHQ1 and 6-FAM were used as detection system. Each assay was tested with 86 BL samples (40 cancer, 46 benign lung disease). Each PCR plate contained several PCR controls. These included 50 ng of bisulfit-treated Sperm DNA (0% BisStd), which is usually unmethylated, 0.5 ng methylated Chemicon DNA in 50 ng Sperm DNA (1% BisStd) and non template controls (NTCs). These controls were used to monitor the general RT-PCR performance and to define concentration limits for sample exclusion (see Data and Statistical analyses).

The 20 μl PCR reactions contained 0.25 μl of bisulfite treated sample DNA (without any prior determination of concentration), 10 μl of QuantiTect Multiplex PCR NoROX mixture (Qiagen, Hilden), 0.3 μM unspecific forward and reverse primer and either 0.3 μM TaqMan probe oder 0.15 μM Scorpion® probe. When a Scorpion® probe was used in the experiment, the concentration of the respective non-probe primer was reduced to 0.15 μM. TaqMan probe concentration was 0.30 μM. For HM-reactions, blockers where added to a final concentration of 1 μM each. For Tsp509I-based assay, 1 U of restriction enzyme was used for the methylation-specific amplification.

Thermocycling conditions were as follows: an initial denaturation at 95° C. for 15 minutes followed by 50 cycles of 95° C. for 15 seconds and a annealing/denaturation step at 56° C. for 30 seconds. Single fluorescent detection was performed during the annealing/elongation step.

### Clinical Samples

Number of clinical samples: 86
Cancer samples: 40
Benign samples: 46

1-21. (canceled)

22. A nucleic acid comprising at least 9 contiguous nucleotides of a treated genomic DNA sequence selected from the group consisting of SEQ ID NO: 16, 17, 30, 31, and sequences complementary thereto.

23. The nucleic acid of claim 22, wherein said nucleic acid is directly or indirectly linked to a detectable label or is a peptide nucleic acid (PNA) oligomer, a 3′-deoxyoligonucleotide or an oligonucleotide derivatized at the 3′ position with other than a free hydroxyl group.

24. The nucleic acid of claim 22, wherein the contiguous base sequence comprises at least one CpG, TpG or CpA dinucleotide sequence.

25. The nucleic acid of claim 22, wherein said nucleic acid is an oligonucleotide.

26. The nucleic acid of claim 22, wherein said nucleic acid comprises at least 9 contiguous nucleotides of the sequence according to
(i) SEQ ID NO: 16 or a sequence complementary thereto, wherein said at least 9 contiguous nucleotides are comprised in the sequence according to SEQ ID NO: 30 or a sequence complementary thereto, or
(ii) SEQ ID NO: 17 or a sequence complementary thereto, wherein said at least 9 contiguous nucleotides are comprised in the sequence according to SEQ ID NO: 31 or a sequence complementary thereto.

27. The nucleic acid of claim 26, wherein said nucleic acid is a primer oligonucleotide.

28. The nucleic acid of claim 27, wherein said primer oligonucleotide has a sequence according to SEQ ID NO: 44 or 45.

29. The nucleic acid of claim 22, wherein said nucleic acid comprises at least 9 contiguous nucleotides of the sequence according to SEQ ID NO: 16 or 17, or a sequence complementary thereto.

30. The nucleic acid of claim 29, wherein said at least 9 contiguous nucleotides are not comprised in a sequence according to SEQ ID NO: 30 or 31, or a sequence complementary thereto.

31. The nucleic acid of claim 30, wherein said nucleic acid is a primer or probe oligonucleotide.

32. The nucleic acid of claim 31, wherein said probe oligonucleotide has a sequence according to SEQ ID NO: 48, 49, 50 or 51.

33. The nucleic acid of claim 31, wherein said probe oligonucleotide is directly or indirectly linked to a detectable label, optionally wherein said detectable label is a fluorescence label, a radionuclide or a mass label.

34. The nucleic acid of claim 22, wherein said nucleic acid comprises at least 9 contiguous nucleotides of the sequence according to SEQ ID NO: 30 or 31, or a sequence complementary thereto.

35. The nucleic acid of claim 34, wherein said at least 9 contiguous nucleotides are not comprised in a sequence according to SEQ ID NO: 16 or 17, or a sequence complementary thereto.

36. The nucleic acid of claim 35, wherein said nucleic acid is a blocker oligonucleotide, optionally wherein said blocker oligonucleotide is a peptide nucleic acid (PNA) oligomer, a 3′-deoxyoligonucleotide or an oligonucleotide derivitized at the 3′ position with other than a free hydroxyl group.

37. The nucleic acid of claim 36, wherein said blocker oligonucleotide has a sequence according to SEQ ID NO: 46 or 47.

38. A kit comprising a pair of primer oligonucleotides comprising a first and a second primer oligonucleotide, wherein the first primer oligonucleotide is a nucleic acid according to claim 26 (i) and the second primer oligonucleotide is a nucleic acid according to claim 26 (ii).

39. The kit of claim 38, further comprising a blocker oligonucleotide, a probe oligonucleotide, and/or a bisulfite reagent; wherein
said blocker oligonucleotide comprises at least 9 contiguous nucleotides of the sequence according to SEQ ID NO: 30 or 31, or a sequence complementary thereto, wherein said at least 9 contiguous nucleotides are not comprised in a sequence according to SEQ ID NO: 16 or 17, or a sequence complementary thereto; and
said probe oligonucleotide comprises at least 9 contiguous nucleotides of the sequence according to SEQ ID NO: 16 or 17, or a sequence complementary thereto and wherein said at least 9 contiguous nucleotides are not comprised in a sequence according to SEQ ID NO: 30 or 31, or a sequence complementary thereto.

40. A kit comprising a first primer oligonucleotide and a second primer oligonucleotide,
(i) wherein said first primer oligonucleotide comprises at least 9 contiguous nucleotides of the sequence according to SEQ ID NO: 16 or 17, or a sequence complementary thereto and wherein said at least 9 contiguous nucleotides are not comprised in a sequence according to SEQ ID NO: 30 or 31, or a sequence complementary thereto; and
(ii) wherein said second primer oligonucleotide comprises either:
at least 9 contiguous nucleotides of the sequence according to SEQ ID NO: 16 or 17, or a sequence complementary thereto and wherein said at least 9 contiguous nucleotides are not comprised in a sequence according to SEQ ID NO: 30 or 31, or a sequence complementary thereto; or
at least 9 contiguous nucleotides of the sequence according to
(i) SEQ ID NO: 16 or a sequence complementary thereto, wherein said at least 9 contiguous nucleotides are comprised in the sequence according to SEQ ID NO: 30 or a sequence complementary thereto, or
(ii) SEQ ID NO: 17 or a sequence complementary thereto, wherein said at least 9 contiguous nucleotides are comprised in the sequence according to SEQ ID NO: 31 or a sequence complementary thereto.

41. The kit of claim 41, further comprising a probe oligonucleotide according to claim 30 and/or a bisulfite reagent.


SEQUENCE LISTING (61 sequences)

SEQ ID NO: 1 - DNA, 9739 bp, Homo Sapiens
acggaagcatagaaatgtattcaataaatatgcaacagaaggacaatcaatctaatatcaaagtgactagagagttaatgccagggctccatgggtatgctgacattccagaatagagaggactgaaggtcagagactctgggtaacctgcacattaagaacgtcctcaagttatcggggggaaagatagagtcctttctgttctacacaaaggggcagatctaaaatcacagtccctcaaaatctctaaaaatttttgcaagatttgaagagagctttgttccaaactccagtgcacacaatataaacacacattatcaatacttacaataagagattggaattaaagtgcacaccacctcccaaagtttttcaaaactgcttctaatcacaaaaaggtaaattaaaaatcctctctatgttacagtagtttaaaaggtaaataatggaagtagaaaggagatgtacatttgttgaaacctgttatgtaccaggcaccttacataacttatttttatttgatcctcacaataattggcaaggtgtgtgctgcaggacactaagaggtcatagattttaggaggctaagaaatgacctaaaactttacatctaatagttggtatagcaaagatttgaagtcaagaatatctgcctctaaaaattagccgtatactatgacaacacatcccaaataaagatgtataaaaagtacttggctcttcatagatattatataaaatatgggacaatattagtgatatacctaaatttagacattagaaagaagtccttcttaatttactctgtaattgtaactcagttgttcatcttacaggtccctatagtaataattaaacattcctttctttgttttatatgtgttttcaaactgctattaaagatccttggtcagcagcctttatatcttttggcgttttgctgctaatgctcggtaaacatgggcagccaaggaaaatgcagatggtgctatggaacacaaaccacgacaacatatggtatacatttagtcctaacaaagtgcgcttcttaacctctgggtgtcgcttcaccattcctttataagactactcatttccagggaaggtttagtttccaaaaccagggcaacaggcccacatgtgttatatggccattcagatgcacagattatgtgtcctaatacctttaagcaagtgcatttagcacatgcttcctagcactatcatgtgtatttatgaactgttttgcccagatggtaagctcctcgaggacaagtgccatgcctgaaatctcaccttattaactgtgcatggcagagtaacaatgttatctatcttgcatatagagacacacaacctataaattgctcttctaggtaaacctcctaactacactgctacattgtgacaattgtcacatcttctagatgaggctcacacaggctaagcaattagccagaatcacaaactaacaggcaatgttttcttcttgcccacaagagaaagcttttcttgtcttgcaacactttattgtttttttcttttaaggtttttattacaaaggtcaatgtctattggccttttaataaaagtaattattatttagtacttagtatacatatgcaaggcattgttctaagggttttacaaatattaactcatttaatcctcctaaccattccatgagatagacactatttttatccacattttacagataaggaaactgagggacagagagattaacttgtccaaaatcagaggactggtaaacagtagcagtgaaatttgaatccagcagtgaagcatagctccacagtgtgtgctcttaaacatacactattctgcttgaataagagtctacaaagatccttactcaggacatatgaggagttgttcagcaagaaaagaagcatgtttactcctccctatttgatgtatttgattatattctaaaacctttcctcatgccaatagaactccgcgtattatactaaactttcctgcaccgaaggcatatgtaatggttaccttgactgacatgtaaaatacccaaaacaaaataccccgagactcgaggattttttgtttcatgaattagacaccaaaaaaaaaattgtctaggcatcagaagtgttatacactcacatctgtaagtctttcttgggattttcagatttcttatacaagatttttttttgtctgaaatatttcagtgctcactcaatgcctgagcttgaaaattatctaatcctttttaattttttcattttcagctactttgccttcaggcatagggcagggcaagcacttgctttcaatatgatgtttaacactttttatgtttttattttcgtcatcttgaaaacatgctagtaaacatccaaacatactgtaaagatttaaaatttggggtgtgtatacgtatatcaacaggagttatgaattatgtatattgcaggcctatataaattagattttgaagaacaagcatcacagtaatcaagcgggtcataataagacattccatgtgaaatgtaaaaactaccttgaataaattatctgtaagttaacattctcattagaatgacatatttattatttctgggtttgtgatattgctttaatgtattttggctaactgttttaatggcatattaagaaaccatttcaggccttttttccccgttggaatttgcagacttttatttccgtttgaactaaaataatataatattaaaaacaatcccccacttcccaatgtacaataccattttcttctgcccagaaatatttaattaaagcaggatataacctcagtaattatttttacacagaatgactcttaaattaatagtcagttgtatgactaaaaattgggagactgacaatcaaaacaatcttaaatgctttgtttatgtgatgaaaatgagtgatcctttaattccctcacacacattaaactgatttcacagattttctgtactggtgtttaacatagtcaagtgcttgaggttatgtaaaataaacaatcttgagatcttattgcaaatgtttgcaatttatgatgtaaactgatttgtgaagaaaaaacaggatcctatgtcgctgaagccaaagaggcatttttcagaaatcaaaatagttccgaaatttgagcattgcattatactaaagtattaccatctgaggacccaaaaaagttataaatctggggaaaaactcaaaatagatgtacatcagttcagctccaagcaaaggcaccgatcttactatcttactgtgtctcttgtatcatagggtccttaacactaatgatgtcttaaacaatctcttttaactcagtttttcccagtcattgattttgcaactcgggaaggttttgcatgcccaaaagatttggggagcagaggatgggggtgtcctttattaattatattaggattattgagtttcgacccaaactacaagcatgttgggccttatttaaatttaaagtgaattcctcagaccccttcttgggatggttcggggaactacgagtttggaactcttgttcacacaccgaggcgcctgccccagagtttggacactggcattccgggagagcaggccttgcgggagtctggacccgaagggcgagactccacagggccaaggaaagcggcctctgtcctccgttagtcttgggggagcagacgcaagaggaggcaagggcgccgcgagctccccggatgcactggtcccacaggccgtgcccgagtggagcactgcgaatggggccaagaaattttggcctttctcgccggacctggctgcctccgcgggcctctccgcctaccgcgctcccgccgcggcccgactcccgcgggtctccgcgccgaacccacctggctcctatcgcacgggacattcccgacccacccacgccgcgtcactgagcctctgtaccgatacccggcgcctccgccagcagggcctggacgcaccgcctcctttgacctcgggcttcccccgcgctccgctgcttggggcagactggccccgagagggagccaccatctcccctgctccagggtctccagggtccgaacccgtgttgggatctgggttaggattagggtttggagcttggagcctgcctgttaggacccccggccccggcgccgactggagctcgctggaggccacaggaccacggcggatggcctggctgcttcagggcgtaccatgcccgcaggcagatgtttattattaaaaactaccgacgttcatcaaccaggagacccgcaaggcctgcgcactgaatacggcccaaatcctgttcagtggtctttgaaagttaaaagaaagaaacctatcgcccacgtctatgctgaggaacagctttgaatacgagctaagacctgggagagggccaagtgggggtggtggggaacactgctggaggatgtggggctttggcaggggttttacgcaccctccagcacgagggtggggggtcccaggggacgctcaggactctttcagtctttgcggaaggtcccgtcatcacaagagcccgcgcgggaaggaaagttcctgccttggacatggtcagggccgagtcttcaaagtctccaactatccccacacaggcagcacctttgggctttactagtgggatttcttccagaagggggccacgacatggggagagagagctcctcattattttcaaggccaggcttttcctaaacccgattctggcttccaccttctaaatttaaccaatgaagaagctgcttcagccaaccctaaacaggagtgtcagatggggaatcctccctccacagtgccctggcctgcccgcctttgcggctcttttcccgctccgaagtcagcacctgccccgctccagagagggtcaacgaactgggactgattgtcgattatgccataccaaacccaggttgatttcgctccgcaggatccctccttccttcctcccaaaagtgtccccagataaagactggaattatagcaaaacgaataacgagagtccatcttggggaaggaagttactctatcctatgttattttacttcttatctgttctttcattaatttggtataaccctgtttctatgctgggtggataaaatggagggggccggcggaaatgcgctcagcgctaacgacagccacagagccacccttcttactgccccttgactgcggcacgtaagagcagaggcaagcgcttttccaagttggtatattcgagagagtgatacgcattaatcaaaagggaaagactatcccggatattttaatagtaacaggaacaaagatgactcgaaccccatcaaatgaaacgatattttcattcaattgatcgcaaagtgtcttcaattaataacttggcacttatttaaaaggtttaacagaattggaggcgacacattttgtactgacaacacgtcatgagataacatcttttgtttaaaataacttaatacactagaaaaaaatatgtcaaatataaataattttgttttcatggagaacaaatagttacaaagctcaaccgcataccaaagttcagttagaagagcttttcctctttcttgtaaattagaaggtaaaacaaaaacaaaaacaaaaccccacaaacttaggcttgctagcgaattgggatacagactggcttagtccaaggtatcccagtttaaatagctacatgaattgaaacaaacaaccaaaagaaaaaaaaaaacaaaaaagaaacaaacaaacaaaaaaaaaaggttacttgaatagatacacctttgtgagataaaatgcaaatgttgaaagtcagtccacacattatagtcaaaataaactttgttcgtgtgtatcaacatatcttacacaaacctagtgcctgtatctagctgggtgttatttaaggtgaatttgacgggatagagagggggaaataaaccactgtcttcaaataggacttcaggaaaccaacaagaaggaacacaagaaaaggggaatggtgggaactaatactgattgagcgcctactgtgtctggcacagcgaggcgctgtaccttcattatttcatttggtggtcttgttttctttttcctttccccacatattctgcccacctctgtcttcttaggacaatgagtgtaattttttccctttctgacttttttcttttgttccccaggatcaaagacagggcaaatatatatatatatatatatatatatatatatatatatatatggcaaatatatgatatatatatatggatatatatatatcaatttccagatacttttggtattgtttttcatagtgaagatgaaaatggagtgagtgcatgggagataagggggtgggaggagacacaccatcaatggaatacaagtaacatgcaaaccggaatttactgtcagcagtccagacagttttgccataataatttctcagagaaatcattgtctgtggcatttttttgtgttaatattatttttctctctcctcgttttaatattttctcttttcttgctcttccatacatggtgggtttaaactcaaagcatctgattcaacgtaaaaggaggcccgcctctctcatcaaatttctcgtgttaacgtgaacttcgcagaaaaaggtcagtttcaaaacctgtgaacttcccagctgcgcacttttttcatcttagttttttaaataagtatacctcttctttctgctttgccttcttagcgcatttttaaaaacacacatttaagttggtaactccccccagtctctcgagaatcgtctgggttgtttttccatatttttaaatgtataaaatactttaattttaaaatttgtttttgctccttggttctccaatttcagcttctgccaaatagcagttaagaaataagttcccttcctctttttctctcccgtttgtctttcgatttttttgtttgctcattttttcattgttaacaagatttttttttctatgcaagagtccatcgttgcagctttgcggtgagccaaactccgcggttccagcactcccctgtccagtctctctccagactcccccaaacccgctcctacaaaacccaattctaggccctcgagtaggaaaacgggcaggagccacggagcctgcgtgcctcgtgagatccctggtcctgcgtggagtctggctttccgagtccaagatgcgataggggacgagggatggtcagtgaggcgggaagagggccggctcccgaggtctcaaaggggtaacggagaagcagcggggcgcggagggcgtgcaggctgagtgccgcgggacaggcgcgacattggtgctggcgttggcgtcacagacccagggctgcggcgtgctttttggctttcagtctgagatcggcgatgctggagttcttgctggtggtcttggcggctgctgcggccgccactaccgaggcggcggaagccgaatccgcggccagcgtggcgagcggcagtccgaagggcggtgctgggaacatcatgtagggcgcgtgcgcggccaggtgcggatgcaggtggtggtgcgcgtgcgccacagcgctgtccagctgcagctgcgcctgaacctgaaaggacaagggcgtcacgttgcaatgactatcctagggtgacaacagaatagaaacagagcattaacggcgtccagcttggccagagagagagttgggttgtgtttgggcacggggagagggcatttggctgtgcatcttgttccgacagtgaccttctcagcctgttgctgaatctggggttccacatggatactccagtgtcccctgggtctccgaaactcgcttagggtaggcttatttccccggaccccgggaacccccttctgcaggaatgtggccgtctcccctgggaactgggctgaaatggtatattggaaagactgcgggtgaggacccttaccctcttctcttgatgctgccatgcatctcttaccccactaccaaaacaacggagtatcaacaagcaaactacgcaactttttaatgtcattcagggaagttactccatcttcaccacaataggcacaattgtgagtcggtacttctaacctttattgccataacaattacccagggatctcgttaaaattcagattctgattcgcaaggtctaggttggggcctgagattctgaatttctaacaatctcccagacggtgttgttgctgctggcccaaagattaccttttgagtagtaaaggtctccttttaaagtcacccctccctctttcttcagtcattctctcttgtttccaaagccaagcatcaggatgccagagtcctgtggcaccaagagagctaagatctccctaacaccgactctcaattcatggtcacacctatctgtctccccggtccaagtcactggagactgagagctaattcatgacatcacaggcatgccaagcaggggtcctggctgaggatagggaaggtcagacactggttgggaaaggatttcgccctcaggcttggaaagtggggagttcagagcactgggatttcccaggattaaatcattgctgcaggctgtccttttaaaatgtactcttaaaggtttctttatggctaagggattgcaaaggcagggcagccctgggaagaccaagacttctctctttaccagagatgaagctttgtttgcagaaacagatttaaaaacaaaagaacgaaaaaacaaaagtatgagctgggagtacttgtgtttatttttcttctgtcagagttatttaatgtgactaagaggtagaaaatacctagaaaagtatctagagggttgccttagaaataccttaggactaacctctgtattggatttgacaaaatcttaatcaaaaggcttttccttcctctctctctaagggcaatctttagtgtatttttaaaggaccagttactgcccccaggccccttcagggattcccatgaggacagaagggactaggcattcaggcctttaataccgtataggtttttaaagggtagtgggtccactatatcatctcaaaatgattctccaactatgcagacattgacatctgggctcagagacaggtgatgttcaacagttcaagcaaacattcaggtattttcatatttaaaaacagcttgaaactaggctgtacttgcctgctgaaatggcatccttaaagcacctacgttgacataaggtgcgactctacaagcttcaaactggctggcggcccctatgagaacacctgtaaaaagtacaagagaaaaataatgtgaggttaacgtttgtagcatttttcagggttccaaagggtacaagacagaacgaattcctttgaaaatggactattatctttctaaactgcatccaaaatttaaataaatgtaacttactcccaaactttaggactccattaacagcctttgaagattattaagtcaagaactattgtcatttttgaggttcctttttttctgtcattgtaatagtaatattcactacttctctctttgctcagactatcaaatgttccttgtataagaagcatacctttatggagttgattttcttgttttctacatttagctcttcgattttgaaaccaaacctataggttggagggggaaaaaaaataaaacctagatgttatgactaaaaatttttttaaattacaaaagtacaaagagaaaagagtcgtg

SEQ ID NO: 2 - DNA, 4313 bp, Homo Sapiens
gggtttgattttttgagactcggggaggacccctggcagatgtgtgcctagccagaacatttggtaaggactcctccaatgaagaaaaagtggaggaatccagccccagcgagaagaggcttccccaccctgccctagacacaccggacagagggcacactctgaccagagccacgtccagtggccaggaggccagcccagcacctcctcccccaccattcccgtcctgggtgggggggtaattttcctgggagcagctgtgggaactgtcgtttctcatcccagcccagccagcactctgaagcctgcaggggaaggacagcacgtgggatggacactggggaaggagcttcgcaaggccagggtgcaaccttcaggccccaggtggcctggcaggccacgctgcctcggagatgcttgccagactccccaagttcactcagggcctggcagcaacctgctggctgcctctgcgggggtctgggttgctgagcacggtgcagctgcccagggccaatcagccctagggtgtccgtgccaggctgcggcctccccgcctcccccgcactgagggtactcatggcgtgcaaatgctcccgcacccccagagctgccctatcggatgtttccaggaattcacacatttccataaaaatgcattttaaatgatggacaggcgagcctggggtaacaacgggtgtttggtgggtagacaagagcaaatgggaaggagcccgagggaggagggggaagagaagaggaaacagaacttccagttggacattctgacaacagctggaaggaaagtctagaaaagatgaagagagaggaggggagaaaccaactggggctcccacccttgccgttggattcctaattctcgtttcaaatgggccctgctctccggcaaaattagtttaaaggattttaaaacaaagaaaacgagatgaccggtctgggagctcctcaatcagagtagagaagttagaggggggcgggcgacttggttttgaagtcttagctgaacagtcacccctcctctccttggcaaaaaggattcctttagaacctccgaggctcctggatttctcccttcgcaaatggagccgcatactgcattcccccgctctttcggatcgctaagcatgtttcatgagggtcgctgtccccgggtggaatgcggccgtatgcacgcgcctccctgcacacgcacacacacgcacacttacaataagtgtctgcaggaggagtgtcctgcgcgccagctctgcgtttaagacaggaagctgccgggttaccgagtcaaatgggagtgacactattcctctccatcagcaaggaaagcggaccacaaaagtccctttgtatctcggcagctcatttaatattatttatgcattttgtgcaaggaattgtgggatttcgccccacggtaaacaatatggaaatcttaaaaatagcgatcttcctgtgcgtgtccacctacgcgccccggggtgacctggcggggctgtcgccgggtgactcacacccctgaaccgcgaagcgacagggaaagcgcgggcgagcgcaggagacgcggtcgggggtctctccgggttcctgggctcccgcacccggagcgggggacgcggccgctttaaggggaggaggggcggcgggctgctcctgtcacccagcggcggccggagcgtcacgtgggcgcgcggcgccgcggccattggcccgaggcacgtgtccaggagaccggcctgcgacgtcactcgagggggctctgttaaaaataagaacaaaaatccagagtgaaagtgtctcaggttgcgccgagtggcctggaaatttccgagcccgcgcggaggccgaggcggcgagggcggcggacggccggggagcgcgggcggcccagcccggcccggccgggccctggcctcgcgtctctcacccatgcgactcgggccgcggagctctgcggggctcggcgggggcgcggccgcacgccggtggggcgccccggcccgcagcggggcggcggccgcgaggagggggcctccatgtgcgtgcgggcggtggcgggcgcgctgaccgcgggcgcccggcaccctcgagggccggctagggcgtgcgggcggggacggccgggcggcggcggcggccggagccggcccgggcgggcgtgagcgccggggaacgcgctgcctgcatgcgcgcagctctcgccccgggcggcccaggcggcggcgccggagcccgaggcggccggacgcggagaggagcggggagcccgggaggcggcccgcgtccccgccggaccactgcgactgtctagaccccggctgcgcggcgaagtcgaggacttggctctgttgaatctctcatcgtctgggcgagcggggcggctcgtggtgtttctaacccagttcgtggattcaaaggtggctccgcgccgagcgcggccggcgacttgtaggacctcagccctggccgcggccgccgcgcacgccctcggaagactcggcggggtgggggcgcgggggtctccgtgtgcgccgcgggagggccgaaggctgatttggaagggcgtccccggagaaccagtgtgggatttactgtgaacagcatggaggagaatgaccccaagcctggcgaagcagcggcggcggtggagggacagcggcagccggaatccagccccggcggcggctcgggcggcggcggcggtagcagcccgggcgaagcggacaccgggcgccggcgggctctgatgctgcccgcggtcctgcaggcgcccggcaaccaccagcacccgcaccgcatcaccaacttcttcatcgacaacatcctgcggcccgagttcggccggcgaaaggacgcggggacctgctgtgcgggcgcgggaggaggaaggggcggcggagccggcggcgaaggcggcgcgagcggtgcggagggaggcggcggcgcgggcggctcggagcagctcttgggctcgggctcccgagagccccggcagaacccgccatgtgcgcccggcgcgggcgggccgctcccagccgccggcagcgactctccgggtgacggggaaggcggctccaagacgctctcgctgcacggtggcgccaagaaaggcggcgaccccggcggccccctggacgggtcgctcaaggcccgcggcttgggcggcggcgacctgtcggtgagctcggactcggacagctcgcaagccggcgccaacctgggcgcgcagcccatgctctggccggcgtgggtctactgtacgcgctactcggaccggccttcttcaggtgagcccgcggggaccacgcgtcccggctcgccgcggggaggcccgcggagctggggggcggtgctggcgcgggaacttaccgggaggaaaacatctcgaacctcccccgcgcacacgcacaaagactcacgcgacattgtgtgaagctgacgccggcccgggcagcggccaggagtccagcggcaggactgattcgctagggggtacagacttcttaggaccgcagaagggaccttctttctttctctgtctctctctctccttcctctctccctgtctcccctctctgtcttccaccccgccttggcgcatctcttcccagcccctagcccatgtctcccccactgtagttttttttggtgggaacgtggtggctggaagatgggcccggaagtgcacactctcatccccttccctacgatcttccaactcaggccaggccggggacgcatgccccagcccaccccagacttgtcctaccatccggttatcccggctgtgctcggggaagaaaaggcgaggccctttgccgctctgccttctgcccctcgggcctgcgctgaccggtgggactcaggaggatgcacacagggaaggaggaaaataaaggcgccctttccccttggctccactttgtttgccagcgccagcccgcagtggtggggctcagcccccttcctgcacacagcgaggacaagggaggcagccgtccctcccggcacctgccatccccaaatagaaaggaccttctctcagggtttcctgggggctgctgatgggaaagaggcagcattcgcaggggccctgcagagatgctggatatattttttcatagatctgcgattttaaaaaactaagtccatgtccttgtagaaatcatcaactgcattccatgcgggtctgcggctgggaaccgccattagaagtggactgtttgaccccgagctggcagcggatccccgctgcccccaaaccctcaactattttgcgggggtcatttgcccagatc

SEQ ID NO: 3 - DNA, 16197 bp, Homo Sapiens
ccctgcaggtggagggggaaagggcttgggggctggtggaggacgcaggagtatgggggagctgtggaaaagacgtggaggcagagccagcaggccttgctaagggacaggaggtggctctagagagaaaatgagggatcagggatgtctcctggggggtggccagctgggtgactgggagaaactgggaaggcgcagatcagggcagcaggggtagcgggatcgggggtgctctgaagtgaacacgtgaaattgaaggtgctcgtcagccttccagtggaggcatccaggaggcagctggaactggaaggaaatggtgacagtcgtcagcaccgtagaggtggcggtggtggtggttacagctggacccctctgggctggtgggaagtgtggaggtgaaggagcagggagcagggggaggtggagaaggaaacctcaggcttacattttcaccctattcttggctgccatattttcaggaagtccctcttgaggctgggacagagggtggggacagttcagtctccctgagagagtttattctcggaggcctgcggtgggagccagggcgcggcggagagggtttcccacctcttccccagcaagcgggagggaggcgccgggctgaggctgcgctgagctcgagctcgccgcccgggtggacccgccctgttcaagcgcggaggggcggaggcctggttgggttgtagcgtggtgcggagcaggacgccgcctcgtgccatggtcactggagacgcacgcccatcttccctcccgggcccgtcgatcctgccatcttccgctcccggccgctcgtgggtgttcgtcatttagaccttccccgtgcttcatgggacgcaagccctcccccagagttcggttttgcaaaagaggtctggagccctcgctacagatcccctccctgggccacggaggatgagaagggtcaccgagccgagccgtaatcctgcgtaacccctgacctctctccttccctgcccccacggcaccccatcgtcccctccccgtctccgaggtcctccagaaaacagcgcagaattgtgcagatgtttaggagatgtgaagatgctggagatgcttaggaggcgacggttacgcgaggagaattgcttccaggtgcgcttctggaacgcacggagatcccccggcggggaaggggccggggctcgcgtcacttagtgtctgctcaccgatttccctctgaagagggggcccagggcctctgctgagggagcggggaggcggtgcgggcccctcccgggcacacatgggtgtccgctccttcctccccctcccctcccctcgcacggagagcggagagcggagagcggagagcgacagggaggcagccgaagacttgaattttgaaaggggagctggcggcgaatggtgaatgagacagctacttaggaagcgagtccagccagcccgggaggcggtggagacccacgcccggaagtaaccggattaggtctcagaatgtgatcgccccccgggtcccgggggagacgccaaggacgcgcaagcggagggcgcggagacaactgggagtcagagtctccatcacttgggctgggaacgcctctgggcgttccgacggggtggcggtgggggtcgggggaggcccttgagaaccgtgcggcccggggagagcccacccattgctgagccccgacacaggctttgaagttgctgcagtggctcagcccctcccccgtccggccctgcgcagcgcggtgccgcagagtccaggccgtgcctccgtttcgtcgctcagagcttattgcggcggctcattggaccacgtcggtggggcgatcgcagcctctgatctgtgagctgcaaagagctccgaggttcattcacaaatctgcgcccccagccgtccctccacgcacccgagccacgtcccgggacccggagagcccggggcgttgggcgctgcggaggaggcccggcttctgtcgctcccttcacctcccagcccgcgagggacttgggggaagggggagcaagctttcgctccggaagaaacgttcggaaccaaggagtctgactcctggatccgggtgcctgttggatccaggctcccactcccgggccctccggtcgaaccaagggtcccgacagggcccgaggcacccgcattcctaggagacaggagctcggccagggcgcatcaccgggtcctgctccgagccagaggatgtagtgtagacacccacccccacaccgcccctccacagagctcctctccccttggggcgtcgctgggcacagggcaggtcgctaggaatccccagtaaaaccacgctcgctcagaggttcgcggcttcctcagagggcgctggggaaagagaggggacctgatttcttcgaccctcggaggaaaagtgctcggggcctcctagggacacagccctggaagctcacctcatcttagcttaggccgcggcgaggtggggaggagagttaggagagggggagaggggctctgcgccctgcagaggcctttaattctggaggaaaaagactgggacacacccaagcgagccaaggcccgagccccacgcacctccacccacccggggcggcgcacagccagctcctgccgggcgtgagcactcgatcaagggagcaagtggaatgaaaatccagctgggggggtccccatcgacacaactgtcccgcagtcgagcttctggattcctgggagatgtggagagcctggggccggctcccgctccgcagagcagactggactgccctaggtgcctggaatgcgcctgcaccctgtccctcggacccgcgggagacccgtgttccgcaagtcccccttctcaccctagtctgcccctacctacatcccgccggcgagtgtgccaccgcgaggcgcctccttccccgggaagggagctcccttccgcgcagacccgcattgcccttcttttcgctcggttttgttttccaggggtagtttctgcagaaaggagattctcctccgggccgaagggctaccagcctgcagtcagttcagccccggaccctgggagatgctcaccactctgcggatcttgatccgaaatcctttctggccgcccactgcggagagtgtcctcgtagagaggttttcaatcgaaggagcctgggctccacattttgtcttcaagcctgagctttcagggctgctttgcacgaggtgcagatgaacttgtgtctgcaaacaagacagaaaaccctaagtgccgcccgaccttcctcttttggggaacccgcactttgtcctgggagcgtgcccagcgccttagtaccaaactttctggccggggctggcagagctcagagtcccgcttccccctaggcgcggccccctaacatctgtaacccaaatgttgcgccgcggccaaaattagccctggcagtgcgaacagagaactaaaagcaggcagtgaatgagaacagctcgcatttctccttcctggtagacggggaggtgtaaatcccgaggaattctagggcattcgctccaaacgtgggaaatcttcgcgcgtatcccgtttcttcctcccagcctgtgttaatgctccaaatggtgtcgagttgctcaactttgccgtcatcacagaggctgttgtgtcttaggggattaactgatgtgagacacacaaaaccctgcaactccataacataaactatagcacagcctttctggagagggctggaatatttgagtgagtttccgagaggaaaagaggagttttttagaggagaaacagagcacttctataatgtgccctaactgagaaatcttgttctactgagcttttctttaagtggaaccagaagtgctgggatgagagggaaaggatgggagtgcgtccaaaggtggacagcaggtccccatccctggtgggagtgagactggacggcatcccccggaaaggtggtttgggccttggacaaggctagaggcaggagtccatgatgcagagatgacacagtgcccctccgcgtgtgagtccacgaaggtcactactgaggctttgtgcttgtaaaaggtcgccacgcttcacacaagcttctatactcaacacagggattgattgggtacagggatccccccataccatacatgtaagcatgtatgtcaattaaagatgctcgtgctaaagaaacggccaactttgttgaactcagaggaactgatcaatcatttaactaagtagaggaaatgtttgaatttaattcgcaatttagttgcctttttatataaaactatatatctttatttatatttgatgaatgaaaaaagaaatcagttcatgactttaacttaaacatatgtttttaaaaatatatttcccccagcttagccagtatataaactaattgagttctcctggttaagcatgattcagttgtgatatttaagagcgggagtggttgtttagatattttcttcctcacgtgaaacctagactaatgagttatcatctaacaagctgtaggtagtttggctgggctggatccagatggtttgagttaaatctagattgtacttgcctaaaattctgtaaacctctagctacgcaaatcctatttccaaaaatgctgggaagtcactgtataagagtttaagctacactagcctctctgtgattacttgtagcctttggggaaagaatagaagaaaagaaaatgtcagcctctgtgaggtggggctggtgtttcaggggttccttttgaacatcttgttttctttccaaaggtcaaaaggaaggcagtggatacatactagaatttcttccatctgtgaatggtcgcaaggctggagaaggtggctagtgtacttcaaggctcattatccttttctgtgttttccttcctgttttggtaggcttagccagctcaagccttgggtgccattcttcaaattcctttgccaaactaattttacttatttgactggattatttgagaggtgccactttcttctgggttgctgtgattttgagggggcattttcataagagtccaggcattaggtggtgaaacagtctgtgctcccaaacctgcccctcccagggctcccgggagactccagagtgcaggtctgtctggggagtctcaggggtgggccttgagtggaagtgggcccaccttccacaggagtccaaattttataggaataagaacagcagtaaaacaggacaagagagcaggcagggagctgccaaggaaaggcgattcttgggaaggcaggtcacccagaataaggctcccctggcgctggagagccggaagggggagcgggcacagaggacctggtttaggtgtgggggttactagcacaagtagagccatccttcagattctccttcagaagcagctgctttccagagaaaccaggtgagggacagtttctgcatctttacacgccagctctggagatctgcccacctgcccccagccgcccccctccccgggcgagcctgggctaagtatcaagtcaggacagaagggcggtccccagtcggctcctggcccactctgcttccccaacacctagggagcttgggctcagcacaggcgcttctccagcgatcggggcagaaccaggacgtgcaatgcgactgcccccccctccccgctccccggcagagcttctgtccgcgccaacccacccaccaggctctgcccgcgccacgcgcgcccctggcaggcctggggcggggaaaggcgaagcgctgggcacgcgagggcctgtgcaccccagttcctacgacgctcctggcccttttcaggcccgccgctgccgcatttaaccccctccttccgggggttattctgaagaagtctcagaccctagacccagcctcccagaccccgtccccgagctcgcccggtgggcctgtaggccggtcctccttcgcccagaggagagcgcagacacgcaatgtccgcccgttggccccgcccgccccacccctgctccccgcgccccttcgtttcggcctttgtctacaggccgggtcggaacgtcagccccaggagccgacggtggctctctgctcgccccggggaaggtgttgctctcctatcggccccaattccccgcccggcgcctggggcttggctgcggggctcggctcccagccgagggcgcagggctggccaggcctgctttggctgaggtggagatctcgctctcagggattgttgggcgtctctgccccgcgagtaacgagaccgccgcgagcgaacggctttcattgagtccatttttccaagtgtcaccacgtcaagctagagaagcgaggcgagtggaggggacgcagaggggccggaaaagtcacccttctctggcctcgttttcagataaaaacgggagcctggctcggcatccgggcgcccggtgtctccggggcgcttcactgaggttttgtttgcaaaactagcgtccgtgtcctgaagcgcacggcgtctggaagctgctttcctgctcgccctctccgaggcccctctttgtgcagcgagcctgagaaatatggaggaccgtcctccgcaagcgggtggtcgcgggcgctctccgattcctgggtgaagctagagggaaaacggggttcccgggtcagtgccccactttccatcccgggagaaactaatgtccggagagggctcgtctgatccgcacagaaaggccgaccttgagggcggcggtcgttcgggggagaaagcggaggctctgggctcgcgggagcgcggcagccggggctggcatcgcagaggagagacacgccactgccccgcatccccagaaagcgcgaggcgccgtcccagctggggcaggcggccgaggccggtcctcatgcgcgcttctcgaagcttcctgaaacaccttgcggagttccgtgtgtatagaactcagcaccgcccagcccctgggcagctcaactttctgcagccccatcccagcttccccggccctttgaacggtgacccctcttaccgctttcccagagttgtttcgtgtttgggtccactctggggggcccggtaccctctagttatctctctcttcatttatttctctttctcctattcctccctccacctctcaccttcctcctttccaggagagcgacccacgaattcctcttcctccagccaaccccagggcccagcccgcagaccctgcgagggcaggtctctgcccaccggcccgccaggcgccctggaggtgacgctctgcttcccagagtctctgttgcagccacgaattggggtctgggtcgcaggaaagcacagggctgaagcccagcgtcttggggccatttatactgaggcagtcagaggcaaagagtcccaagaattcagaaaatacttctcaggaagccgcctaattggctttcatgggacaggtggagccattaacctgggatggttttgcaggaaccaaagagctcagggcccctcctccccccaatatcatgttcaggagacccagagtcgttggaccctccctttcctgactggtgatcatcagagtttccagagttgcagaaaattctccccccaaaaaaccaagtaagcgtcaataagacttcccacaaactctcaccagccctattttcccggggggcaggtagactgtggggtttgattttttgagactcggggaggacccctggcagatgtgtgcctagccagaacatttggtaaggactcctccaatgaagaaaaagtggaggaatccagccccagcgagaagaggcttccccaccctgccctagacacaccggacagagggcacactctgaccagagccacgtccagtggccaggaggccagcccagcacctcctcccccaccattcccgtcctgggtgggggggtaattttcctgggagcagctgtgggaactgtcgtttctcatcccagcccagccagcactctgaagcctgcaggggaaggacagcacgtgggatggacactggggaaggagcttcgcaaggccagggtgcaaccttcaggccccaggtggcctggcaggccacgctgcctcggagatgcttgccagactccccaagttcactcagggcctggcagcaacctgctggctgcctctgcgggggtctgggttgctgagcacggtgcagctgcccagggccaatcagccctagggtgtccgtgccaggctgcggcctccccgcctcccccgcactgagggtactcatggcgtgcaaatgctcccgcacccccagagctgccctatcggatgtttccaggaattcacacatttccataaaaatgcattttaaatgatggacaggcgagcctggggtaacaacgggtgtttggtgggtagacaagagcaaatgggaaggagcccgagggaggagggggaagagaagaggaaacagaacttccagttggacattctgacaacagctggaaggaaagtctagaaaagatgaagagagaggaggggagaaaccaactggggctcccacccttgccgttggattcctaattctcgtttcaaatgggccctgctctccggcaaaattagtttaaaggattttaaaacaaagaaaacgagatgaccggtctgggagctcctcaatcagagtagagaagttagaggggggcgggcgacttggttttgaagtcttagctgaacagtcacccctcctctccttggcaaaaaggattcctttagaacctccgaggctcctggatttctcccttcgcaaatggagccgcatactgcattcccccgctctttcggatcgctaagcatgtttcatgagggtcgctgtccccgggtggaatgcggccgtatgcacgcgcctccctgcacacgcacacacacgcacacttacaataagtgtctgcaggaggagtgtcctgcgcgccagctctgcgtttaagacaggaagctgccgggttaccgagtcaaatgggagtgacactattcctctccatcagcaaggaaagcggaccacaaaagtccctttgtatctcggcagctcatttaatattatttatgcattttgtgcaaggaattgtgggatttcgccccacggtaaacaatatggaaatcttaaaaatagcgatcttcctgtgcgtgtccacctacgcgccccggggtgacctggcggggctgtcgccgggtgactcacacccctgaaccgcgaagcgacagggaaagcgcgggcgagcgcaggagacgcggtcgggggtctctccgggttcctgggctcccgcacccggagcgggggacgcggccgctttaaggggaggaggggcggcgggctgctcctgtcacccagcggcggccggagcgtcacgtgggcgcgcggcgccgcggccattggcccgaggcacgtgtccaggagaccggcctgcgacgtcactcgagggggctctgttaaaaataagaacaaaaatccagagtgaaagtgtctcaggttgcgccgagtggcctggaaatttccgagcccgcgcggaggccgaggcggcgagggcggcggacggccggggagcgcgggcggcccagcccggcccggccgggccctggcctcgcgtctctcacccatgcgactcgggccgcggagctctgcggggctcggcgggggcgcggccgcacgccggtggggcgccccggcccgcagcggggcggcggccgcgaggagggggcctccatgtgcgtgcgggcggtggcgggcgcgctgaccgcgggcgcccggcaccctcgagggccggctagggcgtgcgggcggggacggccgggcggcggcggcggccggagccggcccgggcgggcgtgagcgccggggaacgcgctgcctgcatgcgcgcagctctcgccccgggcggcccaggcggcggcgccggagcccgaggcggccggacgcggagaggagcggggagcccgggaggcggcccgcgtccccgccggaccactgcgactgtctagaccccggctgcgcggcgaagtcgaggacttggctctgttgaatctctcatcgtctgggcgagcggggcggctcgtggtgtttctaacccagttcgtggattcaaaggtggctccgcgccgagcgcggccggcgacttgtaggacctcagccctggccgcggccgccgcgcacgccctcggaagactcggcggggtgggggcgcgggggtctccgtgtgcgccgcgggagggccgaaggctgatttggaagggcgtccccggagaaccagtgtgggatttactgtgaacagcatggaggagaatgaccccaagcctggcgaagcagcggcggcggtggagggacagcggcagccggaatccagccccggcggcggctcgggcggcggcggcggtagcagcccgggcgaagcggacaccgggcgccggcgggctctgatgctgcccgcggtcctgcaggcgcccggcaaccaccagcacccgcaccgcatcaccaacttcttcatcgacaacatcctgcggcccgagttcggccggcgaaaggacgcggggacctgctgtgcgggcgcgggaggaggaaggggcggcggagccggcggcgaaggcggcgcgagcggtgcggagggaggcggcggcgcgggcggctcggagcagctcttgggctcgggctcccgagagccccggcagaacccgccatgtgcgcccggcgcgggcgggccgctcccagccgccggcagcgactctccgggtgacggggaaggcggctccaagacgctctcgctgcacggtggcgccaagaaaggcggcgaccccggcggccccctggacgggtcgctcaaggcccgcggcttgggcggcggcgacctgtcggtgagctcggactcggacagctcgcaagccggcgccaacctgggcgcgcagcccatgctctggccggcgtgggtctactgtacgcgctactcggaccggccttcttcaggtgagcccgcggggaccacgcgtcccggctcgccgcggggaggcccgcggagctggggggcggtgctggcgcgggaacttaccgggaggaaaacatctcgaacctcccccgcgcacacgcacaaagactcacgcgacattgtgtgaagctgacgccggcccgggcagcggccaggagtccagcggcaggactgattcgctagggggtacagacttcttaggaccgcagaagggaccttctttctttctctgtctctctctctccttcctctctccctgtctcccctctctgtcttccaccccgccttggcgcatctcttcccagcccctagcccatgtctcccccactgtagttttttttggtgggaacgtggtggctggaagatgggcccggaagtgcacactctcatccccttccctacgatcttccaactcaggccaggccggggacgcatgccccagcccaccccagacttgtcctaccatccggttatcccggctgtgctcggggaagaaaaggcgaggccctttgccgctctgccttctgcccctcgggcctgcgctgaccggtgggactcaggaggatgcacacagggaaggaggaaaataaaggcgccctttccccttggctccactttgtttgccagcgccagcccgcagtggtggggctcagcccccttcctgcacacagcgaggacaagggaggcagccgtccctcccggcacctgccatccccaaatagaaaggaccttctctcagggtttcctgggggctgctgatgggaaagaggcagcattcgcaggggccctgcagagatgctggatatattttttcatagatctgcgattttaaaaaactaagtccatgtccttgtagaaatcatcaactgcattccatgcgggtctgcggctgggaaccgccattagaagtggactgtttgaccccgagctggcagcggatccccgctgcccccaaaccctcaactattttgcgggggtcatttgcccagatcacagcaggagtgagccaacccttgggccgccatcccgcagaactatgcgtgcatatttctgatgaaattcagatttctcagctagatctgaaatttgctccattgttctcgtcttcctcctttgttaatatttaattaacatacaggctcacaatgccgggcgaggagactcggccgggctttgtgcggcgcgggagttcgctgagccagcccccaacggcccgggagctgggcagcaccgcccggcccggcctggcccggcccagctcagcccagcccaagtcgcctatcttcatgggctttaaaatatccctgcaagataatgtttctgttctttggtttctccgaaagaaaggggagagagagttttcttggggaggtttgattctgtttctgagactcctaagtatttgtcttttgaaagaaaatcaagaaaaaaacctaaaaattattattttagggaaatttattgccataaaatggtgtccttttgcgggctgcttcatgagtgcattaacaagagccccaggaccagaagagtttgggggtagagttctggggaagggagtggttggaaacccagacagagatgggctctgggagcaggaggctggggcctctcctggagccttgtgccccatcccccaccaccgttccggagggccaaccccatctccaaatctgcacctacccctaccaaagccaggcccactggcctggagccctgggcgtgagcaagacaggcactgagtgtgtacgtgtgcatggggtgggtgcccaagtatagggtgtgtgctctcatgggtggtgagcctgcctatgggttgctttaaaagctgcccttggtgctcctgaggtggtgcccacagatcctctttctccaggcctgtctcctggagagagcacaagactcacttggccatgagggagtgcttggcattcaccctgggcctccagttcgtcccccacctcttgctgggcacagccccagcaccctagttgactctcctgacctgggcagggtgcagtcccagggcctccaaggagatccacattcctcttctcctcagtgtgcccggcagctctccggccctgaagggtggggggcccccagccttctccagccacagggacctgtgatgaagctggggccagatgctccctaaagccgattcatacaccgcacaaattgaaacccagaggcgaggtcaccactccctgccagtggccttgcccccttcttcccccacagggaacgccagggggttgagcctcttatcaccaaaaagaaactgatgacacttccctccttctgctctcctccctctgccctttccccatggatagcaggtcctagaagccttacagcgaccctgtccaaaacctggggcaggtccacagggagaaggccaggtcaggttcataagtctgaatcccagttgggaggcacagtggggagggtcagaagtggacctggacaaggtcagctgggctaccctgctgcccacagtgaagcagtcccatgtctggggaaagggtggtgcagtcaacacccttgtagagccaggtcccctctcctggacaggaaacctgggagatttccagtgggtgaaggactcacccactgtgagtagctcagtgcccctccccaccaaggagggaagtacatgcactgactcttccttaaaggaatgaacttgggtccacagagcccttggctgggagtcatagaggagcttgggtggaggcagacactctgggcccctcctgtcctcagggcccacctgcccctgattcccacagcccttggcaccctgtggggtatccccatgagggtccccatcatagccctcagggcgcctcctgcttctgtgaccgccctgcagcctttcccagtcttctctcctcccctctctctcagcacctaccgccatccctgttcctgaacagagagcctcagaaaggactaggaaaaaccaggccagaaagtgtggggagttttgcttacatccaggagttttacttttatctagagacccctcatttgtggtcagcctgcccactaggcctgccccatagtcccacttacatcacacgcagtcctccctcaccaagtggtggaggtccgcgttgagcccatgcccagtccgaagtctagccccacatctggcggttcagcctcgagtggccttgggcgagtcacttccctcttggggccccagaatccttacccggtgcctattgggtggcacacctctgggtaacctgacttctctctgtccaccgtatctacccaggtcccaggtctcgaaaaccaaagaagaagaacccgaacaaagaggacaagcggccgcgcacggcctttaccgccgagcagctgcagaggctcaaggccgagttccagaccaacaggtacctgacggagcagcggcgccagagcctggcgcaggagctgagcctcaacgagtcacagatcaagatttggttccagaacaagcgcgccaagatcaagaaggccacgggcaacaagaacacgctggccgtgcacctcatggcacagggcttgtacaaccactccaccacagccaaggagggcaagtcggacagcgagtagggcggggggcatggaggccaggtctcagtccgcgctaaacaatgcaataatttaaaatcataaagggccagtgtataaagattataccagcattaatagtgaaaatattgtgtattagctaaggttctgaaatattctatgtatatatcatttacaggtggtataaaatccaaaatatctgactataaaatatttttttgagttttttgtgtttatgagattatgctaattttatgggtttttttcttttttgcgaagggggctgcttagggtttcacctttttttaatcccctaagctccattatatgacattggacacttttttattattccaaaagaagaaaaaattaaaacaacttgctgaagtccaaagattttttattgctgcatttcacacaactgtgaaccgaataaatagctcctatttggtctatgacttctgccactttgtttgtgttggcttggtgaggacagcaggaggggcccacacctcaagcctggaccagccacctcaaggccttggggagcttaggggacctggtgggagagaggggacttccagggtccttgggccagttctgggatttggccctgggaagcagcccagcgtaccccaggcctgctctgggaagtcggctccatgctcaccagcagccgcccaggcccgcagcctcacccggctccctctcctcaccctcctgcacctaactccctcctccttctcctttttcctcctcttcctccttcctccttcctcctgctcctcctttcttcttctttttcttctcctcctcctccttccttcctcctcctccttctctttcctcctcctcctcaccaagggcccaaccgtgtgcatacatcgtctgcgtctgtggtctgtgtcgctgtccccagtcccaccgcagtcctgccgcaggcctaaccctcctgccctgggcactgcctccatgcagaagcgcttcgaggttctggggctaaaggcctggggtgtgtggcctaaagcccaagagcggtggggcgaccctccttttggcttggccccaggaatttcctgtgactccaccagccatcatgggtgccagccagggtcccagaaatgaggccatggctcactgtttctgggcgggcagaaggctctgtagagggagatggcatcatctatcttcctttcctttttcttttcttccctatttttttctttttttcctttatttttttcttttcttggagtggctgcttctgctatagagaacattcttccaagataaatatgtgtgtttacacatatgtctgcatgcatgtgaacacacacacacacacacacacaccaggcgtgtttgagtccacagttctgaaacatgtggctaccttgtctttcaaaagaactcagaatcctccaggatctagaagaaggaagaaagtgtgtaaataatcatttcttatcatcactttttgtcttttcttgttttttaaaatatacattttatttttgaaggtgtggtacagtgtaaattaaatatattcaatatatttcccaccaagtacctatatatgtatataaacaaacacattatctatatataacgccac

SEQ ID NO: 4 - DNA, 2609 bp, Homo Sapiens
cgtagattagagatgactatagattttccccagcgcggatcaaagggactgaattgaacgttttagcttaatgatccaacccctgtcacacccacagggacgcgaattccgattttataagcaggtgtgcacgtgcactcagatactcacacaaagccgcgtggagggacgaaaagatcaaccacccgatcgacgaggataggtttgatcttttcgattacctcagtgtgccagtgtatattcccggctgggcctagcgccctaagaaacttcggaactttagctgttaatttttgttttcttactatgacttcccaaagacattctatctgcctaccggggcgaagagaaatgggaccaggcgtcagggcggtgggaccctgtccagggtcctgactccgcgccagggctccagaccagtcggcctccgaaggccctttcacccaccccacacaagaggaaacaaagacttctcagctcaaggcctcagggctgcctcctgactccggtcagtttgcaggaagaggaaacaacaaaacaaaggaaccgccaatccgccgggtattatatccctcagctccaacctccgacttcggaccgccagggtcaccttctctacgctgaccccgcttttcttaaatgaaaacacgccaacaaaagcatacttcggatacaaaatccaagtacgcatcttttttggggaggttagagctgaggtgtacttcggaagatgagaattttgttttcatgaattgggtaacacccaggcattgttaggtaccccgacagaccctctagatatccttctcttcctccttcacactttcttcctatcaaaatagttattgttttgaaattcactagaacaacgacgttctaaaaacaaaggcgcagcaagcatccctttcttcgctgccgcgggctgaaccacggacgctcgcgggtcgcccagccccgacggcccgcagggggcgcgcgccgcagccgcagcacagcccggctacccccagaaagggagccgaatggagggaagcagggagcgcggagggctcgaggcttgcagataaggagaggcgcatcctgggatttgggtcctctgctgctacaacacaaccgtgctattgttggcactgtccgacccaagtgtcggtggtaagcggcgatgtcggggctgggtctctagcaaccgctgtgccctgggtcaggctggtcgccccagctaccgggatccctctccggatgcttccagggcgataggtgctgcacccattgacgggatagccgcatacctccgaacaggtagtggagttcgtctctggcaggcatcctagctgcgctgacaccaaggtcgccacacaatagccatcagccccccttaagccccagaagtaggtttcccctgccccagccatagcggttccagtcgtcgccacgagcccacttcttatccccaagcgcacctccctctcctcacccgggtttatgcccgttacacagagagaactacacagggggaactatggtcctacaccctcgaggggacagacaccggccgtgagacaggcaccacgcagagccttctggtgactgtccgcaggagcgagaccttttttggttttgcagtcggctaggtgtgtgtgtgtgagggtccccagttgactaccgggatgcactgccacttttcggcctggcgggctctgggactccttggtctccgtaggaggccatcctaggccttcggaggaggcgctcccagctggcggcgcccctcgccccgggctcagaggcggacacggtcggtcgcgccctgctggccctttgttcgcgccgcagcgggctgggagcagctgcgcgacaccagacccacagcgccaagacgcgaagcgcgaggaaaccgctgcgcttgactccttctcccccaactcttggacccaggaacgcttccagctcctgcgtcccacacgcccagtcctggcttctctcccggcccagaagtcttcaaggattgaagggctctgcctagggccccgcactcctgcctgactcccatggcccagaaaagcagggggacatttgaaatgtcaccccgggataaatattaacaaaaaagcaaatggacttgtgcaggggccagttatcaatcaaccaggccgcaaggccactcagggcaaacacagcccaggttgggctgggcgagactttctcatggccgccccccgaccgaacccctccccccttggctcaggtccttcttaggtcgttctggggcaaacaccggacgggaagggggcgccgccaactcccccgcggggtttggaggtttcctcgcctccaagtcccgcagggcagggtcggagtgcccaacacccacccccgcccgaacctcgggcctgcgcgccgccttcccctgggtccgcggtgctgcgcttgctgttgggtgtgtgtcgctgtctctttccgagccggcagctcctgctgtgtggccgaagccctctggaatctttaattggaaactaatcttggtcttgatagacgcccacgtcagaggcgcgccacccacccacaccccccgcctcatcccggaggagacgcggcgagaaccctgtcgc

SEQ ID NO: 5 - DNA, 11667 bp, Homo Sapiens
cgggacccgtgggcgccaatcaatgctatggtggcggagagtaaaggggacgaacacagcctggggccatccccggagtcccccagcgtccgcctgtggccgtgcctggttggccgcggatcccggcgggcgccgcaaagcggcgggattgccagcgcagagctccggctctctgccttgtccctgggtccgagcaccggagcctctggtgtctgcggggagaagtctcggattgagaaatacgggagggtctcgtcagtggctgcaggtgcggcagccactctggggacccagtgagaatggggtcgcctggctctgcgcgaacccctcacgtgggtgcagctcctgagccgccgagggaggcggtggtaatgtcgcccagtgccagcagagggcagcctcgaggccgtgagcccgaacggcgacctcgccaaaccgcgggctcccttccagctcccggattctgcggggtagaggcggccttggagcccagagaccagcgacctcagtctgcaggagcccggcgcagaggcccaagggcacccccgggatgtggtcaagctacagcccttaggcagcctcactctgcgacggcaagggcttagagggtggagggggaccagatgccttaggaggggttagaaagccaacgcacacagggaatttgttttcaagcaacagattccaagcatgtggaaaccttttaaatgatgctggtgagagctatgatagttttcgcattcttggcgagggaagtcggaggctagtagcgggatggctcctgggcgcgcgggagacagagggaccagcgatccttgctgggggcagagggactgtggaccaaggatccagacacacttaattggggattccagtcccgactgtggccactcaactggcgttgaacttgagcaactcactataccgctctagcctccgactaatcacttctgtaaaatgggcattgtgggctccgagtctcgtaagggcgctagggattcgagacagcaggaatttcctcactgctccagcaaccctgggttcctgggtttgtaggtgggctgaaaggatcctttcatcgcacgtttgcctggcgtggctgctttagagaccgaggcccgccaggctttgcaacccagttgcgcgtggtcagccatggccttggaaccgccgggatcgcctcagcggcactccggccagctcccgacttcccgccctgggcactagggtcacccagctccagaagatagtgcttatcagcacaggaattgagaccccacacccggcgcagtgggtctcccaggaagcctggcgaagagccaaggcccgccggacctgaggtcgcctggggcgctaaacagacccatctatgagcacattcggccaacacacccaagttcaacagtgggcgggatctttggctggcctcgatctctctcacagtgtgtgcaaacgggtacccattagattctttttgcttgtttcctcatctgtaaactttgaaaggagagtatttcccggggtgattctgaaaatcatgccagaactgaaaaggcctgtgtaaatgcgaggtgtaatgatgacttattagaagcagccagccctgtacgtgcctgtaggatagatgctagaacgttctacaaatgtgtgcacatacacatcacgtatgtgtgaaggtgcacacctgtccctagacacttgacaaatgcacacacgtatgcgtgtacctctggctgagagcaaaggggctaatacagggctcatacaggacatcatcctgcagtcttgtgcgtgcacagtacacacgcgagcacgactgtctgtgcctggagacaccgtatggcaccggaggagctgctcagccgtcggtgccacctgggaagtagggtttcgggtgctgctctggagcgcgggaaagtccggatccgggtctgctggtcagcgcttcggcgccgccgggctcctttatctctcatccgccggccggtgagcggctccagtttcaagacccccgatcctgccggtgaggggagggagcgagaaggagtgcgctccgggcccaagaggctgcagggccccactccccaggtctgtcgtccttcctttatctcctcaggtcacacccccacttaaaataaagaagggtgcctgactcattcaggtcagggaagtcggcccccgggagcctcctgccctcacaagccatggtgaagggaggcgaagccagggtttgccacggcctgggagaaaatgcggctgcagggtctctttgggctgcagcgctggcccggggccccaagactgttaaggtgtgtgtgggaggcgcgcagtgtggctaccgaagagccctccatcaccgcaccggcaggccgccgggctcgtcctcctctcatgcttccaagggttctgagctgcgcagcactcgcatcacccagaagtgtctgtggagaaaacgacctcaggtgtaagcccaaggctgctgcctgcaaaggcaatgccgtggagactgggttccacagcgacctgggtttttaagtcaccgaaagctacagggcagggtcacaaacactctcccgccttctctgcagaaccgtgcggctgacaggagagcgttgcgcagaaaattcgaggccgggcgctggaggagtctccgcggcccggagaaggcacagaggcgcccctgagaggcgcagctggaacaggcgatgcacgggttcggatctggccggccaaccgagcccagcggtcgcgaaaaccagagtcgccacagaggttgagcacgattccatttctggggatcgggtccggtggggagccactgtccctaacgcctccagagactttaagtaggacaatataatgctggtaggagcaaggtgcaaggaatttagcggcagaagcctttcgggggggtggggaaaggcagatccgtgcctcgtctgagcctgggggtggggacagcccctggccttgtagcccctgttccggggatcagctgggcccacctaacccccctacatggggttgaaaggggccaatgggacggccccgccgcccctccctcgggattccacagggccctgacccggcctttatctctgcacggtcagcagtcgcgggggcttcaggaaggaggacaaaggcccggtgtcaccgcgggcgggggctcggtttcctggctctctgctcacactcacagcctttagcggttgttgggggaagatttttaaaaatatgtgtcgaatttcctttttttctcttctagaaacaaacaaacaaaaaaggcaaaaggcgaattccccttcactcctcgtccatagagattaaagtttcctgggatcctgccctttttttttctttgactgcctagaaatacttgttctcccttgtacatagaggaaaatgcggggaaaggttttttaaaactcggtttcatactattattattactaaggacaaccgggcaggctgaggtcccaacgtggatgatccgagttggcctcgcgccggggctctgcagccactgccctgtgcgctcagcacctctgggggcgatcagggcccctgcgcttccgcccgccgcccggcagtcgagagcaccctgtgcccagactggccgactcattctcccccgaattttgtttagagctggcaagggggacttagctcgcgccccaagacctgggcttgcagcgccgccaacaggcccggggacacgaggcgctccaggccggggtcttcccggctgctggcccctctcgctccccacccgctggcggcgcctcggtcgcccgcaattgacccaacccgcttcctgcgtttgcccctcaggtttcccgtttctccacaaaggcctaggggagcctcgcccacaggctgaccctgcaacctctggcccggtggctaccttctgcttttctgaaaaagaaaaggaaaaaaaaaaaaaaaaagaaaaaatcaaccagtcgagggaggcgcgtgaggactggaggcgccagccggacagcctatgagtaggtccctgggtcggtgccgcttcgcgggtcagcacggcctttctcagagaaaacctcctaaacgtgtgaagaccgctttgggggaagcgagagggaggttggaggagccccgggcggggtctcagcgcccaccagctgtgccttcagggcttgggtgttcgctgcaacggcaaccgcgtgagcctcactcccacggccaaggggctagggcagggtggatgcaatcgcgtgcgcctggccccggaaggtgctcgcagggggtgcctgtggccagctgggttaggaagtcaaatcctagaaagttattactaaaggttttttttcccccttcctttcttctttccttttttgcttccttccttccttttttttttttaataggggaagggaaaaaacattattgaacatctactataaactgggcacttaaaaattagaaaatttaattattttaataattctgatacacgcacgtatttctgttttatagaggagaacactgaaatttgaatgggtattttgcccaaggttacatagctgttaaagaggacaccaggctccaacccagttcttcctgactaaacaggccaggtctttgcctcatgctgcacaggccacctccgcgaagtgacaagatcccagaacatgcttttcacagctcagcaccaagcctgtgcggcaaacagcctgtgcagcagcagtttctgtggtacagaaacaaacaaaaaaggcaaaaggcacagctcagactgtgcttccttcttctcctgggaggctctcccttactactcctgggaggctctcccttactacgcagctcccttgagcatcagtggcagctaggctcagctgctgtcaagtctcctttaaaaatttcggaggccatttgcctcacagatacctggaagacctctggttggtctcttctcttcttaggacctgagattcctcaaatgtaatgtaattttgggttcctagacccaagacactctttttatgacagctgggagtcctctctccagaaagtggaaagttgctaagactgctcggttttaagaatccttaattcctggttgtcccgaaggcatggggctcttagtcccatcctttccccatcttttctatgggtcccacagtctactccaagaagtaggggctgccacacagtggtggtaggcctgacgggcagggccccagggtcaccaccaggttagatctgcagaatggggacactgcagggatgctgagcctgatcaagatgtttcctggccttagacccccacccagaagccaggagtctggtccggtgatcctgtccatgaagcctggcccagaccttcataaagggaagagctcagttgagagcaaactatcttgggccagactcagattcctggcccatattctgcttgtctggctgcatgaagcctgggtctggccccaatttccacccgccgccagggggctccttgcagccccatctccagccttggcttccaggaaaccctggcaaaggctggatgcacctaacctcggatgaaaggtccagggcctcttgctcatctaagcctcagatctggagcagtgaactatttactgatgcaagctggaaacaggacgtcaataccctctggagtagggtacaggaccatctccagaactccgtaggtccctttcccacagtcccttggtgcatgtacttcaatggcgaagatttctccaggtccctcttgagaacaggacacctccccctgctttctccaaaccaacccacttactctgactgggcaattttagtgaggtggaggtgactctaagaatttctgtgtaaatgtgaggagagggctcatactgtcagcagtgtgtgagatgtttacacccaccacacacataatcttcatgcaagaacgactcattacccaggacattcagaaattggctgggtatttgtattaaactctatttacattgtatttacacactggccaatatatatacacacactcacatggcccagccatctaggccagactgtatatgctaaggcataattgctatataactcaaatacttttgccctcaggaattgcttaaatcagcttctataatagaaactcaaaagtgcgaagagggagagaagagaaaggaaatgagagatgaagaaagcctgccattgaactaaactttcggccctggggaaaatgaccaaacttaagagctttggagaaatcccgagggctggagttagacagatttttcctgtgggcatccatggggaagtggggtatgatttggctcccttctctcacctccttcaatccctgtgtagacccaggaaaccctcactgattgcttcattttgtcttccttacttggtgaggctgctgctctcttcccagccttgggtgccttttgcctttcagttctcggggaggagacaggctggtccttcattgctggccccggccaacttctccgtctcactggaggtgggctgctacctgcccaccagggcttggtcctggaagcttccctcccttgctttctctgcttgagacggcagttgggggagtgctgacagccagcttcagaacagggagcctctgcctgatgaagcaaaggctctgtccatattactttatttttcagcaagccaggaatacacacccacaaatgcacacatcaccaatgaggtatgaatgccacacacactgtggtcccagtaagcacttcctaagtgttagccatatgattattacaatgccaaaaatatatgcccactctgcaggcgaatgacagacacacacacctcccaccctgtcaccaatacacaactgacactgtacacacacatttgcagcagatacagactacacaattacttgcccatgggctaaattttcacgctagacaaagcacaaacagacatgcacacagcaggctgctttctgttctgcaactccccttagccagatagtggagcagccgggcaccaaaggcacccagacaccaggcctgaccagcctctagggcttcctgctccagctgagcctccctctccatcctgctcctgccggttcctggtaagctccagtcccagtcttgaggtgatgccccctgagaatccccgtggcttggtctcctgccttgtagcctctcgcaggatgcctgtggggggcggggggcattctgagcagaagccacttgggcccaactatacataacaggataccaccctgaggttcctaccgcctgaccactggagggctgaagtgagaggcctgcaggctcattcccgtggcgcaaaaccgctcctggcccaccagccggcctggacctgccgccacattgctggccaggccccgagatagacggggggggggcagtgagggctgggtgcggggtctagtctcagctgagcagcagcccgcaagaaccggcggatctatcctatcagaagtcggaagtcatctatcccatcagaagagtgaaagccgaccaccccctaatccgcctgggagaaccagactgcaaccaggtcaaacgcctgtgtgcctgcgcggaagcatggatcttaattcgttatttggggatctcagtaaaggaagagagagaaagaaaaaagactaaatttctgcagccgcgaagtatgttcaatccttactgaaacctccaaatctcctacccatatctcaaggtaaatgaattctgactattaaaaataataaagcagtcacttctcacatttgtacagaggttagcagtttgcagcgcatttccacaaccagaattttatctattaatgtttggaacaaactcttggctgttatagttgactcaaaatcgaaaagtttgagaacaataataaaaacacagccagcccatacacctagtacaactgtgtatgaggtagaatacaaacttgaataggtacaaaaataaaccaagtcctacctacagtcgcgccttgtgaacgaaccacggcagaaaaagtctttctgctgacgttaaacagccgcaacgggccagtgatgacacagaaaacaaacaatcccaggaaaacaaagcagcagcgacagccacaaacgcctgccaccccggttgctctcaatctttgtttaactcattctgaaaaagataaaattttgctgaaaatcttcctcttttaactagctgctgaactaaggagcgacagaaataaagaagtccgtgttaaaagaaggaacacaaaatttattcgggaatcgacaaggcaaaaaacaaaaacaaaacaaaaaaacacaacaaaagtgctctagttctctctagaaaatttggtcccccaaacaacaagctacagtacaaccaggtctatggttttcgggccgggccggctccgggccacaagaccgcctaggtcggccgctgccgggtttcacatttctccttcccaaggtgggagaagcaggaggtttgaaaaacaaaaagcagggaggacgagccgtcccagcagcgtgggccaggcaggcagtgatctccctgcctccaggacttgcatcgaaagatccggggacatttgtttttgatttttttcttcagatagggagagggtgaaacttccccaattggtagacgaaaccatcttttattggatatattctcctaactctgaacacagctttttgaaggcccaacaaaaactctaacgcaaatcttccagaagagaagatgaggaatactaaagcttttgattttcatatttggatttagcaaactccaaggccacaatacttccccggttgcccggtgaattttttttcctttttttttttttaaatactctcttaaaaagttccaccgatctttcaaacaaaacattagccagccgccggggcccggaggtgcccgaagcgacgggactgatagcggaggaaacgcagcctccctccgcgcccgcccgcggtgtaaaccgagtacaggccgtggagccaggctgccccggctcccgctgggtcccaacccccgccccgcctagtgggccccgcggcaagcggcttctgaacagcttcaagagggttcgcggagcaaacacacgtattggtccgtccctttctcgggcagcgcggtcccgccatcagtcctgtccggcgcgtctagccatggactgcacggcagtcgggcggggaacgcggagagcgagcgcaccgacctgtgagagaaggccaagaggtctgcgctgccgacgcccggtcgcacctccgccccgggccctttccgcggtgaatttgggcaggagacgctggggctccggaaagagacgagcccagtagaaagcgcgcagagaggcagcttcaggccaggggagtgcaaggtcacagaggtcagggaggtgagcacaggaggacataaactgaggggacaaagaggagcgacaggagcttaggaaagcgaaaaagcacagagggaccctgggcgctggctccagaggcgggcccagagggtgtgaggtcaggctggcggcggcgtcgtcggctgcgaccggggccggcgtcgcgcgtccctgcatcctcgcatccgtctgcaccggcatgcggtgggctctcagagatcgaggcgcgaatgcagcgcgccggtcttgctgtcgtggtcccagtaagagcaatgcatcatggcgagctcgggctgccgggcacaagcgaactgcaggcccggcgcactggtgggcgcgggcgccgggggcgcggcggtggctgggctggcagggctgagctggcccggcggcggcgcggcggccccgtggtgcggtggggcaggcggcggtgcggcggccgcgtgcagatggtgtgcgtgcggatgcgggtgggggtgcggcggaggcgggggtgcggccggcgggcctcccaggccattgtacgagttcactacgccggggggcagcgccatgctctgcacgcgtgtgtacggcccgtacgaggcggccgggcccgccagccccttgaccacagcggccgcgccagggctaccggggcccgcggctgcagccgcagctgctgcagccgctgcggctgccgccatctggcaggaggcatagggcatgggtgagggaggctgcggtagcggccacgagttgttgaggaagccagactgcaggtacttggggggcgccaggtagccgtagccgtcggccccggcgcccgccacgccgcacccgcctgcggcgcctccggccccgaagagccccttgccgggctggaagtgcgcgggcggcggccggaagggcctcttcatgcggcggcggcgccggtagttgcccttctcgaacatgtcttcgcaggccgggtccagcgtccagtagttgcccttgcgctcgccgccgccctcgcgcggcaccttgatgaagcactcgttgaggctgaggttgtggcggatgctattttgccagcccttcttattcttctcgtagaacgggaacttcgcgatgatgtactggtagatgccggacagcgtgagcctcttctccgcgctctcgcggatcgccatggcgatgagcgccacgtacgagtacgggggcttctgcgccgggtccggcttctccggggctgtcccgccgccacccccaccgcccttgcctgggctcggcggcggcccttctggctccttgactgtgcgaccggtctctggggccagcagggcccccgccgcgtcctcgggctcggggtagctggccatcatgacaaagccggcgcgccgcggccgggccgcctctgctctccgctccaggcgctggcgcggcaaagagttggggcgcacgagtccgcttacggccaagtctcaaacttctggagactgcggatgccgcccgcgcttgcttgctggaggcctgtcgctgctctcccctctccttccccttcccctagggagcggccggcgggagtggagctcagcctctggccatggggagtccgcccaacagagaggggctccggcctcgccgcccctccccgctcaggccagtccccgccttggtgggttttcttttctgcgctcttcccctccccccgccccccggtttcccgaagcacgacccgcgtctctggcggagctgcctcctggagtccctagtgcgccaggagcctcgctctgttctgattcgtatgggctccaccgagttccgcttgcgtcaggcgccttcgcccctatagcggggcggccagccgcgcacgggcgagttcatctccaagtcactttttgtaaacgccccgcacagcctggaccggcctgcccccgcccagcgagcctcaggggcccagccgacagccaggctcacgcgcccttgaaatctgccggtactcgctctgcgggctgggctgggagatgacgaggaccccggtggggtctgcccgcacccggccaaagcccaggaagctcgggccccagcgaggaaaggcgctccaagcctcctcgcggctttcaggtgaaagaaaacgactcctttgctctgccgtttgctgccgtcttgaggctgaacttctagctcggggctggggaggggcgagacggcgagggggctggacggggtagggtggggagagctgctctgaggctttgggaaagtcagcccagaaacgggtgtgactgtacgaagaagcctcggcctggcctgtccctcgcg

SEQ ID NO: 6 - DNA, 1394 bp, Homo Sapiens
cggaaccccggtccgaagccgagacaggagactggatgcgaggccctcccagagctggtttctctcaaacaacttccaaaactcctagatcctaggggtacgccgaaatcccccaaagcagtccaaagaacacaacgagagtcctaacatcccaggtggcggcgcgctggctccctggagcggggcgggacgcggccgcgcggactcacgtgcacaaccgcgcgggacggggccacgcggactcacgtgcacaaccgcgggaccccagcgccagcgggaccccagcgccagcgggaccccagcgccagcgggaccccagcgccagcgggaccccagcgccagcgggaccccagcgccagcgggaccccagcgccagcgggtctgtggcccagtggagcgagtggagcgctggcgacctgagcggagactgcgccctggacgccccagcctagacgtcaagttacagcccgcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcggtattgggcaggaaatggcagggctgaggccgaccctaggagtataagggagccctccatttcctgcccacatttgtcacctccagttttgcaacctatcccagacacacagaaagcaagcaggactggtggggagacggagcttaacaggaatattttccagcagtgagcaggggctgtatgggacgcgggaggagctcagaggaggcgcggagagtgcccg

SEQ ID NO: 7 - DNA, 6357 bp, Homo Sapiens
gtgtggagattgggaaggtgacaaggtgaaggcaattgaaggaagagccgagggggacatggggaaggattttgtttcacccctcctaagttgaaccattgtcctttgaaggccggctcctggagaaattaaagggcccctgtgtgacacagccatgtcatacataaacagaactctgaagcctatcaactcctgaggctaagtaagagggaatgtaggggccaaggcagaagagaaaccaaaacctcagagcgctgagcaaagatgccaatcagagaaagagaaattcatttgcgatgttaattaacaagcggctaattaaaacggcactttgagtgctaatcaatcgccttattaagttacagccatcactggaacaaattgaaacctccccgccccgttttctgcctttggtgcaggcggggccgcgttcccagataccgtgagaggccttggggcgcggaggttgggggcagcctcggtcagctttctcagtctctcccaggtctacagaatacgccactggacaagtgcctaagcagcgacttctggtccagacacaccgcccggggagtaagtagttgcgtcgaagaacaactcattcagcagcagttaacaccgacgtttcctcctagaaagagctcccgcaaagcggggggatgtgacctgtgggcccccagcaggggtaggaggcagttcagcccgagagggggcgctctagggcctggatcctgcatccctatttcctggaacacacccaacgcctcattctgaaaaccctgcttaggccctggccctggtgccgctcagcaaccaggaaagagctggacctgccttcaggcagcaagaacaggactgccagcctcctgtggctctgtctcccgaggctccatgagaaggggatgggggtgcaagaagggaagagtgaggtggtgtgctgggcgtcggggacgaggacgcacgccagccaagacgtgcctcccacccagcccacgcgcgcttccccacccccctggccctccaaaatcggtaagagaattaagatttcgaatccctattttgaggagccttccgcatttcctaattgttaaattcctgcttttcaccaaattcccgggggagaaacatttggcaataagaagggactgtgaatttaaatgctaattgagtgggtcctttttccgcagctccacctgcctggcagcctctgttgaaaccaaacacactcggagcgcccagtgcaacattcttggggtgccgagtagaagcgcagtaaagagagaccctagcggactcctgtctggtttgctttttaccgactcttacagaaaaaaagagaatgccattggaagaagctcttttgcgtggtgggcgatgtgtgggtgggggacttgtggcatggcccacggtgttgtttctgtgcctgcgatgacacacgtatgtcttgagctgtgggctcgccttcctggaggtgcgcccgaccgcatctgctggtgggtctgagcgtgcttggggtgtcccaggagaactgagagaacggctcccacgtgcaaagttccaaagcattaatattttcatcatattatcattattcaatataataatatttgttcggttagcggcactaattaggccacattaaaaccgtagtgtgtccctaatggtgcgtaatgtgctcacactcacatttttctctctgaggatgggcggctgcaggctggtaggggaggagagacaggcaagcggcgggctggattagggcgtgacgccccccaccacgcacacaaacatacacagcccactggatgtctgccgggtgggagccgcaatctccgcgcggtcgatggggccctccgctgcgcactcggccctgcgccgagcaccctgcagcctcctcccgcgacacggcgctttgaactcggcggattgattttgcttcccttcccccttttgtgtgtgtttgcgttcaattggttaggtttttaagatttgggagggctggtgtgaaagaattaaaatactcttaactggagcccctccgccgagaactggaggtcccgcctcctagttcggcgctttcaggaccctcttcccagagggaatttctttcagaaattccagggtgggcttgtaaaagacgcttccgcagagcaggtcccgtcagggtctttttcctgttcctggtgccagcggtcggcccgggcgccccgcagacctcggcgaggtagatgttaagctcggagagtgcccctcccgcaggcgccgtggcgagatcactctgaatatgtaacatatttgtaacgtgcgccgaggtgtgatgtgtgtgctgaaatagggggatgggggaattcgaagccggattgggaaggcgggggggaggcgcacagaactcacaatgtacttcgcaatctaacaatctgaacattcatttattaaaagctgctgcgtgacatttacactgagccaccagtctctgcctctaatccgggcgaaaacgattgtactgccgagttatggctgcagcgtatggggacgctgctgtccgcggccggacagagcccatcagctacaacgcggaaggcctctgcacccccttgggggcgggaggaaagtactgccagtcctgcctgggggccgagggtaacaagcaccgagcctctcgctccacgcagggccagctgcccagctcagcgaagctcttgtgatctggtgcgtgtctctcgctcttccctccccatcaaagaagtaaactttctacctactccccctaatccgatcgtttagagctgctgttttccttttgtcagattcctcctccccgatcagtctgagtacacgatcagaactgctcagagagcaggaagcacattgatttcagcttgttctgtccacagacaggccctgacaaggttgttagaacagccggagaggtctatacaatcacttaattaccaaaactgtcagtcaggcgggacgcggatccgcgtcccgggctgcgctaggcattccagcactgggccgcgcgcgtgattgatcggtgctgatagcaccgcaaaataattacggcgaattttctgatgtgtgattttatcccaagttcatgcttcagagaggtaatcggagaatgagaagggtcagtgccatttcggattacctggaatctgcgagaaagggtaaaatgggggaaggagctccgaggaaaacgggagagatgggggtgcagagagagagggaagaagaaagcgagttatggattgctggagggactgcaagcaattcgtcaaactgtgcaagtgatttccttcagagccagcatatggcagattgattttgtccaacgtcggttttagccacatttaaaatgatccagcggttattactgcgattggcttaggaactgacaggcagttttaggcgcaaggagtatagatcctgtttaccggagatgtgttcgtaactgctgtcaaatacagttaagtaaatatcattagcgaagagctctgttaagagaaatgccaatccaataaatatgcttttcctccccgccctccgcatggctgcctgcgcttcctccagaggttctccttcctgctcctttgctgcttgggtcagacgtcccaggcatggtgctgactcccgccaccttggagccccgagctgagcctcgggcagaagatgacaggccagccgtggggcaaggaggccgcggaaacgcggaacggcttcggggagacggaagcgcccaatgagattcaccctgcagcccgggtccagcccaccttcctcggagattgccgcggccctcgaacccgggcctaggtcttcatgtcccggcggccagaggacgttgcggggaccactggggagctgccctcagtcagctctctgccccacgccggaggtcctggcgcggcttctttcccgaactagactggcgactctgggccaggccccaaggaccgccccggcctctccggctttgcggggagaatctgaggaaccgagtccaagatagccgacctaggctgttttcacccagaccctgcgtccccgacccgctggagtgaatctgacactgccaggttctctctcatggcatggagtgaatgaagagggccatagatccccttacccagcacagtccctcggcaggccctggaaatccacagggagcagaagcacagtattttctgaaccgctccctctccctgggcctgtggccatttgaaggcagagctctgtgcctccaagacagtaggttttcggtcaagtttggagcctggggccccaacacatttacacagggttggcatcaccgtttccttggactaaaggcaggctcctatatcctttttaaaggaacagaaggaaggaaaaggaaaccaacacgggttatgttcagatagtaggcctatggcaattcttcacagccatagagtcctaatccgagtatcttcccagagaggaaaaacccaaaaaacttttaaaagggggaaagctgggtagatcatagcacccattcttcatgcctaggcagaaaaactaacccagagggagcaaaggggtaagaaatatgaagagatcccctctgggagctgaggagcaccctagtttataatttggtcaaaggagaaagtcactggcctcctcctttgatagaggcgtgtcatctatcttcccagggaacatgatggttcacaaatgaagaggctagccctcctgcagcttttttctacagagtgtaaaacacacaccgccttcatcagtgtttgggatgtaaagaaccctgtctatttaaaagagatactgcatttttaaagtcaaacagtaccaatgtatgtggcgaatcaagtaggtaaacaacttacatatggttgctgcacttgaaggaaccatccattctcatgcacagcaaattgaagaaacaatggcactaatgagccttgcaaaatgcaactgtgaataatgaaagacaacactgcattttgcaacagaaagaataaaggtgaaataatcagctagcaaagaggaaaagaaagcgagcaatgattaaatgatcaaaagctggcagagtgaattcaatgtcactgccagacgcagccatctacccacaagtgaaagttaggtttcaagcacagtgtaattatagctggggttgtcagtttgacattaatgcagccagcagaaatttcctaattggcctcagaggagaaagtgaaccagaaaatatattaacattttaaaaaagcatattttgcctaatcctttcactttcgaacaatatttgaagaccaaaatgccccaggcataagaatttaaatgagcaattttgtttttgaaggaaacggccaatgagacagaaaatagactaaagggaaatcattagtggatgagagatactgacaggcttgccttgctgactggctggcctgtcacttgcagtctgtgttctttagttccacgctatgagctaagttgataacatgaaaagacccataaacgtgcagccagaagtcacagcctattatctggaaattcaaatgcaaggggagggggtggcagagaaggcatcggcgaggttgggagggagaggtgtgcatcgagggaggaggaggaggaggaaggggaggagggaaaggaggaggaggaggagaaaagaagccctcattctttggcataaaatcggccatatcagagaacaataataagctattatctgtcataaatgtgctatggactgccaaaaaatgtagtcccgaatcgacaacattgttcgaactgaagatagcaacaaaatgcttaaagttgcggatgtaatttcacatgcgtccgggttgatgtgatatgaccgtatcagggaaacaaagctaagtgcagtcaggatctgttagtacagtggcttttgatggaacagctgaggcacacatcgcccgtggcatggactccggggccgaacgctcacgaccaagacttttgcccttttgaaatgaaatagaaataggggagctgcaggaaaaccgaatcgcgcttagggtcaggagcaagacaggagctttcagcgaagatctgaacattcagaactggaacgggtaattagcagatagccaggaaaaaataaataaataaataaaaaagcctggatggacctctgtaaacaatcattaagaaaaataaaaatgaaccttcttattagcctgccttggaggtagtcagaaacaaacaaccaaagcaagagaggatgaagatttaaataaaataattatgtgcatcattaaaataatcatatatgtttgtacagacacgtatacaccaaggaacgtaatgggggctcctcgcacagtcccaggagatgcagga

SEQ ID NO: 8 - DNA, 9739 bp, Artificial Sequence
  chemically treated genomic DNA (Homo sapiens)
acggaagtatagaaatgtatttaataaatatgtaatagaaggataattaatttaatattaaagtgattagagagttaatgttagggttttatgggtatgttgatattttagaatagagaggattgaaggttagagattttgggtaatttgtatattaagaacgtttttaagttatcggggggaaagatagagttttttttgttttatataaaggggtagatttaaaattatagttttttaaaatttttaaaaatttttgtaagatttgaagagagttttgttttaaattttagtgtatataatataaatatatattattaatatttataataagagattggaattaaagtgtatattattttttaaagttttttaaaattgtttttaattataaaaaggtaaattaaaaattttttttatgttatagtagtttaaaaggtaaataatggaagtagaaaggagatgtatatttgttgaaatttgttatgtattaggtattttatataatttatttttatttgatttttataataattggtaaggtgtgtgttgtaggatattaagaggttatagattttaggaggttaagaaatgatttaaaattttatatttaatagttggtatagtaaagatttgaagttaagaatatttgtttttaaaaattagtcgtatattatgataatatattttaaataaagatgtataaaaagtatttggttttttatagatattatataaaatatgggataatattagtgatatatttaaatttagatattagaaagaagttttttttaatttattttgtaattgtaatttagttgtttattttataggtttttatagtaataattaaatatttttttttttgttttatatgtgtttttaaattgttattaaagatttttggttagtagtttttatattttttggcgttttgttgttaatgttcggtaaatatgggtagttaaggaaaatgtagatggtgttatggaatataaattacgataatatatggtatatatttagttttaataaagtgcgttttttaatttttgggtgtcgttttattatttttttataagattatttatttttagggaaggtttagtttttaaaattagggtaataggtttatatgtgttatatggttatttagatgtatagattatgtgttttaatatttttaagtaagtgtatttagtatatgttttttagtattattatgtgtatttatgaattgttttgtttagatggtaagtttttcgaggataagtgttatgtttgaaattttattttattaattgtgtatggtagagtaataatgttatttattttgtatatagagatatataatttataaattgtttttttaggtaaattttttaattatattgttatattgtgataattgttatattttttagatgaggtttatataggttaagtaattagttagaattataaattaataggtaatgttttttttttgtttataagagaaagttttttttgttttgtaatattttattgttttttttttttaaggtttttattataaaggttaatgtttattggttttttaataaaagtaattattatttagtatttagtatatatatgtaaggtattgttttaagggttttataaatattaatttatttaatttttttaattattttatgagatagatattatttttatttatattttatagataaggaaattgagggatagagagattaatttgtttaaaattagaggattggtaaatagtagtagtgaaatttgaatttagtagtgaagtatagttttatagtgtgtgtttttaaatatatattattttgtttgaataagagtttataaagatttttatttaggatatatgaggagttgtttagtaagaaaagaagtatgtttatttttttttatttgatgtatttgattatattttaaaatttttttttatgttaatagaatttcgcgtattatattaaatttttttgtatcgaaggtatatgtaatggttattttgattgatatgtaaaatatttaaaataaaatatttcgagattcgaggattttttgttttatgaattagatattaaaaaaaaaattgtttaggtattagaagtgttatatatttatatttgtaagttttttttgggatttttagattttttatataagatttttttttgtttgaaatattttagtgtttatttaatgtttgagtttgaaaattatttaattttttttaatttttttatttttagttattttgtttttaggtatagggtagggtaagtatttgtttttaatatgatgtttaatattttttatgtttttattttcgttattttgaaaatatgttagtaaatatttaaatatattgtaaagatttaaaatttggggtgtgtatacgtatattaataggagttatgaattatgtatattgtaggtttatataaattagattttgaagaataagtattatagtaattaagcgggttataataagatattttatgtgaaatgtaaaaattattttgaataaattatttgtaagttaatatttttattagaatgatatatttattatttttgggtttgtgatattgttttaatgtattttggttaattgttttaatggtatattaagaaattattttaggtttttttttttcgttggaatttgtagatttttattttcgtttgaattaaaataatataatattaaaaataattttttattttttaatgtataatattatttttttttgtttagaaatatttaattaaagtaggatataattttagtaattatttttatatagaatgatttttaaattaatagttagttgtatgattaaaaattgggagattgataattaaaataattttaaatgttttgtttatgtgatgaaaatgagtgattttttaatttttttatatatattaaattgattttatagattttttgtattggtgtttaatatagttaagtgtttgaggttatgtaaaataaataattttgagattttattgtaaatgtttgtaatttatgatgtaaattgatttgtgaagaaaaaataggattttatgtcgttgaagttaaagaggtattttttagaaattaaaatagtttcgaaatttgagtattgtattatattaaagtattattatttgaggatttaaaaaagttataaatttggggaaaaatttaaaatagatgtatattagtttagttttaagtaaaggtatcgattttattattttattgtgttttttgtattatagggtttttaatattaatgatgttttaaataattttttttaatttagttttttttagttattgattttgtaattcgggaaggttttgtatgtttaaaagatttggggagtagaggatgggggtgttttttattaattatattaggattattgagtttcgatttaaattataagtatgttgggttttatttaaatttaaagtgaattttttagatttttttttgggatggttcggggaattacgagtttggaatttttgtttatatatcgaggcgtttgttttagagtttggatattggtatttcgggagagtaggttttgcgggagtttggattcgaagggcgagattttatagggttaaggaaagcggtttttgtttttcgttagttttgggggagtagacgtaagaggaggtaagggcgtcgcgagtttttcggatgtattggttttataggtcgtgttcgagtggagtattgcgaatggggttaagaaattttggtttttttcgtcggatttggttgttttcgcgggttttttcgtttatcgcgttttcgtcgcggttcgattttcgcgggttttcgcgtcgaatttatttggtttttatcgtacgggatattttcgatttatttacgtcgcgttattgagtttttgtatcgatattcggcgttttcgttagtagggtttggacgtatcgttttttttgatttcgggtttttttcgcgtttcgttgtttggggtagattggtttcgagagggagttattattttttttgttttagggtttttagggttcgaattcgtgttgggatttgggttaggattagggtttggagtttggagtttgtttgttaggattttcggtttcggcgtcgattggagttcgttggaggttataggattacggcggatggtttggttgttttagggcgtattatgttcgtaggtagatgtttattattaaaaattatcgacgtttattaattaggagattcgtaaggtttgcgtattgaatacggtttaaattttgtttagtggtttttgaaagttaaaagaaagaaatttatcgtttacgtttatgttgaggaatagttttgaatacgagttaagatttgggagagggttaagtgggggtggtggggaatattgttggaggatgtggggttttggtaggggttttacgtattttttagtacgagggtggggggttttaggggacgtttaggatttttttagtttttgcggaaggtttcgttattataagagttcgcgcgggaaggaaagtttttgttttggatatggttagggtcgagtttttaaagtttttaattatttttatataggtagtatttttgggttttattagtgggattttttttagaagggggttacgatatggggagagagagttttttattatttttaaggttaggttttttttaaattcgattttggtttttattttttaaatttaattaatgaagaagttgttttagttaattttaaataggagtgttagatggggaattttttttttatagtgttttggtttgttcgtttttgcggtttttttttcgtttcgaagttagtatttgtttcgttttagagagggttaacgaattgggattgattgtcgattatgttatattaaatttaggttgatttcgtttcgtaggatttttttttttttttttttaaaagtgtttttagataaagattggaattatagtaaaacgaataacgagagtttattttggggaaggaagttattttattttatgttattttattttttatttgtttttttattaatttggtataattttgtttttatgttgggtggataaaatggagggggtcggcggaaatgcgtttagcgttaacgatagttatagagttattttttttattgttttttgattgcggtacgtaagagtagaggtaagcgtttttttaagttggtatattcgagagagtgatacgtattaattaaaagggaaagattatttcggatattttaatagtaataggaataaagatgattcgaattttattaaatgaaacgatatttttatttaattgatcgtaaagtgtttttaattaataatttggtatttatttaaaaggtttaatagaattggaggcgatatattttgtattgataatacgttatgagataatattttttgtttaaaataatttaatatattagaaaaaaatatgttaaatataaataattttgtttttatggagaataaatagttataaagtttaatcgtatattaaagtttagttagaagagtttttttttttttttgtaaattagaaggtaaaataaaaataaaaataaaattttataaatttaggtttgttagcgaattgggatatagattggtttagtttaaggtattttagtttaaatagttatatgaattgaaataaataattaaaagaaaaaaaaaaataaaaaagaaataaataaataaaaaaaaaaggttatttgaatagatatatttttgtgagataaaatgtaaatgttgaaagttagtttatatattatagttaaaataaattttgttcgtgtgtattaatatattttatataaatttagtgtttgtatttagttgggtgttatttaaggtgaatttgacgggatagagagggggaaataaattattgtttttaaataggattttaggaaattaataagaaggaatataagaaaaggggaatggtgggaattaatattgattgagcgtttattgtgtttggtatagcgaggcgttgtatttttattattttatttggtggttttgtttttttttttttttttttatatattttgtttatttttgtttttttaggataatgagtgtaattttttttttttttgattttttttttttgttttttaggattaaagatagggtaaatatatatatatatatatatatatatatatatatatatatatggtaaatatatgatatatatatatggatatatatatattaatttttagatatttttggtattgttttttatagtgaagatgaaaatggagtgagtgtatgggagataagggggtgggaggagatatattattaatggaatataagtaatatgtaaatcggaatttattgttagtagtttagatagttttgttataataattttttagagaaattattgtttgtggtatttttttgtgttaatattattttttttttttttcgttttaatattttttttttttttgtttttttatatatggtgggtttaaatttaaagtatttgatttaacgtaaaaggaggttcgttttttttattaaatttttcgtgttaacgtgaatttcgtagaaaaaggttagttttaaaatttgtgaattttttagttgcgtattttttttattttagttttttaaataagtatattttttttttttgttttgtttttttagcgtatttttaaaaatatatatttaagttggtaattttttttagtttttcgagaatcgtttgggttgtttttttatatttttaaatgtataaaatattttaattttaaaatttgtttttgttttttggttttttaattttagtttttgttaaatagtagttaagaaataagttttttttttttttttttttttcgtttgtttttcgatttttttgtttgtttatttttttattgttaataagatttttttttttatgtaagagtttatcgttgtagttttgcggtgagttaaatttcgcggttttagtatttttttgtttagtttttttttagatttttttaaattcgtttttataaaatttaattttaggttttcgagtaggaaaacgggtaggagttacggagtttgcgtgtttcgtgagatttttggttttgcgtggagtttggtttttcgagtttaagatgcgataggggacgagggatggttagtgaggcgggaagagggtcggttttcgaggttttaaaggggtaacggagaagtagcggggcgcggagggcgtgtaggttgagtgtcgcgggataggcgcgatattggtgttggcgttggcgttatagatttagggttgcggcgtgttttttggtttttagtttgagatcggcgatgttggagtttttgttggtggttttggcggttgttgcggtcgttattatcgaggcggcggaagtcgaattcgcggttagcgtggcgagcggtagttcgaagggcggtgttgggaatattatgtagggcgcgtgcgcggttaggtgcggatgtaggtggtggtgcgcgtgcgttatagcgttgtttagttgtagttgcgtttgaatttgaaaggataagggcgttacgttgtaatgattattttagggtgataatagaatagaaatagagtattaacggcgtttagtttggttagagagagagttgggttgtgtttgggtacggggagagggtatttggttgtgtattttgtttcgatagtgatttttttagtttgttgttgaatttggggttttatatggatattttagtgttttttgggttttcgaaattcgtttagggtaggtttattttttcggatttcgggaatttttttttgtaggaatgtggtcgttttttttgggaattgggttgaaatggtatattggaaagattgcgggtgaggatttttattttttttttttgatgttgttatgtattttttattttattattaaaataacggagtattaataagtaaattacgtaattttttaatgttatttagggaagttattttatttttattataataggtataattgtgagtcggtatttttaatttttattgttataataattatttagggatttcgttaaaatttagattttgattcgtaaggtttaggttggggtttgagattttgaatttttaataattttttagacggtgttgttgttgttggtttaaagattattttttgagtagtaaaggtttttttttaaagttatttttttttttttttttagttatttttttttgtttttaaagttaagtattaggatgttagagttttgtggtattaagagagttaagatttttttaatatcgatttttaatttatggttatatttatttgttttttcggtttaagttattggagattgagagttaatttatgatattataggtatgttaagtaggggttttggttgaggatagggaaggttagatattggttgggaaaggatttcgtttttaggtttggaaagtggggagtttagagtattgggattttttaggattaaattattgttgtaggttgtttttttaaaatgtatttttaaaggtttttttatggttaagggattgtaaaggtagggtagttttgggaagattaagatttttttttttattagagatgaagttttgtttgtagaaatagatttaaaaataaaagaacgaaaaaataaaagtatgagttgggagtatttgtgtttattttttttttgttagagttatttaatgtgattaagaggtagaaaatatttagaaaagtatttagagggttgttttagaaatattttaggattaatttttgtattggatttgataaaattttaattaaaaggttttttttttttttttttttaagggtaatttttagtgtatttttaaaggattagttattgtttttaggtttttttagggatttttatgaggatagaagggattaggtatttaggtttttaatatcgtataggtttttaaagggtagtgggtttattatattattttaaaatgattttttaattatgtagatattgatatttgggtttagagataggtgatgtttaatagtttaagtaaatatttaggtatttttatatttaaaaatagtttgaaattaggttgtatttgtttgttgaaatggtatttttaaagtatttacgttgatataaggtgcgattttataagttttaaattggttggcggtttttatgagaatatttgtaaaaagtataagagaaaaataatgtgaggttaacgtttgtagtattttttagggttttaaagggtataagatagaacgaatttttttgaaaatggattattatttttttaaattgtatttaaaatttaaataaatgtaatttatttttaaattttaggattttattaatagtttttgaagattattaagttaagaattattgttatttttgaggtttttttttttttgttattgtaatagtaatatttattatttttttttttgtttagattattaaatgttttttgtataagaagtatatttttatggagttgatttttttgttttttatatttagtttttcgattttgaaattaaatttataggttggagggggaaaaaaaataaaatttagatgttatgattaaaaatttttttaaattataaaagtataaagagaaaagagtcgtg

SEQ ID NO: 9 - DNA, 9739 bp, Artificial Sequence
  chemically treated genomic DNA (Homo sapiens)
tacgatttttttttttttgtatttttgtaatttaaaaaaatttttagttataatatttaggttttattttttttttttttttaatttataggtttggttttaaaatcgaagagttaaatgtagaaaataagaaaattaattttataaaggtatgttttttatataaggaatatttgatagtttgagtaaagagagaagtagtgaatattattattataatgatagaaaaaaaggaattttaaaaatgataatagtttttgatttaataatttttaaaggttgttaatggagttttaaagtttgggagtaagttatatttatttaaattttggatgtagtttagaaagataatagtttatttttaaaggaattcgttttgttttgtattttttggaattttgaaaaatgttataaacgttaattttatattattttttttttgtattttttataggtgtttttataggggtcgttagttagtttgaagtttgtagagtcgtattttatgttaacgtaggtgttttaaggatgttattttagtaggtaagtatagtttagttttaagttgtttttaaatatgaaaatatttgaatgtttgtttgaattgttgaatattatttgtttttgagtttagatgttaatgtttgtatagttggagaattattttgagatgatatagtggatttattattttttaaaaatttatacggtattaaaggtttgaatgtttagttttttttgtttttatgggaatttttgaaggggtttgggggtagtaattggttttttaaaaatatattaaagattgtttttagagagagaggaaggaaaagttttttgattaagattttgttaaatttaatatagaggttagttttaaggtatttttaaggtaattttttagatatttttttaggtattttttattttttagttatattaaataattttgatagaagaaaaataaatataagtatttttagtttatatttttgttttttcgtttttttgtttttaaatttgtttttgtaaataaagttttatttttggtaaagagagaagttttggtttttttagggttgttttgtttttgtaattttttagttataaagaaatttttaagagtatattttaaaaggatagtttgtagtaatgatttaattttgggaaattttagtgttttgaattttttattttttaagtttgagggcgaaatttttttttaattagtgtttgattttttttatttttagttaggatttttgtttggtatgtttgtgatgttatgaattagtttttagtttttagtgatttggatcggggagatagataggtgtgattatgaattgagagtcggtgttagggagattttagtttttttggtgttataggattttggtattttgatgtttggttttggaaataagagagaatgattgaagaaagagggaggggtgattttaaaaggagatttttattatttaaaaggtaatttttgggttagtagtaataatatcgtttgggagattgttagaaatttagaattttaggttttaatttagattttgcgaattagaatttgaattttaacgagatttttgggtaattgttatggtaataaaggttagaagtatcgatttataattgtgtttattgtggtgaagatggagtaatttttttgaatgatattaaaaagttgcgtagtttgtttgttgatatttcgttgttttggtagtggggtaagagatgtatggtagtattaagagaagagggtaagggtttttattcgtagtttttttaatatattattttagtttagtttttaggggagacggttatatttttgtagaagggggttttcggggttcggggaaataagtttattttaagcgagtttcggagatttaggggatattggagtatttatgtggaattttagatttagtaataggttgagaaggttattgtcggaataagatgtatagttaaatgtttttttttcgtgtttaaatataatttaattttttttttggttaagttggacgtcgttaatgttttgtttttattttgttgttattttaggatagttattgtaacgtgacgtttttgtttttttaggtttaggcgtagttgtagttggatagcgttgtggcgtacgcgtattattatttgtattcgtatttggtcgcgtacgcgttttatatgatgtttttagtatcgtttttcggattgtcgttcgttacgttggtcgcggattcggttttcgtcgtttcggtagtggcggtcgtagtagtcgttaagattattagtaagaattttagtatcgtcgattttagattgaaagttaaaaagtacgtcgtagttttgggtttgtgacgttaacgttagtattaatgtcgcgtttgtttcgcggtatttagtttgtacgtttttcgcgtttcgttgttttttcgttatttttttgagatttcgggagtcggtttttttttcgttttattgattatttttcgttttttatcgtattttggattcggaaagttagattttacgtaggattagggattttacgaggtacgtaggtttcgtggtttttgttcgtttttttattcgagggtttagaattgggttttgtaggagcgggtttgggggagtttggagagagattggataggggagtgttggaatcgcggagtttggtttatcgtaaagttgtaacgatggatttttgtatagaaaaaaaaattttgttaataatgaaaaaatgagtaaataaaaaaatcgaaagataaacgggagagaaaaagaggaagggaatttattttttaattgttatttggtagaagttgaaattggagaattaaggagtaaaaataaattttaaaattaaagtattttatatatttaaaaatatggaaaaataatttagacgattttcgagagattggggggagttattaatttaaatgtgtgtttttaaaaatgcgttaagaaggtaaagtagaaagaagaggtatatttatttaaaaaattaagatgaaaaaagtgcgtagttgggaagtttataggttttgaaattgatttttttttgcgaagtttacgttaatacgagaaatttgatgagagaggcgggtttttttttacgttgaattagatgttttgagtttaaatttattatgtatggaagagtaagaaaagagaaaatattaaaacgaggagagagaaaaataatattaatataaaaaaatgttatagataatgatttttttgagaaattattatggtaaaattgtttggattgttgatagtaaatttcggtttgtatgttatttgtattttattgatggtgtgtttttttttatttttttattttttatgtatttattttatttttatttttattatgaaaaataatattaaaagtatttggaaattgatatatatatatttatatatatatattatatatttgttatatatatatatatatatatatatatatatatatatatatatttgttttgtttttgattttggggaataaaagaaaaaagttagaaagggaaaaaattatatttattgttttaagaagatagaggtgggtagaatatgtggggaaaggaaaaagaaaataagattattaaatgaaataatgaaggtatagcgtttcgttgtgttagatatagtaggcgtttaattagtattagtttttattatttttttttttttgtgtttttttttgttggttttttgaagttttatttgaagatagtggtttatttttttttttttatttcgttaaatttattttaaataatatttagttagatataggtattaggtttgtgtaagatatgttgatatatacgaataaagtttattttgattataatgtgtggattgatttttaatatttgtattttattttataaaggtgtatttatttaagtaattttttttttttgtttgtttgttttttttttgttttttttttttttttggttgtttgttttaatttatgtagttatttaaattgggatattttggattaagttagtttgtattttaattcgttagtaagtttaagtttgtggggttttgtttttgtttttgttttattttttaatttataagaaagaggaaaagtttttttaattgaattttggtatgcggttgagttttgtaattatttgttttttatgaaaataaaattatttatatttgatatatttttttttagtgtattaagttattttaaataaaagatgttattttatgacgtgttgttagtataaaatgtgtcgtttttaattttgttaaattttttaaataagtgttaagttattaattgaagatattttgcgattaattgaatgaaaatatcgttttatttgatggggttcgagttatttttgtttttgttattattaaaatattcgggatagttttttttttttgattaatgcgtattattttttcgaatatattaatttggaaaagcgtttgtttttgtttttacgtgtcgtagttaaggggtagtaagaagggtggttttgtggttgtcgttagcgttgagcgtattttcgtcggttttttttattttatttatttagtatagaaatagggttatattaaattaatgaaagaatagataagaagtaaaataatataggatagagtaatttttttttttaagatggattttcgttattcgttttgttataattttagtttttatttggggatatttttgggaggaaggaaggagggattttgcggagcgaaattaatttgggtttggtatggtataatcgataattagttttagttcgttgattttttttggagcggggtaggtgttgatttcggagcgggaaaagagtcgtaaaggcgggtaggttagggtattgtggagggaggattttttatttgatatttttgtttagggttggttgaagtagtttttttattggttaaatttagaaggtggaagttagaatcgggtttaggaaaagtttggttttgaaaataatgaggagttttttttttttatgtcgtggtttttttttggaagaaattttattagtaaagtttaaaggtgttgtttgtgtggggatagttggagattttgaagattcggttttgattatgtttaaggtaggaattttttttttcgcgcgggtttttgtgatgacgggatttttcgtaaagattgaaagagttttgagcgttttttgggattttttattttcgtgttggagggtgcgtaaaatttttgttaaagttttatattttttagtagtgttttttattatttttatttggtttttttttaggttttagttcgtatttaaagttgttttttagtatagacgtgggcgataggttttttttttttaatttttaaagattattgaataggatttgggtcgtatttagtgcgtaggttttgcgggttttttggttgatgaacgtcggtagtttttaataataaatatttgtttgcgggtatggtacgttttgaagtagttaggttattcgtcgtggttttgtggtttttagcgagttttagtcggcgtcggggtcgggggttttaataggtaggttttaagttttaaattttaattttaatttagattttaatacgggttcggattttggagattttggagtaggggagatggtggttttttttcggggttagtttgttttaagtagcggagcgcgggggaagttcgaggttaaaggaggcggtgcgtttaggttttgttggcggaggcgtcgggtatcggtatagaggtttagtgacgcggcgtgggtgggtcgggaatgtttcgtgcgataggagttaggtgggttcggcgcggagattcgcgggagtcgggtcgcggcgggagcgcggtaggcggagaggttcgcggaggtagttaggttcggcgagaaaggttaaaattttttggttttattcgtagtgttttattcgggtacggtttgtgggattagtgtattcggggagttcgcggcgtttttgtttttttttgcgtttgtttttttaagattaacggaggatagaggtcgttttttttggttttgtggagtttcgtttttcgggtttagattttcgtaaggtttgttttttcggaatgttagtgtttaaattttggggtaggcgtttcggtgtgtgaataagagttttaaattcgtagtttttcgaattattttaagaaggggtttgaggaatttattttaaatttaaataaggtttaatatgtttgtagtttgggtcgaaatttaataattttaatataattaataaaggatatttttattttttgttttttaaattttttgggtatgtaaaattttttcgagttgtaaaattaatgattgggaaaaattgagttaaaagagattgtttaagatattattagtgttaaggattttatgatataagagatatagtaagatagtaagatcggtgtttttgtttggagttgaattgatgtatatttattttgagtttttttttagatttataatttttttgggtttttagatggtaatattttagtataatgtaatgtttaaatttcggaattattttgatttttgaaaaatgtttttttggttttagcgatataggattttgttttttttttataaattagtttatattataaattgtaaatatttgtaataagattttaagattgtttattttatataattttaagtatttgattatgttaaatattagtatagaaaatttgtgaaattagtttaatgtgtgtgagggaattaaaggattatttatttttattatataaataaagtatttaagattgttttgattgttagttttttaatttttagttatataattgattattaatttaagagttattttgtgtaaaaataattattgaggttatattttgttttaattaaatatttttgggtagaagaaaatggtattgtatattgggaagtgggggattgtttttaatattatattattttagtttaaacggaaataaaagtttgtaaattttaacggggaaaaaaggtttgaaatggttttttaatatgttattaaaatagttagttaaaatatattaaagtaatattataaatttagaaataataaatatgttattttaatgagaatgttaatttatagataatttatttaaggtagtttttatattttatatggaatgttttattatgattcgtttgattattgtgatgtttgttttttaaaatttaatttatataggtttgtaatatatataatttataatttttgttgatatacgtatatatattttaaattttaaatttttatagtatgtttggatgtttattagtatgtttttaagatgacgaaaataaaaatataaaaagtgttaaatattatattgaaagtaagtgtttgttttgttttatgtttgaaggtaaagtagttgaaaatgaaaaaattaaaaaggattagataatttttaagtttaggtattgagtgagtattgaaatattttagataaaaaaaaattttgtataagaaatttgaaaattttaagaaagatttatagatgtgagtgtataatatttttgatgtttagataattttttttttggtgtttaatttatgaaataaaaaattttcgagtttcggggtattttgttttgggtattttatatgttagttaaggtaattattatatatgttttcggtgtaggaaagtttagtataatacgcggagttttattggtatgaggaaaggttttagaatataattaaatatattaaatagggaggagtaaatatgtttttttttttgttgaataattttttatatgttttgagtaaggatttttgtagatttttatttaagtagaatagtgtatgtttaagagtatatattgtggagttatgttttattgttggatttaaattttattgttattgtttattagttttttgattttggataagttaatttttttgttttttagtttttttatttgtaaaatgtggataaaaatagtgtttattttatggaatggttaggaggattaaatgagttaatatttgtaaaatttttagaataatgttttgtatatgtatattaagtattaaataataattatttttattaaaaggttaatagatattgatttttgtaataaaaattttaaaagaaaaaaataataaagtgttgtaagataagaaaagtttttttttgtgggtaagaagaaaatattgtttgttagtttgtgattttggttaattgtttagtttgtgtgagttttatttagaagatgtgataattgttataatgtagtagtgtagttaggaggtttatttagaagagtaatttataggttgtgtgtttttatatgtaagatagataatattgttattttgttatgtatagttaataaggtgagattttaggtatggtatttgttttcgaggagtttattatttgggtaaaatagtttataaatatatatgatagtgttaggaagtatgtgttaaatgtatttgtttaaaggtattaggatatataatttgtgtatttgaatggttatataatatatgtgggtttgttgttttggttttggaaattaaattttttttggaaatgagtagttttataaaggaatggtgaagcgatatttagaggttaagaagcgtattttgttaggattaaatgtatattatatgttgtcgtggtttgtgttttatagtattatttgtattttttttggttgtttatgtttatcgagtattagtagtaaaacgttaaaagatataaaggttgttgattaaggatttttaatagtagtttgaaaatatatataaaataaagaaaggaatgtttaattattattatagggatttgtaagatgaataattgagttataattatagagtaaattaagaaggatttttttttaatgtttaaatttaggtatattattaatattgttttatattttatataatatttatgaagagttaagtattttttatatatttttatttgggatgtgttgttatagtatacggttaatttttagaggtagatatttttgattttaaatttttgttatattaattattagatgtaaagttttaggttattttttagttttttaaaatttatgattttttagtgttttgtagtatatattttgttaattattgtgaggattaaataaaaataagttatgtaaggtgtttggtatataataggttttaataaatgtatattttttttttatttttattatttattttttaaattattgtaatatagagaggatttttaatttatttttttgtgattagaagtagttttgaaaaattttgggaggtggtgtgtattttaattttaattttttattgtaagtattgataatgtgtgtttatattgtgtgtattggagtttggaataaagttttttttaaattttgtaaaaatttttagagattttgagggattgtgattttagatttgtttttttgtgtagaatagaaaggattttattttttttttcgataatttgaggacgtttttaatgtgtaggttatttagagtttttgatttttagttttttttattttggaatgttagtatatttatggagttttggtattaattttttagttattttgatattagattgattgtttttttgttgtatatttattgaatatatttttatgttttcgt

SEQ ID NO: 10 - DNA, 4313 bp, Artificial Sequence
  chemically treated genomic DNA (Homo sapiens)
gggtttgattttttgagattcggggaggatttttggtagatgtgtgtttagttagaatatttggtaaggatttttttaatgaagaaaaagtggaggaatttagttttagcgagaagaggtttttttattttgttttagatatatcggatagagggtatattttgattagagttacgtttagtggttaggaggttagtttagtatttttttttttattattttcgttttgggtgggggggtaatttttttgggagtagttgtgggaattgtcgttttttattttagtttagttagtattttgaagtttgtaggggaaggatagtacgtgggatggatattggggaaggagtttcgtaaggttagggtgtaatttttaggttttaggtggtttggtaggttacgttgtttcggagatgtttgttagattttttaagtttatttagggtttggtagtaatttgttggttgtttttgcgggggtttgggttgttgagtacggtgtagttgtttagggttaattagttttagggtgttcgtgttaggttgcggttttttcgtttttttcgtattgagggtatttatggcgtgtaaatgttttcgtatttttagagttgttttatcggatgtttttaggaatttatatatttttataaaaatgtattttaaatgatggataggcgagtttggggtaataacgggtgtttggtgggtagataagagtaaatgggaaggagttcgagggaggagggggaagagaagaggaaatagaatttttagttggatattttgataatagttggaaggaaagtttagaaaagatgaagagagaggaggggagaaattaattggggtttttatttttgtcgttggatttttaattttcgttttaaatgggttttgtttttcggtaaaattagtttaaaggattttaaaataaagaaaacgagatgatcggtttgggagttttttaattagagtagagaagttagaggggggcgggcgatttggttttgaagttttagttgaatagttattttttttttttttggtaaaaaggatttttttagaattttcgaggtttttggattttttttttcgtaaatggagtcgtatattgtattttttcgttttttcggatcgttaagtatgttttatgagggtcgttgttttcgggtggaatgcggtcgtatgtacgcgtttttttgtatacgtatatatacgtatatttataataagtgtttgtaggaggagtgttttgcgcgttagttttgcgtttaagataggaagttgtcgggttatcgagttaaatgggagtgatattatttttttttattagtaaggaaagcggattataaaagtttttttgtatttcggtagtttatttaatattatttatgtattttgtgtaaggaattgtgggatttcgttttacggtaaataatatggaaattttaaaaatagcgatttttttgtgcgtgtttatttacgcgtttcggggtgatttggcggggttgtcgtcgggtgatttatatttttgaatcgcgaagcgatagggaaagcgcgggcgagcgtaggagacgcggtcgggggttttttcgggtttttgggttttcgtattcggagcgggggacgcggtcgttttaaggggaggaggggcggcgggttgtttttgttatttagcggcggtcggagcgttacgtgggcgcgcggcgtcgcggttattggttcgaggtacgtgtttaggagatcggtttgcgacgttattcgagggggttttgttaaaaataagaataaaaatttagagtgaaagtgttttaggttgcgtcgagtggtttggaaattttcgagttcgcgcggaggtcgaggcggcgagggcggcggacggtcggggagcgcgggcggtttagttcggttcggtcgggttttggtttcgcgttttttatttatgcgattcgggtcgcggagttttgcggggttcggcgggggcgcggtcgtacgtcggtggggcgtttcggttcgtagcggggcggcggtcgcgaggagggggtttttatgtgcgtgcgggcggtggcgggcgcgttgatcgcgggcgttcggtattttcgagggtcggttagggcgtgcgggcggggacggtcgggcggcggcggcggtcggagtcggttcgggcgggcgtgagcgtcggggaacgcgttgtttgtatgcgcgtagttttcgtttcgggcggtttaggcggcggcgtcggagttcgaggcggtcggacgcggagaggagcggggagttcgggaggcggttcgcgttttcgtcggattattgcgattgtttagatttcggttgcgcggcgaagtcgaggatttggttttgttgaattttttatcgtttgggcgagcggggcggttcgtggtgtttttaatttagttcgtggatttaaaggtggtttcgcgtcgagcgcggtcggcgatttgtaggattttagttttggtcgcggtcgtcgcgtacgttttcggaagattcggcggggtgggggcgcgggggttttcgtgtgcgtcgcgggagggtcgaaggttgatttggaagggcgttttcggagaattagtgtgggatttattgtgaatagtatggaggagaatgattttaagtttggcgaagtagcggcggcggtggagggatagcggtagtcggaatttagtttcggcggcggttcgggcggcggcggcggtagtagttcgggcgaagcggatatcgggcgtcggcgggttttgatgttgttcgcggttttgtaggcgttcggtaattattagtattcgtatcgtattattaatttttttatcgataatattttgcggttcgagttcggtcggcgaaaggacgcggggatttgttgtgcgggcgcgggaggaggaaggggcggcggagtcggcggcgaaggcggcgcgagcggtgcggagggaggcggcggcgcgggcggttcggagtagtttttgggttcgggttttcgagagtttcggtagaattcgttatgtgcgttcggcgcgggcgggtcgtttttagtcgtcggtagcgatttttcgggtgacggggaaggcggttttaagacgttttcgttgtacggtggcgttaagaaaggcggcgatttcggcggttttttggacgggtcgtttaaggttcgcggtttgggcggcggcgatttgtcggtgagttcggattcggatagttcgtaagtcggcgttaatttgggcgcgtagtttatgttttggtcggcgtgggtttattgtacgcgttattcggatcggttttttttaggtgagttcgcggggattacgcgtttcggttcgtcgcggggaggttcgcggagttggggggcggtgttggcgcgggaatttatcgggaggaaaatatttcgaatttttttcgcgtatacgtataaagatttacgcgatattgtgtgaagttgacgtcggttcgggtagcggttaggagtttagcggtaggattgattcgttagggggtatagattttttaggatcgtagaagggattttttttttttttttgttttttttttttttttttttttttttgttttttttttttgttttttatttcgttttggcgtatttttttttagtttttagtttatgttttttttattgtagttttttttggtgggaacgtggtggttggaagatgggttcggaagtgtatatttttatttttttttttacgattttttaatttaggttaggtcggggacgtatgttttagtttattttagatttgttttattattcggttatttcggttgtgttcggggaagaaaaggcgaggttttttgtcgttttgttttttgtttttcgggtttgcgttgatcggtgggatttaggaggatgtatatagggaaggaggaaaataaaggcgttttttttttttggttttattttgtttgttagcgttagttcgtagtggtggggtttagttttttttttgtatatagcgaggataagggaggtagtcgtttttttcggtatttgttatttttaaatagaaaggatttttttttagggttttttgggggttgttgatgggaaagaggtagtattcgtaggggttttgtagagatgttggatatatttttttatagatttgcgattttaaaaaattaagtttatgtttttgtagaaattattaattgtattttatgcgggtttgcggttgggaatcgttattagaagtggattgtttgatttcgagttggtagcggattttcgttgtttttaaatttttaattattttgcgggggttatttgtttagatt

SEQ ID NO: 11 - DNA, 4313 bp, Artificial Sequence
  chemically treated genomic DNA (Homo sapiens)
gatttgggtaaatgattttcgtaaaatagttgagggtttgggggtagcggggattcgttgttagttcggggttaaatagtttatttttaatggcggtttttagtcgtagattcgtatggaatgtagttgatgatttttataaggatatggatttagttttttaaaatcgtagatttatgaaaaaatatatttagtatttttgtagggtttttgcgaatgttgttttttttttattagtagtttttaggaaattttgagagaaggttttttttatttggggatggtaggtgtcgggagggacggttgttttttttgttttcgttgtgtgtaggaagggggttgagttttattattgcgggttggcgttggtaaataaagtggagttaaggggaaagggcgtttttatttttttttttttttgtgtgtatttttttgagttttatcggttagcgtaggttcgaggggtagaaggtagagcggtaaagggtttcgttttttttttttcgagtatagtcgggataatcggatggtaggataagtttggggtgggttggggtatgcgttttcggtttggtttgagttggaagatcgtagggaaggggatgagagtgtgtattttcgggtttattttttagttattacgtttttattaaaaaaaattatagtgggggagatatgggttaggggttgggaagagatgcgttaaggcggggtggaagatagagaggggagatagggagagaggaaggagagagagagatagagaaagaaagaaggttttttttgcggttttaagaagtttgtattttttagcgaattagttttgtcgttggatttttggtcgttgttcgggtcggcgttagttttatataatgtcgcgtgagtttttgtgcgtgtgcgcgggggaggttcgagatgtttttttttcggtaagttttcgcgttagtatcgttttttagtttcgcgggttttttcgcggcgagtcgggacgcgtggttttcgcgggtttatttgaagaaggtcggttcgagtagcgcgtatagtagatttacgtcggttagagtatgggttgcgcgtttaggttggcgtcggtttgcgagttgttcgagttcgagtttatcgataggtcgtcgtcgtttaagtcgcgggttttgagcgattcgtttagggggtcgtcggggtcgtcgttttttttggcgttatcgtgtagcgagagcgttttggagtcgtttttttcgttattcggagagtcgttgtcggcggttgggagcggttcgttcgcgtcgggcgtatatggcgggttttgtcggggttttcgggagttcgagtttaagagttgtttcgagtcgttcgcgtcgtcgttttttttcgtatcgttcgcgtcgttttcgtcgtcggtttcgtcgttttttttttttttcgcgttcgtatagtaggttttcgcgttttttcgtcggtcgaattcgggtcgtaggatgttgtcgatgaagaagttggtgatgcggtgcgggtgttggtggttgtcgggcgtttgtaggatcgcgggtagtattagagttcgtcggcgttcggtgttcgtttcgttcgggttgttatcgtcgtcgtcgttcgagtcgtcgtcggggttggatttcggttgtcgttgtttttttatcgtcgtcgttgtttcgttaggtttggggttatttttttttatgttgtttatagtaaattttatattggtttttcggggacgtttttttaaattagttttcggttttttcgcggcgtatacggagattttcgcgtttttatttcgtcgagtttttcgagggcgtgcgcggcggtcgcggttagggttgaggttttataagtcgtcggtcgcgttcggcgcggagttatttttgaatttacgaattgggttagaaatattacgagtcgtttcgttcgtttagacgatgagagatttaatagagttaagttttcgatttcgtcgcgtagtcggggtttagatagtcgtagtggttcggcggggacgcgggtcgtttttcgggtttttcgttttttttcgcgttcggtcgtttcgggtttcggcgtcgtcgtttgggtcgttcggggcgagagttgcgcgtatgtaggtagcgcgtttttcggcgtttacgttcgttcgggtcggtttcggtcgtcgtcgtcgttcggtcgttttcgttcgtacgttttagtcggttttcgagggtgtcgggcgttcgcggttagcgcgttcgttatcgttcgtacgtatatggaggtttttttttcgcggtcgtcgtttcgttgcgggtcggggcgttttatcggcgtgcggtcgcgttttcgtcgagtttcgtagagtttcgcggttcgagtcgtatgggtgagagacgcgaggttagggttcggtcgggtcgggttgggtcgttcgcgtttttcggtcgttcgtcgttttcgtcgtttcggttttcgcgcgggttcggaaatttttaggttattcggcgtaatttgagatatttttattttggatttttgtttttatttttaatagagttttttcgagtgacgtcgtaggtcggttttttggatacgtgtttcgggttaatggtcgcggcgtcgcgcgtttacgtgacgtttcggtcgtcgttgggtgataggagtagttcgtcgttttttttttttttaaagcggtcgcgtttttcgtttcgggtgcgggagtttaggaattcggagagattttcgatcgcgttttttgcgttcgttcgcgttttttttgtcgtttcgcggtttaggggtgtgagttattcggcgatagtttcgttaggttatttcggggcgcgtaggtggatacgtataggaagatcgttatttttaagatttttatattgtttatcgtggggcgaaattttataattttttgtataaaatgtataaataatattaaatgagttgtcgagatataaagggatttttgtggttcgttttttttgttgatggagaggaatagtgttatttttatttgattcggtaattcggtagttttttgttttaaacgtagagttggcgcgtaggatattttttttgtagatatttattgtaagtgtgcgtgtgtgtgcgtgtgtagggaggcgcgtgtatacggtcgtattttattcggggatagcgatttttatgaaatatgtttagcgattcgaaagagcgggggaatgtagtatgcggttttatttgcgaagggagaaatttaggagtttcggaggttttaaaggaattttttttgttaaggagaggaggggtgattgtttagttaagattttaaaattaagtcgttcgtttttttttaatttttttattttgattgaggagtttttagatcggttatttcgttttttttgttttaaaattttttaaattaattttgtcggagagtagggtttatttgaaacgagaattaggaatttaacggtaagggtgggagttttagttggtttttttttttttttttttttattttttttagattttttttttagttgttgttagaatgtttaattggaagttttgtttttttttttttttttttttttttttcgggtttttttttatttgtttttgtttatttattaaatattcgttgttattttaggttcgtttgtttattatttaaaatgtatttttatggaaatgtgtgaatttttggaaatattcgatagggtagttttgggggtgcgggagtatttgtacgttatgagtatttttagtgcgggggaggcggggaggtcgtagtttggtacggatattttagggttgattggttttgggtagttgtatcgtgtttagtaatttagattttcgtagaggtagttagtaggttgttgttaggttttgagtgaatttggggagtttggtaagtattttcgaggtagcgtggtttgttaggttatttggggtttgaaggttgtattttggttttgcgaagttttttttttagtgtttattttacgtgttgttttttttttgtaggttttagagtgttggttgggttgggatgagaaacgatagtttttatagttgtttttaggaaaattatttttttatttaggacgggaatggtgggggaggaggtgttgggttggttttttggttattggacgtggttttggttagagtgtgttttttgttcggtgtgtttagggtagggtggggaagttttttttcgttggggttggatttttttattttttttttattggaggagtttttattaaatgttttggttaggtatatatttgttaggggtttttttcgagttttaaaaaattaaattt

SEQ ID NO: 12 - DNA, 16197 bp, Artificial Sequence
  chemically treated genomic DNA (Homo sapiens)
ttttgtaggtggagggggaaagggtttgggggttggtggaggacgtaggagtatgggggagttgtggaaaagacgtggaggtagagttagtaggttttgttaagggataggaggtggttttagagagaaaatgagggattagggatgttttttggggggtggttagttgggtgattgggagaaattgggaaggcgtagattagggtagtaggggtagcgggatcgggggtgttttgaagtgaatacgtgaaattgaaggtgttcgttagttttttagtggaggtatttaggaggtagttggaattggaaggaaatggtgatagtcgttagtatcgtagaggtggcggtggtggtggttatagttggatttttttgggttggtgggaagtgtggaggtgaaggagtagggagtagggggaggtggagaaggaaattttaggtttatatttttattttatttttggttgttatatttttaggaagttttttttgaggttgggatagagggtggggatagtttagtttttttgagagagtttattttcggaggtttgcggtgggagttagggcgcggcggagagggttttttattttttttttagtaagcgggagggaggcgtcgggttgaggttgcgttgagttcgagttcgtcgttcgggtggattcgttttgtttaagcgcggaggggcggaggtttggttgggttgtagcgtggtgcggagtaggacgtcgtttcgtgttatggttattggagacgtacgtttattttttttttcgggttcgtcgattttgttatttttcgttttcggtcgttcgtgggtgttcgttatttagatttttttcgtgttttatgggacgtaagtttttttttagagttcggttttgtaaaagaggtttggagttttcgttatagattttttttttgggttacggaggatgagaagggttatcgagtcgagtcgtaattttgcgtaatttttgatttttttttttttttgtttttacggtattttatcgtttttttttcgttttcgaggttttttagaaaatagcgtagaattgtgtagatgtttaggagatgtgaagatgttggagatgtttaggaggcgacggttacgcgaggagaattgtttttaggtgcgtttttggaacgtacggagatttttcggcggggaaggggtcggggttcgcgttatttagtgtttgtttatcgatttttttttgaagagggggtttagggtttttgttgagggagcggggaggcggtgcgggtttttttcgggtatatatgggtgttcgtttttttttttttttttttttttttcgtacggagagcggagagcggagagcggagagcgatagggaggtagtcgaagatttgaattttgaaaggggagttggcggcgaatggtgaatgagatagttatttaggaagcgagtttagttagttcgggaggcggtggagatttacgttcggaagtaatcggattaggttttagaatgtgatcgtttttcgggtttcgggggagacgttaaggacgcgtaagcggagggcgcggagataattgggagttagagtttttattatttgggttgggaacgtttttgggcgtttcgacggggtggcggtgggggtcgggggaggtttttgagaatcgtgcggttcggggagagtttatttattgttgagtttcgatataggttttgaagttgttgtagtggtttagtttttttttcgttcggttttgcgtagcgcggtgtcgtagagtttaggtcgtgttttcgtttcgtcgtttagagtttattgcggcggtttattggattacgtcggtggggcgatcgtagtttttgatttgtgagttgtaaagagtttcgaggtttatttataaatttgcgtttttagtcgtttttttacgtattcgagttacgtttcgggattcggagagttcggggcgttgggcgttgcggaggaggttcggtttttgtcgttttttttattttttagttcgcgagggatttgggggaagggggagtaagttttcgtttcggaagaaacgttcggaattaaggagtttgatttttggattcgggtgtttgttggatttaggtttttattttcgggtttttcggtcgaattaagggtttcgatagggttcgaggtattcgtatttttaggagataggagttcggttagggcgtattatcgggttttgtttcgagttagaggatgtagtgtagatatttatttttatatcgtttttttatagagtttttttttttttggggcgtcgttgggtatagggtaggtcgttaggaatttttagtaaaattacgttcgtttagaggttcgcggtttttttagagggcgttggggaaagagaggggatttgattttttcgattttcggaggaaaagtgttcggggttttttagggatatagttttggaagtttattttattttagtttaggtcgcggcgaggtggggaggagagttaggagagggggagaggggttttgcgttttgtagaggtttttaattttggaggaaaaagattgggatatatttaagcgagttaaggttcgagttttacgtatttttatttattcggggcggcgtatagttagtttttgtcgggcgtgagtattcgattaagggagtaagtggaatgaaaatttagttgggggggtttttatcgatataattgtttcgtagtcgagtttttggatttttgggagatgtggagagtttggggtcggttttcgtttcgtagagtagattggattgttttaggtgtttggaatgcgtttgtattttgtttttcggattcgcgggagattcgtgtttcgtaagtttttttttttattttagtttgtttttatttatatttcgtcggcgagtgtgttatcgcgaggcgttttttttttcgggaagggagttttttttcgcgtagattcgtattgttttttttttcgttcggttttgttttttaggggtagtttttgtagaaaggagattttttttcgggtcgaagggttattagtttgtagttagtttagtttcggattttgggagatgtttattattttgcggattttgattcgaaattttttttggtcgtttattgcggagagtgttttcgtagagaggtttttaatcgaaggagtttgggttttatattttgtttttaagtttgagtttttagggttgttttgtacgaggtgtagatgaatttgtgtttgtaaataagatagaaaattttaagtgtcgttcgattttttttttttggggaattcgtattttgttttgggagcgtgtttagcgttttagtattaaattttttggtcggggttggtagagtttagagtttcgtttttttttaggcgcggttttttaatatttgtaatttaaatgttgcgtcgcggttaaaattagttttggtagtgcgaatagagaattaaaagtaggtagtgaatgagaatagttcgtattttttttttttggtagacggggaggtgtaaatttcgaggaattttagggtattcgttttaaacgtgggaaattttcgcgcgtatttcgttttttttttttagtttgtgttaatgttttaaatggtgtcgagttgtttaattttgtcgttattatagaggttgttgtgttttaggggattaattgatgtgagatatataaaattttgtaattttataatataaattatagtatagtttttttggagagggttggaatatttgagtgagttttcgagaggaaaagaggagttttttagaggagaaatagagtatttttataatgtgttttaattgagaaattttgttttattgagtttttttttaagtggaattagaagtgttgggatgagagggaaaggatgggagtgcgtttaaaggtggatagtaggtttttatttttggtgggagtgagattggacggtatttttcggaaaggtggtttgggttttggataaggttagaggtaggagtttatgatgtagagatgatatagtgttttttcgcgtgtgagtttacgaaggttattattgaggttttgtgtttgtaaaaggtcgttacgttttatataagtttttatatttaatatagggattgattgggtatagggatttttttatattatatatgtaagtatgtatgttaattaaagatgttcgtgttaaagaaacggttaattttgttgaatttagaggaattgattaattatttaattaagtagaggaaatgtttgaatttaattcgtaatttagttgtttttttatataaaattatatatttttatttatatttgatgaatgaaaaaagaaattagtttatgattttaatttaaatatatgtttttaaaaatatattttttttagtttagttagtatataaattaattgagtttttttggttaagtatgatttagttgtgatatttaagagcgggagtggttgtttagatatttttttttttacgtgaaatttagattaatgagttattatttaataagttgtaggtagtttggttgggttggatttagatggtttgagttaaatttagattgtatttgtttaaaattttgtaaatttttagttacgtaaattttatttttaaaaatgttgggaagttattgtataagagtttaagttatattagtttttttgtgattatttgtagtttttggggaaagaatagaagaaaagaaaatgttagtttttgtgaggtggggttggtgttttaggggttttttttgaatattttgttttttttttaaaggttaaaaggaaggtagtggatatatattagaattttttttatttgtgaatggtcgtaaggttggagaaggtggttagtgtattttaaggtttattatttttttttgtgtttttttttttgttttggtaggtttagttagtttaagttttgggtgttattttttaaatttttttgttaaattaattttatttatttgattggattatttgagaggtgttatttttttttgggttgttgtgattttgagggggtatttttataagagtttaggtattaggtggtgaaatagtttgtgtttttaaatttgttttttttagggttttcgggagattttagagtgtaggtttgtttggggagttttaggggtgggttttgagtggaagtgggtttattttttataggagtttaaattttataggaataagaatagtagtaaaataggataagagagtaggtagggagttgttaaggaaaggcgatttttgggaaggtaggttatttagaataaggtttttttggcgttggagagtcggaagggggagcgggtatagaggatttggtttaggtgtgggggttattagtataagtagagttattttttagatttttttttagaagtagttgttttttagagaaattaggtgagggatagtttttgtatttttatacgttagttttggagatttgtttatttgtttttagtcgttttttttttcgggcgagtttgggttaagtattaagttaggatagaagggcggtttttagtcggtttttggtttattttgtttttttaatatttagggagtttgggtttagtataggcgtttttttagcgatcggggtagaattaggacgtgtaatgcgattgttttttttttttcgtttttcggtagagtttttgttcgcgttaatttatttattaggttttgttcgcgttacgcgcgtttttggtaggtttggggcggggaaaggcgaagcgttgggtacgcgagggtttgtgtattttagtttttacgacgtttttggttttttttaggttcgtcgttgtcgtatttaatttttttttttcgggggttattttgaagaagttttagattttagatttagttttttagatttcgttttcgagttcgttcggtgggtttgtaggtcggttttttttcgtttagaggagagcgtagatacgtaatgttcgttcgttggtttcgttcgttttatttttgtttttcgcgttttttcgtttcggtttttgtttataggtcgggtcggaacgttagttttaggagtcgacggtggttttttgttcgtttcggggaaggtgttgtttttttatcggttttaatttttcgttcggcgtttggggtttggttgcggggttcggtttttagtcgagggcgtagggttggttaggtttgttttggttgaggtggagatttcgtttttagggattgttgggcgtttttgtttcgcgagtaacgagatcgtcgcgagcgaacggtttttattgagtttatttttttaagtgttattacgttaagttagagaagcgaggcgagtggaggggacgtagaggggtcggaaaagttatttttttttggtttcgtttttagataaaaacgggagtttggttcggtattcgggcgttcggtgttttcggggcgttttattgaggttttgtttgtaaaattagcgttcgtgttttgaagcgtacggcgtttggaagttgtttttttgttcgtttttttcgaggtttttttttgtgtagcgagtttgagaaatatggaggatcgtttttcgtaagcgggtggtcgcgggcgtttttcgatttttgggtgaagttagagggaaaacggggttttcgggttagtgttttattttttatttcgggagaaattaatgttcggagagggttcgtttgattcgtatagaaaggtcgattttgagggcggcggtcgttcgggggagaaagcggaggttttgggttcgcgggagcgcggtagtcggggttggtatcgtagaggagagatacgttattgtttcgtatttttagaaagcgcgaggcgtcgttttagttggggtaggcggtcgaggtcggtttttatgcgcgtttttcgaagttttttgaaatattttgcggagtttcgtgtgtatagaatttagtatcgtttagtttttgggtagtttaattttttgtagttttattttagttttttcggttttttgaacggtgattttttttatcgtttttttagagttgtttcgtgtttgggtttattttggggggttcggtattttttagttatttttttttttatttattttttttttttttatttttttttttattttttattttttttttttttaggagagcgatttacgaatttttttttttttagttaattttagggtttagttcgtagattttgcgagggtaggtttttgtttatcggttcgttaggcgttttggaggtgacgttttgttttttagagtttttgttgtagttacgaattggggtttgggtcgtaggaaagtatagggttgaagtttagcgttttggggttatttatattgaggtagttagaggtaaagagttttaagaatttagaaaatattttttaggaagtcgtttaattggtttttatgggataggtggagttattaatttgggatggttttgtaggaattaaagagtttagggttttttttttttttaatattatgtttaggagatttagagtcgttggatttttttttttttgattggtgattattagagtttttagagttgtagaaaattttttttttaaaaaattaagtaagcgttaataagattttttataaatttttattagttttattttttcggggggtaggtagattgtggggtttgattttttgagattcggggaggatttttggtagatgtgtgtttagttagaatatttggtaaggatttttttaatgaagaaaaagtggaggaatttagttttagcgagaagaggtttttttattttgttttagatatatcggatagagggtatattttgattagagttacgtttagtggttaggaggttagtttagtatttttttttttattattttcgttttgggtgggggggtaatttttttgggagtagttgtgggaattgtcgttttttattttagtttagttagtattttgaagtttgtaggggaaggatagtacgtgggatggatattggggaaggagtttcgtaaggttagggtgtaatttttaggttttaggtggtttggtaggttacgttgtttcggagatgtttgttagattttttaagtttatttagggtttggtagtaatttgttggttgtttttgcgggggtttgggttgttgagtacggtgtagttgtttagggttaattagttttagggtgttcgtgttaggttgcggttttttcgtttttttcgtattgagggtatttatggcgtgtaaatgttttcgtatttttagagttgttttatcggatgtttttaggaatttatatatttttataaaaatgtattttaaatgatggataggcgagtttggggtaataacgggtgtttggtgggtagataagagtaaatgggaaggagttcgagggaggagggggaagagaagaggaaatagaatttttagttggatattttgataatagttggaaggaaagtttagaaaagatgaagagagaggaggggagaaattaattggggtttttatttttgtcgttggatttttaattttcgttttaaatgggttttgtttttcggtaaaattagtttaaaggattttaaaataaagaaaacgagatgatcggtttgggagttttttaattagagtagagaagttagaggggggcgggcgatttggttttgaagttttagttgaatagttattttttttttttttggtaaaaaggatttttttagaattttcgaggtttttggattttttttttcgtaaatggagtcgtatattgtattttttcgttttttcggatcgttaagtatgttttatgagggtcgttgttttcgggtggaatgcggtcgtatgtacgcgtttttttgtatacgtatatatacgtatatttataataagtgtttgtaggaggagtgttttgcgcgttagttttgcgtttaagataggaagttgtcgggttatcgagttaaatgggagtgatattatttttttttattagtaaggaaagcggattataaaagtttttttgtatttcggtagtttatttaatattatttatgtattttgtgtaaggaattgtgggatttcgttttacggtaaataatatggaaattttaaaaatagcgatttttttgtgcgtgtttatttacgcgtttcggggtgatttggcggggttgtcgtcgggtgatttatatttttgaatcgcgaagcgatagggaaagcgcgggcgagcgtaggagacgcggtcgggggttttttcgggtttttgggttttcgtattcggagcgggggacgcggtcgttttaaggggaggaggggcggcgggttgtttttgttatttagcggcggtcggagcgttacgtgggcgcgcggcgtcgcggttattggttcgaggtacgtgtttaggagatcggtttgcgacgttattcgagggggttttgttaaaaataagaataaaaatttagagtgaaagtgttttaggttgcgtcgagtggtttggaaattttcgagttcgcgcggaggtcgaggcggcgagggcggcggacggtcggggagcgcgggcggtttagttcggttcggtcgggttttggtttcgcgttttttatttatgcgattcgggtcgcggagttttgcggggttcggcgggggcgcggtcgtacgtcggtggggcgtttcggttcgtagcggggcggcggtcgcgaggagggggtttttatgtgcgtgcgggcggtggcgggcgcgttgatcgcgggcgttcggtattttcgagggtcggttagggcgtgcgggcggggacggtcgggcggcggcggcggtcggagtcggttcgggcgggcgtgagcgtcggggaacgcgttgtttgtatgcgcgtagttttcgtttcgggcggtttaggcggcggcgtcggagttcgaggcggtcggacgcggagaggagcggggagttcgggaggcggttcgcgttttcgtcggattattgcgattgtttagatttcggttgcgcggcgaagtcgaggatttggttttgttgaattttttatcgtttgggcgagcggggcggttcgtggtgtttttaatttagttcgtggatttaaaggtggtttcgcgtcgagcgcggtcggcgatttgtaggattttagttttggtcgcggtcgtcgcgtacgttttcggaagattcggcggggtgggggcgcgggggttttcgtgtgcgtcgcgggagggtcgaaggttgatttggaagggcgttttcggagaattagtgtgggatttattgtgaatagtatggaggagaatgattttaagtttggcgaagtagcggcggcggtggagggatagcggtagtcggaatttagtttcggcggcggttcgggcggcggcggcggtagtagttcgggcgaagcggatatcgggcgtcggcgggttttgatgttgttcgcggttttgtaggcgttcggtaattattagtattcgtatcgtattattaatttttttatcgataatattttgcggttcgagttcggtcggcgaaaggacgcggggatttgttgtgcgggcgcgggaggaggaaggggcggcggagtcggcggcgaaggcggcgcgagcggtgcggagggaggcggcggcgcgggcggttcggagtagtttttgggttcgggttttcgagagtttcggtagaattcgttatgtgcgttcggcgcgggcgggtcgtttttagtcgtcggtagcgatttttcgggtgacggggaaggcggttttaagacgttttcgttgtacggtggcgttaagaaaggcggcgatttcggcggttttttggacgggtcgtttaaggttcgcggtttgggcggcggcgatttgtcggtgagttcggattcggatagttcgtaagtcggcgttaatttgggcgcgtagtttatgttttggtcggcgtgggtttattgtacgcgttattcggatcggttttttttaggtgagttcgcggggattacgcgtttcggttcgtcgcggggaggttcgcggagttggggggcggtgttggcgcgggaatttatcgggaggaaaatatttcgaatttttttcgcgtatacgtataaagatttacgcgatattgtgtgaagttgacgtcggttcgggtagcggttaggagtttagcggtaggattgattcgttagggggtatagattttttaggatcgtagaagggattttttttttttttttgttttttttttttttttttttttttttgttttttttttttgttttttatttcgttttggcgtatttttttttagtttttagtttatgttttttttattgtagttttttttggtgggaacgtggtggttggaagatgggttcggaagtgtatatttttatttttttttttacgattttttaatttaggttaggtcggggacgtatgttttagtttattttagatttgttttattattcggttatttcggttgtgttcggggaagaaaaggcgaggttttttgtcgttttgttttttgtttttcgggtttgcgttgatcggtgggatttaggaggatgtatatagggaaggaggaaaataaaggcgttttttttttttggttttattttgtttgttagcgttagttcgtagtggtggggtttagttttttttttgtatatagcgaggataagggaggtagtcgtttttttcggtatttgttatttttaaatagaaaggatttttttttagggttttttgggggttgttgatgggaaagaggtagtattcgtaggggttttgtagagatgttggatatatttttttatagatttgcgattttaaaaaattaagtttatgtttttgtagaaattattaattgtattttatgcgggtttgcggttgggaatcgttattagaagtggattgtttgatttcgagttggtagcggattttcgttgtttttaaatttttaattattttgcgggggttatttgtttagattatagtaggagtgagttaatttttgggtcgttatttcgtagaattatgcgtgtatatttttgatgaaatttagattttttagttagatttgaaatttgttttattgttttcgttttttttttttgttaatatttaattaatatataggtttataatgtcgggcgaggagattcggtcgggttttgtgcggcgcgggagttcgttgagttagtttttaacggttcgggagttgggtagtatcgttcggttcggtttggttcggtttagtttagtttagtttaagtcgtttatttttatgggttttaaaatatttttgtaagataatgtttttgttttttggttttttcgaaagaaaggggagagagagtttttttggggaggtttgattttgtttttgagatttttaagtatttgttttttgaaagaaaattaagaaaaaaatttaaaaattattattttagggaaatttattgttataaaatggtgtttttttgcgggttgttttatgagtgtattaataagagttttaggattagaagagtttgggggtagagttttggggaagggagtggttggaaatttagatagagatgggttttgggagtaggaggttggggttttttttggagttttgtgttttattttttattatcgtttcggagggttaattttatttttaaatttgtatttatttttattaaagttaggtttattggtttggagttttgggcgtgagtaagataggtattgagtgtgtacgtgtgtatggggtgggtgtttaagtatagggtgtgtgtttttatgggtggtgagtttgtttatgggttgttttaaaagttgtttttggtgtttttgaggtggtgtttatagattttttttttttaggtttgttttttggagagagtataagatttatttggttatgagggagtgtttggtatttattttgggtttttagttcgttttttattttttgttgggtatagttttagtattttagttgatttttttgatttgggtagggtgtagttttagggtttttaaggagatttatattttttttttttttagtgtgttcggtagtttttcggttttgaagggtggggggtttttagttttttttagttatagggatttgtgatgaagttggggttagatgttttttaaagtcgatttatatatcgtataaattgaaatttagaggcgaggttattattttttgttagtggttttgtttttttttttttttatagggaacgttagggggttgagttttttattattaaaaagaaattgatgatatttttttttttttgtttttttttttttgttttttttttatggatagtaggttttagaagttttatagcgattttgtttaaaatttggggtaggtttatagggagaaggttaggttaggtttataagtttgaattttagttgggaggtatagtggggagggttagaagtggatttggataaggttagttgggttattttgttgtttatagtgaagtagttttatgtttggggaaagggtggtgtagttaatatttttgtagagttaggtttttttttttggataggaaatttgggagatttttagtgggtgaaggatttatttattgtgagtagtttagtgtttttttttattaaggagggaagtatatgtattgatttttttttaaaggaatgaatttgggtttatagagtttttggttgggagttatagaggagtttgggtggaggtagatattttgggttttttttgtttttagggtttatttgtttttgatttttatagtttttggtattttgtggggtatttttatgagggtttttattatagtttttagggcgttttttgtttttgtgatcgttttgtagttttttttagtttttttttttttttttttttttagtatttatcgttatttttgtttttgaatagagagttttagaaaggattaggaaaaattaggttagaaagtgtggggagttttgtttatatttaggagttttatttttatttagagattttttatttgtggttagtttgtttattaggtttgttttatagttttatttatattatacgtagtttttttttattaagtggtggaggttcgcgttgagtttatgtttagttcgaagtttagttttatatttggcggtttagtttcgagtggttttgggcgagttattttttttttggggttttagaatttttattcggtgtttattgggtggtatatttttgggtaatttgatttttttttgtttatcgtatttatttaggttttaggtttcgaaaattaaagaagaagaattcgaataaagaggataagcggtcgcgtacggtttttatcgtcgagtagttgtagaggtttaaggtcgagttttagattaataggtatttgacggagtagcggcgttagagtttggcgtaggagttgagttttaacgagttatagattaagatttggttttagaataagcgcgttaagattaagaaggttacgggtaataagaatacgttggtcgtgtattttatggtatagggtttgtataattattttattatagttaaggagggtaagtcggatagcgagtagggcggggggtatggaggttaggttttagttcgcgttaaataatgtaataatttaaaattataaagggttagtgtataaagattatattagtattaatagtgaaaatattgtgtattagttaaggttttgaaatattttatgtatatattatttataggtggtataaaatttaaaatatttgattataaaatatttttttgagttttttgtgtttatgagattatgttaattttatgggttttttttttttttgcgaagggggttgtttagggttttatttttttttaattttttaagttttattatatgatattggatatttttttattattttaaaagaagaaaaaattaaaataatttgttgaagtttaaagattttttattgttgtattttatataattgtgaatcgaataaatagtttttatttggtttatgatttttgttattttgtttgtgttggtttggtgaggatagtaggaggggtttatattttaagtttggattagttattttaaggttttggggagtttaggggatttggtgggagagaggggatttttagggtttttgggttagttttgggatttggttttgggaagtagtttagcgtattttaggtttgttttgggaagtcggttttatgtttattagtagtcgtttaggttcgtagttttattcggttttttttttttatttttttgtatttaatttttttttttttttttttttttttttttttttttttttttttttttttttgttttttttttttttttttttttttttttttttttttttttttttttttttttttttttttttttttttttttttttattaagggtttaatcgtgtgtatatatcgtttgcgtttgtggtttgtgtcgttgtttttagttttatcgtagttttgtcgtaggtttaatttttttgttttgggtattgtttttatgtagaagcgtttcgaggttttggggttaaaggtttggggtgtgtggtttaaagtttaagagcggtggggcgattttttttttggtttggttttaggaattttttgtgattttattagttattatgggtgttagttagggttttagaaatgaggttatggtttattgtttttgggcgggtagaaggttttgtagagggagatggtattatttattttttttttttttttttttttttttttattttttttttttttttttttatttttttttttttttggagtggttgtttttgttatagagaatatttttttaagataaatatgtgtgtttatatatatgtttgtatgtatgtgaatatatatatatatatatatatattaggcgtgtttgagtttatagttttgaaatatgtggttattttgttttttaaaagaatttagaattttttaggatttagaagaaggaagaaagtgtgtaaataattattttttattattattttttgtttttttttgttttttaaaatatatattttatttttgaaggtgtggtatagtgtaaattaaatatatttaatatattttttattaagtatttatatatgtatataaataaatatattatttatatataacgttat

SEQ ID NO: 13 - DNA, 16197 bp, Artificial Sequence
  chemically treated genomic DNA (Homo sapiens)
gtggcgttatatatagataatgtgtttgtttatatatatatataggtatttggtgggaaatatattgaatatatttaatttatattgtattatatttttaaaaataaaatgtatattttaaaaaataagaaaagataaaaagtgatgataagaaatgattatttatatattttttttttttttttagattttggaggattttgagtttttttgaaagataaggtagttatatgttttagaattgtggatttaaatacgtttggtgtgtgtgtgtgtgtgtgtgtgtttatatgtatgtagatatatgtgtaaatatatatatttattttggaagaatgttttttatagtagaagtagttattttaagaaaagaaaaaaataaaggaaaaaaagaaaaaaatagggaagaaaagaaaaaggaaaggaagatagatgatgttatttttttttatagagttttttgttcgtttagaaatagtgagttatggttttatttttgggattttggttggtatttatgatggttggtggagttataggaaatttttggggttaagttaaaaggagggtcgttttatcgtttttgggttttaggttatatattttaggtttttagttttagaatttcgaagcgtttttgtatggaggtagtgtttagggtaggagggttaggtttgcggtaggattgcggtgggattggggatagcgatatagattatagacgtagacgatgtatgtatacggttgggtttttggtgaggaggaggaggaaagagaaggaggaggaggaaggaaggaggaggaggagaagaaaaagaagaagaaaggaggagtaggaggaaggaggaaggaggaagaggaggaaaaaggagaaggaggagggagttaggtgtaggagggtgaggagagggagtcgggtgaggttgcgggtttgggcggttgttggtgagtatggagtcgattttttagagtaggtttggggtacgttgggttgttttttagggttaaattttagaattggtttaaggattttggaagtttttttttttttattaggttttttaagttttttaaggttttgaggtggttggtttaggtttgaggtgtgggttttttttgttgtttttattaagttaatataaataaagtggtagaagttatagattaaataggagttatttattcggtttatagttgtgtgaaatgtagtaataaaaaatttttggattttagtaagttgttttaattttttttttttttggaataataaaaaagtgtttaatgttatataatggagtttaggggattaaaaaaaggtgaaattttaagtagtttttttcgtaaaaaagaaaaaaatttataaaattagtataattttataaatataaaaaatttaaaaaaatattttatagttagatattttggattttatattatttgtaaatgatatatatatagaatattttagaattttagttaatatataatatttttattattaatgttggtataatttttatatattggttttttatgattttaaattattgtattgtttagcgcggattgagatttggtttttatgtttttcgttttattcgttgttcgatttgttttttttggttgtggtggagtggttgtataagttttgtgttatgaggtgtacggttagcgtgtttttgttgttcgtggtttttttgattttggcgcgtttgttttggaattaaattttgatttgtgattcgttgaggtttagtttttgcgttaggttttggcgtcgttgtttcgttaggtatttgttggtttggaattcggttttgagtttttgtagttgttcggcggtaaaggtcgtgcgcggtcgtttgttttttttgttcgggttttttttttttggttttcgagatttgggatttgggtagatacggtggatagagagaagttaggttatttagaggtgtgttatttaataggtatcgggtaaggattttggggttttaagagggaagtgattcgtttaaggttattcgaggttgaatcgttagatgtggggttagatttcggattgggtatgggtttaacgcggatttttattatttggtgagggaggattgcgtgtgatgtaagtgggattatggggtaggtttagtgggtaggttgattataaatgaggggtttttagataaaagtaaaatttttggatgtaagtaaaattttttatattttttggtttggttttttttagttttttttgaggttttttgtttaggaatagggatggcggtaggtgttgagagagaggggaggagagaagattgggaaaggttgtagggcggttatagaagtaggaggcgttttgagggttatgatggggatttttatggggatattttatagggtgttaagggttgtgggaattaggggtaggtgggttttgaggataggaggggtttagagtgtttgtttttatttaagtttttttatgatttttagttaagggttttgtggatttaagtttatttttttaaggaagagttagtgtatgtatttttttttttggtggggaggggtattgagttatttatagtgggtgagttttttatttattggaaattttttaggttttttgtttaggagaggggatttggttttataagggtgttgattgtattattttttttttagatatgggattgttttattgtgggtagtagggtagtttagttgattttgtttaggtttatttttgattttttttattgtgttttttaattgggatttagatttatgaatttgatttggttttttttttgtggatttgttttaggttttggatagggtcgttgtaaggtttttaggatttgttatttatggggaaagggtagagggaggagagtagaaggagggaagtgttattagtttttttttggtgataagaggtttaattttttggcgttttttgtgggggaagaagggggtaaggttattggtagggagtggtgatttcgtttttgggttttaatttgtgcggtgtatgaatcggttttagggagtatttggttttagttttattataggtttttgtggttggagaaggttgggggttttttattttttagggtcggagagttgtcgggtatattgaggagaagaggaatgtggatttttttggaggttttgggattgtattttgtttaggttaggagagttaattagggtgttggggttgtgtttagtaagaggtgggggacgaattggaggtttagggtgaatgttaagtatttttttatggttaagtgagttttgtgttttttttaggagataggtttggagaaagaggatttgtgggtattattttaggagtattaagggtagtttttaaagtaatttataggtaggtttattatttatgagagtatatattttatatttgggtatttattttatgtatacgtatatatttagtgtttgttttgtttacgtttagggttttaggttagtgggtttggttttggtaggggtaggtgtagatttggagatggggttggtttttcggaacggtggtgggggatggggtataaggttttaggagaggttttagttttttgtttttagagtttatttttgtttgggtttttaattatttttttttttagaattttatttttaaatttttttggttttggggtttttgttaatgtatttatgaagtagttcgtaaaaggatattattttatggtaataaatttttttaaaataataatttttaggttttttttttgatttttttttaaaagataaatatttaggagttttagaaatagaattaaatttttttaagaaaatttttttttttttttttttcggagaaattaaagaatagaaatattattttgtagggatattttaaagtttatgaagataggcgatttgggttgggttgagttgggtcgggttaggtcgggtcgggcggtgttgtttagttttcgggtcgttgggggttggtttagcgaattttcgcgtcgtataaagttcggtcgagttttttcgttcggtattgtgagtttgtatgttaattaaatattaataaaggaggaagacgagaataatggagtaaattttagatttagttgagaaatttgaattttattagaaatatgtacgtatagttttgcgggatggcggtttaagggttggtttatttttgttgtgatttgggtaaatgattttcgtaaaatagttgagggtttgggggtagcggggattcgttgttagttcggggttaaatagtttatttttaatggcggtttttagtcgtagattcgtatggaatgtagttgatgatttttataaggatatggatttagttttttaaaatcgtagatttatgaaaaaatatatttagtatttttgtagggtttttgcgaatgttgttttttttttattagtagtttttaggaaattttgagagaaggttttttttatttggggatggtaggtgtcgggagggacggttgttttttttgttttcgttgtgtgtaggaagggggttgagttttattattgcgggttggcgttggtaaataaagtggagttaaggggaaagggcgtttttatttttttttttttttgtgtgtatttttttgagttttatcggttagcgtaggttcgaggggtagaaggtagagcggtaaagggtttcgttttttttttttcgagtatagtcgggataatcggatggtaggataagtttggggtgggttggggtatgcgttttcggtttggtttgagttggaagatcgtagggaaggggatgagagtgtgtattttcgggtttattttttagttattacgtttttattaaaaaaaattatagtgggggagatatgggttaggggttgggaagagatgcgttaaggcggggtggaagatagagaggggagatagggagagaggaaggagagagagagatagagaaagaaagaaggttttttttgcggttttaagaagtttgtattttttagcgaattagttttgtcgttggatttttggtcgttgttcgggtcggcgttagttttatataatgtcgcgtgagtttttgtgcgtgtgcgcgggggaggttcgagatgtttttttttcggtaagttttcgcgttagtatcgttttttagtttcgcgggttttttcgcggcgagtcgggacgcgtggttttcgcgggtttatttgaagaaggtcggttcgagtagcgcgtatagtagatttacgtcggttagagtatgggttgcgcgtttaggttggcgtcggtttgcgagttgttcgagttcgagtttatcgataggtcgtcgtcgtttaagtcgcgggttttgagcgattcgtttagggggtcgtcggggtcgtcgttttttttggcgttatcgtgtagcgagagcgttttggagtcgtttttttcgttattcggagagtcgttgtcggcggttgggagcggttcgttcgcgtcgggcgtatatggcgggttttgtcggggttttcgggagttcgagtttaagagttgtttcgagtcgttcgcgtcgtcgttttttttcgtatcgttcgcgtcgttttcgtcgtcggtttcgtcgttttttttttttttcgcgttcgtatagtaggttttcgcgttttttcgtcggtcgaattcgggtcgtaggatgttgtcgatgaagaagttggtgatgcggtgcgggtgttggtggttgtcgggcgtttgtaggatcgcgggtagtattagagttcgtcggcgttcggtgttcgtttcgttcgggttgttatcgtcgtcgtcgttcgagtcgtcgtcggggttggatttcggttgtcgttgtttttttatcgtcgtcgttgtttcgttaggtttggggttatttttttttatgttgtttatagtaaattttatattggtttttcggggacgtttttttaaattagttttcggttttttcgcggcgtatacggagattttcgcgtttttatttcgtcgagtttttcgagggcgtgcgcggcggtcgcggttagggttgaggttttataagtcgtcggtcgcgttcggcgcggagttatttttgaatttacgaattgggttagaaatattacgagtcgtttcgttcgtttagacgatgagagatttaatagagttaagttttcgatttcgtcgcgtagtcggggtttagatagtcgtagtggttcggcggggacgcgggtcgtttttcgggtttttcgttttttttcgcgttcggtcgtttcgggtttcggcgtcgtcgtttgggtcgttcggggcgagagttgcgcgtatgtaggtagcgcgtttttcggcgtttacgttcgttcgggtcggtttcggtcgtcgtcgtcgttcggtcgttttcgttcgtacgttttagtcggttttcgagggtgtcgggcgttcgcggttagcgcgttcgttatcgttcgtacgtatatggaggtttttttttcgcggtcgtcgtttcgttgcgggtcggggcgttttatcggcgtgcggtcgcgttttcgtcgagtttcgtagagtttcgcggttcgagtcgtatgggtgagagacgcgaggttagggttcggtcgggtcgggttgggtcgttcgcgtttttcggtcgttcgtcgttttcgtcgtttcggttttcgcgcgggttcggaaatttttaggttattcggcgtaatttgagatatttttattttggatttttgtttttatttttaatagagttttttcgagtgacgtcgtaggtcggttttttggatacgtgtttcgggttaatggtcgcggcgtcgcgcgtttacgtgacgtttcggtcgtcgttgggtgataggagtagttcgtcgttttttttttttttaaagcggtcgcgtttttcgtttcgggtgcgggagtttaggaattcggagagattttcgatcgcgttttttgcgttcgttcgcgttttttttgtcgtttcgcggtttaggggtgtgagttattcggcgatagtttcgttaggttatttcggggcgcgtaggtggatacgtataggaagatcgttatttttaagatttttatattgtttatcgtggggcgaaattttataattttttgtataaaatgtataaataatattaaatgagttgtcgagatataaagggatttttgtggttcgttttttttgttgatggagaggaatagtgttatttttatttgattcggtaattcggtagttttttgttttaaacgtagagttggcgcgtaggatattttttttgtagatatttattgtaagtgtgcgtgtgtgtgcgtgtgtagggaggcgcgtgtatacggtcgtattttattcggggatagcgatttttatgaaatatgtttagcgattcgaaagagcgggggaatgtagtatgcggttttatttgcgaagggagaaatttaggagtttcggaggttttaaaggaattttttttgttaaggagaggaggggtgattgtttagttaagattttaaaattaagtcgttcgtttttttttaatttttttattttgattgaggagtttttagatcggttatttcgttttttttgttttaaaattttttaaattaattttgtcggagagtagggtttatttgaaacgagaattaggaatttaacggtaagggtgggagttttagttggtttttttttttttttttttttattttttttagattttttttttagttgttgttagaatgtttaattggaagttttgtttttttttttttttttttttttttttcgggtttttttttatttgtttttgtttatttattaaatattcgttgttattttaggttcgtttgtttattatttaaaatgtatttttatggaaatgtgtgaatttttggaaatattcgatagggtagttttgggggtgcgggagtatttgtacgttatgagtatttttagtgcgggggaggcggggaggtcgtagtttggtacggatattttagggttgattggttttgggtagttgtatcgtgtttagtaatttagattttcgtagaggtagttagtaggttgttgttaggttttgagtgaatttggggagtttggtaagtattttcgaggtagcgtggtttgttaggttatttggggtttgaaggttgtattttggttttgcgaagttttttttttagtgtttattttacgtgttgttttttttttgtaggttttagagtgttggttgggttgggatgagaaacgatagtttttatagttgtttttaggaaaattatttttttatttaggacgggaatggtgggggaggaggtgttgggttggttttttggttattggacgtggttttggttagagtgtgttttttgttcggtgtgtttagggtagggtggggaagttttttttcgttggggttggatttttttattttttttttattggaggagtttttattaaatgttttggttaggtatatatttgttaggggtttttttcgagttttaaaaaattaaattttatagtttatttgtttttcgggaaaatagggttggtgagagtttgtgggaagttttattgacgtttatttggttttttggggggagaattttttgtaattttggaaattttgatgattattagttaggaaagggagggtttaacgattttgggttttttgaatatgatattggggggaggaggggttttgagttttttggtttttgtaaaattattttaggttaatggttttatttgttttatgaaagttaattaggcggttttttgagaagtattttttgaatttttgggattttttgtttttgattgttttagtataaatggttttaagacgttgggttttagttttgtgtttttttgcgatttagattttaattcgtggttgtaatagagattttgggaagtagagcgttatttttagggcgtttggcgggtcggtgggtagagatttgttttcgtagggtttgcgggttgggttttggggttggttggaggaagaggaattcgtgggtcgtttttttggaaaggaggaaggtgagaggtggagggaggaataggagaaagagaaataaatgaagagagagataattagagggtatcgggttttttagagtggatttaaatacgaaataattttgggaaagcggtaagaggggttatcgtttaaagggtcggggaagttgggatggggttgtagaaagttgagttgtttaggggttgggcggtgttgagttttatatatacggaatttcgtaaggtgttttaggaagtttcgagaagcgcgtatgaggatcggtttcggtcgtttgttttagttgggacggcgtttcgcgttttttggggatgcggggtagtggcgtgttttttttttgcgatgttagtttcggttgtcgcgttttcgcgagtttagagttttcgttttttttttcgaacgatcgtcgtttttaaggtcggtttttttgtgcggattagacgagtttttttcggatattagtttttttcgggatggaaagtggggtattgattcgggaatttcgttttttttttagttttatttaggaatcggagagcgttcgcgattattcgtttgcggaggacggttttttatattttttaggttcgttgtataaagaggggtttcggagagggcgagtaggaaagtagtttttagacgtcgtgcgttttaggatacggacgttagttttgtaaataaaattttagtgaagcgtttcggagatatcgggcgttcggatgtcgagttaggttttcgtttttatttgaaaacgaggttagagaagggtgattttttcggtttttttgcgttttttttattcgtttcgtttttttagtttgacgtggtgatatttggaaaaatggatttaatgaaagtcgttcgttcgcggcggtttcgttattcgcggggtagagacgtttaataatttttgagagcgagatttttattttagttaaagtaggtttggttagttttgcgttttcggttgggagtcgagtttcgtagttaagttttaggcgtcgggcggggaattggggtcgataggagagtaatatttttttcggggcgagtagagagttatcgtcggtttttggggttgacgtttcgattcggtttgtagataaaggtcgaaacgaaggggcgcggggagtaggggtggggcgggcggggttaacgggcggatattgcgtgtttgcgtttttttttgggcgaaggaggatcggtttataggtttatcgggcgagttcggggacggggtttgggaggttgggtttagggtttgagatttttttagaataattttcggaaggagggggttaaatgcggtagcggcgggtttgaaaagggttaggagcgtcgtaggaattggggtgtataggttttcgcgtgtttagcgtttcgttttttttcgttttaggtttgttaggggcgcgcgtggcgcgggtagagtttggtgggtgggttggcgcggatagaagttttgtcggggagcggggaggggggggtagtcgtattgtacgttttggttttgtttcgatcgttggagaagcgtttgtgttgagtttaagttttttaggtgttggggaagtagagtgggttaggagtcgattggggatcgtttttttgttttgatttgatatttagtttaggttcgttcggggaggggggcggttgggggtaggtgggtagatttttagagttggcgtgtaaagatgtagaaattgttttttatttggtttttttggaaagtagttgtttttgaaggagaatttgaaggatggttttatttgtgttagtaatttttatatttaaattaggttttttgtgttcgttttttttttcggttttttagcgttaggggagttttattttgggtgatttgtttttttaagaatcgtttttttttggtagttttttgtttgtttttttgttttgttttattgttgtttttatttttataaaatttggatttttgtggaaggtgggtttatttttatttaaggtttatttttgagattttttagatagatttgtattttggagtttttcgggagttttgggaggggtaggtttgggagtatagattgttttattatttaatgtttggatttttatgaaaatgtttttttaaaattatagtaatttagaagaaagtggtattttttaaataatttagttaaataagtaaaattagtttggtaaaggaatttgaagaatggtatttaaggtttgagttggttaagtttattaaaataggaaggaaaatatagaaaaggataatgagttttgaagtatattagttattttttttagttttgcgattatttatagatggaagaaattttagtatgtatttattgttttttttttgatttttggaaagaaaataagatgtttaaaaggaatttttgaaatattagttttattttatagaggttgatattttttttttttttattttttttttaaaggttataagtaattatagagaggttagtgtagtttaaatttttatatagtgattttttagtatttttggaaataggatttgcgtagttagaggtttatagaattttaggtaagtataatttagatttaatttaaattatttggatttagtttagttaaattatttatagtttgttagatgataatttattagtttaggttttacgtgaggaagaaaatatttaaataattattttcgtttttaaatattataattgaattatgtttaattaggagaatttaattagtttatatattggttaagttgggggaaatatatttttaaaaatatatgtttaagttaaagttatgaattgatttttttttttatttattaaatataaataaagatatatagttttatataaaaaggtaattaaattgcgaattaaatttaaatattttttttatttagttaaatgattgattagtttttttgagtttaataaagttggtcgtttttttagtacgagtatttttaattgatatatatgtttatatgtatggtatggggggatttttgtatttaattaatttttgtgttgagtatagaagtttgtgtgaagcgtggcgattttttataagtataaagttttagtagtgattttcgtggatttatacgcggaggggtattgtgttatttttgtattatggatttttgtttttagttttgtttaaggtttaaattattttttcgggggatgtcgtttagttttatttttattagggatggggatttgttgtttatttttggacgtatttttatttttttttttttattttagtatttttggttttatttaaagaaaagtttagtagaataagattttttagttagggtatattatagaagtgttttgttttttttttaaaaaatttttttttttttttcggaaatttatttaaatattttagttttttttagaaaggttgtgttatagtttatgttatggagttgtagggttttgtgtgttttatattagttaattttttaagatataatagtttttgtgatgacggtaaagttgagtaattcgatattatttggagtattaatataggttgggaggaagaaacgggatacgcgcgaagattttttacgtttggagcgaatgttttagaatttttcgggatttatattttttcgtttattaggaaggagaaatgcgagttgtttttatttattgtttgtttttagttttttgttcgtattgttagggttaattttggtcgcggcgtaatatttgggttatagatgttagggggtcgcgtttagggggaagcgggattttgagttttgttagtttcggttagaaagtttggtattaaggcgttgggtacgtttttaggataaagtgcgggttttttaaaagaggaaggtcgggcggtatttagggttttttgttttgtttgtagatataagtttatttgtatttcgtgtaaagtagttttgaaagtttaggtttgaagataaaatgtggagtttaggttttttcgattgaaaatttttttacgaggatatttttcgtagtgggcggttagaaaggatttcggattaagattcgtagagtggtgagtattttttagggttcggggttgaattgattgtaggttggtagtttttcggttcggaggagaattttttttttgtagaaattatttttggaaaataaaatcgagcgaaaagaagggtaatgcgggtttgcgcggaagggagtttttttttcggggaaggaggcgtttcgcggtggtatattcgtcggcgggatgtaggtaggggtagattagggtgagaagggggatttgcggaatacgggtttttcgcgggttcgagggatagggtgtaggcgtattttaggtatttagggtagtttagtttgttttgcggagcgggagtcggttttaggttttttatattttttaggaatttagaagttcgattgcgggatagttgtgtcgatggggatttttttagttggatttttattttatttgtttttttgatcgagtgtttacgttcggtaggagttggttgtgcgtcgtttcgggtgggtggaggtgcgtggggttcgggttttggttcgtttgggtgtgttttagttttttttttttagaattaaaggtttttgtagggcgtagagttttttttttttttttttaatttttttttttatttcgtcgcggtttaagttaagatgaggtgagtttttagggttgtgtttttaggaggtttcgagtatttttttttcgagggtcgaagaaattaggttttttttttttttttagcgttttttgaggaagtcgcgaatttttgagcgagcgtggttttattggggatttttagcgatttgttttgtgtttagcgacgttttaaggggagaggagttttgtggaggggcggtgtgggggtgggtgtttatattatattttttggttcggagtaggattcggtgatgcgttttggtcgagtttttgttttttaggaatgcgggtgtttcgggttttgtcgggatttttggttcgatcggagggttcgggagtgggagtttggatttaataggtattcggatttaggagttagattttttggtttcgaacgtttttttcggagcgaaagtttgttttttttttttttaagtttttcgcgggttgggaggtgaagggagcgatagaagtcgggtttttttcgtagcgtttaacgtttcgggtttttcgggtttcgggacgtggttcgggtgcgtggagggacggttgggggcgtagatttgtgaatgaatttcggagttttttgtagtttatagattagaggttgcgatcgttttatcgacgtggtttaatgagtcgtcgtaataagttttgagcgacgaaacggaggtacggtttggattttgcggtatcgcgttgcgtagggtcggacgggggaggggttgagttattgtagtaattttaaagtttgtgtcggggtttagtaatgggtgggtttttttcgggtcgtacggtttttaagggtttttttcgatttttatcgttatttcgtcggaacgtttagaggcgtttttagtttaagtgatggagattttgatttttagttgttttcgcgtttttcgtttgcgcgtttttggcgtttttttcgggattcggggggcgattatattttgagatttaattcggttattttcgggcgtgggtttttatcgtttttcgggttggttggattcgttttttaagtagttgttttatttattattcgtcgttagttttttttttaaaatttaagttttcggttgtttttttgtcgtttttcgtttttcgtttttcgtttttcgtgcgaggggaggggagggggaggaaggagcggatatttatgtgtgttcgggaggggttcgtatcgttttttcgtttttttagtagaggttttgggttttttttttagagggaaatcggtgagtagatattaagtgacgcgagtttcggtttttttttcgtcgggggattttcgtgcgttttagaagcgtatttggaagtaatttttttcgcgtaatcgtcgttttttaagtatttttagtatttttatattttttaaatatttgtataattttgcgttgttttttggaggatttcggagacggggaggggacgatggggtgtcgtgggggtagggaaggagagaggttaggggttacgtaggattacggttcggttcggtgattttttttatttttcgtggtttagggaggggatttgtagcgagggttttagattttttttgtaaaatcgaattttgggggagggtttgcgttttatgaagtacggggaaggtttaaatgacgaatatttacgagcggtcgggagcggaagatggtaggatcgacgggttcgggagggaagatgggcgtgcgtttttagtgattatggtacgaggcggcgttttgtttcgtattacgttataatttaattaggttttcgttttttcgcgtttgaatagggcgggtttattcgggcggcgagttcgagtttagcgtagttttagttcggcgtttttttttcgtttgttggggaagaggtgggaaatttttttcgtcgcgttttggtttttatcgtaggttttcgagaataaatttttttagggagattgaattgtttttattttttgttttagttttaagagggattttttgaaaatatggtagttaagaatagggtgaaaatgtaagtttgaggtttttttttttatttttttttgttttttgtttttttatttttatattttttattagtttagaggggtttagttgtaattattattatcgttatttttacggtgttgacgattgttattatttttttttagttttagttgttttttggatgtttttattggaaggttgacgagtatttttaattttacgtgtttattttagagtattttcgatttcgttatttttgttgttttgatttgcgtttttttagttttttttagttatttagttggttattttttaggagatatttttgattttttattttttttttagagttattttttgttttttagtaaggtttgttggttttgtttttacgttttttttatagtttttttatatttttgcgttttttattagtttttaagttttttttttttttatttgtaggg

SEQ ID NO: 14 - DNA, 2609 bp, Artificial Sequence
  chemically treated genomic DNA (Homo sapiens)
cgtagattagagatgattatagattttttttagcgcggattaaagggattgaattgaacgttttagtttaatgatttaatttttgttatatttatagggacgcgaatttcgattttataagtaggtgtgtacgtgtatttagatatttatataaagtcgcgtggagggacgaaaagattaattattcgatcgacgaggataggtttgattttttcgattattttagtgtgttagtgtatattttcggttgggtttagcgttttaagaaatttcggaattttagttgttaatttttgtttttttattatgattttttaaagatattttatttgtttatcggggcgaagagaaatgggattaggcgttagggcggtgggattttgtttagggttttgatttcgcgttagggttttagattagtcggttttcgaaggtttttttatttattttatataagaggaaataaagattttttagtttaaggttttagggttgttttttgatttcggttagtttgtaggaagaggaaataataaaataaaggaatcgttaattcgtcgggtattatattttttagttttaattttcgatttcggatcgttagggttattttttttacgttgatttcgttttttttaaatgaaaatacgttaataaaagtatatttcggatataaaatttaagtacgtattttttttggggaggttagagttgaggtgtatttcggaagatgagaattttgtttttatgaattgggtaatatttaggtattgttaggtatttcgatagattttttagatattttttttttttttttttatattttttttttattaaaatagttattgttttgaaatttattagaataacgacgttttaaaaataaaggcgtagtaagtattttttttttcgttgtcgcgggttgaattacggacgttcgcgggtcgtttagtttcgacggttcgtagggggcgcgcgtcgtagtcgtagtatagttcggttatttttagaaagggagtcgaatggagggaagtagggagcgcggagggttcgaggtttgtagataaggagaggcgtattttgggatttgggttttttgttgttataatataatcgtgttattgttggtattgttcgatttaagtgtcggtggtaagcggcgatgtcggggttgggtttttagtaatcgttgtgttttgggttaggttggtcgttttagttatcgggattttttttcggatgtttttagggcgataggtgttgtatttattgacgggatagtcgtatattttcgaataggtagtggagttcgtttttggtaggtattttagttgcgttgatattaaggtcgttatataatagttattagttttttttaagttttagaagtaggttttttttgttttagttatagcggttttagtcgtcgttacgagtttattttttatttttaagcgtatttttttttttttattcgggtttatgttcgttatatagagagaattatatagggggaattatggttttatattttcgaggggatagatatcggtcgtgagataggtattacgtagagttttttggtgattgttcgtaggagcgagattttttttggttttgtagtcggttaggtgtgtgtgtgtgagggtttttagttgattatcgggatgtattgttatttttcggtttggcgggttttgggattttttggttttcgtaggaggttattttaggttttcggaggaggcgtttttagttggcggcgtttttcgtttcgggtttagaggcggatacggtcggtcgcgttttgttggttttttgttcgcgtcgtagcgggttgggagtagttgcgcgatattagatttatagcgttaagacgcgaagcgcgaggaaatcgttgcgtttgatttttttttttttaatttttggatttaggaacgtttttagtttttgcgttttatacgtttagttttggtttttttttcggtttagaagtttttaaggattgaagggttttgtttagggtttcgtatttttgtttgatttttatggtttagaaaagtagggggatatttgaaatgttatttcgggataaatattaataaaaaagtaaatggatttgtgtaggggttagttattaattaattaggtcgtaaggttatttagggtaaatatagtttaggttgggttgggcgagatttttttatggtcgtttttcgatcgaatttttttttttttggtttaggttttttttaggtcgttttggggtaaatatcggacgggaagggggcgtcgttaattttttcgcggggtttggaggttttttcgtttttaagtttcgtagggtagggtcggagtgtttaatatttattttcgttcgaatttcgggtttgcgcgtcgtttttttttgggttcgcggtgttgcgtttgttgttgggtgtgtgtcgttgttttttttcgagtcggtagtttttgttgtgtggtcgaagttttttggaatttttaattggaaattaattttggttttgatagacgtttacgttagaggcgcgttatttatttatatttttcgttttatttcggaggagacgcggcgagaattttgtcgt

SEQ ID NO: 15 - DNA, 2609 bp, Artificial Sequence
  chemically treated genomic DNA (Homo sapiens)
gcgatagggttttcgtcgcgtttttttcgggatgaggcggggggtgtgggtgggtggcgcgtttttgacgtgggcgtttattaagattaagattagtttttaattaaagattttagagggtttcggttatatagtaggagttgtcggttcggaaagagatagcgatatatatttaatagtaagcgtagtatcgcggatttaggggaaggcggcgcgtaggttcgaggttcgggcgggggtgggtgttgggtatttcgattttgttttgcgggatttggaggcgaggaaatttttaaatttcgcgggggagttggcggcgtttttttttcgttcggtgtttgttttagaacgatttaagaaggatttgagttaaggggggaggggttcggtcggggggcggttatgagaaagtttcgtttagtttaatttgggttgtgtttgttttgagtggttttgcggtttggttgattgataattggtttttgtataagtttatttgtttttttgttaatatttatttcggggtgatattttaaatgtttttttgtttttttgggttatgggagttaggtaggagtgcggggttttaggtagagttttttaatttttgaagatttttgggtcgggagagaagttaggattgggcgtgtgggacgtaggagttggaagcgtttttgggtttaagagttgggggagaaggagttaagcgtagcggttttttcgcgtttcgcgttttggcgttgtgggtttggtgtcgcgtagttgtttttagttcgttgcggcgcgaataaagggttagtagggcgcgatcgatcgtgttcgtttttgagttcggggcgaggggcgtcgttagttgggagcgtttttttcgaaggtttaggatggttttttacggagattaaggagttttagagttcgttaggtcgaaaagtggtagtgtatttcggtagttaattggggatttttatatatatatatttagtcgattgtaaaattaaaaaaggtttcgtttttgcggatagttattagaaggttttgcgtggtgtttgttttacggtcggtgtttgttttttcgagggtgtaggattatagttttttttgtgtagttttttttgtgtaacgggtataaattcgggtgaggagagggaggtgcgtttggggataagaagtgggttcgtggcgacgattggaatcgttatggttggggtaggggaaatttatttttggggtttaaggggggttgatggttattgtgtggcgattttggtgttagcgtagttaggatgtttgttagagacgaattttattatttgttcggaggtatgcggttatttcgttaatgggtgtagtatttatcgttttggaagtattcggagagggatttcggtagttggggcgattagtttgatttagggtatagcggttgttagagatttagtttcgatatcgtcgtttattatcgatatttgggtcggatagtgttaataatagtacggttgtgttgtagtagtagaggatttaaattttaggatgcgtttttttttatttgtaagtttcgagtttttcgcgttttttgtttttttttattcggttttttttttgggggtagtcgggttgtgttgcggttgcggcgcgcgttttttgcgggtcgtcggggttgggcgattcgcgagcgttcgtggtttagttcgcggtagcgaagaaagggatgtttgttgcgtttttgtttttagaacgtcgttgttttagtgaattttaaaataataattattttgataggaagaaagtgtgaaggaggaagagaaggatatttagagggtttgtcggggtatttaataatgtttgggtgttatttaatttatgaaaataaaatttttatttttcgaagtatattttagttttaatttttttaaaaaagatgcgtatttggattttgtattcgaagtatgtttttgttggcgtgtttttatttaagaaaagcggggttagcgtagagaaggtgattttggcggttcgaagtcggaggttggagttgagggatataatattcggcggattggcggtttttttgttttgttgttttttttttttgtaaattgatcggagttaggaggtagttttgaggttttgagttgagaagtttttgtttttttttgtgtggggtgggtgaaagggttttcggaggtcgattggtttggagttttggcgcggagttaggattttggatagggttttatcgttttgacgtttggttttattttttttcgtttcggtaggtagatagaatgtttttgggaagttatagtaagaaaataaaaattaatagttaaagtttcgaagttttttagggcgttaggtttagtcgggaatatatattggtatattgaggtaatcgaaaagattaaatttattttcgtcgatcgggtggttgattttttcgtttttttacgcggttttgtgtgagtatttgagtgtacgtgtatatttgtttataaaatcggaattcgcgtttttgtgggtgtgataggggttggattattaagttaaaacgtttaatttagtttttttgattcgcgttggggaaaatttatagttatttttaatttacg

SEQ ID NO: 16 - DNA, 11667 bp, Artificial Sequence
  chemically treated genomic DNA (Homo sapiens)
cgggattcgtgggcgttaattaatgttatggtggcggagagtaaaggggacgaatatagtttggggttattttcggagttttttagcgttcgtttgtggtcgtgtttggttggtcgcggatttcggcgggcgtcgtaaagcggcgggattgttagcgtagagtttcggttttttgttttgtttttgggttcgagtatcggagtttttggtgtttgcggggagaagtttcggattgagaaatacgggagggtttcgttagtggttgtaggtgcggtagttattttggggatttagtgagaatggggtcgtttggttttgcgcgaattttttacgtgggtgtagtttttgagtcgtcgagggaggcggtggtaatgtcgtttagtgttagtagagggtagtttcgaggtcgtgagttcgaacggcgatttcgttaaatcgcgggtttttttttagttttcggattttgcggggtagaggcggttttggagtttagagattagcgattttagtttgtaggagttcggcgtagaggtttaagggtattttcgggatgtggttaagttatagtttttaggtagttttattttgcgacggtaagggtttagagggtggagggggattagatgttttaggaggggttagaaagttaacgtatatagggaatttgtttttaagtaatagattttaagtatgtggaaattttttaaatgatgttggtgagagttatgatagttttcgtatttttggcgagggaagtcggaggttagtagcgggatggtttttgggcgcgcgggagatagagggattagcgatttttgttgggggtagagggattgtggattaaggatttagatatatttaattggggattttagtttcgattgtggttatttaattggcgttgaatttgagtaatttattatatcgttttagttttcgattaattatttttgtaaaatgggtattgtgggtttcgagtttcgtaagggcgttagggattcgagatagtaggaatttttttattgttttagtaattttgggtttttgggtttgtaggtgggttgaaaggatttttttatcgtacgtttgtttggcgtggttgttttagagatcgaggttcgttaggttttgtaatttagttgcgcgtggttagttatggttttggaatcgtcgggatcgttttagcggtatttcggttagttttcgatttttcgttttgggtattagggttatttagttttagaagatagtgtttattagtataggaattgagattttatattcggcgtagtgggttttttaggaagtttggcgaagagttaaggttcgtcggatttgaggtcgtttggggcgttaaatagatttatttatgagtatattcggttaatatatttaagtttaatagtgggcgggatttttggttggtttcgattttttttatagtgtgtgtaaacgggtatttattagattttttttgtttgtttttttatttgtaaattttgaaaggagagtatttttcggggtgattttgaaaattatgttagaattgaaaaggtttgtgtaaatgcgaggtgtaatgatgatttattagaagtagttagttttgtacgtgtttgtaggatagatgttagaacgttttataaatgtgtgtatatatatattacgtatgtgtgaaggtgtatatttgtttttagatatttgataaatgtatatacgtatgcgtgtatttttggttgagagtaaaggggttaatatagggtttatataggatattattttgtagttttgtgcgtgtatagtatatacgcgagtacgattgtttgtgtttggagatatcgtatggtatcggaggagttgtttagtcgtcggtgttatttgggaagtagggtttcgggtgttgttttggagcgcgggaaagttcggattcgggtttgttggttagcgtttcggcgtcgtcgggtttttttattttttattcgtcggtcggtgagcggttttagttttaagattttcgattttgtcggtgaggggagggagcgagaaggagtgcgtttcgggtttaagaggttgtagggttttattttttaggtttgtcgttttttttttatttttttaggttatatttttatttaaaataaagaagggtgtttgatttatttaggttagggaagtcggttttcgggagttttttgtttttataagttatggtgaagggaggcgaagttagggtttgttacggtttgggagaaaatgcggttgtagggtttttttgggttgtagcgttggttcggggttttaagattgttaaggtgtgtgtgggaggcgcgtagtgtggttatcgaagagttttttattatcgtatcggtaggtcgtcgggttcgttttttttttatgtttttaagggttttgagttgcgtagtattcgtattatttagaagtgtttgtggagaaaacgattttaggtgtaagtttaaggttgttgtttgtaaaggtaatgtcgtggagattgggttttatagcgatttgggtttttaagttatcgaaagttatagggtagggttataaatattttttcgttttttttgtagaatcgtgcggttgataggagagcgttgcgtagaaaattcgaggtcgggcgttggaggagttttcgcggttcggagaaggtatagaggcgtttttgagaggcgtagttggaataggcgatgtacgggttcggatttggtcggttaatcgagtttagcggtcgcgaaaattagagtcgttatagaggttgagtacgattttatttttggggatcgggttcggtggggagttattgtttttaacgtttttagagattttaagtaggataatataatgttggtaggagtaaggtgtaaggaatttagcggtagaagtttttcgggggggtggggaaaggtagattcgtgtttcgtttgagtttgggggtggggatagtttttggttttgtagtttttgtttcggggattagttgggtttatttaatttttttatatggggttgaaaggggttaatgggacggtttcgtcgtttttttttcgggattttatagggttttgattcggtttttatttttgtacggttagtagtcgcgggggttttaggaaggaggataaaggttcggtgttatcgcgggcgggggttcggttttttggttttttgtttatatttatagtttttagcggttgttgggggaagatttttaaaaatatgtgtcgaatttttttttttttttttttagaaataaataaataaaaaaggtaaaaggcgaatttttttttatttttcgtttatagagattaaagttttttgggattttgttttttttttttttttgattgtttagaaatatttgtttttttttgtatatagaggaaaatgcggggaaaggttttttaaaattcggttttatattattattattattaaggataatcgggtaggttgaggttttaacgtggatgattcgagttggtttcgcgtcggggttttgtagttattgttttgtgcgtttagtatttttgggggcgattagggtttttgcgttttcgttcgtcgttcggtagtcgagagtattttgtgtttagattggtcgatttattttttttcgaattttgtttagagttggtaagggggatttagttcgcgttttaagatttgggtttgtagcgtcgttaataggttcggggatacgaggcgttttaggtcggggttttttcggttgttggtttttttcgttttttattcgttggcggcgtttcggtcgttcgtaattgatttaattcgttttttgcgtttgttttttaggtttttcgtttttttataaaggtttaggggagtttcgtttataggttgattttgtaatttttggttcggtggttattttttgtttttttgaaaaagaaaaggaaaaaaaaaaaaaaaaagaaaaaattaattagtcgagggaggcgcgtgaggattggaggcgttagtcggatagtttatgagtaggtttttgggtcggtgtcgtttcgcgggttagtacggttttttttagagaaaattttttaaacgtgtgaagatcgttttgggggaagcgagagggaggttggaggagtttcgggcggggttttagcgtttattagttgtgtttttagggtttgggtgttcgttgtaacggtaatcgcgtgagttttatttttacggttaaggggttagggtagggtggatgtaatcgcgtgcgtttggtttcggaaggtgttcgtagggggtgtttgtggttagttgggttaggaagttaaattttagaaagttattattaaaggtttttttttttttttttttttttttttttttttttgtttttttttttttttttttttttttaataggggaagggaaaaaatattattgaatatttattataaattgggtatttaaaaattagaaaatttaattattttaataattttgatatacgtacgtatttttgttttatagaggagaatattgaaatttgaatgggtattttgtttaaggttatatagttgttaaagaggatattaggttttaatttagttttttttgattaaataggttaggtttttgttttatgttgtataggttattttcgcgaagtgataagattttagaatatgttttttatagtttagtattaagtttgtgcggtaaatagtttgtgtagtagtagtttttgtggtatagaaataaataaaaaaggtaaaaggtatagtttagattgtgtttttttttttttttgggaggtttttttttattatttttgggaggtttttttttattacgtagtttttttgagtattagtggtagttaggtttagttgttgttaagttttttttaaaaatttcggaggttatttgttttatagatatttggaagatttttggttggttttttttttttttaggatttgagattttttaaatgtaatgtaattttgggtttttagatttaagatattttttttatgatagttgggagttttttttttagaaagtggaaagttgttaagattgttcggttttaagaatttttaatttttggttgtttcgaaggtatggggtttttagttttattttttttttattttttttatgggttttatagtttattttaagaagtaggggttgttatatagtggtggtaggtttgacgggtagggttttagggttattattaggttagatttgtagaatggggatattgtagggatgttgagtttgattaagatgttttttggttttagatttttatttagaagttaggagtttggttcggtgattttgtttatgaagtttggtttagatttttataaagggaagagtttagttgagagtaaattattttgggttagatttagatttttggtttatattttgtttgtttggttgtatgaagtttgggtttggttttaatttttattcgtcgttagggggttttttgtagttttatttttagttttggtttttaggaaattttggtaaaggttggatgtatttaatttcggatgaaaggtttagggttttttgtttatttaagttttagatttggagtagtgaattatttattgatgtaagttggaaataggacgttaatattttttggagtagggtataggattatttttagaatttcgtaggttttttttttatagttttttggtgtatgtattttaatggcgaagatttttttaggttttttttgagaataggatatttttttttgttttttttaaattaatttatttattttgattgggtaattttagtgaggtggaggtgattttaagaatttttgtgtaaatgtgaggagagggtttatattgttagtagtgtgtgagatgtttatatttattatatatataatttttatgtaagaacgatttattatttaggatatttagaaattggttgggtatttgtattaaattttatttatattgtatttatatattggttaatatatatatatatatttatatggtttagttatttaggttagattgtatatgttaaggtataattgttatataatttaaatatttttgtttttaggaattgtttaaattagtttttataatagaaatttaaaagtgcgaagagggagagaagagaaaggaaatgagagatgaagaaagtttgttattgaattaaattttcggttttggggaaaatgattaaatttaagagttttggagaaatttcgagggttggagttagatagattttttttgtgggtatttatggggaagtggggtatgatttggttttttttttttattttttttaatttttgtgtagatttaggaaatttttattgattgttttattttgttttttttatttggtgaggttgttgttttttttttagttttgggtgttttttgttttttagttttcggggaggagataggttggttttttattgttggtttcggttaattttttcgttttattggaggtgggttgttatttgtttattagggtttggttttggaagttttttttttttgttttttttgtttgagacggtagttgggggagtgttgatagttagttttagaatagggagtttttgtttgatgaagtaaaggttttgtttatattattttattttttagtaagttaggaatatatatttataaatgtatatattattaatgaggtatgaatgttatatatattgtggttttagtaagtattttttaagtgttagttatatgattattataatgttaaaaatatatgtttattttgtaggcgaatgatagatatatatattttttattttgttattaatatataattgatattgtatatatatatttgtagtagatatagattatataattatttgtttatgggttaaatttttacgttagataaagtataaatagatatgtatatagtaggttgttttttgttttgtaattttttttagttagatagtggagtagtcgggtattaaaggtatttagatattaggtttgattagtttttagggttttttgttttagttgagtttttttttttattttgtttttgtcggtttttggtaagttttagttttagttttgaggtgatgttttttgagaattttcgtggtttggttttttgttttgtagtttttcgtaggatgtttgtggggggcggggggtattttgagtagaagttatttgggtttaattatatataataggatattattttgaggtttttatcgtttgattattggagggttgaagtgagaggtttgtaggtttattttcgtggcgtaaaatcgtttttggtttattagtcggtttggatttgtcgttatattgttggttaggtttcgagatagacggggggggggtagtgagggttgggtgcggggtttagttttagttgagtagtagttcgtaagaatcggcggatttattttattagaagtcggaagttatttattttattagaagagtgaaagtcgattattttttaattcgtttgggagaattagattgtaattaggttaaacgtttgtgtgtttgcgcggaagtatggattttaattcgttatttggggattttagtaaaggaagagagagaaagaaaaaagattaaatttttgtagtcgcgaagtatgtttaatttttattgaaatttttaaattttttatttatattttaaggtaaatgaattttgattattaaaaataataaagtagttattttttatatttgtatagaggttagtagtttgtagcgtatttttataattagaattttatttattaatgtttggaataaatttttggttgttatagttgatttaaaatcgaaaagtttgagaataataataaaaatatagttagtttatatatttagtataattgtgtatgaggtagaatataaatttgaataggtataaaaataaattaagttttatttatagtcgcgttttgtgaacgaattacggtagaaaaagtttttttgttgacgttaaatagtcgtaacgggttagtgatgatatagaaaataaataattttaggaaaataaagtagtagcgatagttataaacgtttgttatttcggttgtttttaatttttgtttaatttattttgaaaaagataaaattttgttgaaaattttttttttttaattagttgttgaattaaggagcgatagaaataaagaagttcgtgttaaaagaaggaatataaaatttattcgggaatcgataaggtaaaaaataaaaataaaataaaaaaatataataaaagtgttttagttttttttagaaaatttggttttttaaataataagttatagtataattaggtttatggttttcgggtcgggtcggtttcgggttataagatcgtttaggtcggtcgttgtcgggttttatattttttttttttaaggtgggagaagtaggaggtttgaaaaataaaaagtagggaggacgagtcgttttagtagcgtgggttaggtaggtagtgatttttttgtttttaggatttgtatcgaaagattcggggatatttgtttttgatttttttttttagatagggagagggtgaaatttttttaattggtagacgaaattattttttattggatatatttttttaattttgaatatagttttttgaaggtttaataaaaattttaacgtaaattttttagaagagaagatgaggaatattaaagtttttgatttttatatttggatttagtaaattttaaggttataatattttttcggttgttcggtgaatttttttttttttttttttttttaaatatttttttaaaaagttttatcgattttttaaataaaatattagttagtcgtcggggttcggaggtgttcgaagcgacgggattgatagcggaggaaacgtagttttttttcgcgttcgttcgcggtgtaaatcgagtataggtcgtggagttaggttgtttcggttttcgttgggttttaattttcgtttcgtttagtgggtttcgcggtaagcggtttttgaatagttttaagagggttcgcggagtaaatatacgtattggttcgtttttttttcgggtagcgcggtttcgttattagttttgttcggcgcgtttagttatggattgtacggtagtcgggcggggaacgcggagagcgagcgtatcgatttgtgagagaaggttaagaggtttgcgttgtcgacgttcggtcgtattttcgtttcgggtttttttcgcggtgaatttgggtaggagacgttggggtttcggaaagagacgagtttagtagaaagcgcgtagagaggtagttttaggttaggggagtgtaaggttatagaggttagggaggtgagtataggaggatataaattgaggggataaagaggagcgataggagtttaggaaagcgaaaaagtatagagggattttgggcgttggttttagaggcgggtttagagggtgtgaggttaggttggcggcggcgtcgtcggttgcgatcggggtcggcgtcgcgcgtttttgtattttcgtattcgtttgtatcggtatgcggtgggtttttagagatcgaggcgcgaatgtagcgcgtcggttttgttgtcgtggttttagtaagagtaatgtattatggcgagttcgggttgtcgggtataagcgaattgtaggttcggcgtattggtgggcgcgggcgtcgggggcgcggcggtggttgggttggtagggttgagttggttcggcggcggcgcggcggtttcgtggtgcggtggggtaggcggcggtgcggcggtcgcgtgtagatggtgtgcgtgcggatgcgggtgggggtgcggcggaggcgggggtgcggtcggcgggttttttaggttattgtacgagtttattacgtcggggggtagcgttatgttttgtacgcgtgtgtacggttcgtacgaggcggtcgggttcgttagttttttgattatagcggtcgcgttagggttatcggggttcgcggttgtagtcgtagttgttgtagtcgttgcggttgtcgttatttggtaggaggtatagggtatgggtgagggaggttgcggtagcggttacgagttgttgaggaagttagattgtaggtatttggggggcgttaggtagtcgtagtcgtcggtttcggcgttcgttacgtcgtattcgtttgcggcgttttcggtttcgaagagttttttgtcgggttggaagtgcgcgggcggcggtcggaagggtttttttatgcggcggcggcgtcggtagttgtttttttcgaatatgttttcgtaggtcgggtttagcgtttagtagttgtttttgcgttcgtcgtcgttttcgcgcggtattttgatgaagtattcgttgaggttgaggttgtggcggatgttattttgttagttttttttatttttttcgtagaacgggaatttcgcgatgatgtattggtagatgtcggatagcgtgagtttttttttcgcgttttcgcggatcgttatggcgatgagcgttacgtacgagtacgggggtttttgcgtcgggttcggttttttcggggttgtttcgtcgttatttttatcgtttttgtttgggttcggcggcggtttttttggttttttgattgtgcgatcggtttttggggttagtagggttttcgtcgcgttttcgggttcggggtagttggttattatgataaagtcggcgcgtcgcggtcgggtcgtttttgtttttcgttttaggcgttggcgcggtaaagagttggggcgtacgagttcgtttacggttaagttttaaatttttggagattgcggatgtcgttcgcgtttgtttgttggaggtttgtcgttgttttttttttttttttttttttttttagggagcggtcggcgggagtggagtttagtttttggttatggggagttcgtttaatagagaggggtttcggtttcgtcgttttttttcgtttaggttagttttcgttttggtgggtttttttttttgcgtttttttttttttttcgtttttcggtttttcgaagtacgattcgcgtttttggcggagttgttttttggagtttttagtgcgttaggagtttcgttttgttttgattcgtatgggttttatcgagtttcgtttgcgttaggcgttttcgtttttatagcggggcggttagtcgcgtacgggcgagtttatttttaagttattttttgtaaacgtttcgtatagtttggatcggtttgttttcgtttagcgagttttaggggtttagtcgatagttaggtttacgcgtttttgaaatttgtcggtattcgttttgcgggttgggttgggagatgacgaggatttcggtggggtttgttcgtattcggttaaagtttaggaagttcgggttttagcgaggaaaggcgttttaagttttttcgcggtttttaggtgaaagaaaacgatttttttgttttgtcgtttgttgtcgttttgaggttgaatttttagttcggggttggggaggggcgagacggcgagggggttggacggggtagggtggggagagttgttttgaggttttgggaaagttagtttagaaacgggtgtgattgtacgaagaagtttcggtttggtttgtttttcgcg

SEQ ID NO: 17 - DNA, 11667 bp, Artificial Sequence
  chemically treated genomic DNA (Homo sapiens)
cgcgagggataggttaggtcgaggttttttcgtatagttatattcgtttttgggttgatttttttaaagttttagagtagttttttttattttatttcgtttagttttttcgtcgtttcgtttttttttagtttcgagttagaagtttagttttaagacggtagtaaacggtagagtaaaggagtcgtttttttttatttgaaagtcgcgaggaggtttggagcgttttttttcgttggggttcgagttttttgggttttggtcgggtgcgggtagattttatcggggttttcgttattttttagtttagttcgtagagcgagtatcggtagattttaagggcgcgtgagtttggttgtcggttgggtttttgaggttcgttgggcgggggtaggtcggtttaggttgtgcggggcgtttataaaaagtgatttggagatgaattcgttcgtgcgcggttggtcgtttcgttataggggcgaaggcgtttgacgtaagcggaattcggtggagtttatacgaattagaatagagcgaggtttttggcgtattagggattttaggaggtagtttcgttagagacgcgggtcgtgtttcgggaaatcggggggcggggggaggggaagagcgtagaaaagaaaatttattaaggcggggattggtttgagcggggaggggcggcgaggtcggagtttttttttgttgggcggattttttatggttagaggttgagttttattttcgtcggtcgttttttaggggaaggggaaggagaggggagagtagcgataggtttttagtaagtaagcgcgggcggtattcgtagtttttagaagtttgagatttggtcgtaagcggattcgtgcgttttaattttttgtcgcgttagcgtttggagcggagagtagaggcggttcggtcgcggcgcgtcggttttgttatgatggttagttatttcgagttcgaggacgcggcgggggttttgttggttttagagatcggtcgtatagttaaggagttagaagggtcgtcgtcgagtttaggtaagggcggtgggggtggcggcgggatagtttcggagaagtcggattcggcgtagaagttttcgtattcgtacgtggcgtttatcgttatggcgattcgcgagagcgcggagaagaggtttacgttgttcggtatttattagtatattatcgcgaagttttcgttttacgagaagaataagaagggttggtaaaatagtattcgttataattttagttttaacgagtgttttattaaggtgtcgcgcgagggcggcggcgagcgtaagggtaattattggacgttggattcggtttgcgaagatatgttcgagaagggtaattatcggcgtcgtcgtcgtatgaagaggttttttcggtcgtcgttcgcgtatttttagttcggtaaggggtttttcggggtcggaggcgtcgtaggcgggtgcggcgtggcgggcgtcggggtcgacggttacggttatttggcgttttttaagtatttgtagtttggtttttttaataattcgtggtcgttatcgtagttttttttatttatgttttatgttttttgttagatggcggtagtcgtagcggttgtagtagttgcggttgtagtcgcgggtttcggtagttttggcgcggtcgttgtggttaaggggttggcgggttcggtcgtttcgtacgggtcgtatatacgcgtgtagagtatggcgttgtttttcggcgtagtgaattcgtataatggtttgggaggttcgtcggtcgtattttcgttttcgtcgtatttttattcgtattcgtacgtatattatttgtacgcggtcgtcgtatcgtcgtttgttttatcgtattacggggtcgtcgcgtcgtcgtcgggttagtttagttttgttagtttagttatcgtcgcgttttcggcgttcgcgtttattagtgcgtcgggtttgtagttcgtttgtgttcggtagttcgagttcgttatgatgtattgtttttattgggattacgatagtaagatcggcgcgttgtattcgcgtttcgatttttgagagtttatcgtatgtcggtgtagacggatgcgaggatgtagggacgcgcgacgtcggtttcggtcgtagtcgacgacgtcgtcgttagtttgattttatattttttgggttcgtttttggagttagcgtttagggtttttttgtgttttttcgtttttttaagtttttgtcgtttttttttgtttttttagtttatgtttttttgtgtttatttttttgatttttgtgattttgtatttttttggtttgaagttgtttttttgcgcgttttttattgggttcgttttttttcggagttttagcgttttttgtttaaatttatcgcggaaagggttcggggcggaggtgcgatcgggcgtcggtagcgtagattttttggtttttttttataggtcggtgcgttcgtttttcgcgtttttcgttcgattgtcgtgtagtttatggttagacgcgtcggataggattgatggcgggatcgcgttgttcgagaaagggacggattaatacgtgtgtttgtttcgcgaatttttttgaagttgtttagaagtcgtttgtcgcggggtttattaggcggggcgggggttgggatttagcgggagtcggggtagtttggttttacggtttgtattcggtttatatcgcgggcgggcgcggagggaggttgcgtttttttcgttattagtttcgtcgtttcgggtattttcgggtttcggcggttggttaatgttttgtttgaaagatcggtggaattttttaagagagtatttaaaaaaaaaaaaaggaaaaaaaatttatcgggtaatcggggaagtattgtggttttggagtttgttaaatttaaatatgaaaattaaaagttttagtattttttatttttttttttggaagatttgcgttagagtttttgttgggtttttaaaaagttgtgtttagagttaggagaatatatttaataaaagatggtttcgtttattaattggggaagttttattttttttttatttgaagaaaaaaattaaaaataaatgttttcggatttttcgatgtaagttttggaggtagggagattattgtttgtttggtttacgttgttgggacggttcgttttttttgttttttgttttttaaattttttgttttttttattttgggaaggagaaatgtgaaattcggtagcggtcgatttaggcggttttgtggttcggagtcggttcggttcgaaaattatagatttggttgtattgtagtttgttgtttgggggattaaattttttagagagaattagagtatttttgttgtgtttttttgttttgtttttgttttttgttttgtcgattttcgaataaattttgtgtttttttttttaatacggatttttttatttttgtcgttttttagtttagtagttagttaaaagaggaagatttttagtaaaattttattttttttagaatgagttaaataaagattgagagtaatcggggtggtaggcgtttgtggttgtcgttgttgttttgtttttttgggattgtttgttttttgtgttattattggttcgttgcggttgtttaacgttagtagaaagattttttttgtcgtggttcgtttataaggcgcgattgtaggtaggatttggtttatttttgtatttatttaagtttgtattttattttatatatagttgtattaggtgtatgggttggttgtgtttttattattgtttttaaatttttcgattttgagttaattataatagttaagagtttgttttaaatattaatagataaaattttggttgtggaaatgcgttgtaaattgttaatttttgtataaatgtgagaagtgattgttttattatttttaatagttagaatttatttattttgagatatgggtaggagatttggaggttttagtaaggattgaatatatttcgcggttgtagaaatttagttttttttttttttttttttttttttattgagatttttaaataacgaattaagatttatgttttcgcgtaggtatataggcgtttgatttggttgtagtttggtttttttaggcggattagggggtggtcggtttttatttttttgatgggatagatgattttcgatttttgataggatagattcgtcggtttttgcgggttgttgtttagttgagattagatttcgtatttagtttttattgtttttttttcgtttatttcggggtttggttagtaatgtggcggtaggtttaggtcggttggtgggttaggagcggttttgcgttacgggaatgagtttgtaggttttttattttagttttttagtggttaggcggtaggaattttagggtggtattttgttatgtatagttgggtttaagtggtttttgtttagaatgtttttcgttttttataggtattttgcgagaggttataaggtaggagattaagttacggggatttttagggggtattattttaagattgggattggagtttattaggaatcggtaggagtaggatggagagggaggtttagttggagtaggaagttttagaggttggttaggtttggtgtttgggtgtttttggtgttcggttgttttattatttggttaaggggagttgtagaatagaaagtagtttgttgtgtgtatgtttgtttgtgttttgtttagcgtgaaaatttagtttatgggtaagtaattgtgtagtttgtatttgttgtaaatgtgtgtgtatagtgttagttgtgtattggtgatagggtgggaggtgtgtgtgtttgttattcgtttgtagagtgggtatatatttttggtattgtaataattatatggttaatatttaggaagtgtttattgggattatagtgtgtgtggtatttatattttattggtgatgtgtgtatttgtgggtgtgtatttttggtttgttgaaaaataaagtaatatggatagagtttttgttttattaggtagaggttttttgttttgaagttggttgttagtatttttttaattgtcgttttaagtagagaaagtaagggagggaagtttttaggattaagttttggtgggtaggtagtagtttatttttagtgagacggagaagttggtcggggttagtaatgaaggattagtttgtttttttttcgagaattgaaaggtaaaaggtatttaaggttgggaagagagtagtagttttattaagtaaggaagataaaatgaagtaattagtgagggttttttgggtttatatagggattgaaggaggtgagagaagggagttaaattatattttatttttttatggatgtttataggaaaaatttgtttaattttagttttcgggatttttttaaagtttttaagtttggttattttttttagggtcgaaagtttagtttaatggtaggttttttttattttttattttttttttttttttttttttttcgtatttttgagtttttattatagaagttgatttaagtaatttttgagggtaaaagtatttgagttatatagtaattatgttttagtatatatagtttggtttagatggttgggttatgtgagtgtgtgtatatatattggttagtgtgtaaatataatgtaaatagagtttaatataaatatttagttaatttttgaatgttttgggtaatgagtcgtttttgtatgaagattatgtgtgtggtgggtgtaaatattttatatattgttgatagtatgagttttttttttatatttatatagaaatttttagagttatttttattttattaaaattgtttagttagagtaagtgggttggtttggagaaagtagggggaggtgttttgtttttaagagggatttggagaaattttcgttattgaagtatatgtattaagggattgtgggaaagggatttacggagttttggagatggttttgtattttattttagagggtattgacgttttgtttttagtttgtattagtaaatagtttattgttttagatttgaggtttagatgagtaagaggttttggattttttattcgaggttaggtgtatttagtttttgttagggttttttggaagttaaggttggagatggggttgtaaggagttttttggcggcgggtggaaattggggttagatttaggttttatgtagttagataagtagaatatgggttaggaatttgagtttggtttaagatagtttgtttttaattgagtttttttttttatgaaggtttgggttaggttttatggataggattatcggattagatttttggtttttgggtgggggtttaaggttaggaaatattttgattaggtttagtatttttgtagtgtttttattttgtagatttaatttggtggtgattttggggttttgttcgttaggtttattattattgtgtggtagtttttattttttggagtagattgtgggatttatagaaaagatggggaaaggatgggattaagagttttatgttttcgggataattaggaattaaggatttttaaaatcgagtagttttagtaattttttattttttggagagaggatttttagttgttataaaaagagtgttttgggtttaggaatttaaaattatattatatttgaggaattttaggttttaagaagagaagagattaattagaggttttttaggtatttgtgaggtaaatggttttcgaaatttttaaaggagatttgatagtagttgagtttagttgttattgatgtttaagggagttgcgtagtaagggagagttttttaggagtagtaagggagagttttttaggagaagaaggaagtatagtttgagttgtgttttttgttttttttgtttgtttttgtattatagaaattgttgttgtataggttgtttgtcgtataggtttggtgttgagttgtgaaaagtatgttttgggattttgttatttcgcggaggtggtttgtgtagtatgaggtaaagatttggtttgtttagttaggaagaattgggttggagtttggtgttttttttaatagttatgtaattttgggtaaaatatttatttaaattttagtgtttttttttataaaatagaaatacgtgcgtgtattagaattattaaaataattaaattttttaatttttaagtgtttagtttatagtagatgtttaataatgttttttttttttttttattaaaaaaaaaaaaggaaggaaggaagtaaaaaaggaaagaagaaaggaagggggaaaaaaaatttttagtaataattttttaggatttgattttttaatttagttggttataggtattttttgcgagtatttttcggggttaggcgtacgcgattgtatttattttgttttagttttttggtcgtgggagtgaggtttacgcggttgtcgttgtagcgaatatttaagttttgaaggtatagttggtgggcgttgagatttcgttcggggtttttttaatttttttttcgttttttttaaagcggtttttatacgtttaggaggttttttttgagaaaggtcgtgttgattcgcgaagcggtatcgatttagggatttatttataggttgttcggttggcgtttttagtttttacgcgtttttttcgattggttgatttttttttttttttttttttttttttttttttttttagaaaagtagaaggtagttatcgggttagaggttgtagggttagtttgtgggcgaggtttttttaggtttttgtggagaaacgggaaatttgaggggtaaacgtaggaagcgggttgggttaattgcgggcgatcgaggcgtcgttagcgggtggggagcgagaggggttagtagtcgggaagatttcggtttggagcgtttcgtgttttcgggtttgttggcggcgttgtaagtttaggttttggggcgcgagttaagttttttttgttagttttaaataaaattcgggggagaatgagtcggttagtttgggtatagggtgttttcgattgtcgggcggcgggcggaagcgtaggggttttgatcgtttttagaggtgttgagcgtatagggtagtggttgtagagtttcggcgcgaggttaattcggattatttacgttgggattttagtttgttcggttgtttttagtaataataatagtatgaaatcgagttttaaaaaattttttttcgtatttttttttatgtataagggagaataagtatttttaggtagttaaagaaaaaaaaagggtaggattttaggaaattttaatttttatggacgaggagtgaaggggaattcgttttttgttttttttgtttgtttgtttttagaagagaaaaaaaggaaattcgatatatatttttaaaaatttttttttaataatcgttaaaggttgtgagtgtgagtagagagttaggaaatcgagttttcgttcgcggtgatatcgggtttttgtttttttttttgaagttttcgcgattgttgatcgtgtagagataaaggtcgggttagggttttgtggaatttcgagggaggggcggcggggtcgttttattggttttttttaattttatgtaggggggttaggtgggtttagttgattttcggaataggggttataaggttaggggttgtttttatttttaggtttagacgaggtacggatttgtttttttttattttttcgaaaggtttttgtcgttaaattttttgtattttgtttttattagtattatattgttttatttaaagtttttggaggcgttagggatagtggttttttatcggattcgatttttagaaatggaatcgtgtttaatttttgtggcgattttggttttcgcgatcgttgggttcggttggtcggttagattcgaattcgtgtatcgtttgttttagttgcgttttttaggggcgtttttgtgtttttttcgggtcgcggagatttttttagcgttcggtttcgaattttttgcgtaacgtttttttgttagtcgtacggttttgtagagaaggcgggagagtgtttgtgattttgttttgtagttttcggtgatttaaaaatttaggtcgttgtggaatttagtttttacggtattgtttttgtaggtagtagttttgggtttatatttgaggtcgttttttttatagatatttttgggtgatgcgagtgttgcgtagtttagaatttttggaagtatgagaggaggacgagttcggcggtttgtcggtgcggtgatggagggtttttcggtagttatattgcgcgttttttatatatattttaatagttttggggtttcgggttagcgttgtagtttaaagagattttgtagtcgtatttttttttaggtcgtggtaaattttggtttcgtttttttttattatggtttgtgagggtaggaggttttcgggggtcgatttttttgatttgaatgagttaggtatttttttttattttaagtgggggtgtgatttgaggagataaaggaaggacgatagatttggggagtggggttttgtagttttttgggttcggagcgtattttttttcgttttttttttttatcggtaggatcgggggttttgaaattggagtcgtttatcggtcggcggatgagagataaaggagttcggcggcgtcgaagcgttgattagtagattcggattcggattttttcgcgttttagagtagtattcgaaattttattttttaggtggtatcgacggttgagtagttttttcggtgttatacggtgtttttaggtatagatagtcgtgttcgcgtgtgtattgtgtacgtataagattgtaggatgatgttttgtatgagttttgtattagtttttttgtttttagttagaggtatacgtatacgtgtgtgtatttgttaagtgtttagggataggtgtgtatttttatatatacgtgatgtgtatgtgtatatatttgtagaacgttttagtatttattttataggtacgtatagggttggttgtttttaataagttattattatatttcgtatttatataggtttttttagttttggtatgatttttagaattatttcgggaaatattttttttttaaagtttatagatgaggaaataagtaaaaagaatttaatgggtattcgtttgtatatattgtgagagagatcgaggttagttaaagatttcgtttattgttgaatttgggtgtgttggtcgaatgtgtttatagatgggtttgtttagcgttttaggcgattttaggttcggcgggttttggtttttcgttaggttttttgggagatttattgcgtcgggtgtggggttttaatttttgtgttgataagtattattttttggagttgggtgattttagtgtttagggcgggaagtcgggagttggtcggagtgtcgttgaggcgatttcggcggttttaaggttatggttgattacgcgtaattgggttgtaaagtttggcgggtttcggtttttaaagtagttacgttaggtaaacgtgcgatgaaaggatttttttagtttatttataaatttaggaatttagggttgttggagtagtgaggaaatttttgttgtttcgaatttttagcgtttttacgagattcggagtttataatgtttattttatagaagtgattagtcggaggttagagcggtatagtgagttgtttaagtttaacgttagttgagtggttatagtcgggattggaatttttaattaagtgtgtttggatttttggtttatagtttttttgtttttagtaaggatcgttggtttttttgtttttcgcgcgtttaggagttatttcgttattagttttcgatttttttcgttaagaatgcgaaaattattatagtttttattagtattatttaaaaggtttttatatgtttggaatttgttgtttgaaaataaattttttgtgtgcgttggttttttaattttttttaaggtatttggtttttttttattttttaagtttttgtcgtcgtagagtgaggttgtttaagggttgtagtttgattatatttcgggggtgtttttgggtttttgcgtcgggtttttgtagattgaggtcgttggtttttgggttttaaggtcgtttttatttcgtagaattcgggagttggaagggagttcgcggtttggcgaggtcgtcgttcgggtttacggtttcgaggttgttttttgttggtattgggcgatattattatcgtttttttcggcggtttaggagttgtatttacgtgaggggttcgcgtagagttaggcgattttatttttattgggtttttagagtggttgtcgtatttgtagttattgacgagattttttcgtattttttaattcgagattttttttcgtagatattagaggtttcggtgttcggatttagggataaggtagagagtcggagttttgcgttggtaatttcgtcgttttgcggcgttcgtcgggattcgcggttaattaggtacggttataggcggacgttgggggatttcggggatggttttaggttgtgttcgttttttttatttttcgttattatagtattgattggcgtttacgggtttcg

SEQ ID NO: 18 - DNA, 1394 bp, Artificial Sequence
  chemically treated genomic DNA (Homo sapiens)
cggaatttcggttcgaagtcgagataggagattggatgcgaggtttttttagagttggttttttttaaataatttttaaaatttttagattttaggggtacgtcgaaattttttaaagtagtttaaagaatataacgagagttttaatattttaggtggcggcgcgttggttttttggagcggggcgggacgcggtcgcgcggatttacgtgtataatcgcgcgggacggggttacgcggatttacgtgtataatcgcgggattttagcgttagcgggattttagcgttagcgggattttagcgttagcgggattttagcgttagcgggattttagcgttagcgggattttagcgttagcgggattttagcgttagcgggtttgtggtttagtggagcgagtggagcgttggcgatttgagcggagattgcgttttggacgttttagtttagacgttaagttatagttcgcgtagtagtagtaaaggggaaggggtaggagtcgggtatagttggattcggaggtcgtgatttaggggaaagcgtgggcggtcgatttagggtagttgcggcggcgaggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcggtattgggtaggaaatggtagggttgaggtcgattttaggagtataagggagttttttattttttgtttatatttgttatttttagttttgtaatttattttagatatatagaaagtaagtaggattggtggggagacggagtttaataggaatattttttagtagtgagtaggggttgtatgggacgcgggaggagtttagaggaggcgcggagagtgttcg

SEQ ID NO: 19 - DNA, 1394 bp, Artificial Sequence
  chemically treated genomic DNA (Homo sapiens)
cgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgttatttgggatgttaggattttcgttgtgttttttggattgttttgggggatttcggcgtatttttaggatttaggagttttggaagttgtttgagagaaattagttttgggagggtttcgtatttagttttttgtttcggtttcggatcggggtttcg

SEQ ID NO: 20 - DNA, 6357 bp, Artificial Sequence
  chemically treated genomic DNA (Homo sapiens)
gtgtggagattgggaaggtgataaggtgaaggtaattgaaggaagagtcgagggggatatggggaaggattttgttttattttttttaagttgaattattgttttttgaaggtcggtttttggagaaattaaagggtttttgtgtgatatagttatgttatatataaatagaattttgaagtttattaatttttgaggttaagtaagagggaatgtaggggttaaggtagaagagaaattaaaattttagagcgttgagtaaagatgttaattagagaaagagaaatttatttgcgatgttaattaataagcggttaattaaaacggtattttgagtgttaattaatcgttttattaagttatagttattattggaataaattgaaattttttcgtttcgttttttgtttttggtgtaggcggggtcgcgtttttagatatcgtgagaggttttggggcgcggaggttgggggtagtttcggttagtttttttagttttttttaggtttatagaatacgttattggataagtgtttaagtagcgatttttggtttagatatatcgttcggggagtaagtagttgcgtcgaagaataatttatttagtagtagttaatatcgacgtttttttttagaaagagttttcgtaaagcggggggatgtgatttgtgggtttttagtaggggtaggaggtagtttagttcgagagggggcgttttagggtttggattttgtatttttattttttggaatatatttaacgttttattttgaaaattttgtttaggttttggttttggtgtcgtttagtaattaggaaagagttggatttgtttttaggtagtaagaataggattgttagttttttgtggttttgtttttcgaggttttatgagaaggggatgggggtgtaagaagggaagagtgaggtggtgtgttgggcgtcggggacgaggacgtacgttagttaagacgtgttttttatttagtttacgcgcgtttttttatttttttggttttttaaaatcggtaagagaattaagatttcgaatttttattttgaggagtttttcgtattttttaattgttaaatttttgttttttattaaattttcgggggagaaatatttggtaataagaagggattgtgaatttaaatgttaattgagtgggtttttttttcgtagttttatttgtttggtagtttttgttgaaattaaatatattcggagcgtttagtgtaatatttttggggtgtcgagtagaagcgtagtaaagagagattttagcggatttttgtttggtttgttttttatcgatttttatagaaaaaaagagaatgttattggaagaagtttttttgcgtggtgggcgatgtgtgggtgggggatttgtggtatggtttacggtgttgtttttgtgtttgcgatgatatacgtatgttttgagttgtgggttcgtttttttggaggtgcgttcgatcgtatttgttggtgggtttgagcgtgtttggggtgttttaggagaattgagagaacggtttttacgtgtaaagttttaaagtattaatatttttattatattattattatttaatataataatatttgttcggttagcggtattaattaggttatattaaaatcgtagtgtgtttttaatggtgcgtaatgtgtttatatttatatttttttttttgaggatgggcggttgtaggttggtaggggaggagagataggtaagcggcgggttggattagggcgtgacgttttttattacgtatataaatatatatagtttattggatgtttgtcgggtgggagtcgtaattttcgcgcggtcgatggggtttttcgttgcgtattcggttttgcgtcgagtattttgtagttttttttcgcgatacggcgttttgaattcggcggattgattttgtttttttttttttttttgtgtgtgtttgcgtttaattggttaggtttttaagatttgggagggttggtgtgaaagaattaaaatatttttaattggagttttttcgtcgagaattggaggtttcgttttttagttcggcgtttttaggattttttttttagagggaattttttttagaaattttagggtgggtttgtaaaagacgttttcgtagagtaggtttcgttagggttttttttttgtttttggtgttagcggtcggttcgggcgtttcgtagatttcggcgaggtagatgttaagttcggagagtgtttttttcgtaggcgtcgtggcgagattattttgaatatgtaatatatttgtaacgtgcgtcgaggtgtgatgtgtgtgttgaaatagggggatgggggaattcgaagtcggattgggaaggcgggggggaggcgtatagaatttataatgtatttcgtaatttaataatttgaatatttatttattaaaagttgttgcgtgatatttatattgagttattagtttttgtttttaattcgggcgaaaacgattgtattgtcgagttatggttgtagcgtatggggacgttgttgttcgcggtcggatagagtttattagttataacgcggaaggtttttgtatttttttgggggcgggaggaaagtattgttagttttgtttgggggtcgagggtaataagtatcgagtttttcgttttacgtagggttagttgtttagtttagcgaagtttttgtgatttggtgcgtgtttttcgtttttttttttttattaaagaagtaaattttttatttattttttttaattcgatcgtttagagttgttgttttttttttgttagatttttttttttcgattagtttgagtatacgattagaattgtttagagagtaggaagtatattgattttagtttgttttgtttatagataggttttgataaggttgttagaatagtcggagaggtttatataattatttaattattaaaattgttagttaggcgggacgcggattcgcgtttcgggttgcgttaggtattttagtattgggtcgcgcgcgtgattgatcggtgttgatagtatcgtaaaataattacggcgaattttttgatgtgtgattttattttaagtttatgttttagagaggtaatcggagaatgagaagggttagtgttatttcggattatttggaatttgcgagaaagggtaaaatgggggaaggagtttcgaggaaaacgggagagatgggggtgtagagagagagggaagaagaaagcgagttatggattgttggagggattgtaagtaattcgttaaattgtgtaagtgattttttttagagttagtatatggtagattgattttgtttaacgtcggttttagttatatttaaaatgatttagcggttattattgcgattggtttaggaattgataggtagttttaggcgtaaggagtatagattttgtttatcggagatgtgttcgtaattgttgttaaatatagttaagtaaatattattagcgaagagttttgttaagagaaatgttaatttaataaatatgtttttttttttcgtttttcgtatggttgtttgcgttttttttagaggtttttttttttgtttttttgttgtttgggttagacgttttaggtatggtgttgattttcgttattttggagtttcgagttgagtttcgggtagaagatgataggttagtcgtggggtaaggaggtcgcggaaacgcggaacggtttcggggagacggaagcgtttaatgagatttattttgtagttcgggtttagtttatttttttcggagattgtcgcggttttcgaattcgggtttaggtttttatgtttcggcggttagaggacgttgcggggattattggggagttgtttttagttagttttttgttttacgtcggaggttttggcgcggtttttttttcgaattagattggcgattttgggttaggttttaaggatcgtttcggttttttcggttttgcggggagaatttgaggaatcgagtttaagatagtcgatttaggttgtttttatttagattttgcgttttcgattcgttggagtgaatttgatattgttaggtttttttttatggtatggagtgaatgaagagggttatagatttttttatttagtatagtttttcggtaggttttggaaatttatagggagtagaagtatagtattttttgaatcgttttttttttttgggtttgtggttatttgaaggtagagttttgtgtttttaagatagtaggttttcggttaagtttggagtttggggttttaatatatttatatagggttggtattatcgtttttttggattaaaggtaggtttttatattttttttaaaggaatagaaggaaggaaaaggaaattaatacgggttatgtttagatagtaggtttatggtaattttttatagttatagagttttaattcgagtatttttttagagaggaaaaatttaaaaaatttttaaaagggggaaagttgggtagattatagtatttattttttatgtttaggtagaaaaattaatttagagggagtaaaggggtaagaaatatgaagagattttttttgggagttgaggagtattttagtttataatttggttaaaggagaaagttattggtttttttttttgatagaggcgtgttatttatttttttagggaatatgatggtttataaatgaagaggttagtttttttgtagtttttttttatagagtgtaaaatatatatcgtttttattagtgtttgggatgtaaagaattttgtttatttaaaagagatattgtatttttaaagttaaatagtattaatgtatgtggcgaattaagtaggtaaataatttatatatggttgttgtatttgaaggaattatttatttttatgtatagtaaattgaagaaataatggtattaatgagttttgtaaaatgtaattgtgaataatgaaagataatattgtattttgtaatagaaagaataaaggtgaaataattagttagtaaagaggaaaagaaagcgagtaatgattaaatgattaaaagttggtagagtgaatttaatgttattgttagacgtagttatttatttataagtgaaagttaggttttaagtatagtgtaattatagttggggttgttagtttgatattaatgtagttagtagaaattttttaattggttttagaggagaaagtgaattagaaaatatattaatattttaaaaaagtatattttgtttaatttttttattttcgaataatatttgaagattaaaatgttttaggtataagaatttaaatgagtaattttgtttttgaaggaaacggttaatgagatagaaaatagattaaagggaaattattagtggatgagagatattgataggtttgttttgttgattggttggtttgttatttgtagtttgtgttttttagttttacgttatgagttaagttgataatatgaaaagatttataaacgtgtagttagaagttatagtttattatttggaaatttaaatgtaaggggagggggtggtagagaaggtatcggcgaggttgggagggagaggtgtgtatcgagggaggaggaggaggaggaaggggaggagggaaaggaggaggaggaggagaaaagaagtttttattttttggtataaaatcggttatattagagaataataataagttattatttgttataaatgtgttatggattgttaaaaaatgtagtttcgaatcgataatattgttcgaattgaagatagtaataaaatgtttaaagttgcggatgtaattttatatgcgttcgggttgatgtgatatgatcgtattagggaaataaagttaagtgtagttaggatttgttagtatagtggtttttgatggaatagttgaggtatatatcgttcgtggtatggatttcggggtcgaacgtttacgattaagatttttgtttttttgaaatgaaatagaaataggggagttgtaggaaaatcgaatcgcgtttagggttaggagtaagataggagtttttagcgaagatttgaatatttagaattggaacgggtaattagtagatagttaggaaaaaataaataaataaataaaaaagtttggatggatttttgtaaataattattaagaaaaataaaaatgaatttttttattagtttgttttggaggtagttagaaataaataattaaagtaagagaggatgaagatttaaataaaataattatgtgtattattaaaataattatatatgtttgtatagatacgtatatattaaggaacgtaatgggggtttttcgtatagttttaggagatgtagga

SEQ ID NO: 21 - DNA, 6357 bp, Artificial Sequence
  chemically treated genomic DNA (Homo sapiens)
ttttgtattttttgggattgtgcgaggagtttttattacgttttttggtgtatacgtgtttgtataaatatatatgattattttaatgatgtatataattattttatttaaatttttatttttttttgttttggttgtttgtttttgattatttttaaggtaggttaataagaaggtttatttttattttttttaatgattgtttatagaggtttatttaggtttttttatttatttatttatttttttttggttatttgttaattattcgttttagttttgaatgtttagattttcgttgaaagtttttgttttgtttttgattttaagcgcgattcggtttttttgtagtttttttatttttattttattttaaaagggtaaaagttttggtcgtgagcgttcggtttcggagtttatgttacgggcgatgtgtgttttagttgttttattaaaagttattgtattaatagattttgattgtatttagttttgtttttttgatacggttatattatattaattcggacgtatgtgaaattatattcgtaattttaagtattttgttgttatttttagttcgaataatgttgtcgattcgggattatattttttggtagtttatagtatatttatgatagataatagtttattattgttttttgatatggtcgattttatgttaaagaatgagggtttttttttttttttttttttttttttttttttttttttttttttttttttttttttttcgatgtatattttttttttttaatttcgtcgatgttttttttgttattttttttttttgtatttgaatttttagataataggttgtgatttttggttgtacgtttatgggtttttttatgttattaatttagtttatagcgtggaattaaagaatatagattgtaagtgataggttagttagttagtaaggtaagtttgttagtattttttatttattaatgatttttttttagtttattttttgttttattggtcgttttttttaaaaataaaattgtttatttaaatttttatgtttggggtattttggtttttaaatattgttcgaaagtgaaaggattaggtaaaatatgtttttttaaaatgttaatatattttttggtttatttttttttttgaggttaattaggaaatttttgttggttgtattaatgttaaattgataattttagttataattatattgtgtttgaaatttaatttttatttgtgggtagatggttgcgtttggtagtgatattgaatttattttgttagtttttgattatttaattattgttcgttttttttttttttttgttagttgattattttatttttattttttttgttgtaaaatgtagtgttgttttttattatttatagttgtattttgtaaggtttattagtgttattgtttttttaatttgttgtgtatgagaatggatggtttttttaagtgtagtaattatatgtaagttgtttatttatttgattcgttatatatattggtattgtttgattttaaaaatgtagtattttttttaaatagatagggttttttatattttaaatattgatgaaggcggtgtgtgttttatattttgtagaaaaaagttgtaggagggttagtttttttatttgtgaattattatgttttttgggaagatagatgatacgtttttattaaaggaggaggttagtgatttttttttttgattaaattataaattagggtgttttttagtttttagaggggatttttttatattttttatttttttgttttttttgggttagtttttttgtttaggtatgaagaatgggtgttatgatttatttagtttttttttttttaaaagttttttgggttttttttttttgggaagatattcggattaggattttatggttgtgaagaattgttataggtttattatttgaatataattcgtgttggtttttttttttttttttttgtttttttaaaaaggatataggagtttgtttttagtttaaggaaacggtgatgttaattttgtgtaaatgtgttggggttttaggttttaaatttgatcgaaaatttattgttttggaggtatagagttttgtttttaaatggttataggtttagggagagggagcggtttagaaaatattgtgtttttgttttttgtggatttttagggtttgtcgagggattgtgttgggtaaggggatttatggttttttttatttattttatgttatgagagagaatttggtagtgttagatttattttagcgggtcggggacgtagggtttgggtgaaaatagtttaggtcggttattttggattcggttttttagatttttttcgtaaagtcggagaggtcggggcggtttttggggtttggtttagagtcgttagtttagttcgggaaagaagtcgcgttaggattttcggcgtggggtagagagttgattgagggtagttttttagtggttttcgtaacgttttttggtcgtcgggatatgaagatttaggttcgggttcgagggtcgcggtaattttcgaggaaggtgggttggattcgggttgtagggtgaattttattgggcgttttcgttttttcgaagtcgtttcgcgttttcgcggtttttttgttttacggttggtttgttattttttgttcgaggtttagttcggggttttaaggtggcgggagttagtattatgtttgggacgtttgatttaagtagtaaaggagtaggaaggagaatttttggaggaagcgtaggtagttatgcggagggcggggaggaaaagtatatttattggattggtattttttttaatagagtttttcgttaatgatatttatttaattgtatttgatagtagttacgaatatattttcggtaaataggatttatattttttgcgtttaaaattgtttgttagtttttaagttaatcgtagtaataatcgttggattattttaaatgtggttaaaatcgacgttggataaaattaatttgttatatgttggttttgaaggaaattatttgtatagtttgacgaattgtttgtagtttttttagtaatttataattcgttttttttttttttttttttttgtatttttatttttttcgtttttttcggagtttttttttttattttattttttttcgtagattttaggtaattcgaaatggtattgattttttttatttttcgattatttttttgaagtatgaatttgggataaaattatatattagaaaattcgtcgtaattattttgcggtgttattagtatcgattaattacgcgcgcggtttagtgttggaatgtttagcgtagttcgggacgcggattcgcgtttcgtttgattgatagttttggtaattaagtgattgtatagattttttcggttgttttaataattttgttagggtttgtttgtggatagaataagttgaaattaatgtgttttttgttttttgagtagttttgatcgtgtatttagattgatcggggaggaggaatttgataaaaggaaaatagtagttttaaacgatcggattagggggagtaggtagaaagtttatttttttgatggggagggaagagcgagagatacgtattagattataagagtttcgttgagttgggtagttggttttgcgtggagcgagaggttcggtgtttgttattttcggtttttaggtaggattggtagtatttttttttcgtttttaagggggtgtagaggtttttcgcgttgtagttgatgggttttgttcggtcgcggatagtagcgtttttatacgttgtagttataattcggtagtataatcgttttcgttcggattagaggtagagattggtggtttagtgtaaatgttacgtagtagtttttaataaatgaatgtttagattgttagattgcgaagtatattgtgagttttgtgcgtttttttttcgtttttttaattcggtttcgaatttttttatttttttattttagtatatatattatatttcggcgtacgttataaatatgttatatatttagagtgatttcgttacggcgtttgcgggaggggtatttttcgagtttaatatttatttcgtcgaggtttgcggggcgttcgggtcgatcgttggtattaggaataggaaaaagattttgacgggatttgttttgcggaagcgttttttataagtttattttggaatttttgaaagaaattttttttgggaagagggttttgaaagcgtcgaattaggaggcgggatttttagttttcggcggaggggttttagttaagagtattttaatttttttatattagtttttttaaattttaaaaatttaattaattgaacgtaaatatatataaaagggggaagggaagtaaaattaattcgtcgagtttaaagcgtcgtgtcgcgggaggaggttgtagggtgttcggcgtagggtcgagtgcgtagcggagggttttatcgatcgcgcggagattgcggtttttattcggtagatatttagtgggttgtgtatgtttgtgtgcgtggtggggggcgttacgttttaatttagttcgtcgtttgtttgtttttttttttttattagtttgtagtcgtttatttttagagagaaaaatgtgagtgtgagtatattacgtattattagggatatattacggttttaatgtggtttaattagtgtcgttaatcgaataaatattattatattgaataatgataatatgatgaaaatattaatgttttggaattttgtacgtgggagtcgtttttttagtttttttgggatattttaagtacgtttagatttattagtagatgcggtcgggcgtatttttaggaaggcgagtttatagtttaagatatacgtgtgttatcgtaggtatagaaataatatcgtgggttatgttataagttttttatttatatatcgtttattacgtaaaagagttttttttaatggtattttttttttttttgtaagagtcggtaaaaagtaaattagataggagttcgttagggtttttttttattgcgtttttattcggtattttaagaatgttgtattgggcgtttcgagtgtgtttggttttaatagaggttgttaggtaggtggagttgcggaaaaaggatttatttaattagtatttaaatttatagtttttttttattgttaaatgtttttttttcgggaatttggtgaaaagtaggaatttaataattaggaaatgcggaaggttttttaaaatagggattcgaaattttaatttttttatcgattttggagggttaggggggtggggaagcgcgcgtgggttgggtgggaggtacgttttggttggcgtgcgttttcgttttcgacgtttagtatattattttatttttttttttttgtatttttattttttttttatggagtttcgggagatagagttataggaggttggtagttttgtttttgttgtttgaaggtaggtttagtttttttttggttgttgagcggtattagggttagggtttaagtagggtttttagaatgaggcgttgggtgtgttttaggaaatagggatgtaggatttaggttttagagcgttttttttcgggttgaattgttttttatttttgttgggggtttataggttatattttttcgttttgcgggagttttttttaggaggaaacgtcggtgttaattgttgttgaatgagttgtttttcgacgtaattatttatttttcgggcggtgtgtttggattagaagtcgttgtttaggtatttgtttagtggcgtattttgtagatttgggagagattgagaaagttgatcgaggttgtttttaattttcgcgttttaaggttttttacggtatttgggaacgcggtttcgtttgtattaaaggtagaaaacggggcggggaggttttaatttgttttagtgatggttgtaatttaataaggcgattgattagtatttaaagtgtcgttttaattagtcgtttgttaattaatatcgtaaatgaatttttttttttttgattggtatttttgtttagcgttttgaggttttggttttttttttgttttggtttttatatttttttttatttagttttaggagttgataggttttagagttttgtttatgtatgatatggttgtgttatataggggttttttaatttttttaggagtcggtttttaaaggataatggtttaatttaggaggggtgaaataaaatttttttttatgtttttttcggttttttttttaattgtttttattttgttatttttttaatttttatat

SEQ ID NO: 22 - DNA, 9739 bp, Artificial Sequence
  chemically treated genomic DNA (Homo sapiens)
atggaagtatagaaatgtatttaataaatatgtaatagaaggataattaatttaatattaaagtgattagagagttaatgttagggttttatgggtatgttgatattttagaatagagaggattgaaggttagagattttgggtaatttgtatattaagaatgtttttaagttattggggggaaagatagagttttttttgttttatataaaggggtagatttaaaattatagttttttaaaatttttaaaaatttttgtaagatttgaagagagttttgttttaaattttagtgtatataatataaatatatattattaatatttataataagagattggaattaaagtgtatattattttttaaagttttttaaaattgtttttaattataaaaaggtaaattaaaaattttttttatgttatagtagtttaaaaggtaaataatggaagtagaaaggagatgtatatttgttgaaatttgttatgtattaggtattttatataatttatttttatttgatttttataataattggtaaggtgtgtgttgtaggatattaagaggttatagattttaggaggttaagaaatgatttaaaattttatatttaatagttggtatagtaaagatttgaagttaagaatatttgtttttaaaaattagttgtatattatgataatatattttaaataaagatgtataaaaagtatttggttttttatagatattatataaaatatgggataatattagtgatatatttaaatttagatattagaaagaagttttttttaatttattttgtaattgtaatttagttgtttattttataggtttttatagtaataattaaatatttttttttttgttttatatgtgtttttaaattgttattaaagatttttggttagtagtttttatattttttggtgttttgttgttaatgtttggtaaatatgggtagttaaggaaaatgtagatggtgttatggaatataaattatgataatatatggtatatatttagttttaataaagtgtgttttttaatttttgggtgttgttttattatttttttataagattatttatttttagggaaggtttagtttttaaaattagggtaataggtttatatgtgttatatggttatttagatgtatagattatgtgttttaatatttttaagtaagtgtatttagtatatgttttttagtattattatgtgtatttatgaattgttttgtttagatggtaagttttttgaggataagtgttatgtttgaaattttattttattaattgtgtatggtagagtaataatgttatttattttgtatatagagatatataatttataaattgtttttttaggtaaattttttaattatattgttatattgtgataattgttatattttttagatgaggtttatataggttaagtaattagttagaattataaattaataggtaatgttttttttttgtttataagagaaagttttttttgttttgtaatattttattgttttttttttttaaggtttttattataaaggttaatgtttattggttttttaataaaagtaattattatttagtatttagtatatatatgtaaggtattgttttaagggttttataaatattaatttatttaatttttttaattattttatgagatagatattatttttatttatattttatagataaggaaattgagggatagagagattaatttgtttaaaattagaggattggtaaatagtagtagtgaaatttgaatttagtagtgaagtatagttttatagtgtgtgtttttaaatatatattattttgtttgaataagagtttataaagatttttatttaggatatatgaggagttgtttagtaagaaaagaagtatgtttatttttttttatttgatgtatttgattatattttaaaatttttttttatgttaatagaattttgtgtattatattaaatttttttgtattgaaggtatatgtaatggttattttgattgatatgtaaaatatttaaaataaaatattttgagatttgaggattttttgttttatgaattagatattaaaaaaaaaattgtttaggtattagaagtgttatatatttatatttgtaagttttttttgggatttttagattttttatataagatttttttttgtttgaaatattttagtgtttatttaatgtttgagtttgaaaattatttaattttttttaatttttttatttttagttattttgtttttaggtatagggtagggtaagtatttgtttttaatatgatgtttaatattttttatgtttttatttttgttattttgaaaatatgttagtaaatatttaaatatattgtaaagatttaaaatttggggtgtgtatatgtatattaataggagttatgaattatgtatattgtaggtttatataaattagattttgaagaataagtattatagtaattaagtgggttataataagatattttatgtgaaatgtaaaaattattttgaataaattatttgtaagttaatatttttattagaatgatatatttattatttttgggtttgtgatattgttttaatgtattttggttaattgttttaatggtatattaagaaattattttaggttttttttttttgttggaatttgtagatttttatttttgtttgaattaaaataatataatattaaaaataattttttattttttaatgtataatattatttttttttgtttagaaatatttaattaaagtaggatataattttagtaattatttttatatagaatgatttttaaattaatagttagttgtatgattaaaaattgggagattgataattaaaataattttaaatgttttgtttatgtgatgaaaatgagtgattttttaatttttttatatatattaaattgattttatagattttttgtattggtgtttaatatagttaagtgtttgaggttatgtaaaataaataattttgagattttattgtaaatgtttgtaatttatgatgtaaattgatttgtgaagaaaaaataggattttatgttgttgaagttaaagaggtattttttagaaattaaaatagttttgaaatttgagtattgtattatattaaagtattattatttgaggatttaaaaaagttataaatttggggaaaaatttaaaatagatgtatattagtttagttttaagtaaaggtattgattttattattttattgtgttttttgtattatagggtttttaatattaatgatgttttaaataattttttttaatttagttttttttagttattgattttgtaatttgggaaggttttgtatgtttaaaagatttggggagtagaggatgggggtgttttttattaattatattaggattattgagttttgatttaaattataagtatgttgggttttatttaaatttaaagtgaattttttagatttttttttgggatggtttggggaattatgagtttggaatttttgtttatatattgaggtgtttgttttagagtttggatattggtattttgggagagtaggttttgtgggagtttggatttgaagggtgagattttatagggttaaggaaagtggtttttgttttttgttagttttgggggagtagatgtaagaggaggtaagggtgttgtgagttttttggatgtattggttttataggttgtgtttgagtggagtattgtgaatggggttaagaaattttggttttttttgttggatttggttgtttttgtgggtttttttgtttattgtgtttttgttgtggtttgatttttgtgggtttttgtgttgaatttatttggtttttattgtatgggatatttttgatttatttatgttgtgttattgagtttttgtattgatatttggtgtttttgttagtagggtttggatgtattgttttttttgattttgggttttttttgtgttttgttgtttggggtagattggttttgagagggagttattattttttttgttttagggtttttagggtttgaatttgtgttgggatttgggttaggattagggtttggagtttggagtttgtttgttaggatttttggttttggtgttgattggagtttgttggaggttataggattatggtggatggtttggttgttttagggtgtattatgtttgtaggtagatgtttattattaaaaattattgatgtttattaattaggagatttgtaaggtttgtgtattgaatatggtttaaattttgtttagtggtttttgaaagttaaaagaaagaaatttattgtttatgtttatgttgaggaatagttttgaatatgagttaagatttgggagagggttaagtgggggtggtggggaatattgttggaggatgtggggttttggtaggggttttatgtattttttagtatgagggtggggggttttaggggatgtttaggatttttttagtttttgtggaaggttttgttattataagagtttgtgtgggaaggaaagtttttgttttggatatggttagggttgagtttttaaagtttttaattatttttatataggtagtatttttgggttttattagtgggattttttttagaagggggttatgatatggggagagagagttttttattatttttaaggttaggttttttttaaatttgattttggtttttattttttaaatttaattaatgaagaagttgttttagttaattttaaataggagtgttagatggggaattttttttttatagtgttttggtttgtttgtttttgtggttttttttttgttttgaagttagtatttgttttgttttagagagggttaatgaattgggattgattgttgattatgttatattaaatttaggttgattttgttttgtaggatttttttttttttttttttaaaagtgtttttagataaagattggaattatagtaaaatgaataatgagagtttattttggggaaggaagttattttattttatgttattttattttttatttgtttttttattaatttggtataattttgtttttatgttgggtggataaaatggagggggttggtggaaatgtgtttagtgttaatgatagttatagagttattttttttattgttttttgattgtggtatgtaagagtagaggtaagtgtttttttaagttggtatatttgagagagtgatatgtattaattaaaagggaaagattattttggatattttaatagtaataggaataaagatgatttgaattttattaaatgaaatgatatttttatttaattgattgtaaagtgtttttaattaataatttggtatttatttaaaaggtttaatagaattggaggtgatatattttgtattgataatatgttatgagataatattttttgtttaaaataatttaatatattagaaaaaaatatgttaaatataaataattttgtttttatggagaataaatagttataaagtttaattgtatattaaagtttagttagaagagtttttttttttttttgtaaattagaaggtaaaataaaaataaaaataaaattttataaatttaggtttgttagtgaattgggatatagattggtttagtttaaggtattttagtttaaatagttatatgaattgaaataaataattaaaagaaaaaaaaaaataaaaaagaaataaataaataaaaaaaaaaggttatttgaatagatatatttttgtgagataaaatgtaaatgttgaaagttagtttatatattatagttaaaataaattttgtttgtgtgtattaatatattttatataaatttagtgtttgtatttagttgggtgttatttaaggtgaatttgatgggatagagagggggaaataaattattgtttttaaataggattttaggaaattaataagaaggaatataagaaaaggggaatggtgggaattaatattgattgagtgtttattgtgtttggtatagtgaggtgttgtatttttattattttatttggtggttttgtttttttttttttttttttatatattttgtttatttttgtttttttaggataatgagtgtaattttttttttttttgattttttttttttgttttttaggattaaagatagggtaaatatatatatatatatatatatatatatatatatatatatatggtaaatatatgatatatatatatggatatatatatattaatttttagatatttttggtattgttttttatagtgaagatgaaaatggagtgagtgtatgggagataagggggtgggaggagatatattattaatggaatataagtaatatgtaaattggaatttattgttagtagtttagatagttttgttataataattttttagagaaattattgtttgtggtatttttttgtgttaatattatttttttttttttttgttttaatattttttttttttttgtttttttatatatggtgggtttaaatttaaagtatttgatttaatgtaaaaggaggtttgttttttttattaaattttttgtgttaatgtgaattttgtagaaaaaggttagttttaaaatttgtgaattttttagttgtgtattttttttattttagttttttaaataagtatattttttttttttgttttgtttttttagtgtatttttaaaaatatatatttaagttggtaattttttttagttttttgagaattgtttgggttgtttttttatatttttaaatgtataaaatattttaattttaaaatttgtttttgttttttggttttttaattttagtttttgttaaatagtagttaagaaataagtttttttttttttttttttttttgtttgttttttgatttttttgtttgtttatttttttattgttaataagatttttttttttatgtaagagtttattgttgtagttttgtggtgagttaaattttgtggttttagtatttttttgtttagtttttttttagatttttttaaatttgtttttataaaatttaattttaggtttttgagtaggaaaatgggtaggagttatggagtttgtgtgttttgtgagatttttggttttgtgtggagtttggttttttgagtttaagatgtgataggggatgagggatggttagtgaggtgggaagagggttggtttttgaggttttaaaggggtaatggagaagtagtggggtgtggagggtgtgtaggttgagtgttgtgggataggtgtgatattggtgttggtgttggtgttatagatttagggttgtggtgtgttttttggtttttagtttgagattggtgatgttggagtttttgttggtggttttggtggttgttgtggttgttattattgaggtggtggaagttgaatttgtggttagtgtggtgagtggtagtttgaagggtggtgttgggaatattatgtagggtgtgtgtgtggttaggtgtggatgtaggtggtggtgtgtgtgtgttatagtgttgtttagttgtagttgtgtttgaatttgaaaggataagggtgttatgttgtaatgattattttagggtgataatagaatagaaatagagtattaatggtgtttagtttggttagagagagagttgggttgtgtttgggtatggggagagggtatttggttgtgtattttgttttgatagtgatttttttagtttgttgttgaatttggggttttatatggatattttagtgttttttgggtttttgaaatttgtttagggtaggtttatttttttggattttgggaatttttttttgtaggaatgtggttgttttttttgggaattgggttgaaatggtatattggaaagattgtgggtgaggatttttattttttttttttgatgttgttatgtattttttattttattattaaaataatggagtattaataagtaaattatgtaattttttaatgttatttagggaagttattttatttttattataataggtataattgtgagttggtatttttaatttttattgttataataattatttagggattttgttaaaatttagattttgatttgtaaggtttaggttggggtttgagattttgaatttttaataattttttagatggtgttgttgttgttggtttaaagattattttttgagtagtaaaggtttttttttaaagttatttttttttttttttttagttatttttttttgtttttaaagttaagtattaggatgttagagttttgtggtattaagagagttaagatttttttaatattgatttttaatttatggttatatttatttgtttttttggtttaagttattggagattgagagttaatttatgatattataggtatgttaagtaggggttttggttgaggatagggaaggttagatattggttgggaaaggattttgtttttaggtttggaaagtggggagtttagagtattgggattttttaggattaaattattgttgtaggttgtttttttaaaatgtatttttaaaggtttttttatggttaagggattgtaaaggtagggtagttttgggaagattaagatttttttttttattagagatgaagttttgtttgtagaaatagatttaaaaataaaagaatgaaaaaataaaagtatgagttgggagtatttgtgtttattttttttttgttagagttatttaatgtgattaagaggtagaaaatatttagaaaagtatttagagggttgttttagaaatattttaggattaatttttgtattggatttgataaaattttaattaaaaggttttttttttttttttttttaagggtaatttttagtgtatttttaaaggattagttattgtttttaggtttttttagggatttttatgaggatagaagggattaggtatttaggtttttaatattgtataggtttttaaagggtagtgggtttattatattattttaaaatgattttttaattatgtagatattgatatttgggtttagagataggtgatgtttaatagtttaagtaaatatttaggtatttttatatttaaaaatagtttgaaattaggttgtatttgtttgttgaaatggtatttttaaagtatttatgttgatataaggtgtgattttataagttttaaattggttggtggtttttatgagaatatttgtaaaaagtataagagaaaaataatgtgaggttaatgtttgtagtattttttagggttttaaagggtataagatagaatgaatttttttgaaaatggattattatttttttaaattgtatttaaaatttaaataaatgtaatttatttttaaattttaggattttattaatagtttttgaagattattaagttaagaattattgttatttttgaggtttttttttttttgttattgtaatagtaatatttattatttttttttttgtttagattattaaatgttttttgtataagaagtatatttttatggagttgatttttttgttttttatatttagttttttgattttgaaattaaatttataggttggagggggaaaaaaaataaaatttagatgttatgattaaaaatttttttaaattataaaagtataaagagaaaagagttgtg

SEQ ID NO: 23 - DNA, 9739 bp, Artificial Sequence
  chemically treated genomic DNA (Homo sapiens)
tatgatttttttttttttgtatttttgtaatttaaaaaaatttttagttataatatttaggttttattttttttttttttttaatttataggtttggttttaaaattgaagagttaaatgtagaaaataagaaaattaattttataaaggtatgttttttatataaggaatatttgatagtttgagtaaagagagaagtagtgaatattattattataatgatagaaaaaaaggaattttaaaaatgataatagtttttgatttaataatttttaaaggttgttaatggagttttaaagtttgggagtaagttatatttatttaaattttggatgtagtttagaaagataatagtttatttttaaaggaatttgttttgttttgtattttttggaattttgaaaaatgttataaatgttaattttatattattttttttttgtattttttataggtgtttttataggggttgttagttagtttgaagtttgtagagttgtattttatgttaatgtaggtgttttaaggatgttattttagtaggtaagtatagtttagttttaagttgtttttaaatatgaaaatatttgaatgtttgtttgaattgttgaatattatttgtttttgagtttagatgttaatgtttgtatagttggagaattattttgagatgatatagtggatttattattttttaaaaatttatatggtattaaaggtttgaatgtttagttttttttgtttttatgggaatttttgaaggggtttgggggtagtaattggttttttaaaaatatattaaagattgtttttagagagagaggaaggaaaagttttttgattaagattttgttaaatttaatatagaggttagttttaaggtatttttaaggtaattttttagatatttttttaggtattttttattttttagttatattaaataattttgatagaagaaaaataaatataagtatttttagtttatatttttgtttttttgtttttttgtttttaaatttgtttttgtaaataaagttttatttttggtaaagagagaagttttggtttttttagggttgttttgtttttgtaattttttagttataaagaaatttttaagagtatattttaaaaggatagtttgtagtaatgatttaattttgggaaattttagtgttttgaattttttattttttaagtttgagggtgaaatttttttttaattagtgtttgattttttttatttttagttaggatttttgtttggtatgtttgtgatgttatgaattagtttttagtttttagtgatttggattggggagatagataggtgtgattatgaattgagagttggtgttagggagattttagtttttttggtgttataggattttggtattttgatgtttggttttggaaataagagagaatgattgaagaaagagggaggggtgattttaaaaggagatttttattatttaaaaggtaatttttgggttagtagtaataatattgtttgggagattgttagaaatttagaattttaggttttaatttagattttgtgaattagaatttgaattttaatgagatttttgggtaattgttatggtaataaaggttagaagtattgatttataattgtgtttattgtggtgaagatggagtaatttttttgaatgatattaaaaagttgtgtagtttgtttgttgatattttgttgttttggtagtggggtaagagatgtatggtagtattaagagaagagggtaagggtttttatttgtagtttttttaatatattattttagtttagtttttaggggagatggttatatttttgtagaagggggtttttggggtttggggaaataagtttattttaagtgagttttggagatttaggggatattggagtatttatgtggaattttagatttagtaataggttgagaaggttattgttggaataagatgtatagttaaatgttttttttttgtgtttaaatataatttaattttttttttggttaagttggatgttgttaatgttttgtttttattttgttgttattttaggatagttattgtaatgtgatgtttttgtttttttaggtttaggtgtagttgtagttggatagtgttgtggtgtatgtgtattattatttgtatttgtatttggttgtgtatgtgttttatatgatgtttttagtattgttttttggattgttgtttgttatgttggttgtggatttggtttttgttgttttggtagtggtggttgtagtagttgttaagattattagtaagaattttagtattgttgattttagattgaaagttaaaaagtatgttgtagttttgggtttgtgatgttaatgttagtattaatgttgtgtttgttttgtggtatttagtttgtatgttttttgtgttttgttgtttttttgttatttttttgagattttgggagttggttttttttttgttttattgattattttttgttttttattgtattttggatttggaaagttagattttatgtaggattagggattttatgaggtatgtaggttttgtggtttttgtttgtttttttatttgagggtttagaattgggttttgtaggagtgggtttgggggagtttggagagagattggataggggagtgttggaattgtggagtttggtttattgtaaagttgtaatgatggatttttgtatagaaaaaaaaattttgttaataatgaaaaaatgagtaaataaaaaaattgaaagataaatgggagagaaaaagaggaagggaatttattttttaattgttatttggtagaagttgaaattggagaattaaggagtaaaaataaattttaaaattaaagtattttatatatttaaaaatatggaaaaataatttagatgatttttgagagattggggggagttattaatttaaatgtgtgtttttaaaaatgtgttaagaaggtaaagtagaaagaagaggtatatttatttaaaaaattaagatgaaaaaagtgtgtagttgggaagtttataggttttgaaattgatttttttttgtgaagtttatgttaatatgagaaatttgatgagagaggtgggtttttttttatgttgaattagatgttttgagtttaaatttattatgtatggaagagtaagaaaagagaaaatattaaaatgaggagagagaaaaataatattaatataaaaaaatgttatagataatgatttttttgagaaattattatggtaaaattgtttggattgttgatagtaaattttggtttgtatgttatttgtattttattgatggtgtgtttttttttatttttttattttttatgtatttattttatttttatttttattatgaaaaataatattaaaagtatttggaaattgatatatatatatttatatatatatattatatatttgttatatatatatatatatatatatatatatatatatatatatatttgttttgtttttgattttggggaataaaagaaaaaagttagaaagggaaaaaattatatttattgttttaagaagatagaggtgggtagaatatgtggggaaaggaaaaagaaaataagattattaaatgaaataatgaaggtatagtgttttgttgtgttagatatagtaggtgtttaattagtattagtttttattatttttttttttttgtgtttttttttgttggttttttgaagttttatttgaagatagtggtttatttttttttttttattttgttaaatttattttaaataatatttagttagatataggtattaggtttgtgtaagatatgttgatatatatgaataaagtttattttgattataatgtgtggattgatttttaatatttgtattttattttataaaggtgtatttatttaagtaattttttttttttgtttgtttgttttttttttgttttttttttttttttggttgtttgttttaatttatgtagttatttaaattgggatattttggattaagttagtttgtattttaatttgttagtaagtttaagtttgtggggttttgtttttgtttttgttttattttttaatttataagaaagaggaaaagtttttttaattgaattttggtatgtggttgagttttgtaattatttgttttttatgaaaataaaattatttatatttgatatatttttttttagtgtattaagttattttaaataaaagatgttattttatgatgtgttgttagtataaaatgtgttgtttttaattttgttaaattttttaaataagtgttaagttattaattgaagatattttgtgattaattgaatgaaaatattgttttatttgatggggtttgagttatttttgtttttgttattattaaaatatttgggatagttttttttttttgattaatgtgtattatttttttgaatatattaatttggaaaagtgtttgtttttgtttttatgtgttgtagttaaggggtagtaagaagggtggttttgtggttgttgttagtgttgagtgtatttttgttggttttttttattttatttatttagtatagaaatagggttatattaaattaatgaaagaatagataagaagtaaaataatataggatagagtaatttttttttttaagatggatttttgttatttgttttgttataattttagtttttatttggggatatttttgggaggaaggaaggagggattttgtggagtgaaattaatttgggtttggtatggtataattgataattagttttagtttgttgattttttttggagtggggtaggtgttgattttggagtgggaaaagagttgtaaaggtgggtaggttagggtattgtggagggaggattttttatttgatatttttgtttagggttggttgaagtagtttttttattggttaaatttagaaggtggaagttagaattgggtttaggaaaagtttggttttgaaaataatgaggagttttttttttttatgttgtggtttttttttggaagaaattttattagtaaagtttaaaggtgttgtttgtgtggggatagttggagattttgaagatttggttttgattatgtttaaggtaggaatttttttttttgtgtgggtttttgtgatgatgggattttttgtaaagattgaaagagttttgagtgttttttgggattttttatttttgtgttggagggtgtgtaaaatttttgttaaagttttatattttttagtagtgttttttattatttttatttggtttttttttaggttttagtttgtatttaaagttgttttttagtatagatgtgggtgataggttttttttttttaatttttaaagattattgaataggatttgggttgtatttagtgtgtaggttttgtgggttttttggttgatgaatgttggtagtttttaataataaatatttgtttgtgggtatggtatgttttgaagtagttaggttatttgttgtggttttgtggtttttagtgagttttagttggtgttggggttgggggttttaataggtaggttttaagttttaaattttaattttaatttagattttaatatgggtttggattttggagattttggagtaggggagatggtggtttttttttggggttagtttgttttaagtagtggagtgtgggggaagtttgaggttaaaggaggtggtgtgtttaggttttgttggtggaggtgttgggtattggtatagaggtttagtgatgtggtgtgggtgggttgggaatgttttgtgtgataggagttaggtgggtttggtgtggagatttgtgggagttgggttgtggtgggagtgtggtaggtggagaggtttgtggaggtagttaggtttggtgagaaaggttaaaattttttggttttatttgtagtgttttatttgggtatggtttgtgggattagtgtatttggggagtttgtggtgtttttgtttttttttgtgtttgtttttttaagattaatggaggatagaggttgttttttttggttttgtggagttttgttttttgggtttagatttttgtaaggtttgtttttttggaatgttagtgtttaaattttggggtaggtgttttggtgtgtgaataagagttttaaatttgtagttttttgaattattttaagaaggggtttgaggaatttattttaaatttaaataaggtttaatatgtttgtagtttgggttgaaatttaataattttaatataattaataaaggatatttttattttttgttttttaaattttttgggtatgtaaaatttttttgagttgtaaaattaatgattgggaaaaattgagttaaaagagattgtttaagatattattagtgttaaggattttatgatataagagatatagtaagatagtaagattggtgtttttgtttggagttgaattgatgtatatttattttgagtttttttttagatttataatttttttgggtttttagatggtaatattttagtataatgtaatgtttaaattttggaattattttgatttttgaaaaatgtttttttggttttagtgatataggattttgttttttttttataaattagtttatattataaattgtaaatatttgtaataagattttaagattgtttattttatataattttaagtatttgattatgttaaatattagtatagaaaatttgtgaaattagtttaatgtgtgtgagggaattaaaggattatttatttttattatataaataaagtatttaagattgttttgattgttagttttttaatttttagttatataattgattattaatttaagagttattttgtgtaaaaataattattgaggttatattttgttttaattaaatatttttgggtagaagaaaatggtattgtatattgggaagtgggggattgtttttaatattatattattttagtttaaatggaaataaaagtttgtaaattttaatggggaaaaaaggtttgaaatggttttttaatatgttattaaaatagttagttaaaatatattaaagtaatattataaatttagaaataataaatatgttattttaatgagaatgttaatttatagataatttatttaaggtagtttttatattttatatggaatgttttattatgatttgtttgattattgtgatgtttgttttttaaaatttaatttatataggtttgtaatatatataatttataatttttgttgatatatgtatatatattttaaattttaaatttttatagtatgtttggatgtttattagtatgtttttaagatgatgaaaataaaaatataaaaagtgttaaatattatattgaaagtaagtgtttgttttgttttatgtttgaaggtaaagtagttgaaaatgaaaaaattaaaaaggattagataatttttaagtttaggtattgagtgagtattgaaatattttagataaaaaaaaattttgtataagaaatttgaaaattttaagaaagatttatagatgtgagtgtataatatttttgatgtttagataattttttttttggtgtttaatttatgaaataaaaaatttttgagttttggggtattttgttttgggtattttatatgttagttaaggtaattattatatatgtttttggtgtaggaaagtttagtataatatgtggagttttattggtatgaggaaaggttttagaatataattaaatatattaaatagggaggagtaaatatgtttttttttttgttgaataattttttatatgttttgagtaaggatttttgtagatttttatttaagtagaatagtgtatgtttaagagtatatattgtggagttatgttttattgttggatttaaattttattgttattgtttattagttttttgattttggataagttaatttttttgttttttagtttttttatttgtaaaatgtggataaaaatagtgtttattttatggaatggttaggaggattaaatgagttaatatttgtaaaatttttagaataatgttttgtatatgtatattaagtattaaataataattatttttattaaaaggttaatagatattgatttttgtaataaaaattttaaaagaaaaaaataataaagtgttgtaagataagaaaagtttttttttgtgggtaagaagaaaatattgtttgttagtttgtgattttggttaattgtttagtttgtgtgagttttatttagaagatgtgataattgttataatgtagtagtgtagttaggaggtttatttagaagagtaatttataggttgtgtgtttttatatgtaagatagataatattgttattttgttatgtatagttaataaggtgagattttaggtatggtatttgtttttgaggagtttattatttgggtaaaatagtttataaatatatatgatagtgttaggaagtatgtgttaaatgtatttgtttaaaggtattaggatatataatttgtgtatttgaatggttatataatatatgtgggtttgttgttttggttttggaaattaaattttttttggaaatgagtagttttataaaggaatggtgaagtgatatttagaggttaagaagtgtattttgttaggattaaatgtatattatatgttgttgtggtttgtgttttatagtattatttgtattttttttggttgtttatgtttattgagtattagtagtaaaatgttaaaagatataaaggttgttgattaaggatttttaatagtagtttgaaaatatatataaaataaagaaaggaatgtttaattattattatagggatttgtaagatgaataattgagttataattatagagtaaattaagaaggatttttttttaatgtttaaatttaggtatattattaatattgttttatattttatataatatttatgaagagttaagtattttttatatatttttatttgggatgtgttgttatagtatatggttaatttttagaggtagatatttttgattttaaatttttgttatattaattattagatgtaaagttttaggttattttttagttttttaaaatttatgattttttagtgttttgtagtatatattttgttaattattgtgaggattaaataaaaataagttatgtaaggtgtttggtatataataggttttaataaatgtatattttttttttatttttattatttattttttaaattattgtaatatagagaggatttttaatttatttttttgtgattagaagtagttttgaaaaattttgggaggtggtgtgtattttaattttaattttttattgtaagtattgataatgtgtgtttatattgtgtgtattggagtttggaataaagttttttttaaattttgtaaaaatttttagagattttgagggattgtgattttagatttgtttttttgtgtagaatagaaaggattttatttttttttttgataatttgaggatgtttttaatgtgtaggttatttagagtttttgatttttagttttttttattttggaatgttagtatatttatggagttttggtattaattttttagttattttgatattagattgattgtttttttgttgtatatttattgaatatatttttatgtttttgt

SEQ ID NO: 24 - DNA, 4313 bp, Artificial Sequence
  chemically treated genomic DNA (Homo sapiens)
gggtttgattttttgagatttggggaggatttttggtagatgtgtgtttagttagaatatttggtaaggatttttttaatgaagaaaaagtggaggaatttagttttagtgagaagaggtttttttattttgttttagatatattggatagagggtatattttgattagagttatgtttagtggttaggaggttagtttagtatttttttttttattatttttgttttgggtgggggggtaatttttttgggagtagttgtgggaattgttgttttttattttagtttagttagtattttgaagtttgtaggggaaggatagtatgtgggatggatattggggaaggagttttgtaaggttagggtgtaatttttaggttttaggtggtttggtaggttatgttgttttggagatgtttgttagattttttaagtttatttagggtttggtagtaatttgttggttgtttttgtgggggtttgggttgttgagtatggtgtagttgtttagggttaattagttttagggtgtttgtgttaggttgtggtttttttgttttttttgtattgagggtatttatggtgtgtaaatgtttttgtatttttagagttgttttattggatgtttttaggaatttatatatttttataaaaatgtattttaaatgatggataggtgagtttggggtaataatgggtgtttggtgggtagataagagtaaatgggaaggagtttgagggaggagggggaagagaagaggaaatagaatttttagttggatattttgataatagttggaaggaaagtttagaaaagatgaagagagaggaggggagaaattaattggggtttttatttttgttgttggatttttaatttttgttttaaatgggttttgttttttggtaaaattagtttaaaggattttaaaataaagaaaatgagatgattggtttgggagttttttaattagagtagagaagttagaggggggtgggtgatttggttttgaagttttagttgaatagttattttttttttttttggtaaaaaggatttttttagaatttttgaggtttttggatttttttttttgtaaatggagttgtatattgtatttttttgtttttttggattgttaagtatgttttatgagggttgttgtttttgggtggaatgtggttgtatgtatgtgtttttttgtatatgtatatatatgtatatttataataagtgtttgtaggaggagtgttttgtgtgttagttttgtgtttaagataggaagttgttgggttattgagttaaatgggagtgatattatttttttttattagtaaggaaagtggattataaaagtttttttgtattttggtagtttatttaatattatttatgtattttgtgtaaggaattgtgggattttgttttatggtaaataatatggaaattttaaaaatagtgatttttttgtgtgtgtttatttatgtgttttggggtgatttggtggggttgttgttgggtgatttatatttttgaattgtgaagtgatagggaaagtgtgggtgagtgtaggagatgtggttgggggtttttttgggtttttgggtttttgtatttggagtgggggatgtggttgttttaaggggaggaggggtggtgggttgtttttgttatttagtggtggttggagtgttatgtgggtgtgtggtgttgtggttattggtttgaggtatgtgtttaggagattggtttgtgatgttatttgagggggttttgttaaaaataagaataaaaatttagagtgaaagtgttttaggttgtgttgagtggtttggaaatttttgagtttgtgtggaggttgaggtggtgagggtggtggatggttggggagtgtgggtggtttagtttggtttggttgggttttggttttgtgttttttatttatgtgatttgggttgtggagttttgtggggtttggtgggggtgtggttgtatgttggtggggtgttttggtttgtagtggggtggtggttgtgaggagggggtttttatgtgtgtgtgggtggtggtgggtgtgttgattgtgggtgtttggtatttttgagggttggttagggtgtgtgggtggggatggttgggtggtggtggtggttggagttggtttgggtgggtgtgagtgttggggaatgtgttgtttgtatgtgtgtagtttttgttttgggtggtttaggtggtggtgttggagtttgaggtggttggatgtggagaggagtggggagtttgggaggtggtttgtgtttttgttggattattgtgattgtttagattttggttgtgtggtgaagttgaggatttggttttgttgaattttttattgtttgggtgagtggggtggtttgtggtgtttttaatttagtttgtggatttaaaggtggttttgtgttgagtgtggttggtgatttgtaggattttagttttggttgtggttgttgtgtatgtttttggaagatttggtggggtgggggtgtgggggtttttgtgtgtgttgtgggagggttgaaggttgatttggaagggtgtttttggagaattagtgtgggatttattgtgaatagtatggaggagaatgattttaagtttggtgaagtagtggtggtggtggagggatagtggtagttggaatttagttttggtggtggtttgggtggtggtggtggtagtagtttgggtgaagtggatattgggtgttggtgggttttgatgttgtttgtggttttgtaggtgtttggtaattattagtatttgtattgtattattaatttttttattgataatattttgtggtttgagtttggttggtgaaaggatgtggggatttgttgtgtgggtgtgggaggaggaaggggtggtggagttggtggtgaaggtggtgtgagtggtgtggagggaggtggtggtgtgggtggtttggagtagtttttgggtttgggtttttgagagttttggtagaatttgttatgtgtgtttggtgtgggtgggttgtttttagttgttggtagtgattttttgggtgatggggaaggtggttttaagatgtttttgttgtatggtggtgttaagaaaggtggtgattttggtggttttttggatgggttgtttaaggtttgtggtttgggtggtggtgatttgttggtgagtttggatttggatagtttgtaagttggtgttaatttgggtgtgtagtttatgttttggttggtgtgggtttattgtatgtgttatttggattggttttttttaggtgagtttgtggggattatgtgttttggtttgttgtggggaggtttgtggagttggggggtggtgttggtgtgggaatttattgggaggaaaatattttgaattttttttgtgtatatgtataaagatttatgtgatattgtgtgaagttgatgttggtttgggtagtggttaggagtttagtggtaggattgatttgttagggggtatagattttttaggattgtagaagggattttttttttttttttgttttttttttttttttttttttttttgttttttttttttgttttttattttgttttggtgtatttttttttagtttttagtttatgttttttttattgtagttttttttggtgggaatgtggtggttggaagatgggtttggaagtgtatatttttatttttttttttatgattttttaatttaggttaggttggggatgtatgttttagtttattttagatttgttttattatttggttattttggttgtgtttggggaagaaaaggtgaggttttttgttgttttgttttttgttttttgggtttgtgttgattggtgggatttaggaggatgtatatagggaaggaggaaaataaaggtgttttttttttttggttttattttgtttgttagtgttagtttgtagtggtggggtttagttttttttttgtatatagtgaggataagggaggtagttgttttttttggtatttgttatttttaaatagaaaggatttttttttagggttttttgggggttgttgatgggaaagaggtagtatttgtaggggttttgtagagatgttggatatatttttttatagatttgtgattttaaaaaattaagtttatgtttttgtagaaattattaattgtattttatgtgggtttgtggttgggaattgttattagaagtggattgtttgattttgagttggtagtggatttttgttgtttttaaatttttaattattttgtgggggttatttgtttagatt

SEQ ID NO: 25 - DNA, 4313 bp, Artificial Sequence
  chemically treated genomic DNA (Homo sapiens)
gatttgggtaaatgatttttgtaaaatagttgagggtttgggggtagtggggatttgttgttagtttggggttaaatagtttatttttaatggtggtttttagttgtagatttgtatggaatgtagttgatgatttttataaggatatggatttagttttttaaaattgtagatttatgaaaaaatatatttagtatttttgtagggtttttgtgaatgttgttttttttttattagtagtttttaggaaattttgagagaaggttttttttatttggggatggtaggtgttgggagggatggttgttttttttgtttttgttgtgtgtaggaagggggttgagttttattattgtgggttggtgttggtaaataaagtggagttaaggggaaagggtgtttttatttttttttttttttgtgtgtatttttttgagttttattggttagtgtaggtttgaggggtagaaggtagagtggtaaagggttttgtttttttttttttgagtatagttgggataattggatggtaggataagtttggggtgggttggggtatgtgtttttggtttggtttgagttggaagattgtagggaaggggatgagagtgtgtatttttgggtttattttttagttattatgtttttattaaaaaaaattatagtgggggagatatgggttaggggttgggaagagatgtgttaaggtggggtggaagatagagaggggagatagggagagaggaaggagagagagagatagagaaagaaagaaggttttttttgtggttttaagaagtttgtattttttagtgaattagttttgttgttggatttttggttgttgtttgggttggtgttagttttatataatgttgtgtgagtttttgtgtgtgtgtgtgggggaggtttgagatgttttttttttggtaagtttttgtgttagtattgttttttagttttgtgggtttttttgtggtgagttgggatgtgtggtttttgtgggtttatttgaagaaggttggtttgagtagtgtgtatagtagatttatgttggttagagtatgggttgtgtgtttaggttggtgttggtttgtgagttgtttgagtttgagtttattgataggttgttgttgtttaagttgtgggttttgagtgatttgtttagggggttgttggggttgttgttttttttggtgttattgtgtagtgagagtgttttggagttgttttttttgttatttggagagttgttgttggtggttgggagtggtttgtttgtgttgggtgtatatggtgggttttgttggggtttttgggagtttgagtttaagagttgttttgagttgtttgtgttgttgtttttttttgtattgtttgtgttgtttttgttgttggttttgttgtttttttttttttttgtgtttgtatagtaggtttttgtgtttttttgttggttgaatttgggttgtaggatgttgttgatgaagaagttggtgatgtggtgtgggtgttggtggttgttgggtgtttgtaggattgtgggtagtattagagtttgttggtgtttggtgtttgttttgtttgggttgttattgttgttgttgtttgagttgttgttggggttggattttggttgttgttgtttttttattgttgttgttgttttgttaggtttggggttatttttttttatgttgtttatagtaaattttatattggttttttggggatgtttttttaaattagtttttggtttttttgtggtgtatatggagatttttgtgtttttattttgttgagttttttgagggtgtgtgtggtggttgtggttagggttgaggttttataagttgttggttgtgtttggtgtggagttatttttgaatttatgaattgggttagaaatattatgagttgttttgtttgtttagatgatgagagatttaatagagttaagtttttgattttgttgtgtagttggggtttagatagttgtagtggtttggtggggatgtgggttgttttttgggttttttgtttttttttgtgtttggttgttttgggttttggtgttgttgtttgggttgtttggggtgagagttgtgtgtatgtaggtagtgtgttttttggtgtttatgtttgtttgggttggttttggttgttgttgttgtttggttgtttttgtttgtatgttttagttggtttttgagggtgttgggtgtttgtggttagtgtgtttgttattgtttgtatgtatatggaggttttttttttgtggttgttgttttgttgtgggttggggtgttttattggtgtgtggttgtgtttttgttgagttttgtagagttttgtggtttgagttgtatgggtgagagatgtgaggttagggtttggttgggttgggttgggttgtttgtgttttttggttgtttgttgtttttgttgttttggtttttgtgtgggtttggaaatttttaggttatttggtgtaatttgagatatttttattttggatttttgtttttatttttaatagagtttttttgagtgatgttgtaggttggttttttggatatgtgttttgggttaatggttgtggtgttgtgtgtttatgtgatgttttggttgttgttgggtgataggagtagtttgttgttttttttttttttaaagtggttgtgttttttgttttgggtgtgggagtttaggaatttggagagatttttgattgtgttttttgtgtttgtttgtgttttttttgttgttttgtggtttaggggtgtgagttatttggtgatagttttgttaggttattttggggtgtgtaggtggatatgtataggaagattgttatttttaagatttttatattgtttattgtggggtgaaattttataattttttgtataaaatgtataaataatattaaatgagttgttgagatataaagggatttttgtggtttgttttttttgttgatggagaggaatagtgttatttttatttgatttggtaatttggtagttttttgttttaaatgtagagttggtgtgtaggatattttttttgtagatatttattgtaagtgtgtgtgtgtgtgtgtgtgtagggaggtgtgtgtatatggttgtattttatttggggatagtgatttttatgaaatatgtttagtgatttgaaagagtgggggaatgtagtatgtggttttatttgtgaagggagaaatttaggagttttggaggttttaaaggaattttttttgttaaggagaggaggggtgattgtttagttaagattttaaaattaagttgtttgtttttttttaatttttttattttgattgaggagtttttagattggttattttgttttttttgttttaaaattttttaaattaattttgttggagagtagggtttatttgaaatgagaattaggaatttaatggtaagggtgggagttttagttggtttttttttttttttttttttattttttttagattttttttttagttgttgttagaatgtttaattggaagttttgttttttttttttttttttttttttttttgggtttttttttatttgtttttgtttatttattaaatatttgttgttattttaggtttgtttgtttattatttaaaatgtatttttatggaaatgtgtgaatttttggaaatatttgatagggtagttttgggggtgtgggagtatttgtatgttatgagtatttttagtgtgggggaggtggggaggttgtagtttggtatggatattttagggttgattggttttgggtagttgtattgtgtttagtaatttagatttttgtagaggtagttagtaggttgttgttaggttttgagtgaatttggggagtttggtaagtatttttgaggtagtgtggtttgttaggttatttggggtttgaaggttgtattttggttttgtgaagttttttttttagtgtttattttatgtgttgttttttttttgtaggttttagagtgttggttgggttgggatgagaaatgatagtttttatagttgtttttaggaaaattatttttttatttaggatgggaatggtgggggaggaggtgttgggttggttttttggttattggatgtggttttggttagagtgtgttttttgtttggtgtgtttagggtagggtggggaagtttttttttgttggggttggatttttttattttttttttattggaggagtttttattaaatgttttggttaggtatatatttgttaggggttttttttgagttttaaaaaattaaattt

SEQ ID NO: 26 - DNA, 16197 bp, Artificial Sequence
  chemically treated genomic DNA (Homo sapiens)
ttttgtaggtggagggggaaagggtttgggggttggtggaggatgtaggagtatgggggagttgtggaaaagatgtggaggtagagttagtaggttttgttaagggataggaggtggttttagagagaaaatgagggattagggatgttttttggggggtggttagttgggtgattgggagaaattgggaaggtgtagattagggtagtaggggtagtgggattgggggtgttttgaagtgaatatgtgaaattgaaggtgtttgttagttttttagtggaggtatttaggaggtagttggaattggaaggaaatggtgatagttgttagtattgtagaggtggtggtggtggtggttatagttggatttttttgggttggtgggaagtgtggaggtgaaggagtagggagtagggggaggtggagaaggaaattttaggtttatatttttattttatttttggttgttatatttttaggaagttttttttgaggttgggatagagggtggggatagtttagtttttttgagagagtttatttttggaggtttgtggtgggagttagggtgtggtggagagggttttttattttttttttagtaagtgggagggaggtgttgggttgaggttgtgttgagtttgagtttgttgtttgggtggatttgttttgtttaagtgtggaggggtggaggtttggttgggttgtagtgtggtgtggagtaggatgttgttttgtgttatggttattggagatgtatgtttatttttttttttgggtttgttgattttgttattttttgtttttggttgtttgtgggtgtttgttatttagattttttttgtgttttatgggatgtaagtttttttttagagtttggttttgtaaaagaggtttggagtttttgttatagattttttttttgggttatggaggatgagaagggttattgagttgagttgtaattttgtgtaatttttgatttttttttttttttgtttttatggtattttattgttttttttttgtttttgaggttttttagaaaatagtgtagaattgtgtagatgtttaggagatgtgaagatgttggagatgtttaggaggtgatggttatgtgaggagaattgtttttaggtgtgtttttggaatgtatggagattttttggtggggaaggggttggggtttgtgttatttagtgtttgtttattgatttttttttgaagagggggtttagggtttttgttgagggagtggggaggtggtgtgggttttttttgggtatatatgggtgtttgttttttttttttttttttttttttttgtatggagagtggagagtggagagtggagagtgatagggaggtagttgaagatttgaattttgaaaggggagttggtggtgaatggtgaatgagatagttatttaggaagtgagtttagttagtttgggaggtggtggagatttatgtttggaagtaattggattaggttttagaatgtgattgttttttgggttttgggggagatgttaaggatgtgtaagtggagggtgtggagataattgggagttagagtttttattatttgggttgggaatgtttttgggtgttttgatggggtggtggtgggggttgggggaggtttttgagaattgtgtggtttggggagagtttatttattgttgagttttgatataggttttgaagttgttgtagtggtttagttttttttttgtttggttttgtgtagtgtggtgttgtagagtttaggttgtgtttttgttttgttgtttagagtttattgtggtggtttattggattatgttggtggggtgattgtagtttttgatttgtgagttgtaaagagttttgaggtttatttataaatttgtgtttttagttgtttttttatgtatttgagttatgttttgggatttggagagtttggggtgttgggtgttgtggaggaggtttggtttttgttgttttttttattttttagtttgtgagggatttgggggaagggggagtaagtttttgttttggaagaaatgtttggaattaaggagtttgatttttggatttgggtgtttgttggatttaggtttttatttttgggttttttggttgaattaagggttttgatagggtttgaggtatttgtatttttaggagataggagtttggttagggtgtattattgggttttgttttgagttagaggatgtagtgtagatatttatttttatattgtttttttatagagtttttttttttttggggtgttgttgggtatagggtaggttgttaggaatttttagtaaaattatgtttgtttagaggtttgtggtttttttagagggtgttggggaaagagaggggatttgatttttttgatttttggaggaaaagtgtttggggttttttagggatatagttttggaagtttattttattttagtttaggttgtggtgaggtggggaggagagttaggagagggggagaggggttttgtgttttgtagaggtttttaattttggaggaaaaagattgggatatatttaagtgagttaaggtttgagttttatgtatttttatttatttggggtggtgtatagttagtttttgttgggtgtgagtatttgattaagggagtaagtggaatgaaaatttagttgggggggtttttattgatataattgttttgtagttgagtttttggatttttgggagatgtggagagtttggggttggtttttgttttgtagagtagattggattgttttaggtgtttggaatgtgtttgtattttgttttttggatttgtgggagatttgtgttttgtaagtttttttttttattttagtttgtttttatttatattttgttggtgagtgtgttattgtgaggtgtttttttttttgggaagggagtttttttttgtgtagatttgtattgtttttttttttgtttggttttgttttttaggggtagtttttgtagaaaggagatttttttttgggttgaagggttattagtttgtagttagtttagttttggattttgggagatgtttattattttgtggattttgatttgaaattttttttggttgtttattgtggagagtgtttttgtagagaggtttttaattgaaggagtttgggttttatattttgtttttaagtttgagtttttagggttgttttgtatgaggtgtagatgaatttgtgtttgtaaataagatagaaaattttaagtgttgtttgattttttttttttggggaatttgtattttgttttgggagtgtgtttagtgttttagtattaaattttttggttggggttggtagagtttagagttttgtttttttttaggtgtggttttttaatatttgtaatttaaatgttgtgttgtggttaaaattagttttggtagtgtgaatagagaattaaaagtaggtagtgaatgagaatagtttgtattttttttttttggtagatggggaggtgtaaattttgaggaattttagggtatttgttttaaatgtgggaaatttttgtgtgtattttgttttttttttttagtttgtgttaatgttttaaatggtgttgagttgtttaattttgttgttattatagaggttgttgtgttttaggggattaattgatgtgagatatataaaattttgtaattttataatataaattatagtatagtttttttggagagggttggaatatttgagtgagtttttgagaggaaaagaggagttttttagaggagaaatagagtatttttataatgtgttttaattgagaaattttgttttattgagtttttttttaagtggaattagaagtgttgggatgagagggaaaggatgggagtgtgtttaaaggtggatagtaggtttttatttttggtgggagtgagattggatggtattttttggaaaggtggtttgggttttggataaggttagaggtaggagtttatgatgtagagatgatatagtgtttttttgtgtgtgagtttatgaaggttattattgaggttttgtgtttgtaaaaggttgttatgttttatataagtttttatatttaatatagggattgattgggtatagggatttttttatattatatatgtaagtatgtatgttaattaaagatgtttgtgttaaagaaatggttaattttgttgaatttagaggaattgattaattatttaattaagtagaggaaatgtttgaatttaatttgtaatttagttgtttttttatataaaattatatatttttatttatatttgatgaatgaaaaaagaaattagtttatgattttaatttaaatatatgtttttaaaaatatattttttttagtttagttagtatataaattaattgagtttttttggttaagtatgatttagttgtgatatttaagagtgggagtggttgtttagatatttttttttttatgtgaaatttagattaatgagttattatttaataagttgtaggtagtttggttgggttggatttagatggtttgagttaaatttagattgtatttgtttaaaattttgtaaatttttagttatgtaaattttatttttaaaaatgttgggaagttattgtataagagtttaagttatattagtttttttgtgattatttgtagtttttggggaaagaatagaagaaaagaaaatgttagtttttgtgaggtggggttggtgttttaggggttttttttgaatattttgttttttttttaaaggttaaaaggaaggtagtggatatatattagaattttttttatttgtgaatggttgtaaggttggagaaggtggttagtgtattttaaggtttattatttttttttgtgtttttttttttgttttggtaggtttagttagtttaagttttgggtgttattttttaaatttttttgttaaattaattttatttatttgattggattatttgagaggtgttatttttttttgggttgttgtgattttgagggggtatttttataagagtttaggtattaggtggtgaaatagtttgtgtttttaaatttgttttttttagggtttttgggagattttagagtgtaggtttgtttggggagttttaggggtgggttttgagtggaagtgggtttattttttataggagtttaaattttataggaataagaatagtagtaaaataggataagagagtaggtagggagttgttaaggaaaggtgatttttgggaaggtaggttatttagaataaggtttttttggtgttggagagttggaagggggagtgggtatagaggatttggtttaggtgtgggggttattagtataagtagagttattttttagatttttttttagaagtagttgttttttagagaaattaggtgagggatagtttttgtatttttatatgttagttttggagatttgtttatttgtttttagttgtttttttttttgggtgagtttgggttaagtattaagttaggatagaagggtggtttttagttggtttttggtttattttgtttttttaatatttagggagtttgggtttagtataggtgtttttttagtgattggggtagaattaggatgtgtaatgtgattgtttttttttttttgttttttggtagagtttttgtttgtgttaatttatttattaggttttgtttgtgttatgtgtgtttttggtaggtttggggtggggaaaggtgaagtgttgggtatgtgagggtttgtgtattttagtttttatgatgtttttggttttttttaggtttgttgttgttgtatttaattttttttttttgggggttattttgaagaagttttagattttagatttagttttttagattttgtttttgagtttgtttggtgggtttgtaggttggtttttttttgtttagaggagagtgtagatatgtaatgtttgtttgttggttttgtttgttttatttttgttttttgtgtttttttgttttggtttttgtttataggttgggttggaatgttagttttaggagttgatggtggttttttgtttgttttggggaaggtgttgtttttttattggttttaattttttgtttggtgtttggggtttggttgtggggtttggtttttagttgagggtgtagggttggttaggtttgttttggttgaggtggagattttgtttttagggattgttgggtgtttttgttttgtgagtaatgagattgttgtgagtgaatggtttttattgagtttatttttttaagtgttattatgttaagttagagaagtgaggtgagtggaggggatgtagaggggttggaaaagttatttttttttggttttgtttttagataaaaatgggagtttggtttggtatttgggtgtttggtgtttttggggtgttttattgaggttttgtttgtaaaattagtgtttgtgttttgaagtgtatggtgtttggaagttgtttttttgtttgttttttttgaggtttttttttgtgtagtgagtttgagaaatatggaggattgttttttgtaagtgggtggttgtgggtgttttttgatttttgggtgaagttagagggaaaatggggtttttgggttagtgttttattttttattttgggagaaattaatgtttggagagggtttgtttgatttgtatagaaaggttgattttgagggtggtggttgtttgggggagaaagtggaggttttgggtttgtgggagtgtggtagttggggttggtattgtagaggagagatatgttattgttttgtatttttagaaagtgtgaggtgttgttttagttggggtaggtggttgaggttggtttttatgtgtgttttttgaagttttttgaaatattttgtggagttttgtgtgtatagaatttagtattgtttagtttttgggtagtttaattttttgtagttttattttagtttttttggttttttgaatggtgattttttttattgtttttttagagttgttttgtgtttgggtttattttggggggtttggtattttttagttatttttttttttatttattttttttttttttatttttttttttattttttattttttttttttttaggagagtgatttatgaatttttttttttttagttaattttagggtttagtttgtagattttgtgagggtaggtttttgtttattggtttgttaggtgttttggaggtgatgttttgttttttagagtttttgttgtagttatgaattggggtttgggttgtaggaaagtatagggttgaagtttagtgttttggggttatttatattgaggtagttagaggtaaagagttttaagaatttagaaaatattttttaggaagttgtttaattggtttttatgggataggtggagttattaatttgggatggttttgtaggaattaaagagtttagggttttttttttttttaatattatgtttaggagatttagagttgttggatttttttttttttgattggtgattattagagtttttagagttgtagaaaattttttttttaaaaaattaagtaagtgttaataagattttttataaatttttattagttttatttttttggggggtaggtagattgtggggtttgattttttgagatttggggaggatttttggtagatgtgtgtttagttagaatatttggtaaggatttttttaatgaagaaaaagtggaggaatttagttttagtgagaagaggtttttttattttgttttagatatattggatagagggtatattttgattagagttatgtttagtggttaggaggttagtttagtatttttttttttattatttttgttttgggtgggggggtaatttttttgggagtagttgtgggaattgttgttttttattttagtttagttagtattttgaagtttgtaggggaaggatagtatgtgggatggatattggggaaggagttttgtaaggttagggtgtaatttttaggttttaggtggtttggtaggttatgttgttttggagatgtttgttagattttttaagtttatttagggtttggtagtaatttgttggttgtttttgtgggggtttgggttgttgagtatggtgtagttgtttagggttaattagttttagggtgtttgtgttaggttgtggtttttttgttttttttgtattgagggtatttatggtgtgtaaatgtttttgtatttttagagttgttttattggatgtttttaggaatttatatatttttataaaaatgtattttaaatgatggataggtgagtttggggtaataatgggtgtttggtgggtagataagagtaaatgggaaggagtttgagggaggagggggaagagaagaggaaatagaatttttagttggatattttgataatagttggaaggaaagtttagaaaagatgaagagagaggaggggagaaattaattggggtttttatttttgttgttggatttttaatttttgttttaaatgggttttgttttttggtaaaattagtttaaaggattttaaaataaagaaaatgagatgattggtttgggagttttttaattagagtagagaagttagaggggggtgggtgatttggttttgaagttttagttgaatagttattttttttttttttggtaaaaaggatttttttagaatttttgaggtttttggatttttttttttgtaaatggagttgtatattgtatttttttgtttttttggattgttaagtatgttttatgagggttgttgtttttgggtggaatgtggttgtatgtatgtgtttttttgtatatgtatatatatgtatatttataataagtgtttgtaggaggagtgttttgtgtgttagttttgtgtttaagataggaagttgttgggttattgagttaaatgggagtgatattatttttttttattagtaaggaaagtggattataaaagtttttttgtattttggtagtttatttaatattatttatgtattttgtgtaaggaattgtgggattttgttttatggtaaataatatggaaattttaaaaatagtgatttttttgtgtgtgtttatttatgtgttttggggtgatttggtggggttgttgttgggtgatttatatttttgaattgtgaagtgatagggaaagtgtgggtgagtgtaggagatgtggttgggggtttttttgggtttttgggtttttgtatttggagtgggggatgtggttgttttaaggggaggaggggtggtgggttgtttttgttatttagtggtggttggagtgttatgtgggtgtgtggtgttgtggttattggtttgaggtatgtgtttaggagattggtttgtgatgttatttgagggggttttgttaaaaataagaataaaaatttagagtgaaagtgttttaggttgtgttgagtggtttggaaatttttgagtttgtgtggaggttgaggtggtgagggtggtggatggttggggagtgtgggtggtttagtttggtttggttgggttttggttttgtgttttttatttatgtgatttgggttgtggagttttgtggggtttggtgggggtgtggttgtatgttggtggggtgttttggtttgtagtggggtggtggttgtgaggagggggtttttatgtgtgtgtgggtggtggtgggtgtgttgattgtgggtgtttggtatttttgagggttggttagggtgtgtgggtggggatggttgggtggtggtggtggttggagttggtttgggtgggtgtgagtgttggggaatgtgttgtttgtatgtgtgtagtttttgttttgggtggtttaggtggtggtgttggagtttgaggtggttggatgtggagaggagtggggagtttgggaggtggtttgtgtttttgttggattattgtgattgtttagattttggttgtgtggtgaagttgaggatttggttttgttgaattttttattgtttgggtgagtggggtggtttgtggtgtttttaatttagtttgtggatttaaaggtggttttgtgttgagtgtggttggtgatttgtaggattttagttttggttgtggttgttgtgtatgtttttggaagatttggtggggtgggggtgtgggggtttttgtgtgtgttgtgggagggttgaaggttgatttggaagggtgtttttggagaattagtgtgggatttattgtgaatagtatggaggagaatgattttaagtttggtgaagtagtggtggtggtggagggatagtggtagttggaatttagttttggtggtggtttgggtggtggtggtggtagtagtttgggtgaagtggatattgggtgttggtgggttttgatgttgtttgtggttttgtaggtgtttggtaattattagtatttgtattgtattattaatttttttattgataatattttgtggtttgagtttggttggtgaaaggatgtggggatttgttgtgtgggtgtgggaggaggaaggggtggtggagttggtggtgaaggtggtgtgagtggtgtggagggaggtggtggtgtgggtggtttggagtagtttttgggtttgggtttttgagagttttggtagaatttgttatgtgtgtttggtgtgggtgggttgtttttagttgttggtagtgattttttgggtgatggggaaggtggttttaagatgtttttgttgtatggtggtgttaagaaaggtggtgattttggtggttttttggatgggttgtttaaggtttgtggtttgggtggtggtgatttgttggtgagtttggatttggatagtttgtaagttggtgttaatttgggtgtgtagtttatgttttggttggtgtgggtttattgtatgtgttatttggattggttttttttaggtgagtttgtggggattatgtgttttggtttgttgtggggaggtttgtggagttggggggtggtgttggtgtgggaatttattgggaggaaaatattttgaattttttttgtgtatatgtataaagatttatgtgatattgtgtgaagttgatgttggtttgggtagtggttaggagtttagtggtaggattgatttgttagggggtatagattttttaggattgtagaagggattttttttttttttttgttttttttttttttttttttttttttgttttttttttttgttttttattttgttttggtgtatttttttttagtttttagtttatgttttttttattgtagttttttttggtgggaatgtggtggttggaagatgggtttggaagtgtatatttttatttttttttttatgattttttaatttaggttaggttggggatgtatgttttagtttattttagatttgttttattatttggttattttggttgtgtttggggaagaaaaggtgaggttttttgttgttttgttttttgttttttgggtttgtgttgattggtgggatttaggaggatgtatatagggaaggaggaaaataaaggtgttttttttttttggttttattttgtttgttagtgttagtttgtagtggtggggtttagttttttttttgtatatagtgaggataagggaggtagttgttttttttggtatttgttatttttaaatagaaaggatttttttttagggttttttgggggttgttgatgggaaagaggtagtatttgtaggggttttgtagagatgttggatatatttttttatagatttgtgattttaaaaaattaagtttatgtttttgtagaaattattaattgtattttatgtgggtttgtggttgggaattgttattagaagtggattgtttgattttgagttggtagtggatttttgttgtttttaaatttttaattattttgtgggggttatttgtttagattatagtaggagtgagttaatttttgggttgttattttgtagaattatgtgtgtatatttttgatgaaatttagattttttagttagatttgaaatttgttttattgtttttgttttttttttttgttaatatttaattaatatataggtttataatgttgggtgaggagatttggttgggttttgtgtggtgtgggagtttgttgagttagtttttaatggtttgggagttgggtagtattgtttggtttggtttggtttggtttagtttagtttagtttaagttgtttatttttatgggttttaaaatatttttgtaagataatgtttttgttttttggtttttttgaaagaaaggggagagagagtttttttggggaggtttgattttgtttttgagatttttaagtatttgttttttgaaagaaaattaagaaaaaaatttaaaaattattattttagggaaatttattgttataaaatggtgtttttttgtgggttgttttatgagtgtattaataagagttttaggattagaagagtttgggggtagagttttggggaagggagtggttggaaatttagatagagatgggttttgggagtaggaggttggggttttttttggagttttgtgttttattttttattattgttttggagggttaattttatttttaaatttgtatttatttttattaaagttaggtttattggtttggagttttgggtgtgagtaagataggtattgagtgtgtatgtgtgtatggggtgggtgtttaagtatagggtgtgtgtttttatgggtggtgagtttgtttatgggttgttttaaaagttgtttttggtgtttttgaggtggtgtttatagattttttttttttaggtttgttttttggagagagtataagatttatttggttatgagggagtgtttggtatttattttgggtttttagtttgttttttattttttgttgggtatagttttagtattttagttgatttttttgatttgggtagggtgtagttttagggtttttaaggagatttatattttttttttttttagtgtgtttggtagttttttggttttgaagggtggggggtttttagttttttttagttatagggatttgtgatgaagttggggttagatgttttttaaagttgatttatatattgtataaattgaaatttagaggtgaggttattattttttgttagtggttttgtttttttttttttttatagggaatgttagggggttgagttttttattattaaaaagaaattgatgatatttttttttttttgtttttttttttttgttttttttttatggatagtaggttttagaagttttatagtgattttgtttaaaatttggggtaggtttatagggagaaggttaggttaggtttataagtttgaattttagttgggaggtatagtggggagggttagaagtggatttggataaggttagttgggttattttgttgtttatagtgaagtagttttatgtttggggaaagggtggtgtagttaatatttttgtagagttaggtttttttttttggataggaaatttgggagatttttagtgggtgaaggatttatttattgtgagtagtttagtgtttttttttattaaggagggaagtatatgtattgatttttttttaaaggaatgaatttgggtttatagagtttttggttgggagttatagaggagtttgggtggaggtagatattttgggttttttttgtttttagggtttatttgtttttgatttttatagtttttggtattttgtggggtatttttatgagggtttttattatagtttttagggtgttttttgtttttgtgattgttttgtagttttttttagtttttttttttttttttttttttagtatttattgttatttttgtttttgaatagagagttttagaaaggattaggaaaaattaggttagaaagtgtggggagttttgtttatatttaggagttttatttttatttagagattttttatttgtggttagtttgtttattaggtttgttttatagttttatttatattatatgtagtttttttttattaagtggtggaggtttgtgttgagtttatgtttagtttgaagtttagttttatatttggtggtttagttttgagtggttttgggtgagttattttttttttggggttttagaatttttatttggtgtttattgggtggtatatttttgggtaatttgatttttttttgtttattgtatttatttaggttttaggttttgaaaattaaagaagaagaatttgaataaagaggataagtggttgtgtatggtttttattgttgagtagttgtagaggtttaaggttgagttttagattaataggtatttgatggagtagtggtgttagagtttggtgtaggagttgagttttaatgagttatagattaagatttggttttagaataagtgtgttaagattaagaaggttatgggtaataagaatatgttggttgtgtattttatggtatagggtttgtataattattttattatagttaaggagggtaagttggatagtgagtagggtggggggtatggaggttaggttttagtttgtgttaaataatgtaataatttaaaattataaagggttagtgtataaagattatattagtattaatagtgaaaatattgtgtattagttaaggttttgaaatattttatgtatatattatttataggtggtataaaatttaaaatatttgattataaaatatttttttgagttttttgtgtttatgagattatgttaattttatgggttttttttttttttgtgaagggggttgtttagggttttatttttttttaattttttaagttttattatatgatattggatatttttttattattttaaaagaagaaaaaattaaaataatttgttgaagtttaaagattttttattgttgtattttatataattgtgaattgaataaatagtttttatttggtttatgatttttgttattttgtttgtgttggtttggtgaggatagtaggaggggtttatattttaagtttggattagttattttaaggttttggggagtttaggggatttggtgggagagaggggatttttagggtttttgggttagttttgggatttggttttgggaagtagtttagtgtattttaggtttgttttgggaagttggttttatgtttattagtagttgtttaggtttgtagttttatttggttttttttttttatttttttgtatttaatttttttttttttttttttttttttttttttttttttttttttttttttttgttttttttttttttttttttttttttttttttttttttttttttttttttttttttttttttttttttttttttttattaagggtttaattgtgtgtatatattgtttgtgtttgtggtttgtgttgttgtttttagttttattgtagttttgttgtaggtttaatttttttgttttgggtattgtttttatgtagaagtgttttgaggttttggggttaaaggtttggggtgtgtggtttaaagtttaagagtggtggggtgattttttttttggtttggttttaggaattttttgtgattttattagttattatgggtgttagttagggttttagaaatgaggttatggtttattgtttttgggtgggtagaaggttttgtagagggagatggtattatttattttttttttttttttttttttttttttattttttttttttttttttttatttttttttttttttggagtggttgtttttgttatagagaatatttttttaagataaatatgtgtgtttatatatatgtttgtatgtatgtgaatatatatatatatatatatatattaggtgtgtttgagtttatagttttgaaatatgtggttattttgttttttaaaagaatttagaattttttaggatttagaagaaggaagaaagtgtgtaaataattattttttattattattttttgtttttttttgttttttaaaatatatattttatttttgaaggtgtggtatagtgtaaattaaatatatttaatatattttttattaagtatttatatatgtatataaataaatatattatttatatataatgttat

SEQ ID NO: 27 - DNA, 16197 bp, Artificial Sequence
  chemically treated genomic DNA (Homo sapiens)
gtggtgttatatatagataatgtgtttgtttatatatatatataggtatttggtgggaaatatattgaatatatttaatttatattgtattatatttttaaaaataaaatgtatattttaaaaaataagaaaagataaaaagtgatgataagaaatgattatttatatattttttttttttttttagattttggaggattttgagtttttttgaaagataaggtagttatatgttttagaattgtggatttaaatatgtttggtgtgtgtgtgtgtgtgtgtgtgtttatatgtatgtagatatatgtgtaaatatatatatttattttggaagaatgttttttatagtagaagtagttattttaagaaaagaaaaaaataaaggaaaaaaagaaaaaaatagggaagaaaagaaaaaggaaaggaagatagatgatgttatttttttttatagagttttttgtttgtttagaaatagtgagttatggttttatttttgggattttggttggtatttatgatggttggtggagttataggaaatttttggggttaagttaaaaggagggttgttttattgtttttgggttttaggttatatattttaggtttttagttttagaattttgaagtgtttttgtatggaggtagtgtttagggtaggagggttaggtttgtggtaggattgtggtgggattggggatagtgatatagattatagatgtagatgatgtatgtatatggttgggtttttggtgaggaggaggaggaaagagaaggaggaggaggaaggaaggaggaggaggagaagaaaaagaagaagaaaggaggagtaggaggaaggaggaaggaggaagaggaggaaaaaggagaaggaggagggagttaggtgtaggagggtgaggagagggagttgggtgaggttgtgggtttgggtggttgttggtgagtatggagttgattttttagagtaggtttggggtatgttgggttgttttttagggttaaattttagaattggtttaaggattttggaagtttttttttttttattaggttttttaagttttttaaggttttgaggtggttggtttaggtttgaggtgtgggttttttttgttgtttttattaagttaatataaataaagtggtagaagttatagattaaataggagttatttatttggtttatagttgtgtgaaatgtagtaataaaaaatttttggattttagtaagttgttttaattttttttttttttggaataataaaaaagtgtttaatgttatataatggagtttaggggattaaaaaaaggtgaaattttaagtagttttttttgtaaaaaagaaaaaaatttataaaattagtataattttataaatataaaaaatttaaaaaaatattttatagttagatattttggattttatattatttgtaaatgatatatatatagaatattttagaattttagttaatatataatatttttattattaatgttggtataatttttatatattggttttttatgattttaaattattgtattgtttagtgtggattgagatttggtttttatgttttttgttttatttgttgtttgatttgttttttttggttgtggtggagtggttgtataagttttgtgttatgaggtgtatggttagtgtgtttttgttgtttgtggtttttttgattttggtgtgtttgttttggaattaaattttgatttgtgatttgttgaggtttagtttttgtgttaggttttggtgttgttgttttgttaggtatttgttggtttggaatttggttttgagtttttgtagttgtttggtggtaaaggttgtgtgtggttgtttgttttttttgtttgggttttttttttttggtttttgagatttgggatttgggtagatatggtggatagagagaagttaggttatttagaggtgtgttatttaataggtattgggtaaggattttggggttttaagagggaagtgatttgtttaaggttatttgaggttgaattgttagatgtggggttagattttggattgggtatgggtttaatgtggatttttattatttggtgagggaggattgtgtgtgatgtaagtgggattatggggtaggtttagtgggtaggttgattataaatgaggggtttttagataaaagtaaaatttttggatgtaagtaaaattttttatattttttggtttggttttttttagttttttttgaggttttttgtttaggaatagggatggtggtaggtgttgagagagaggggaggagagaagattgggaaaggttgtagggtggttatagaagtaggaggtgttttgagggttatgatggggatttttatggggatattttatagggtgttaagggttgtgggaattaggggtaggtgggttttgaggataggaggggtttagagtgtttgtttttatttaagtttttttatgatttttagttaagggttttgtggatttaagtttatttttttaaggaagagttagtgtatgtatttttttttttggtggggaggggtattgagttatttatagtgggtgagttttttatttattggaaattttttaggttttttgtttaggagaggggatttggttttataagggtgttgattgtattattttttttttagatatgggattgttttattgtgggtagtagggtagtttagttgattttgtttaggtttatttttgattttttttattgtgttttttaattgggatttagatttatgaatttgatttggttttttttttgtggatttgttttaggttttggatagggttgttgtaaggtttttaggatttgttatttatggggaaagggtagagggaggagagtagaaggagggaagtgttattagtttttttttggtgataagaggtttaattttttggtgttttttgtgggggaagaagggggtaaggttattggtagggagtggtgattttgtttttgggttttaatttgtgtggtgtatgaattggttttagggagtatttggttttagttttattataggtttttgtggttggagaaggttgggggttttttattttttagggttggagagttgttgggtatattgaggagaagaggaatgtggatttttttggaggttttgggattgtattttgtttaggttaggagagttaattagggtgttggggttgtgtttagtaagaggtgggggatgaattggaggtttagggtgaatgttaagtatttttttatggttaagtgagttttgtgttttttttaggagataggtttggagaaagaggatttgtgggtattattttaggagtattaagggtagtttttaaagtaatttataggtaggtttattatttatgagagtatatattttatatttgggtatttattttatgtatatgtatatatttagtgtttgttttgtttatgtttagggttttaggttagtgggtttggttttggtaggggtaggtgtagatttggagatggggttggttttttggaatggtggtgggggatggggtataaggttttaggagaggttttagttttttgtttttagagtttatttttgtttgggtttttaattatttttttttttagaattttatttttaaatttttttggttttggggtttttgttaatgtatttatgaagtagtttgtaaaaggatattattttatggtaataaatttttttaaaataataatttttaggttttttttttgatttttttttaaaagataaatatttaggagttttagaaatagaattaaatttttttaagaaaattttttttttttttttttttggagaaattaaagaatagaaatattattttgtagggatattttaaagtttatgaagataggtgatttgggttgggttgagttgggttgggttaggttgggttgggtggtgttgtttagtttttgggttgttgggggttggtttagtgaatttttgtgttgtataaagtttggttgagtttttttgtttggtattgtgagtttgtatgttaattaaatattaataaaggaggaagatgagaataatggagtaaattttagatttagttgagaaatttgaattttattagaaatatgtatgtatagttttgtgggatggtggtttaagggttggtttatttttgttgtgatttgggtaaatgatttttgtaaaatagttgagggtttgggggtagtggggatttgttgttagtttggggttaaatagtttatttttaatggtggtttttagttgtagatttgtatggaatgtagttgatgatttttataaggatatggatttagttttttaaaattgtagatttatgaaaaaatatatttagtatttttgtagggtttttgtgaatgttgttttttttttattagtagtttttaggaaattttgagagaaggttttttttatttggggatggtaggtgttgggagggatggttgttttttttgtttttgttgtgtgtaggaagggggttgagttttattattgtgggttggtgttggtaaataaagtggagttaaggggaaagggtgtttttatttttttttttttttgtgtgtatttttttgagttttattggttagtgtaggtttgaggggtagaaggtagagtggtaaagggttttgtttttttttttttgagtatagttgggataattggatggtaggataagtttggggtgggttggggtatgtgtttttggtttggtttgagttggaagattgtagggaaggggatgagagtgtgtatttttgggtttattttttagttattatgtttttattaaaaaaaattatagtgggggagatatgggttaggggttgggaagagatgtgttaaggtggggtggaagatagagaggggagatagggagagaggaaggagagagagagatagagaaagaaagaaggttttttttgtggttttaagaagtttgtattttttagtgaattagttttgttgttggatttttggttgttgtttgggttggtgttagttttatataatgttgtgtgagtttttgtgtgtgtgtgtgggggaggtttgagatgttttttttttggtaagtttttgtgttagtattgttttttagttttgtgggtttttttgtggtgagttgggatgtgtggtttttgtgggtttatttgaagaaggttggtttgagtagtgtgtatagtagatttatgttggttagagtatgggttgtgtgtttaggttggtgttggtttgtgagttgtttgagtttgagtttattgataggttgttgttgtttaagttgtgggttttgagtgatttgtttagggggttgttggggttgttgttttttttggtgttattgtgtagtgagagtgttttggagttgttttttttgttatttggagagttgttgttggtggttgggagtggtttgtttgtgttgggtgtatatggtgggttttgttggggtttttgggagtttgagtttaagagttgttttgagttgtttgtgttgttgtttttttttgtattgtttgtgttgtttttgttgttggttttgttgtttttttttttttttgtgtttgtatagtaggtttttgtgtttttttgttggttgaatttgggttgtaggatgttgttgatgaagaagttggtgatgtggtgtgggtgttggtggttgttgggtgtttgtaggattgtgggtagtattagagtttgttggtgtttggtgtttgttttgtttgggttgttattgttgttgttgtttgagttgttgttggggttggattttggttgttgttgtttttttattgttgttgttgttttgttaggtttggggttatttttttttatgttgtttatagtaaattttatattggttttttggggatgtttttttaaattagtttttggtttttttgtggtgtatatggagatttttgtgtttttattttgttgagttttttgagggtgtgtgtggtggttgtggttagggttgaggttttataagttgttggttgtgtttggtgtggagttatttttgaatttatgaattgggttagaaatattatgagttgttttgtttgtttagatgatgagagatttaatagagttaagtttttgattttgttgtgtagttggggtttagatagttgtagtggtttggtggggatgtgggttgttttttgggttttttgtttttttttgtgtttggttgttttgggttttggtgttgttgtttgggttgtttggggtgagagttgtgtgtatgtaggtagtgtgttttttggtgtttatgtttgtttgggttggttttggttgttgttgttgtttggttgtttttgtttgtatgttttagttggtttttgagggtgttgggtgtttgtggttagtgtgtttgttattgtttgtatgtatatggaggttttttttttgtggttgttgttttgttgtgggttggggtgttttattggtgtgtggttgtgtttttgttgagttttgtagagttttgtggtttgagttgtatgggtgagagatgtgaggttagggtttggttgggttgggttgggttgtttgtgttttttggttgtttgttgtttttgttgttttggtttttgtgtgggtttggaaatttttaggttatttggtgtaatttgagatatttttattttggatttttgtttttatttttaatagagtttttttgagtgatgttgtaggttggttttttggatatgtgttttgggttaatggttgtggtgttgtgtgtttatgtgatgttttggttgttgttgggtgataggagtagtttgttgttttttttttttttaaagtggttgtgttttttgttttgggtgtgggagtttaggaatttggagagatttttgattgtgttttttgtgtttgtttgtgttttttttgttgttttgtggtttaggggtgtgagttatttggtgatagttttgttaggttattttggggtgtgtaggtggatatgtataggaagattgttatttttaagatttttatattgtttattgtggggtgaaattttataattttttgtataaaatgtataaataatattaaatgagttgttgagatataaagggatttttgtggtttgttttttttgttgatggagaggaatagtgttatttttatttgatttggtaatttggtagttttttgttttaaatgtagagttggtgtgtaggatattttttttgtagatatttattgtaagtgtgtgtgtgtgtgtgtgtgtagggaggtgtgtgtatatggttgtattttatttggggatagtgatttttatgaaatatgtttagtgatttgaaagagtgggggaatgtagtatgtggttttatttgtgaagggagaaatttaggagttttggaggttttaaaggaattttttttgttaaggagaggaggggtgattgtttagttaagattttaaaattaagttgtttgtttttttttaatttttttattttgattgaggagtttttagattggttattttgttttttttgttttaaaattttttaaattaattttgttggagagtagggtttatttgaaatgagaattaggaatttaatggtaagggtgggagttttagttggtttttttttttttttttttttattttttttagattttttttttagttgttgttagaatgtttaattggaagttttgttttttttttttttttttttttttttttgggtttttttttatttgtttttgtttatttattaaatatttgttgttattttaggtttgtttgtttattatttaaaatgtatttttatggaaatgtgtgaatttttggaaatatttgatagggtagttttgggggtgtgggagtatttgtatgttatgagtatttttagtgtgggggaggtggggaggttgtagtttggtatggatattttagggttgattggttttgggtagttgtattgtgtttagtaatttagatttttgtagaggtagttagtaggttgttgttaggttttgagtgaatttggggagtttggtaagtatttttgaggtagtgtggtttgttaggttatttggggtttgaaggttgtattttggttttgtgaagttttttttttagtgtttattttatgtgttgttttttttttgtaggttttagagtgttggttgggttgggatgagaaatgatagtttttatagttgtttttaggaaaattatttttttatttaggatgggaatggtgggggaggaggtgttgggttggttttttggttattggatgtggttttggttagagtgtgttttttgtttggtgtgtttagggtagggtggggaagtttttttttgttggggttggatttttttattttttttttattggaggagtttttattaaatgttttggttaggtatatatttgttaggggttttttttgagttttaaaaaattaaattttatagtttatttgttttttgggaaaatagggttggtgagagtttgtgggaagttttattgatgtttatttggttttttggggggagaattttttgtaattttggaaattttgatgattattagttaggaaagggagggtttaatgattttgggttttttgaatatgatattggggggaggaggggttttgagttttttggtttttgtaaaattattttaggttaatggttttatttgttttatgaaagttaattaggtggttttttgagaagtattttttgaatttttgggattttttgtttttgattgttttagtataaatggttttaagatgttgggttttagttttgtgtttttttgtgatttagattttaatttgtggttgtaatagagattttgggaagtagagtgttatttttagggtgtttggtgggttggtgggtagagatttgtttttgtagggtttgtgggttgggttttggggttggttggaggaagaggaatttgtgggttgtttttttggaaaggaggaaggtgagaggtggagggaggaataggagaaagagaaataaatgaagagagagataattagagggtattgggttttttagagtggatttaaatatgaaataattttgggaaagtggtaagaggggttattgtttaaagggttggggaagttgggatggggttgtagaaagttgagttgtttaggggttgggtggtgttgagttttatatatatggaattttgtaaggtgttttaggaagttttgagaagtgtgtatgaggattggttttggttgtttgttttagttgggatggtgttttgtgttttttggggatgtggggtagtggtgtgttttttttttgtgatgttagttttggttgttgtgtttttgtgagtttagagtttttgtttttttttttgaatgattgttgtttttaaggttggtttttttgtgtggattagatgagttttttttggatattagttttttttgggatggaaagtggggtattgatttgggaattttgttttttttttagttttatttaggaattggagagtgtttgtgattatttgtttgtggaggatggttttttatattttttaggtttgttgtataaagaggggttttggagagggtgagtaggaaagtagtttttagatgttgtgtgttttaggatatggatgttagttttgtaaataaaattttagtgaagtgttttggagatattgggtgtttggatgttgagttaggtttttgtttttatttgaaaatgaggttagagaagggtgatttttttggtttttttgtgttttttttatttgttttgtttttttagtttgatgtggtgatatttggaaaaatggatttaatgaaagttgtttgtttgtggtggttttgttatttgtggggtagagatgtttaataatttttgagagtgagatttttattttagttaaagtaggtttggttagttttgtgtttttggttgggagttgagttttgtagttaagttttaggtgttgggtggggaattggggttgataggagagtaatattttttttggggtgagtagagagttattgttggtttttggggttgatgttttgatttggtttgtagataaaggttgaaatgaaggggtgtggggagtaggggtggggtgggtggggttaatgggtggatattgtgtgtttgtgtttttttttgggtgaaggaggattggtttataggtttattgggtgagtttggggatggggtttgggaggttgggtttagggtttgagatttttttagaataatttttggaaggagggggttaaatgtggtagtggtgggtttgaaaagggttaggagtgttgtaggaattggggtgtataggtttttgtgtgtttagtgttttgtttttttttgttttaggtttgttaggggtgtgtgtggtgtgggtagagtttggtgggtgggttggtgtggatagaagttttgttggggagtggggaggggggggtagttgtattgtatgttttggttttgttttgattgttggagaagtgtttgtgttgagtttaagttttttaggtgttggggaagtagagtgggttaggagttgattggggattgtttttttgttttgatttgatatttagtttaggtttgtttggggaggggggtggttgggggtaggtgggtagatttttagagttggtgtgtaaagatgtagaaattgttttttatttggtttttttggaaagtagttgtttttgaaggagaatttgaaggatggttttatttgtgttagtaatttttatatttaaattaggttttttgtgtttgtttttttttttggttttttagtgttaggggagttttattttgggtgatttgtttttttaagaattgtttttttttggtagttttttgtttgtttttttgttttgttttattgttgtttttatttttataaaatttggatttttgtggaaggtgggtttatttttatttaaggtttatttttgagattttttagatagatttgtattttggagttttttgggagttttgggaggggtaggtttgggagtatagattgttttattatttaatgtttggatttttatgaaaatgtttttttaaaattatagtaatttagaagaaagtggtattttttaaataatttagttaaataagtaaaattagtttggtaaaggaatttgaagaatggtatttaaggtttgagttggttaagtttattaaaataggaaggaaaatatagaaaaggataatgagttttgaagtatattagttattttttttagttttgtgattatttatagatggaagaaattttagtatgtatttattgttttttttttgatttttggaaagaaaataagatgtttaaaaggaatttttgaaatattagttttattttatagaggttgatattttttttttttttattttttttttaaaggttataagtaattatagagaggttagtgtagtttaaatttttatatagtgattttttagtatttttggaaataggatttgtgtagttagaggtttatagaattttaggtaagtataatttagatttaatttaaattatttggatttagtttagttaaattatttatagtttgttagatgataatttattagtttaggttttatgtgaggaagaaaatatttaaataattatttttgtttttaaatattataattgaattatgtttaattaggagaatttaattagtttatatattggttaagttgggggaaatatatttttaaaaatatatgtttaagttaaagttatgaattgatttttttttttatttattaaatataaataaagatatatagttttatataaaaaggtaattaaattgtgaattaaatttaaatattttttttatttagttaaatgattgattagtttttttgagtttaataaagttggttgtttttttagtatgagtatttttaattgatatatatgtttatatgtatggtatggggggatttttgtatttaattaatttttgtgttgagtatagaagtttgtgtgaagtgtggtgattttttataagtataaagttttagtagtgatttttgtggatttatatgtggaggggtattgtgttatttttgtattatggatttttgtttttagttttgtttaaggtttaaattatttttttgggggatgttgtttagttttatttttattagggatggggatttgttgtttatttttggatgtatttttatttttttttttttattttagtatttttggttttatttaaagaaaagtttagtagaataagattttttagttagggtatattatagaagtgttttgttttttttttaaaaaattttttttttttttttggaaatttatttaaatattttagttttttttagaaaggttgtgttatagtttatgttatggagttgtagggttttgtgtgttttatattagttaattttttaagatataatagtttttgtgatgatggtaaagttgagtaatttgatattatttggagtattaatataggttgggaggaagaaatgggatatgtgtgaagattttttatgtttggagtgaatgttttagaattttttgggatttatatttttttgtttattaggaaggagaaatgtgagttgtttttatttattgtttgtttttagttttttgtttgtattgttagggttaattttggttgtggtgtaatatttgggttatagatgttagggggttgtgtttagggggaagtgggattttgagttttgttagttttggttagaaagtttggtattaaggtgttgggtatgtttttaggataaagtgtgggttttttaaaagaggaaggttgggtggtatttagggttttttgttttgtttgtagatataagtttatttgtattttgtgtaaagtagttttgaaagtttaggtttgaagataaaatgtggagtttaggtttttttgattgaaaatttttttatgaggatattttttgtagtgggtggttagaaaggattttggattaagatttgtagagtggtgagtattttttagggtttggggttgaattgattgtaggttggtagttttttggtttggaggagaattttttttttgtagaaattatttttggaaaataaaattgagtgaaaagaagggtaatgtgggtttgtgtggaagggagttttttttttggggaaggaggtgttttgtggtggtatatttgttggtgggatgtaggtaggggtagattagggtgagaagggggatttgtggaatatgggttttttgtgggtttgagggatagggtgtaggtgtattttaggtatttagggtagtttagtttgttttgtggagtgggagttggttttaggttttttatattttttaggaatttagaagtttgattgtgggatagttgtgttgatggggatttttttagttggatttttattttatttgtttttttgattgagtgtttatgtttggtaggagttggttgtgtgttgttttgggtgggtggaggtgtgtggggtttgggttttggtttgtttgggtgtgttttagttttttttttttagaattaaaggtttttgtagggtgtagagttttttttttttttttttaatttttttttttattttgttgtggtttaagttaagatgaggtgagtttttagggttgtgtttttaggaggttttgagtattttttttttgagggttgaagaaattaggttttttttttttttttagtgttttttgaggaagttgtgaatttttgagtgagtgtggttttattggggatttttagtgatttgttttgtgtttagtgatgttttaaggggagaggagttttgtggaggggtggtgtgggggtgggtgtttatattatattttttggtttggagtaggatttggtgatgtgttttggttgagtttttgttttttaggaatgtgggtgttttgggttttgttgggatttttggtttgattggagggtttgggagtgggagtttggatttaataggtatttggatttaggagttagattttttggttttgaatgttttttttggagtgaaagtttgttttttttttttttaagttttttgtgggttgggaggtgaagggagtgatagaagttgggttttttttgtagtgtttaatgttttgggttttttgggttttgggatgtggtttgggtgtgtggagggatggttgggggtgtagatttgtgaatgaattttggagttttttgtagtttatagattagaggttgtgattgttttattgatgtggtttaatgagttgttgtaataagttttgagtgatgaaatggaggtatggtttggattttgtggtattgtgttgtgtagggttggatgggggaggggttgagttattgtagtaattttaaagtttgtgttggggtttagtaatgggtgggttttttttgggttgtatggtttttaagggttttttttgatttttattgttattttgttggaatgtttagaggtgtttttagtttaagtgatggagattttgatttttagttgtttttgtgttttttgtttgtgtgtttttggtgttttttttgggatttggggggtgattatattttgagatttaatttggttatttttgggtgtgggtttttattgttttttgggttggttggatttgttttttaagtagttgttttatttattatttgttgttagttttttttttaaaatttaagtttttggttgtttttttgttgttttttgttttttgttttttgttttttgtgtgaggggaggggagggggaggaaggagtggatatttatgtgtgtttgggaggggtttgtattgtttttttgtttttttagtagaggttttgggttttttttttagagggaaattggtgagtagatattaagtgatgtgagttttggttttttttttgttgggggatttttgtgtgttttagaagtgtatttggaagtaattttttttgtgtaattgttgttttttaagtatttttagtatttttatattttttaaatatttgtataattttgtgttgttttttggaggattttggagatggggaggggatgatggggtgttgtgggggtagggaaggagagaggttaggggttatgtaggattatggtttggtttggtgattttttttattttttgtggtttagggaggggatttgtagtgagggttttagattttttttgtaaaattgaattttgggggagggtttgtgttttatgaagtatggggaaggtttaaatgatgaatatttatgagtggttgggagtggaagatggtaggattgatgggtttgggagggaagatgggtgtgtgtttttagtgattatggtatgaggtggtgttttgttttgtattatgttataatttaattaggtttttgtttttttgtgtttgaatagggtgggtttatttgggtggtgagtttgagtttagtgtagttttagtttggtgttttttttttgtttgttggggaagaggtgggaaattttttttgttgtgttttggtttttattgtaggtttttgagaataaatttttttagggagattgaattgtttttattttttgttttagttttaagagggattttttgaaaatatggtagttaagaatagggtgaaaatgtaagtttgaggtttttttttttatttttttttgttttttgtttttttatttttatattttttattagtttagaggggtttagttgtaattattattattgttatttttatggtgttgatgattgttattatttttttttagttttagttgttttttggatgtttttattggaaggttgatgagtatttttaattttatgtgtttattttagagtatttttgattttgttatttttgttgttttgatttgtgtttttttagttttttttagttatttagttggttattttttaggagatatttttgattttttattttttttttagagttattttttgttttttagtaaggtttgttggttttgtttttatgttttttttatagtttttttatatttttgtgttttttattagtttttaagttttttttttttttatttgtaggg

SEQ ID NO: 28 - DNA, 2609 bp, Artificial Sequence
  chemically treated genomic DNA (Homo sapiens)
tgtagattagagatgattatagattttttttagtgtggattaaagggattgaattgaatgttttagtttaatgatttaatttttgttatatttatagggatgtgaattttgattttataagtaggtgtgtatgtgtatttagatatttatataaagttgtgtggagggatgaaaagattaattatttgattgatgaggataggtttgatttttttgattattttagtgtgttagtgtatatttttggttgggtttagtgttttaagaaattttggaattttagttgttaatttttgtttttttattatgattttttaaagatattttatttgtttattggggtgaagagaaatgggattaggtgttagggtggtgggattttgtttagggttttgattttgtgttagggttttagattagttggtttttgaaggtttttttatttattttatataagaggaaataaagattttttagtttaaggttttagggttgttttttgattttggttagtttgtaggaagaggaaataataaaataaaggaattgttaatttgttgggtattatattttttagttttaatttttgattttggattgttagggttattttttttatgttgattttgttttttttaaatgaaaatatgttaataaaagtatattttggatataaaatttaagtatgtattttttttggggaggttagagttgaggtgtattttggaagatgagaattttgtttttatgaattgggtaatatttaggtattgttaggtattttgatagattttttagatattttttttttttttttttatattttttttttattaaaatagttattgttttgaaatttattagaataatgatgttttaaaaataaaggtgtagtaagtatttttttttttgttgttgtgggttgaattatggatgtttgtgggttgtttagttttgatggtttgtagggggtgtgtgttgtagttgtagtatagtttggttatttttagaaagggagttgaatggagggaagtagggagtgtggagggtttgaggtttgtagataaggagaggtgtattttgggatttgggttttttgttgttataatataattgtgttattgttggtattgtttgatttaagtgttggtggtaagtggtgatgttggggttgggtttttagtaattgttgtgttttgggttaggttggttgttttagttattgggatttttttttggatgtttttagggtgataggtgttgtatttattgatgggatagttgtatatttttgaataggtagtggagtttgtttttggtaggtattttagttgtgttgatattaaggttgttatataatagttattagttttttttaagttttagaagtaggttttttttgttttagttatagtggttttagttgttgttatgagtttattttttatttttaagtgtatttttttttttttatttgggtttatgtttgttatatagagagaattatatagggggaattatggttttatatttttgaggggatagatattggttgtgagataggtattatgtagagttttttggtgattgtttgtaggagtgagattttttttggttttgtagttggttaggtgtgtgtgtgtgagggtttttagttgattattgggatgtattgttattttttggtttggtgggttttgggattttttggtttttgtaggaggttattttaggtttttggaggaggtgtttttagttggtggtgttttttgttttgggtttagaggtggatatggttggttgtgttttgttggttttttgtttgtgttgtagtgggttgggagtagttgtgtgatattagatttatagtgttaagatgtgaagtgtgaggaaattgttgtgtttgatttttttttttttaatttttggatttaggaatgtttttagtttttgtgttttatatgtttagttttggttttttttttggtttagaagtttttaaggattgaagggttttgtttagggttttgtatttttgtttgatttttatggtttagaaaagtagggggatatttgaaatgttattttgggataaatattaataaaaaagtaaatggatttgtgtaggggttagttattaattaattaggttgtaaggttatttagggtaaatatagtttaggttgggttgggtgagatttttttatggttgttttttgattgaatttttttttttttggtttaggttttttttaggttgttttggggtaaatattggatgggaagggggtgttgttaatttttttgtggggtttggaggtttttttgtttttaagttttgtagggtagggttggagtgtttaatatttatttttgtttgaattttgggtttgtgtgttgtttttttttgggtttgtggtgttgtgtttgttgttgggtgtgtgttgttgtttttttttgagttggtagtttttgttgtgtggttgaagttttttggaatttttaattggaaattaattttggttttgatagatgtttatgttagaggtgtgttatttatttatattttttgttttattttggaggagatgtggtgagaattttgttgt

SEQ ID NO: 29 - DNA, 2609 bp, Artificial Sequence
  chemically treated genomic DNA (Homo sapiens)
gtgatagggtttttgttgtgttttttttgggatgaggtggggggtgtgggtgggtggtgtgtttttgatgtgggtgtttattaagattaagattagtttttaattaaagattttagagggttttggttatatagtaggagttgttggtttggaaagagatagtgatatatatttaatagtaagtgtagtattgtggatttaggggaaggtggtgtgtaggtttgaggtttgggtgggggtgggtgttgggtattttgattttgttttgtgggatttggaggtgaggaaatttttaaattttgtgggggagttggtggtgttttttttttgtttggtgtttgttttagaatgatttaagaaggatttgagttaaggggggaggggtttggttggggggtggttatgagaaagttttgtttagtttaatttgggttgtgtttgttttgagtggttttgtggtttggttgattgataattggtttttgtataagtttatttgtttttttgttaatatttattttggggtgatattttaaatgtttttttgtttttttgggttatgggagttaggtaggagtgtggggttttaggtagagttttttaatttttgaagatttttgggttgggagagaagttaggattgggtgtgtgggatgtaggagttggaagtgtttttgggtttaagagttgggggagaaggagttaagtgtagtggtttttttgtgttttgtgttttggtgttgtgggtttggtgttgtgtagttgtttttagtttgttgtggtgtgaataaagggttagtagggtgtgattgattgtgtttgtttttgagtttggggtgaggggtgttgttagttgggagtgttttttttgaaggtttaggatggttttttatggagattaaggagttttagagtttgttaggttgaaaagtggtagtgtattttggtagttaattggggatttttatatatatatatttagttgattgtaaaattaaaaaaggttttgtttttgtggatagttattagaaggttttgtgtggtgtttgttttatggttggtgtttgtttttttgagggtgtaggattatagttttttttgtgtagttttttttgtgtaatgggtataaatttgggtgaggagagggaggtgtgtttggggataagaagtgggtttgtggtgatgattggaattgttatggttggggtaggggaaatttatttttggggtttaaggggggttgatggttattgtgtggtgattttggtgttagtgtagttaggatgtttgttagagatgaattttattatttgtttggaggtatgtggttattttgttaatgggtgtagtatttattgttttggaagtatttggagagggattttggtagttggggtgattagtttgatttagggtatagtggttgttagagatttagttttgatattgttgtttattattgatatttgggttggatagtgttaataatagtatggttgtgttgtagtagtagaggatttaaattttaggatgtgtttttttttatttgtaagttttgagttttttgtgttttttgtttttttttatttggttttttttttgggggtagttgggttgtgttgtggttgtggtgtgtgttttttgtgggttgttggggttgggtgatttgtgagtgtttgtggtttagtttgtggtagtgaagaaagggatgtttgttgtgtttttgtttttagaatgttgttgttttagtgaattttaaaataataattattttgataggaagaaagtgtgaaggaggaagagaaggatatttagagggtttgttggggtatttaataatgtttgggtgttatttaatttatgaaaataaaatttttattttttgaagtatattttagttttaatttttttaaaaaagatgtgtatttggattttgtatttgaagtatgtttttgttggtgtgtttttatttaagaaaagtggggttagtgtagagaaggtgattttggtggtttgaagttggaggttggagttgagggatataatatttggtggattggtggtttttttgttttgttgttttttttttttgtaaattgattggagttaggaggtagttttgaggttttgagttgagaagtttttgtttttttttgtgtggggtgggtgaaagggtttttggaggttgattggtttggagttttggtgtggagttaggattttggatagggttttattgttttgatgtttggttttatttttttttgttttggtaggtagatagaatgtttttgggaagttatagtaagaaaataaaaattaatagttaaagttttgaagttttttagggtgttaggtttagttgggaatatatattggtatattgaggtaattgaaaagattaaatttatttttgttgattgggtggttgatttttttgtttttttatgtggttttgtgtgagtatttgagtgtatgtgtatatttgtttataaaattggaatttgtgtttttgtgggtgtgataggggttggattattaagttaaaatgtttaatttagtttttttgatttgtgttggggaaaatttatagttatttttaatttatg

SEQ ID NO: 30 - DNA, 11667 bp, Artificial Sequence
  chemically treated genomic DNA (Homo sapiens)
tgggatttgtgggtgttaattaatgttatggtggtggagagtaaaggggatgaatatagtttggggttatttttggagttttttagtgtttgtttgtggttgtgtttggttggttgtggattttggtgggtgttgtaaagtggtgggattgttagtgtagagttttggttttttgttttgtttttgggtttgagtattggagtttttggtgtttgtggggagaagttttggattgagaaatatgggagggttttgttagtggttgtaggtgtggtagttattttggggatttagtgagaatggggttgtttggttttgtgtgaattttttatgtgggtgtagtttttgagttgttgagggaggtggtggtaatgttgtttagtgttagtagagggtagttttgaggttgtgagtttgaatggtgattttgttaaattgtgggtttttttttagtttttggattttgtggggtagaggtggttttggagtttagagattagtgattttagtttgtaggagtttggtgtagaggtttaagggtatttttgggatgtggttaagttatagtttttaggtagttttattttgtgatggtaagggtttagagggtggagggggattagatgttttaggaggggttagaaagttaatgtatatagggaatttgtttttaagtaatagattttaagtatgtggaaattttttaaatgatgttggtgagagttatgatagtttttgtatttttggtgagggaagttggaggttagtagtgggatggtttttgggtgtgtgggagatagagggattagtgatttttgttgggggtagagggattgtggattaaggatttagatatatttaattggggattttagttttgattgtggttatttaattggtgttgaatttgagtaatttattatattgttttagtttttgattaattatttttgtaaaatgggtattgtgggttttgagttttgtaagggtgttagggatttgagatagtaggaatttttttattgttttagtaattttgggtttttgggtttgtaggtgggttgaaaggatttttttattgtatgtttgtttggtgtggttgttttagagattgaggtttgttaggttttgtaatttagttgtgtgtggttagttatggttttggaattgttgggattgttttagtggtattttggttagtttttgattttttgttttgggtattagggttatttagttttagaagatagtgtttattagtataggaattgagattttatatttggtgtagtgggttttttaggaagtttggtgaagagttaaggtttgttggatttgaggttgtttggggtgttaaatagatttatttatgagtatatttggttaatatatttaagtttaatagtgggtgggatttttggttggttttgattttttttatagtgtgtgtaaatgggtatttattagattttttttgtttgtttttttatttgtaaattttgaaaggagagtattttttggggtgattttgaaaattatgttagaattgaaaaggtttgtgtaaatgtgaggtgtaatgatgatttattagaagtagttagttttgtatgtgtttgtaggatagatgttagaatgttttataaatgtgtgtatatatatattatgtatgtgtgaaggtgtatatttgtttttagatatttgataaatgtatatatgtatgtgtgtatttttggttgagagtaaaggggttaatatagggtttatataggatattattttgtagttttgtgtgtgtatagtatatatgtgagtatgattgtttgtgtttggagatattgtatggtattggaggagttgtttagttgttggtgttatttgggaagtagggttttgggtgttgttttggagtgtgggaaagtttggatttgggtttgttggttagtgttttggtgttgttgggtttttttattttttatttgttggttggtgagtggttttagttttaagatttttgattttgttggtgaggggagggagtgagaaggagtgtgttttgggtttaagaggttgtagggttttattttttaggtttgttgttttttttttatttttttaggttatatttttatttaaaataaagaagggtgtttgatttatttaggttagggaagttggtttttgggagttttttgtttttataagttatggtgaagggaggtgaagttagggtttgttatggtttgggagaaaatgtggttgtagggtttttttgggttgtagtgttggtttggggttttaagattgttaaggtgtgtgtgggaggtgtgtagtgtggttattgaagagttttttattattgtattggtaggttgttgggtttgttttttttttatgtttttaagggttttgagttgtgtagtatttgtattatttagaagtgtttgtggagaaaatgattttaggtgtaagtttaaggttgttgtttgtaaaggtaatgttgtggagattgggttttatagtgatttgggtttttaagttattgaaagttatagggtagggttataaatatttttttgttttttttgtagaattgtgtggttgataggagagtgttgtgtagaaaatttgaggttgggtgttggaggagtttttgtggtttggagaaggtatagaggtgtttttgagaggtgtagttggaataggtgatgtatgggtttggatttggttggttaattgagtttagtggttgtgaaaattagagttgttatagaggttgagtatgattttatttttggggattgggtttggtggggagttattgtttttaatgtttttagagattttaagtaggataatataatgttggtaggagtaaggtgtaaggaatttagtggtagaagttttttgggggggtggggaaaggtagatttgtgttttgtttgagtttgggggtggggatagtttttggttttgtagtttttgttttggggattagttgggtttatttaatttttttatatggggttgaaaggggttaatgggatggttttgttgttttttttttgggattttatagggttttgatttggtttttatttttgtatggttagtagttgtgggggttttaggaaggaggataaaggtttggtgttattgtgggtgggggtttggttttttggttttttgtttatatttatagtttttagtggttgttgggggaagatttttaaaaatatgtgttgaatttttttttttttttttttagaaataaataaataaaaaaggtaaaaggtgaatttttttttattttttgtttatagagattaaagttttttgggattttgttttttttttttttttgattgtttagaaatatttgtttttttttgtatatagaggaaaatgtggggaaaggttttttaaaatttggttttatattattattattattaaggataattgggtaggttgaggttttaatgtggatgatttgagttggttttgtgttggggttttgtagttattgttttgtgtgtttagtatttttgggggtgattagggtttttgtgtttttgtttgttgtttggtagttgagagtattttgtgtttagattggttgatttatttttttttgaattttgtttagagttggtaagggggatttagtttgtgttttaagatttgggtttgtagtgttgttaataggtttggggatatgaggtgttttaggttggggtttttttggttgttggttttttttgttttttatttgttggtggtgttttggttgtttgtaattgatttaatttgttttttgtgtttgttttttaggttttttgtttttttataaaggtttaggggagttttgtttataggttgattttgtaatttttggtttggtggttattttttgtttttttgaaaaagaaaaggaaaaaaaaaaaaaaaaagaaaaaattaattagttgagggaggtgtgtgaggattggaggtgttagttggatagtttatgagtaggtttttgggttggtgttgttttgtgggttagtatggttttttttagagaaaattttttaaatgtgtgaagattgttttgggggaagtgagagggaggttggaggagttttgggtggggttttagtgtttattagttgtgtttttagggtttgggtgtttgttgtaatggtaattgtgtgagttttatttttatggttaaggggttagggtagggtggatgtaattgtgtgtgtttggttttggaaggtgtttgtagggggtgtttgtggttagttgggttaggaagttaaattttagaaagttattattaaaggtttttttttttttttttttttttttttttttttttgtttttttttttttttttttttttttaataggggaagggaaaaaatattattgaatatttattataaattgggtatttaaaaattagaaaatttaattattttaataattttgatatatgtatgtatttttgttttatagaggagaatattgaaatttgaatgggtattttgtttaaggttatatagttgttaaagaggatattaggttttaatttagttttttttgattaaataggttaggtttttgttttatgttgtataggttatttttgtgaagtgataagattttagaatatgttttttatagtttagtattaagtttgtgtggtaaatagtttgtgtagtagtagtttttgtggtatagaaataaataaaaaaggtaaaaggtatagtttagattgtgtttttttttttttttgggaggtttttttttattatttttgggaggtttttttttattatgtagtttttttgagtattagtggtagttaggtttagttgttgttaagttttttttaaaaattttggaggttatttgttttatagatatttggaagatttttggttggttttttttttttttaggatttgagattttttaaatgtaatgtaattttgggtttttagatttaagatattttttttatgatagttgggagttttttttttagaaagtggaaagttgttaagattgtttggttttaagaatttttaatttttggttgttttgaaggtatggggtttttagttttattttttttttattttttttatgggttttatagtttattttaagaagtaggggttgttatatagtggtggtaggtttgatgggtagggttttagggttattattaggttagatttgtagaatggggatattgtagggatgttgagtttgattaagatgttttttggttttagatttttatttagaagttaggagtttggtttggtgattttgtttatgaagtttggtttagatttttataaagggaagagtttagttgagagtaaattattttgggttagatttagatttttggtttatattttgtttgtttggttgtatgaagtttgggtttggttttaatttttatttgttgttagggggttttttgtagttttatttttagttttggtttttaggaaattttggtaaaggttggatgtatttaattttggatgaaaggtttagggttttttgtttatttaagttttagatttggagtagtgaattatttattgatgtaagttggaaataggatgttaatattttttggagtagggtataggattatttttagaattttgtaggttttttttttatagttttttggtgtatgtattttaatggtgaagatttttttaggttttttttgagaataggatatttttttttgttttttttaaattaatttatttattttgattgggtaattttagtgaggtggaggtgattttaagaatttttgtgtaaatgtgaggagagggtttatattgttagtagtgtgtgagatgtttatatttattatatatataatttttatgtaagaatgatttattatttaggatatttagaaattggttgggtatttgtattaaattttatttatattgtatttatatattggttaatatatatatatatatttatatggtttagttatttaggttagattgtatatgttaaggtataattgttatataatttaaatatttttgtttttaggaattgtttaaattagtttttataatagaaatttaaaagtgtgaagagggagagaagagaaaggaaatgagagatgaagaaagtttgttattgaattaaatttttggttttggggaaaatgattaaatttaagagttttggagaaattttgagggttggagttagatagattttttttgtgggtatttatggggaagtggggtatgatttggttttttttttttattttttttaatttttgtgtagatttaggaaatttttattgattgttttattttgttttttttatttggtgaggttgttgttttttttttagttttgggtgttttttgttttttagtttttggggaggagataggttggttttttattgttggttttggttaatttttttgttttattggaggtgggttgttatttgtttattagggtttggttttggaagttttttttttttgttttttttgtttgagatggtagttgggggagtgttgatagttagttttagaatagggagtttttgtttgatgaagtaaaggttttgtttatattattttattttttagtaagttaggaatatatatttataaatgtatatattattaatgaggtatgaatgttatatatattgtggttttagtaagtattttttaagtgttagttatatgattattataatgttaaaaatatatgtttattttgtaggtgaatgatagatatatatattttttattttgttattaatatataattgatattgtatatatatatttgtagtagatatagattatataattatttgtttatgggttaaatttttatgttagataaagtataaatagatatgtatatagtaggttgttttttgttttgtaattttttttagttagatagtggagtagttgggtattaaaggtatttagatattaggtttgattagtttttagggttttttgttttagttgagtttttttttttattttgtttttgttggtttttggtaagttttagttttagttttgaggtgatgttttttgagaatttttgtggtttggttttttgttttgtagttttttgtaggatgtttgtggggggtggggggtattttgagtagaagttatttgggtttaattatatataataggatattattttgaggtttttattgtttgattattggagggttgaagtgagaggtttgtaggtttatttttgtggtgtaaaattgtttttggtttattagttggtttggatttgttgttatattgttggttaggttttgagatagatggggggggggtagtgagggttgggtgtggggtttagttttagttgagtagtagtttgtaagaattggtggatttattttattagaagttggaagttatttattttattagaagagtgaaagttgattattttttaatttgtttgggagaattagattgtaattaggttaaatgtttgtgtgtttgtgtggaagtatggattttaatttgttatttggggattttagtaaaggaagagagagaaagaaaaaagattaaatttttgtagttgtgaagtatgtttaatttttattgaaatttttaaattttttatttatattttaaggtaaatgaattttgattattaaaaataataaagtagttattttttatatttgtatagaggttagtagtttgtagtgtatttttataattagaattttatttattaatgtttggaataaatttttggttgttatagttgatttaaaattgaaaagtttgagaataataataaaaatatagttagtttatatatttagtataattgtgtatgaggtagaatataaatttgaataggtataaaaataaattaagttttatttatagttgtgttttgtgaatgaattatggtagaaaaagtttttttgttgatgttaaatagttgtaatgggttagtgatgatatagaaaataaataattttaggaaaataaagtagtagtgatagttataaatgtttgttattttggttgtttttaatttttgtttaatttattttgaaaaagataaaattttgttgaaaattttttttttttaattagttgttgaattaaggagtgatagaaataaagaagtttgtgttaaaagaaggaatataaaatttatttgggaattgataaggtaaaaaataaaaataaaataaaaaaatataataaaagtgttttagttttttttagaaaatttggttttttaaataataagttatagtataattaggtttatggtttttgggttgggttggttttgggttataagattgtttaggttggttgttgttgggttttatattttttttttttaaggtgggagaagtaggaggtttgaaaaataaaaagtagggaggatgagttgttttagtagtgtgggttaggtaggtagtgatttttttgtttttaggatttgtattgaaagatttggggatatttgtttttgatttttttttttagatagggagagggtgaaatttttttaattggtagatgaaattattttttattggatatatttttttaattttgaatatagttttttgaaggtttaataaaaattttaatgtaaattttttagaagagaagatgaggaatattaaagtttttgatttttatatttggatttagtaaattttaaggttataatatttttttggttgtttggtgaatttttttttttttttttttttttaaatatttttttaaaaagttttattgattttttaaataaaatattagttagttgttggggtttggaggtgtttgaagtgatgggattgatagtggaggaaatgtagtttttttttgtgtttgtttgtggtgtaaattgagtataggttgtggagttaggttgttttggtttttgttgggttttaatttttgttttgtttagtgggttttgtggtaagtggtttttgaatagttttaagagggtttgtggagtaaatatatgtattggtttgttttttttttgggtagtgtggttttgttattagttttgtttggtgtgtttagttatggattgtatggtagttgggtggggaatgtggagagtgagtgtattgatttgtgagagaaggttaagaggtttgtgttgttgatgtttggttgtatttttgttttgggttttttttgtggtgaatttgggtaggagatgttggggttttggaaagagatgagtttagtagaaagtgtgtagagaggtagttttaggttaggggagtgtaaggttatagaggttagggaggtgagtataggaggatataaattgaggggataaagaggagtgataggagtttaggaaagtgaaaaagtatagagggattttgggtgttggttttagaggtgggtttagagggtgtgaggttaggttggtggtggtgttgttggttgtgattggggttggtgttgtgtgtttttgtatttttgtatttgtttgtattggtatgtggtgggtttttagagattgaggtgtgaatgtagtgtgttggttttgttgttgtggttttagtaagagtaatgtattatggtgagtttgggttgttgggtataagtgaattgtaggtttggtgtattggtgggtgtgggtgttgggggtgtggtggtggttgggttggtagggttgagttggtttggtggtggtgtggtggttttgtggtgtggtggggtaggtggtggtgtggtggttgtgtgtagatggtgtgtgtgtggatgtgggtgggggtgtggtggaggtgggggtgtggttggtgggttttttaggttattgtatgagtttattatgttggggggtagtgttatgttttgtatgtgtgtgtatggtttgtatgaggtggttgggtttgttagttttttgattatagtggttgtgttagggttattggggtttgtggttgtagttgtagttgttgtagttgttgtggttgttgttatttggtaggaggtatagggtatgggtgagggaggttgtggtagtggttatgagttgttgaggaagttagattgtaggtatttggggggtgttaggtagttgtagttgttggttttggtgtttgttatgttgtatttgtttgtggtgtttttggttttgaagagttttttgttgggttggaagtgtgtgggtggtggttggaagggtttttttatgtggtggtggtgttggtagttgttttttttgaatatgtttttgtaggttgggtttagtgtttagtagttgtttttgtgtttgttgttgtttttgtgtggtattttgatgaagtatttgttgaggttgaggttgtggtggatgttattttgttagttttttttattttttttgtagaatgggaattttgtgatgatgtattggtagatgttggatagtgtgagttttttttttgtgtttttgtggattgttatggtgatgagtgttatgtatgagtatgggggtttttgtgttgggtttggtttttttggggttgttttgttgttatttttattgtttttgtttgggtttggtggtggtttttttggttttttgattgtgtgattggtttttggggttagtagggtttttgttgtgtttttgggtttggggtagttggttattatgataaagttggtgtgttgtggttgggttgtttttgttttttgttttaggtgttggtgtggtaaagagttggggtgtatgagtttgtttatggttaagttttaaatttttggagattgtggatgttgtttgtgtttgtttgttggaggtttgttgttgttttttttttttttttttttttttttagggagtggttggtgggagtggagtttagtttttggttatggggagtttgtttaatagagaggggttttggttttgttgtttttttttgtttaggttagtttttgttttggtgggtttttttttttgtgttttttttttttttttgttttttggttttttgaagtatgatttgtgtttttggtggagttgttttttggagtttttagtgtgttaggagttttgttttgttttgatttgtatgggttttattgagttttgtttgtgttaggtgtttttgtttttatagtggggtggttagttgtgtatgggtgagtttatttttaagttattttttgtaaatgttttgtatagtttggattggtttgtttttgtttagtgagttttaggggtttagttgatagttaggtttatgtgtttttgaaatttgttggtatttgttttgtgggttgggttgggagatgatgaggattttggtggggtttgtttgtatttggttaaagtttaggaagtttgggttttagtgaggaaaggtgttttaagtttttttgtggtttttaggtgaaagaaaatgatttttttgttttgttgtttgttgttgttttgaggttgaatttttagtttggggttggggaggggtgagatggtgagggggttggatggggtagggtggggagagttgttttgaggttttgggaaagttagtttagaaatgggtgtgattgtatgaagaagttttggtttggtttgttttttgtg

SEQ ID NO: 31 - DNA, 11667 bp, Artificial Sequence
  chemically treated genomic DNA (Homo sapiens)
tgtgagggataggttaggttgaggtttttttgtatagttatatttgtttttgggttgatttttttaaagttttagagtagttttttttattttattttgtttagtttttttgttgttttgtttttttttagttttgagttagaagtttagttttaagatggtagtaaatggtagagtaaaggagttgtttttttttatttgaaagttgtgaggaggtttggagtgtttttttttgttggggtttgagttttttgggttttggttgggtgtgggtagattttattggggtttttgttattttttagtttagtttgtagagtgagtattggtagattttaagggtgtgtgagtttggttgttggttgggtttttgaggtttgttgggtgggggtaggttggtttaggttgtgtggggtgtttataaaaagtgatttggagatgaatttgtttgtgtgtggttggttgttttgttataggggtgaaggtgtttgatgtaagtggaatttggtggagtttatatgaattagaatagagtgaggtttttggtgtattagggattttaggaggtagttttgttagagatgtgggttgtgttttgggaaattggggggtggggggaggggaagagtgtagaaaagaaaatttattaaggtggggattggtttgagtggggaggggtggtgaggttggagtttttttttgttgggtggattttttatggttagaggttgagttttatttttgttggttgttttttaggggaaggggaaggagaggggagagtagtgataggtttttagtaagtaagtgtgggtggtatttgtagtttttagaagtttgagatttggttgtaagtggatttgtgtgttttaattttttgttgtgttagtgtttggagtggagagtagaggtggtttggttgtggtgtgttggttttgttatgatggttagttattttgagtttgaggatgtggtgggggttttgttggttttagagattggttgtatagttaaggagttagaagggttgttgttgagtttaggtaagggtggtgggggtggtggtgggatagttttggagaagttggatttggtgtagaagtttttgtatttgtatgtggtgtttattgttatggtgatttgtgagagtgtggagaagaggtttatgttgtttggtatttattagtatattattgtgaagtttttgttttatgagaagaataagaagggttggtaaaatagtatttgttataattttagttttaatgagtgttttattaaggtgttgtgtgagggtggtggtgagtgtaagggtaattattggatgttggatttggtttgtgaagatatgtttgagaagggtaattattggtgttgttgttgtatgaagaggtttttttggttgttgtttgtgtatttttagtttggtaaggggttttttggggttggaggtgttgtaggtgggtgtggtgtggtgggtgttggggttgatggttatggttatttggtgttttttaagtatttgtagtttggtttttttaataatttgtggttgttattgtagttttttttatttatgttttatgttttttgttagatggtggtagttgtagtggttgtagtagttgtggttgtagttgtgggttttggtagttttggtgtggttgttgtggttaaggggttggtgggtttggttgttttgtatgggttgtatatatgtgtgtagagtatggtgttgttttttggtgtagtgaatttgtataatggtttgggaggtttgttggttgtatttttgtttttgttgtatttttatttgtatttgtatgtatattatttgtatgtggttgttgtattgttgtttgttttattgtattatggggttgttgtgttgttgttgggttagtttagttttgttagtttagttattgttgtgtttttggtgtttgtgtttattagtgtgttgggtttgtagtttgtttgtgtttggtagtttgagtttgttatgatgtattgtttttattgggattatgatagtaagattggtgtgttgtatttgtgttttgatttttgagagtttattgtatgttggtgtagatggatgtgaggatgtagggatgtgtgatgttggttttggttgtagttgatgatgttgttgttagtttgattttatattttttgggtttgtttttggagttagtgtttagggtttttttgtgtttttttgtttttttaagtttttgttgtttttttttgtttttttagtttatgtttttttgtgtttatttttttgatttttgtgattttgtatttttttggtttgaagttgtttttttgtgtgttttttattgggtttgtttttttttggagttttagtgttttttgtttaaatttattgtggaaagggtttggggtggaggtgtgattgggtgttggtagtgtagattttttggtttttttttataggttggtgtgtttgttttttgtgttttttgtttgattgttgtgtagtttatggttagatgtgttggataggattgatggtgggattgtgttgtttgagaaagggatggattaatatgtgtgtttgttttgtgaatttttttgaagttgtttagaagttgtttgttgtggggtttattaggtggggtgggggttgggatttagtgggagttggggtagtttggttttatggtttgtatttggtttatattgtgggtgggtgtggagggaggttgtgttttttttgttattagttttgttgttttgggtatttttgggttttggtggttggttaatgttttgtttgaaagattggtggaattttttaagagagtatttaaaaaaaaaaaaaggaaaaaaaatttattgggtaattggggaagtattgtggttttggagtttgttaaatttaaatatgaaaattaaaagttttagtattttttatttttttttttggaagatttgtgttagagtttttgttgggtttttaaaaagttgtgtttagagttaggagaatatatttaataaaagatggttttgtttattaattggggaagttttattttttttttatttgaagaaaaaaattaaaaataaatgtttttggattttttgatgtaagttttggaggtagggagattattgtttgtttggtttatgttgttgggatggtttgttttttttgttttttgttttttaaattttttgttttttttattttgggaaggagaaatgtgaaatttggtagtggttgatttaggtggttttgtggtttggagttggtttggtttgaaaattatagatttggttgtattgtagtttgttgtttgggggattaaattttttagagagaattagagtatttttgttgtgtttttttgttttgtttttgttttttgttttgttgatttttgaataaattttgtgtttttttttttaatatggatttttttatttttgttgttttttagtttagtagttagttaaaagaggaagatttttagtaaaattttattttttttagaatgagttaaataaagattgagagtaattggggtggtaggtgtttgtggttgttgttgttgttttgtttttttgggattgtttgttttttgtgttattattggtttgttgtggttgtttaatgttagtagaaagattttttttgttgtggtttgtttataaggtgtgattgtaggtaggatttggtttatttttgtatttatttaagtttgtattttattttatatatagttgtattaggtgtatgggttggttgtgtttttattattgtttttaaattttttgattttgagttaattataatagttaagagtttgttttaaatattaatagataaaattttggttgtggaaatgtgttgtaaattgttaatttttgtataaatgtgagaagtgattgttttattatttttaatagttagaatttatttattttgagatatgggtaggagatttggaggttttagtaaggattgaatatattttgtggttgtagaaatttagttttttttttttttttttttttttttattgagatttttaaataatgaattaagatttatgtttttgtgtaggtatataggtgtttgatttggttgtagtttggtttttttaggtggattagggggtggttggtttttatttttttgatgggatagatgatttttgatttttgataggatagatttgttggtttttgtgggttgttgtttagttgagattagattttgtatttagtttttattgttttttttttgtttattttggggtttggttagtaatgtggtggtaggtttaggttggttggtgggttaggagtggttttgtgttatgggaatgagtttgtaggttttttattttagttttttagtggttaggtggtaggaattttagggtggtattttgttatgtatagttgggtttaagtggtttttgtttagaatgttttttgttttttataggtattttgtgagaggttataaggtaggagattaagttatggggatttttagggggtattattttaagattgggattggagtttattaggaattggtaggagtaggatggagagggaggtttagttggagtaggaagttttagaggttggttaggtttggtgtttgggtgtttttggtgtttggttgttttattatttggttaaggggagttgtagaatagaaagtagtttgttgtgtgtatgtttgtttgtgttttgtttagtgtgaaaatttagtttatgggtaagtaattgtgtagtttgtatttgttgtaaatgtgtgtgtatagtgttagttgtgtattggtgatagggtgggaggtgtgtgtgtttgttatttgtttgtagagtgggtatatatttttggtattgtaataattatatggttaatatttaggaagtgtttattgggattatagtgtgtgtggtatttatattttattggtgatgtgtgtatttgtgggtgtgtatttttggtttgttgaaaaataaagtaatatggatagagtttttgttttattaggtagaggttttttgttttgaagttggttgttagtatttttttaattgttgttttaagtagagaaagtaagggagggaagtttttaggattaagttttggtgggtaggtagtagtttatttttagtgagatggagaagttggttggggttagtaatgaaggattagtttgttttttttttgagaattgaaaggtaaaaggtatttaaggttgggaagagagtagtagttttattaagtaaggaagataaaatgaagtaattagtgagggttttttgggtttatatagggattgaaggaggtgagagaagggagttaaattatattttatttttttatggatgtttataggaaaaatttgtttaattttagtttttgggatttttttaaagtttttaagtttggttattttttttagggttgaaagtttagtttaatggtaggttttttttattttttatttttttttttttttttttttttttgtatttttgagtttttattatagaagttgatttaagtaatttttgagggtaaaagtatttgagttatatagtaattatgttttagtatatatagtttggtttagatggttgggttatgtgagtgtgtgtatatatattggttagtgtgtaaatataatgtaaatagagtttaatataaatatttagttaatttttgaatgttttgggtaatgagttgtttttgtatgaagattatgtgtgtggtgggtgtaaatattttatatattgttgatagtatgagttttttttttatatttatatagaaatttttagagttatttttattttattaaaattgtttagttagagtaagtgggttggtttggagaaagtagggggaggtgttttgtttttaagagggatttggagaaatttttgttattgaagtatatgtattaagggattgtgggaaagggatttatggagttttggagatggttttgtattttattttagagggtattgatgttttgtttttagtttgtattagtaaatagtttattgttttagatttgaggtttagatgagtaagaggttttggattttttatttgaggttaggtgtatttagtttttgttagggttttttggaagttaaggttggagatggggttgtaaggagttttttggtggtgggtggaaattggggttagatttaggttttatgtagttagataagtagaatatgggttaggaatttgagtttggtttaagatagtttgtttttaattgagtttttttttttatgaaggtttgggttaggttttatggataggattattggattagatttttggtttttgggtgggggtttaaggttaggaaatattttgattaggtttagtatttttgtagtgtttttattttgtagatttaatttggtggtgattttggggttttgtttgttaggtttattattattgtgtggtagtttttattttttggagtagattgtgggatttatagaaaagatggggaaaggatgggattaagagttttatgtttttgggataattaggaattaaggatttttaaaattgagtagttttagtaattttttattttttggagagaggatttttagttgttataaaaagagtgttttgggtttaggaatttaaaattatattatatttgaggaattttaggttttaagaagagaagagattaattagaggttttttaggtatttgtgaggtaaatggtttttgaaatttttaaaggagatttgatagtagttgagtttagttgttattgatgtttaagggagttgtgtagtaagggagagttttttaggagtagtaagggagagttttttaggagaagaaggaagtatagtttgagttgtgttttttgttttttttgtttgtttttgtattatagaaattgttgttgtataggttgtttgttgtataggtttggtgttgagttgtgaaaagtatgttttgggattttgttattttgtggaggtggtttgtgtagtatgaggtaaagatttggtttgtttagttaggaagaattgggttggagtttggtgttttttttaatagttatgtaattttgggtaaaatatttatttaaattttagtgtttttttttataaaatagaaatatgtgtgtgtattagaattattaaaataattaaattttttaatttttaagtgtttagtttatagtagatgtttaataatgttttttttttttttttattaaaaaaaaaaaaggaaggaaggaagtaaaaaaggaaagaagaaaggaagggggaaaaaaaatttttagtaataattttttaggatttgattttttaatttagttggttataggtattttttgtgagtattttttggggttaggtgtatgtgattgtatttattttgttttagttttttggttgtgggagtgaggtttatgtggttgttgttgtagtgaatatttaagttttgaaggtatagttggtgggtgttgagattttgtttggggtttttttaattttttttttgttttttttaaagtggtttttatatgtttaggaggttttttttgagaaaggttgtgttgatttgtgaagtggtattgatttagggatttatttataggttgtttggttggtgtttttagtttttatgtgttttttttgattggttgatttttttttttttttttttttttttttttttttttttagaaaagtagaaggtagttattgggttagaggttgtagggttagtttgtgggtgaggtttttttaggtttttgtggagaaatgggaaatttgaggggtaaatgtaggaagtgggttgggttaattgtgggtgattgaggtgttgttagtgggtggggagtgagaggggttagtagttgggaagattttggtttggagtgttttgtgtttttgggtttgttggtggtgttgtaagtttaggttttggggtgtgagttaagttttttttgttagttttaaataaaatttgggggagaatgagttggttagtttgggtatagggtgtttttgattgttgggtggtgggtggaagtgtaggggttttgattgtttttagaggtgttgagtgtatagggtagtggttgtagagttttggtgtgaggttaatttggattatttatgttgggattttagtttgtttggttgtttttagtaataataatagtatgaaattgagttttaaaaaatttttttttgtatttttttttatgtataagggagaataagtatttttaggtagttaaagaaaaaaaaagggtaggattttaggaaattttaatttttatggatgaggagtgaaggggaatttgttttttgttttttttgtttgtttgtttttagaagagaaaaaaaggaaatttgatatatatttttaaaaatttttttttaataattgttaaaggttgtgagtgtgagtagagagttaggaaattgagtttttgtttgtggtgatattgggtttttgtttttttttttgaagtttttgtgattgttgattgtgtagagataaaggttgggttagggttttgtggaattttgagggaggggtggtggggttgttttattggttttttttaattttatgtaggggggttaggtgggtttagttgatttttggaataggggttataaggttaggggttgtttttatttttaggtttagatgaggtatggatttgtttttttttatttttttgaaaggtttttgttgttaaattttttgtattttgtttttattagtattatattgttttatttaaagtttttggaggtgttagggatagtggttttttattggatttgatttttagaaatggaattgtgtttaatttttgtggtgattttggtttttgtgattgttgggtttggttggttggttagatttgaatttgtgtattgtttgttttagttgtgttttttaggggtgtttttgtgttttttttgggttgtggagatttttttagtgtttggttttgaattttttgtgtaatgtttttttgttagttgtatggttttgtagagaaggtgggagagtgtttgtgattttgttttgtagtttttggtgatttaaaaatttaggttgttgtggaatttagtttttatggtattgtttttgtaggtagtagttttgggtttatatttgaggttgttttttttatagatatttttgggtgatgtgagtgttgtgtagtttagaatttttggaagtatgagaggaggatgagtttggtggtttgttggtgtggtgatggagggttttttggtagttatattgtgtgttttttatatatattttaatagttttggggttttgggttagtgttgtagtttaaagagattttgtagttgtatttttttttaggttgtggtaaattttggttttgtttttttttattatggtttgtgagggtaggaggtttttgggggttgatttttttgatttgaatgagttaggtatttttttttattttaagtgggggtgtgatttgaggagataaaggaaggatgatagatttggggagtggggttttgtagttttttgggtttggagtgtatttttttttgttttttttttttattggtaggattgggggttttgaaattggagttgtttattggttggtggatgagagataaaggagtttggtggtgttgaagtgttgattagtagatttggatttggatttttttgtgttttagagtagtatttgaaattttattttttaggtggtattgatggttgagtagtttttttggtgttatatggtgtttttaggtatagatagttgtgtttgtgtgtgtattgtgtatgtataagattgtaggatgatgttttgtatgagttttgtattagtttttttgtttttagttagaggtatatgtatatgtgtgtgtatttgttaagtgtttagggataggtgtgtatttttatatatatgtgatgtgtatgtgtatatatttgtagaatgttttagtatttattttataggtatgtatagggttggttgtttttaataagttattattatattttgtatttatataggtttttttagttttggtatgatttttagaattattttgggaaatattttttttttaaagtttatagatgaggaaataagtaaaaagaatttaatgggtatttgtttgtatatattgtgagagagattgaggttagttaaagattttgtttattgttgaatttgggtgtgttggttgaatgtgtttatagatgggtttgtttagtgttttaggtgattttaggtttggtgggttttggttttttgttaggttttttgggagatttattgtgttgggtgtggggttttaatttttgtgttgataagtattattttttggagttgggtgattttagtgtttagggtgggaagttgggagttggttggagtgttgttgaggtgattttggtggttttaaggttatggttgattatgtgtaattgggttgtaaagtttggtgggttttggtttttaaagtagttatgttaggtaaatgtgtgatgaaaggatttttttagtttatttataaatttaggaatttagggttgttggagtagtgaggaaatttttgttgttttgaatttttagtgtttttatgagatttggagtttataatgtttattttatagaagtgattagttggaggttagagtggtatagtgagttgtttaagtttaatgttagttgagtggttatagttgggattggaatttttaattaagtgtgtttggatttttggtttatagtttttttgtttttagtaaggattgttggtttttttgttttttgtgtgtttaggagttattttgttattagtttttgattttttttgttaagaatgtgaaaattattatagtttttattagtattatttaaaaggtttttatatgtttggaatttgttgtttgaaaataaattttttgtgtgtgttggttttttaattttttttaaggtatttggtttttttttattttttaagtttttgttgttgtagagtgaggttgtttaagggttgtagtttgattatattttgggggtgtttttgggtttttgtgttgggtttttgtagattgaggttgttggtttttgggttttaaggttgtttttattttgtagaatttgggagttggaagggagtttgtggtttggtgaggttgttgtttgggtttatggttttgaggttgttttttgttggtattgggtgatattattattgttttttttggtggtttaggagttgtatttatgtgaggggtttgtgtagagttaggtgattttatttttattgggtttttagagtggttgttgtatttgtagttattgatgagatttttttgtattttttaatttgagatttttttttgtagatattagaggttttggtgtttggatttagggataaggtagagagttggagttttgtgttggtaattttgttgttttgtggtgtttgttgggatttgtggttaattaggtatggttataggtggatgttgggggattttggggatggttttaggttgtgtttgttttttttattttttgttattatagtattgattggtgtttatgggttttg

SEQ ID NO: 32 - DNA, 1394 bp, Artificial Sequence
  chemically treated genomic DNA (Homo sapiens)
tggaattttggtttgaagttgagataggagattggatgtgaggtttttttagagttggttttttttaaataatttttaaaatttttagattttaggggtatgttgaaattttttaaagtagtttaaagaatataatgagagttttaatattttaggtggtggtgtgttggttttttggagtggggtgggatgtggttgtgtggatttatgtgtataattgtgtgggatggggttatgtggatttatgtgtataattgtgggattttagtgttagtgggattttagtgttagtgggattttagtgttagtgggattttagtgttagtgggattttagtgttagtgggattttagtgttagtgggattttagtgttagtgggtttgtggtttagtggagtgagtggagtgttggtgatttgagtggagattgtgttttggatgttttagtttagatgttaagttatagtttgtgtagtagtagtaaaggggaaggggtaggagttgggtatagttggatttggaggttgtgatttaggggaaagtgtgggtggttgatttagggtagttgtggtggtgaggtaggtgggttttttgttttttggagttgtttttttttatatttgtttttggtgtttttagtagtttttattttggttttttgtggttattgtgggatttggtgttgttgttagtttagtggggagtgaattagtgtttttttttgtttttggtttttttgatggtatgaggaatttttgttttgttttatagatttttggtttttgttgagtgtggtattggagtttgttttgttagggttttggaattagagaaagttgttttttggttatttgaagtgttggatttttatagtgttttttagtttgggtgggagtggtggttgtgttgttgaaggttggggtttttggtgtgaaagggaggtagttgtagttttagttttattttagaagtggtttttgtattgttgtggtgggtgtttttgggttttgattttgttagtgttgtggggtagaggtatttggagtttgtagggtttagatttgggttggaaaagttttgttgattgtaggtaagtgtttgggaggggtggttaggtgaagttttggtgttttattatatatttttgggttttatgttagttgtatttgtggtattgggtaggaaatggtagggttgaggttgattttaggagtataagggagttttttattttttgtttatatttgttatttttagttttgtaatttattttagatatatagaaagtaagtaggattggtggggagatggagtttaataggaatattttttagtagtgagtaggggttgtatgggatgtgggaggagtttagaggaggtgtggagagtgtttg

SEQ ID NO: 33 - DNA, 1394 bp, Artificial Sequence
  chemically treated genomic DNA (Homo sapiens)
tgggtattttttgtgttttttttgagttttttttgtgttttatatagtttttgtttattgttggaaaatatttttgttaagttttgtttttttattagttttgtttgttttttgtgtgtttgggataggttgtaaaattggaggtgataaatgtgggtaggaaatggagggtttttttatatttttagggttggttttagttttgttattttttgtttaatattgtggatgtaattggtatgggatttggaagtgtgtggtaaagtgttggggttttgtttggttgttttttttggatgtttgtttgtagttagtgaagtttttttaatttaggtttgggttttgtgagttttaggtgtttttgttttgtggtgttggtgaagttgaagtttgagaatgtttattgtagtgatgtgaaggttgtttttggggtggggttgaggttgtagttgttttttttttgtattaaggattttaatttttagtgatgtagttgttgtttttgtttaggttgggaggtattgtagggatttgatgttttaggtggttaaagagtgattttttttgattttagggttttggtggggtaggttttagtattgtatttggtggaggttgaaggtttgtggggtaggataggagttttttgtgttgttggaagggttgaggatgaaggagggtgttaatttattttttattgggttggtggtaatgttgaattttgtagtgattgtggagggttaaggtgaaaattgttgggggtgttgagggtaggtgtggggaggggtggttttagggagtaaggagtttatttgttttgttgttgtagttgttttgggttgattgtttatgtttttttttgggttatgatttttggatttaattgtgtttggtttttgtttttttttttttgttgttgttgtgtgggttgtaatttgatgtttaggttggggtgtttagggtgtagtttttgtttaggttgttagtgttttatttgttttattgggttatagatttgttggtgttggggttttgttggtgttggggttttgttggtgttggggttttgttggtgttggggttttgttggtgttggggttttgttggtgttggggttttgttggtgttggggttttgtggttgtgtatgtgagtttgtgtggttttgttttgtgtggttgtgtatgtgagtttgtgtggttgtgttttgttttgttttagggagttagtgtgttgttatttgggatgttaggatttttgttgtgttttttggattgttttgggggattttggtgtatttttaggatttaggagttttggaagttgtttgagagaaattagttttgggagggttttgtatttagttttttgttttggttttggattggggttttg

SEQ ID NO: 34 - DNA, 6357 bp, Artificial Sequence
  chemically treated genomic DNA (Homo sapiens)
gtgtggagattgggaaggtgataaggtgaaggtaattgaaggaagagttgagggggatatggggaaggattttgttttattttttttaagttgaattattgttttttgaaggttggtttttggagaaattaaagggtttttgtgtgatatagttatgttatatataaatagaattttgaagtttattaatttttgaggttaagtaagagggaatgtaggggttaaggtagaagagaaattaaaattttagagtgttgagtaaagatgttaattagagaaagagaaatttatttgtgatgttaattaataagtggttaattaaaatggtattttgagtgttaattaattgttttattaagttatagttattattggaataaattgaaatttttttgttttgttttttgtttttggtgtaggtggggttgtgtttttagatattgtgagaggttttggggtgtggaggttgggggtagttttggttagtttttttagttttttttaggtttatagaatatgttattggataagtgtttaagtagtgatttttggtttagatatattgtttggggagtaagtagttgtgttgaagaataatttatttagtagtagttaatattgatgtttttttttagaaagagtttttgtaaagtggggggatgtgatttgtgggtttttagtaggggtaggaggtagtttagtttgagagggggtgttttagggtttggattttgtatttttattttttggaatatatttaatgttttattttgaaaattttgtttaggttttggttttggtgttgtttagtaattaggaaagagttggatttgtttttaggtagtaagaataggattgttagttttttgtggttttgttttttgaggttttatgagaaggggatgggggtgtaagaagggaagagtgaggtggtgtgttgggtgttggggatgaggatgtatgttagttaagatgtgttttttatttagtttatgtgtgtttttttatttttttggttttttaaaattggtaagagaattaagattttgaatttttattttgaggagttttttgtattttttaattgttaaatttttgttttttattaaatttttgggggagaaatatttggtaataagaagggattgtgaatttaaatgttaattgagtgggttttttttttgtagttttatttgtttggtagtttttgttgaaattaaatatatttggagtgtttagtgtaatatttttggggtgttgagtagaagtgtagtaaagagagattttagtggatttttgtttggtttgttttttattgatttttatagaaaaaaagagaatgttattggaagaagtttttttgtgtggtgggtgatgtgtgggtgggggatttgtggtatggtttatggtgttgtttttgtgtttgtgatgatatatgtatgttttgagttgtgggtttgtttttttggaggtgtgtttgattgtatttgttggtgggtttgagtgtgtttggggtgttttaggagaattgagagaatggtttttatgtgtaaagttttaaagtattaatatttttattatattattattatttaatataataatatttgtttggttagtggtattaattaggttatattaaaattgtagtgtgtttttaatggtgtgtaatgtgtttatatttatatttttttttttgaggatgggtggttgtaggttggtaggggaggagagataggtaagtggtgggttggattagggtgtgatgttttttattatgtatataaatatatatagtttattggatgtttgttgggtgggagttgtaatttttgtgtggttgatggggttttttgttgtgtatttggttttgtgttgagtattttgtagtttttttttgtgatatggtgttttgaatttggtggattgattttgtttttttttttttttttgtgtgtgtttgtgtttaattggttaggtttttaagatttgggagggttggtgtgaaagaattaaaatatttttaattggagtttttttgttgagaattggaggttttgttttttagtttggtgtttttaggattttttttttagagggaattttttttagaaattttagggtgggtttgtaaaagatgtttttgtagagtaggttttgttagggttttttttttgtttttggtgttagtggttggtttgggtgttttgtagattttggtgaggtagatgttaagtttggagagtgttttttttgtaggtgttgtggtgagattattttgaatatgtaatatatttgtaatgtgtgttgaggtgtgatgtgtgtgttgaaatagggggatgggggaatttgaagttggattgggaaggtgggggggaggtgtatagaatttataatgtattttgtaatttaataatttgaatatttatttattaaaagttgttgtgtgatatttatattgagttattagtttttgtttttaatttgggtgaaaatgattgtattgttgagttatggttgtagtgtatggggatgttgttgtttgtggttggatagagtttattagttataatgtggaaggtttttgtatttttttgggggtgggaggaaagtattgttagttttgtttgggggttgagggtaataagtattgagttttttgttttatgtagggttagttgtttagtttagtgaagtttttgtgatttggtgtgtgttttttgtttttttttttttattaaagaagtaaattttttatttattttttttaatttgattgtttagagttgttgttttttttttgttagattttttttttttgattagtttgagtatatgattagaattgtttagagagtaggaagtatattgattttagtttgttttgtttatagataggttttgataaggttgttagaatagttggagaggtttatataattatttaattattaaaattgttagttaggtgggatgtggatttgtgttttgggttgtgttaggtattttagtattgggttgtgtgtgtgattgattggtgttgatagtattgtaaaataattatggtgaattttttgatgtgtgattttattttaagtttatgttttagagaggtaattggagaatgagaagggttagtgttattttggattatttggaatttgtgagaaagggtaaaatgggggaaggagttttgaggaaaatgggagagatgggggtgtagagagagagggaagaagaaagtgagttatggattgttggagggattgtaagtaatttgttaaattgtgtaagtgattttttttagagttagtatatggtagattgattttgtttaatgttggttttagttatatttaaaatgatttagtggttattattgtgattggtttaggaattgataggtagttttaggtgtaaggagtatagattttgtttattggagatgtgtttgtaattgttgttaaatatagttaagtaaatattattagtgaagagttttgttaagagaaatgttaatttaataaatatgttttttttttttgttttttgtatggttgtttgtgttttttttagaggtttttttttttgtttttttgttgtttgggttagatgttttaggtatggtgttgatttttgttattttggagttttgagttgagttttgggtagaagatgataggttagttgtggggtaaggaggttgtggaaatgtggaatggttttggggagatggaagtgtttaatgagatttattttgtagtttgggtttagtttattttttttggagattgttgtggtttttgaatttgggtttaggtttttatgttttggtggttagaggatgttgtggggattattggggagttgtttttagttagttttttgttttatgttggaggttttggtgtggttttttttttgaattagattggtgattttgggttaggttttaaggattgttttggtttttttggttttgtggggagaatttgaggaattgagtttaagatagttgatttaggttgtttttatttagattttgtgtttttgatttgttggagtgaatttgatattgttaggtttttttttatggtatggagtgaatgaagagggttatagatttttttatttagtatagttttttggtaggttttggaaatttatagggagtagaagtatagtattttttgaattgttttttttttttgggtttgtggttatttgaaggtagagttttgtgtttttaagatagtaggtttttggttaagtttggagtttggggttttaatatatttatatagggttggtattattgtttttttggattaaaggtaggtttttatattttttttaaaggaatagaaggaaggaaaaggaaattaatatgggttatgtttagatagtaggtttatggtaattttttatagttatagagttttaatttgagtatttttttagagaggaaaaatttaaaaaatttttaaaagggggaaagttgggtagattatagtatttattttttatgtttaggtagaaaaattaatttagagggagtaaaggggtaagaaatatgaagagattttttttgggagttgaggagtattttagtttataatttggttaaaggagaaagttattggtttttttttttgatagaggtgtgttatttatttttttagggaatatgatggtttataaatgaagaggttagtttttttgtagtttttttttatagagtgtaaaatatatattgtttttattagtgtttgggatgtaaagaattttgtttatttaaaagagatattgtatttttaaagttaaatagtattaatgtatgtggtgaattaagtaggtaaataatttatatatggttgttgtatttgaaggaattatttatttttatgtatagtaaattgaagaaataatggtattaatgagttttgtaaaatgtaattgtgaataatgaaagataatattgtattttgtaatagaaagaataaaggtgaaataattagttagtaaagaggaaaagaaagtgagtaatgattaaatgattaaaagttggtagagtgaatttaatgttattgttagatgtagttatttatttataagtgaaagttaggttttaagtatagtgtaattatagttggggttgttagtttgatattaatgtagttagtagaaattttttaattggttttagaggagaaagtgaattagaaaatatattaatattttaaaaaagtatattttgtttaatttttttatttttgaataatatttgaagattaaaatgttttaggtataagaatttaaatgagtaattttgtttttgaaggaaatggttaatgagatagaaaatagattaaagggaaattattagtggatgagagatattgataggtttgttttgttgattggttggtttgttatttgtagtttgtgttttttagttttatgttatgagttaagttgataatatgaaaagatttataaatgtgtagttagaagttatagtttattatttggaaatttaaatgtaaggggagggggtggtagagaaggtattggtgaggttgggagggagaggtgtgtattgagggaggaggaggaggaggaaggggaggagggaaaggaggaggaggaggagaaaagaagtttttattttttggtataaaattggttatattagagaataataataagttattatttgttataaatgtgttatggattgttaaaaaatgtagttttgaattgataatattgtttgaattgaagatagtaataaaatgtttaaagttgtggatgtaattttatatgtgtttgggttgatgtgatatgattgtattagggaaataaagttaagtgtagttaggatttgttagtatagtggtttttgatggaatagttgaggtatatattgtttgtggtatggattttggggttgaatgtttatgattaagatttttgtttttttgaaatgaaatagaaataggggagttgtaggaaaattgaattgtgtttagggttaggagtaagataggagtttttagtgaagatttgaatatttagaattggaatgggtaattagtagatagttaggaaaaaataaataaataaataaaaaagtttggatggatttttgtaaataattattaagaaaaataaaaatgaatttttttattagtttgttttggaggtagttagaaataaataattaaagtaagagaggatgaagatttaaataaaataattatgtgtattattaaaataattatatatgtttgtatagatatgtatatattaaggaatgtaatgggggttttttgtatagttttaggagatgtagga

SEQ ID NO: 35 - DNA, 6357 bp, Artificial Sequence
  chemically treated genomic DNA (Homo sapiens)
ttttgtattttttgggattgtgtgaggagtttttattatgttttttggtgtatatgtgtttgtataaatatatatgattattttaatgatgtatataattattttatttaaatttttatttttttttgttttggttgtttgtttttgattatttttaaggtaggttaataagaaggtttatttttattttttttaatgattgtttatagaggtttatttaggtttttttatttatttatttatttttttttggttatttgttaattatttgttttagttttgaatgtttagatttttgttgaaagtttttgttttgtttttgattttaagtgtgatttggtttttttgtagtttttttatttttattttattttaaaagggtaaaagttttggttgtgagtgtttggttttggagtttatgttatgggtgatgtgtgttttagttgttttattaaaagttattgtattaatagattttgattgtatttagttttgtttttttgatatggttatattatattaatttggatgtatgtgaaattatatttgtaattttaagtattttgttgttatttttagtttgaataatgttgttgatttgggattatattttttggtagtttatagtatatttatgatagataatagtttattattgttttttgatatggttgattttatgttaaagaatgagggttttttttttttttttttttttttttttttttttttttttttttttttttttttttttttgatgtatattttttttttttaattttgttgatgttttttttgttattttttttttttgtatttgaatttttagataataggttgtgatttttggttgtatgtttatgggtttttttatgttattaatttagtttatagtgtggaattaaagaatatagattgtaagtgataggttagttagttagtaaggtaagtttgttagtattttttatttattaatgatttttttttagtttattttttgttttattggttgttttttttaaaaataaaattgtttatttaaatttttatgtttggggtattttggtttttaaatattgtttgaaagtgaaaggattaggtaaaatatgtttttttaaaatgttaatatattttttggtttatttttttttttgaggttaattaggaaatttttgttggttgtattaatgttaaattgataattttagttataattatattgtgtttgaaatttaatttttatttgtgggtagatggttgtgtttggtagtgatattgaatttattttgttagtttttgattatttaattattgtttgttttttttttttttttgttagttgattattttatttttattttttttgttgtaaaatgtagtgttgttttttattatttatagttgtattttgtaaggtttattagtgttattgtttttttaatttgttgtgtatgagaatggatggtttttttaagtgtagtaattatatgtaagttgtttatttatttgatttgttatatatattggtattgtttgattttaaaaatgtagtattttttttaaatagatagggttttttatattttaaatattgatgaaggtggtgtgtgttttatattttgtagaaaaaagttgtaggagggttagtttttttatttgtgaattattatgttttttgggaagatagatgatatgtttttattaaaggaggaggttagtgatttttttttttgattaaattataaattagggtgttttttagtttttagaggggatttttttatattttttatttttttgttttttttgggttagtttttttgtttaggtatgaagaatgggtgttatgatttatttagtttttttttttttaaaagttttttgggttttttttttttgggaagatatttggattaggattttatggttgtgaagaattgttataggtttattatttgaatataatttgtgttggtttttttttttttttttttgtttttttaaaaaggatataggagtttgtttttagtttaaggaaatggtgatgttaattttgtgtaaatgtgttggggttttaggttttaaatttgattgaaaatttattgttttggaggtatagagttttgtttttaaatggttataggtttagggagagggagtggtttagaaaatattgtgtttttgttttttgtggatttttagggtttgttgagggattgtgttgggtaaggggatttatggttttttttatttattttatgttatgagagagaatttggtagtgttagatttattttagtgggttggggatgtagggtttgggtgaaaatagtttaggttggttattttggatttggttttttagattttttttgtaaagttggagaggttggggtggtttttggggtttggtttagagttgttagtttagtttgggaaagaagttgtgttaggatttttggtgtggggtagagagttgattgagggtagttttttagtggtttttgtaatgttttttggttgttgggatatgaagatttaggtttgggtttgagggttgtggtaatttttgaggaaggtgggttggatttgggttgtagggtgaattttattgggtgtttttgtttttttgaagttgttttgtgtttttgtggtttttttgttttatggttggtttgttattttttgtttgaggtttagtttggggttttaaggtggtgggagttagtattatgtttgggatgtttgatttaagtagtaaaggagtaggaaggagaatttttggaggaagtgtaggtagttatgtggagggtggggaggaaaagtatatttattggattggtattttttttaatagagttttttgttaatgatatttatttaattgtatttgatagtagttatgaatatatttttggtaaataggatttatattttttgtgtttaaaattgtttgttagtttttaagttaattgtagtaataattgttggattattttaaatgtggttaaaattgatgttggataaaattaatttgttatatgttggttttgaaggaaattatttgtatagtttgatgaattgtttgtagtttttttagtaatttataatttgttttttttttttttttttttttgtatttttattttttttgttttttttggagtttttttttttattttatttttttttgtagattttaggtaatttgaaatggtattgattttttttattttttgattatttttttgaagtatgaatttgggataaaattatatattagaaaatttgttgtaattattttgtggtgttattagtattgattaattatgtgtgtggtttagtgttggaatgtttagtgtagtttgggatgtggatttgtgttttgtttgattgatagttttggtaattaagtgattgtatagatttttttggttgttttaataattttgttagggtttgtttgtggatagaataagttgaaattaatgtgttttttgttttttgagtagttttgattgtgtatttagattgattggggaggaggaatttgataaaaggaaaatagtagttttaaatgattggattagggggagtaggtagaaagtttatttttttgatggggagggaagagtgagagatatgtattagattataagagttttgttgagttgggtagttggttttgtgtggagtgagaggtttggtgtttgttatttttggtttttaggtaggattggtagtattttttttttgtttttaagggggtgtagaggttttttgtgttgtagttgatgggttttgtttggttgtggatagtagtgtttttatatgttgtagttataatttggtagtataattgtttttgtttggattagaggtagagattggtggtttagtgtaaatgttatgtagtagtttttaataaatgaatgtttagattgttagattgtgaagtatattgtgagttttgtgtgttttttttttgtttttttaatttggttttgaatttttttatttttttattttagtatatatattatattttggtgtatgttataaatatgttatatatttagagtgattttgttatggtgtttgtgggaggggtattttttgagtttaatatttattttgttgaggtttgtggggtgtttgggttgattgttggtattaggaataggaaaaagattttgatgggatttgttttgtggaagtgttttttataagtttattttggaatttttgaaagaaattttttttgggaagagggttttgaaagtgttgaattaggaggtgggatttttagtttttggtggaggggttttagttaagagtattttaatttttttatattagtttttttaaattttaaaaatttaattaattgaatgtaaatatatataaaagggggaagggaagtaaaattaatttgttgagtttaaagtgttgtgttgtgggaggaggttgtagggtgtttggtgtagggttgagtgtgtagtggagggttttattgattgtgtggagattgtggtttttatttggtagatatttagtgggttgtgtatgtttgtgtgtgtggtggggggtgttatgttttaatttagtttgttgtttgtttgtttttttttttttattagtttgtagttgtttatttttagagagaaaaatgtgagtgtgagtatattatgtattattagggatatattatggttttaatgtggtttaattagtgttgttaattgaataaatattattatattgaataatgataatatgatgaaaatattaatgttttggaattttgtatgtgggagttgtttttttagtttttttgggatattttaagtatgtttagatttattagtagatgtggttgggtgtatttttaggaaggtgagtttatagtttaagatatatgtgtgttattgtaggtatagaaataatattgtgggttatgttataagttttttatttatatattgtttattatgtaaaagagttttttttaatggtattttttttttttttgtaagagttggtaaaaagtaaattagataggagtttgttagggtttttttttattgtgtttttatttggtattttaagaatgttgtattgggtgttttgagtgtgtttggttttaatagaggttgttaggtaggtggagttgtggaaaaaggatttatttaattagtatttaaatttatagtttttttttattgttaaatgttttttttttgggaatttggtgaaaagtaggaatttaataattaggaaatgtggaaggttttttaaaatagggatttgaaattttaatttttttattgattttggagggttaggggggtggggaagtgtgtgtgggttgggtgggaggtatgttttggttggtgtgtgtttttgtttttgatgtttagtatattattttatttttttttttttgtatttttattttttttttatggagttttgggagatagagttataggaggttggtagttttgtttttgttgtttgaaggtaggtttagtttttttttggttgttgagtggtattagggttagggtttaagtagggtttttagaatgaggtgttgggtgtgttttaggaaatagggatgtaggatttaggttttagagtgtttttttttgggttgaattgttttttatttttgttgggggtttataggttatatttttttgttttgtgggagttttttttaggaggaaatgttggtgttaattgttgttgaatgagttgttttttgatgtaattatttattttttgggtggtgtgtttggattagaagttgttgtttaggtatttgtttagtggtgtattttgtagatttgggagagattgagaaagttgattgaggttgtttttaatttttgtgttttaaggttttttatggtatttgggaatgtggttttgtttgtattaaaggtagaaaatggggtggggaggttttaatttgttttagtgatggttgtaatttaataaggtgattgattagtatttaaagtgttgttttaattagttgtttgttaattaatattgtaaatgaatttttttttttttgattggtatttttgtttagtgttttgaggttttggttttttttttgttttggtttttatatttttttttatttagttttaggagttgataggttttagagttttgtttatgtatgatatggttgtgttatataggggttttttaatttttttaggagttggtttttaaaggataatggtttaatttaggaggggtgaaataaaatttttttttatgttttttttggttttttttttaattgtttttattttgttatttttttaatttttatat

SEQ ID NO: 36 - DNA, 167 bp, Artificial Sequence
  Synthetic construct ONECUT 1 MSP-Amplicon
gttttgaaatttattagaataacgacgttttaaaaataaaggcgtagtaagtattttttttttcgttgtcgcgggttgaattacggacgttcgcgggtcgtttagtttcgacggttcgtagggggcgcgcgtcgtagtcgtagtatagttcggttatttttagaaag

SEQ ID NO: 37 - DNA, 139 bp, Artificial Sequence
  Synthetic construct TFAP2E (76256) MSP-Amplicon (on Bis-1)
tttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgg

SEQ ID NO: 38 - DNA, 97 bp, Artificial Sequence
  Synthetic construct BARHL2 TSP PCR Amplicon
attgtttgttagtttttaagttaatcgtagtaataatcgttggattattttaaatgtggttaaaatcgacgttggataaaattaatttgttatatgt

SEQ ID NO: 39 - DNA, 163 bp, Artificial Sequence
  Synthetic construct FOXL2 HM-Amplicon
ccaagacctgggcttgcagcgccgccaacaggcccggggacacgaggcgctccaggccggggtcttcccggctgctggcccctctcgctccccacccgctggcggcgcctcggtcgcccgcaattgacccaacccgcttcctgcgtttgcccctcaggtttcc

SEQ ID NO: 40 - DNA, 95 bp, Artificial Sequence
  Synthetic construct TFAP2E HM-Amplicon
aaacccaaacctaaattaaaaaaacttcgctaactacaaacaaacgtccgaaaaaaacgaccaaacgaaaccccgacgctttaccacacacttcc

SEQ ID NO: 41 - DNA, 29 bp, Artificial Sequence
  Synthetic construct ONECUT 1 3658891.1Forward
gttttgaaatttattagaataacgacgtt

SEQ ID NO: 42 - DNA, 31 bp, Artificial Sequence
  Synthetic construct ONECUT 13658891.1Reverse
ctttctaaaaataaccgaactatactacgac

SEQ ID NO: 43 - DNA, 21 bp, Artificial Sequence
  Synthetic construct ONECUT 13658891.1Probe
tacggacgttcgcgggtcgtt

SEQ ID NO: 44 - DNA, 20 bp, Artificial Sequence
  Synthetic construct FOXL2 17389-Forward
ccaaaacctaaacttacaac

SEQ ID NO: 45 - DNA, 16 bp, Artificial Sequence
  Synthetic construct FOXL217389-Reverse
gagaggggttagtagt

SEQ ID NO: 46 - DNA, 31 bp, Artificial Sequence
  Synthetic construct FOXL2 17389-Forward Blocker
tacaacaccaccaacaaacccaaaaacacaa

SEQ ID NO: 47 - DNA, 23 bp, Artificial Sequence
  Synthetic construct FOXL2 17389-Reverse Blocker
ttgggaagattttggtttggagt

SEQ ID NO: 48 - DNA, 39 bp, Artificial Sequence
  Synthetic construct FOXL2 17389-SC-CH3 (methylation specific Scorpion probe)
ccgccgaaaacacgaaacggcgggagaggggttagtagt

SEQ ID NO: 49 - DNA, 49 bp, Artificial Sequence
  Synthetic construct FOXL2 17389-SC-total (Scorpion probe specific for total DNA)
cccgggaagattttggtttggagcccgggccaaaacctaaacttacaac

SEQ ID NO: 50 - DNA, 20 bp, Artificial Sequence
  Synthetic construct FOXL2 17389-Taqman probe specific for total DNA
ctccaaaccaaaatcttccc

SEQ ID NO: 51 - DNA, 19 bp, Artificial Sequence
  Synthetic construct FOXL2 17389-Taqman probe specific for methylated DNA
ccgaaaacacgaaacgctc

SEQ ID NO: 52 - DNA, 21 bp, Artificial Sequence
  TFAP2E MSPepsilon-G2 (forward Primer) MSP-Amplicon (on Bis-1)
tttagaagcggttttcgtatc

SEQ ID NO: 53 - DNA, 20 bp, Artificial Sequence
  TFAP2E MSPepsilon-C1 (reverse Primer) MSP-Amplicon (on Bis-1)
ccgaacgcttacctacaatc

SEQ ID NO: 54 - DNA, 22 bp, Artificial Sequence
  Synthetic construct TFAP2E MSPepsilon-P1 (Probe) MSP-Amplicon (on Bis-1)
ttgcggtgggcgttttcgggtt

SEQ ID NO: 55 - DNA, 20 bp, Artificial Sequence
  TFAP2E HM 76256-21 (forward Primer) (on Bis-2)
aaacccaaacctaaattaaa

SEQ ID NO: 56 - DNA, 17 bp, Artificial Sequence
  TFAP2E 76256-22 (reverse Primer) (on Bis-2)
ggaagtgtgtggtaaag

SEQ ID NO: 57 - DNA, 30 bp, Artificial Sequence
  Synthetic construct TFAP2E 76256.2B5 (Blocker) (on Bis-2)
gtaaagtgttggggttttgtttggttgttt

SEQ ID NO: 58 - DNA, 26 bp, Artificial Sequence
  Synthetic construct TFAP2E 76256-28dS (Probe) (on Bis-2)
aaaaacttcgctaactacaaacaaac

SEQ ID NO: 59 - DNA, 25 bp, Artificial Sequence
  BARHL2 Primer
attgtttgttagtttttaagttaat

SEQ ID NO: 60 - DNA, 27 bp, Artificial Sequence
  BARHL2 Primer
acatataacaaatatattttatccaac

SEQ ID NO: 61 - DNA, 26 bp, Artificial Sequence
  Synthetic construct BARHL2 TAQMAN probe
ttggattattttaaatgtggttaaaa